(19) United States
(12) Patent Application Publication
(10) Pub. No.: US 2024/0216432 A1
(43) Pub. Date: Jul. 4, 2024

(54) ANTI-FLT3 ANTIBODIES, CARS, CAR T CELLS AND METHODS OF USE

(71) Applicant: Hemogenyx Pharmaceuticals LLC, Brooklyn, NY (US)

(72) Inventors: Vladislav M. SANDLER, New York, NY (US); Elina SHRESTHA, Long Island City, NY (US); Raymond LIANG, Long Island City, NY (US); Carina Rachel SIROCHINSKY, New York, NY (US); Ronen BEN JEHUDA, New York, NY (US)

(21) Appl. No.: 18/684,056

(22) PCT Filed: Aug. 15, 2022
(86) PCT No.: PCT/US2022/074984

§ 371 Date: Feb. 15, 2024

Related U.S. Application Data
(60) Provisional application No. 63/253,009, filed on Oct. 6, 2021.
(60) Provisional application No. 63/233,530, filed on Aug. 16, 2021.

(51) Int. Cl. A61K 35/17; A61K 39/00; A61P 35/02; C07K 16/28

(52) U.S. Cl. CPC A61K 35/17; A61K 39/001103; A61K 39/4611; A61K 39/4631; A61P 35/02; C07K 16/2863; A61K 2239/13; A61K 2239/21; A61K 2239/22; A61K 2239/48

(57) ABSTRACT

Provided herein are anti-FLT3 antibodies or antigen binding fragments thereof, including heavy chain variable region, light chain variable region and single chain fragments (such as humanized anti-FLT3 antibodies and fragments thereof). In some aspects, the antibodies or fragments specifically bind human FLT3. Also provided herein are recombinant receptors, such as chimeric antigen receptors (CARs), comprising such antibodies or fragments. Also provided herein are immune cells comprising such CARs, such as CAR T cells. Also provided herein are methods of use of such antibodies or fragments, CARs and immune cells.

### CROSS-REFERENCE TO RELATED APPLICATIONS

This application claims the benefit of U.S. Provisional Patent Application No. 63/233,530 filed Aug. 16, 2021, and U.S. Provisional Patent Application No. 63/253,009 filed Oct. 6, 2021, each of which is incorporated by reference herein in its entirety.

### REFERENCE TO AN ELECTRONIC SEQUENCE LISTING

The contents of the electronic sequence listing (HEPH_001_002WO_SeqList_ST26.xml; Size: 228,845 bytes; and Date of Creation: Aug. 11, 2022) are incorporated by reference herein in their entirety.

### FIELD

In some aspects, the present invention relates to anti-FLT3 humanized antibodies or antigen binding fragments thereof, chimeric antigen receptors (CARs) comprising such antibodies or fragments, immune cells expressing such CARs, and uses of such antibodies, CARs and cells.

### BACKGROUND

### FLT3

FLT3 is Fms Related Receptor Tyrosine Kinase 3. FLT3 is also known as fetal liver kinase 2 (FLK2). FLT3, a member of the class III tyrosine kinase receptor family, is expressed in normal hematopoietic progenitors as well as in leukemic blasts, and it plays an important role in cell proliferation, differentiation, and survival. Activation of the FLT3 receptor by the FLT3 ligand leads to receptor dimerization and phosphorylation, and activation of downstream signaling pathways, including the Janus kinase (JAK) 2 signal transducer (JAK2), signal transducer and activator of transcription (STAT) 5, and mitogen-activated protein kinase (MAPK) pathways. Mutations in the FLT3 gene, found in approximately 40% of patients with AML, are believed to promote its autophosphorylation and constitutive activation, leading to ligand-independent proliferation (Frankfurt O et al., Current Opinion in Oncology (2007) 19(6): 635-649).

Normal FLT3 expression is mostly restricted to CD34+ hematopoietic stem cells (HSCs), early hematopoietic progenitors (HPs), and dendritic cells (DCs). Activation of FLT3, through binding of FLT3 ligand (FLT3L), promotes normal differentiation of downstream blood lineages.

FLT3 expression is high in a variety of hematologic malignancies, including in most of AML patients. AML blasts in a majority of patients having AML express FLT3 and this expression is thought to promote survival and proliferation. Tyrosine kinase inhibitors (TKIs) have been developed to specifically target FLT3; however, secondary mutations leading to resistance against FLT3 remain a major obstacle.

### Hematopoietic Stem Cells

The hematopoietic stem cell is the common ancestor of all blood cells. As multipotent cells, they can differentiate into multiple cell lineages, but not all the lineages derived from the three germ layers. Hematopoietic stem cell differentiation gives rise to the lymphoid and myeloid cell lineages, the two major branches of hematopoiesis. (Kondo, M. “Lymphoid and myeloid lineage commitment in multipotent hematopoietic progenitors,” Immunol. Rev. 2010 November; 238(1): 37-46). Lymphoid lineage cells include T, B, and natural killer (NK) cells. The myeloid lineage includes megakaryocytes and erythrocytes (MegE) as well as different subsets of granulocytes (neutrophils, eosinophils and basophils), monocytes, macrophages, and mast cells (GM), which belong to the myeloid lineage (Id. citing Kondo M, et al. Biology of hematopoietic stem cells and progenitors; implications for clinical application. Ann. Rev Immunol. 2003; 21:759-806., Weissman I L. Translating stem and progenitor cell biology to the clinic: barriers and opportunities. Science (New York, NY. 2000 Feb. 25; 287(5457); 1442-6); see also Iwaskaki, H. and Akashi, K. “Myeloid lineage commitment from the hematopoietic stem cell,” Immunity 26(6) June 2007, 726-40).

HSCs present self-renewal potential and differentiation capacity into blood lineages; i.e., when stem cells divide, 50% of the daughter cells, on average, are committed with a cell lineage, while the remaining 50% do not differentiate. The process maintains the same number of stem cells by asymmetric cell division, so that each dividing stem cell originates one new stem cell and one differentiated cell. In contrast, in symmetric division, the stem cells originate 100% of identical stem cells. (Gordon, M. Stem cells and haemopoiesis. In: HofTbrand, V., Catovsky, D., Tuddenham, E. G., 5th ed. Blackwell Publishing, (2005): Differential niche and Wnt requirements during acute myeloid leukemia, pp. 1-12. New York.).

The lymphoid and myeloid lineages are separable at the progenitor level. Common lymphoid progenitors (CLPs) can differentiate into all types of lymphocytes without noticeable myeloid potential under physiological conditions (Kondo M, Scherer D C, Miyamoto T, King A G, Akashi K, Sugamura K. et al. Cell-fate conversion of lymphoid committed progenitors by instructive actions of cytokines. Nature. 2000 Sep. 21; 407(6802):383-6), although some myeloid related genes might be detected in CLPs, depending on the experimental conditions (Delogu A, Schebesta A, Sun Q, Aschenbrenner K, Perlot T, Busslinger M. Gene repression by Pax5 in B cells is essential for blood cell homeostasis and is reversed in plasma cells. Immunity. 2006 March; 24(3):269-81).

Similarly, common myeloid progenitors (CMPs) can give rise to all classes of myeloid cells with no or extensively low levels of B-cell potential (Akashi K, Traver D, Miyamoto T, Weissman I L. A clonogenic common myeloid progenitor that gives rise to all myeloid lineages. Nature. 2000 Mar. 9; 404(6774): 193-7). Another cell type, dendritic cells (DCs), is not clearly grouped either in lymphoid or myeloid lineage, because DC can arise from either CLPs or CMPs (Manz M G, Traver D, Miyamoto T, Weissman I L, Akashi K. Dendritic cell potentials of early lymphoid and myeloid progenitors. Blood. 2001 Jun. 1; 97(11):3333-41, Traver D, Akashi K, Manz M, Merad M, Miyamoto T, Engleman E G, et al. Development of CD8alpha-positive dendritic cells from a common myeloid progenitor. Science (New York, NY. 2000 Dec. 15; 290(5499):2152-4)). CMPs can proliferate and differentiate into megakaryocyte-erythrocyte (MegE) progenitors and granulocyte-monocyte (GM) progenitors, which further give rise to megakaryocytes, erythrocytes, granulocytes, monocytes and others. (Iwasaki H, Akashi K. Myeloid lineage commitment from the hematopoietic stem cell. Immunity. 2007; 26:726-740).

It is likely that differences in the expression levels of transcription factors determine the lineage affiliation of a differentiating cell. The transcription factors PU.1 and GATA-1 have been implicated in myeloid and erythroid/megakaryocyte lineage differentiation, respectively (Gordon, M. Stem cells and haemopoiesis. In: Hoffbrand, V., Catovsky, D., Tuddenham, E. G., 5th ed. Blackwell Publishing, (2005): Differential niche and Wnt requirements during acute myeloid leukemia, pp. 1-12. New York.).

### Characterization of HSCs

HSCs are undifferentiated and resemble small lymphocytes. A large fraction of HSCs is quiescent, in the GO phase of the cell cycle, which protects them from the action of cell cycle-dependent drugs. The quiescent state of stem cells is maintained by transforming growth factor-β (TGF-β). The activity of TGF-β is mediated by p53, a tumor suppressor gene that regulates cell proliferation and targets the cyclin-dependent kinase inhibitor p21 (Gordon, M. Stem cells and haemopoiesis. In: Hoffbrand, V., Catovsky, D., Tuddenham, E. G., 5th ed. Blackwell Publishing, (2005): Differential niche and Wnt requirements during acute myeloid leukemia, pp. 1-12. New York.). Quiescence of HSCs is critical not only for protecting the stem cell compartment and sustaining stem cell pools during long periods of time, but also for minimizing the accumulation of replication associated mutations. Many of the intrinsic transcriptional factors that maintain HSCs quiescence are found to be associated with leukemias. For example, chromosomal translocations resulting in the fusion of FoxOs and myeloid/lymphoid or mixed lineage leukemia have been reported in acute myeloid leukemias (See, e.g., Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1).

The majority of normal HSCs are present among the CD34+/CD38−/CD90+ bone marrow cell fractions with some HSCs also observed among CD34−/Lin− cells. CD34+/CD38+ cell fractions contain some HSCs endowed with short-term repopulating activity. Other recognized markers include the tyrosine kinase receptor c-kit (CD117) coupled with a lack of terminal differentiation markers such as CD4 and CD8 (Rossi et al., Methods in Molecular Biology (2011) 750(2): 47-59).

### Classification of HSCs

The hematopoietic stem cell pool can be subdivided into three main groups: (1) short-term HSCs, capable of generating clones of differentiating cells for only 4-6 weeks; (2) intermediate-term HSCs, capable of sustaining a differentiating cell progeny for 6-8 months before becoming extinct; and (3) long-term HSCs, capable of maintaining hematopoiesis indefinitely. (Testa U. Annals of Hematology (2011) 90(3): 245-271).

### Hematopoiesis

Hematopoiesis is a highly coordinated process wherein HSCs differentiate into mature blood cells supported by a specialized regulatory microenvironment, consisting of components which control the fate specification of stem and progenitor cells, as well as maintaining their development by supplying the requisite factors (“niche”). The term “bone marrow (BM) niche” as used herein refers to a well-organized architecture composed of elements (e.g., osteoblasts, osteoclasts, bone marrow endothelial cells, stromal cells, adipocytes and extracellular matrix proteins (ECM)) that play an essential role in the survival, growth and differentiation of diverse lineages of blood cells. The bone marrow niche is an important post-natal microenvironment in which HSCs proliferate, mature and give rise to myeloid and lymphoid progenitors.

Bone marrow (BM) is present in the medullary cavities of all animal bones. It consists of a variety of precursor and mature cell types, including hematopoietic cells (the precursors of mature blood cells) and stromal cells (the precursors of a broad spectrum of connective tissue cells), both of which appear to be capable of differentiating into other cell types. The mononuclear fraction of bone marrow contains stromal cells, hematopoietic precursors, and endothelial precursors.

Unlike secondary lymphoid organs such as spleen with distinct gross structures including red and white pulp, BM has no clear structural features, except for the endosteum that contains osteoblasts. The endosteum region comes in contact with calcified hard bones and provides a special microenvironment which is necessary for the maintenance of HSC activity (Kondo M, Immunology Reviews (2010) 238(1): 37-46; Bydlowski and de Lara Janz (2012)). Hematopoietic Stem Cell in Acute Myeloid Leukemia Development, Advances in Hematopoietic Stem Cell Research, Dr. Rosana Pelayo (Ed.), ISBN: 978-953-307-930-1).

Within the niche, HSCs are believed to receive support and growth signals originating from several sources, including: fibroblasts, endothelial and reticular cells, adipocytes, osteoblasts and mesenchymal stem cells (MSCs). The main function of the niche is to integrate local changes in nutrients, oxygen, paracrine and autocrine signals and to change HSCs quiescence, trafficking, and/or expansion in response to signals from the systemic circulation (Broner, F. & Carson, M C. Topics in bone biology. Springer. 2009; 4: pp. 2-4. New York, USA.).

Although the nature of true MSCs remains misunderstood, CXC chemokine ligand 12 (CXCL12)-expressing CD146 MSCs were recently reported to be self-renewing progenitors that reside on the sinusoidal surfaces and contribute to organization of the sinusoidal wall structure, produce angiopoietin-1 (Ang-1), and are capable of generating osteoblasts that form the endosteal niche (Konopleva, M Y, & Jordan, C T, Biology and Therapeutic Targeting (2011) 9(5): 591-599). These CXCL12 reticular cells may serve as a transit pathway for shuttling HSCs between the osteoblastic and vascular niches where essential but different maintenance signals are provided.

Cytokines and chemokines produced by bone marrow MSCs concentrate in particular niches secondary to varying local production and through the effects of cytokine binding glycosaminoglycans. Of these, CXCL12/stromal cell-derived factor-1 alpha positively regulates HSCs homing, while transforming growth factors FMS-like tyrosine kinase 3 (Flt3) ligand and Ang-1 act as quiescence factors (See, e.g., Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1). CXCL12-CXCR4 signaling is involved in homing of HSCs into BM during ontogeny as well as survival and proliferation of colony-forming progenitor cells. The CXCR4-selective antagonist-induced mobilization of HSCs into the peripheral blood further indicates a role for CXCL12 in retaining HSCs in hematopoietic organs. BM engraftment involves subsequent cell-to-cell interactions through the BMSC-produced complex extracellular matrix. Thus, vascular cell adhesion molecule-1 (VCAM-1) or fibronectin is critical for adhesion to the BM derived MSCs. In this way, the control of hematopoietic stem cell proliferation kinetics is critically important for the regulation of correct hematopoietic cell production. These control mechanisms could be classified as intrinsic or extrinsic to the stem cells, or a combination of both (See, e.g., Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1).

HSC self-renewal and differentiation can be controlled by external factors (extrinsic control), such as cell-cell interactions in the hematopoietic microenvironment or cytokines, such as SCF (stem cell factor) and its receptor c-kit, Flt-3 ligand, TGF-β, TNF-α and others. Cytokines regulate a variety of hematopoietic cell functions through the activation of multiple signal transduction pathways. The major pathways relevant to cell proliferation and differentiation are the Janus kinase (Jak)/signal transducers and activators of transcription (STATs), the mitogen-activated protein (MAP) kinase and the phosphatidylinositol (PI) 3-kinase pathways (Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1).

In addition, expression of other transcription factors, such as, stem cell leukemia (SCL) hematopoietic transcription factor; GATA-2; and gene products involved in cell cycle control, such as the cyclin dependent kinase inhibitors (CKIs) p16, p21 and p27 have been shown to be essential for hematopoietic cell development from the earliest stages (intrinsic control), (Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1).

Notch-1-Jagged pathway may serve to integrate extracellular signals with intracellular signaling and cell cycle control. Notch-1 is a surface receptor on hematopoietic stem cell membranes that binds to its ligand. Jagged, on stromal cells. This results in cleavage of the cytoplasmic portion of Notch-1, which can then act as a transcription factor (Gordon, M. Stem cells and haemopoiesis. In: Hoffbrand, V., Catovsky, D., Tuddenham, E. G., 5th ed. Blackwell Publishing, (2005): Differential niche and Wnt requirements during acute myeloid leukemia, pp. 1-12. New York.).

Disorders that are Treated Using BM/HSC Transplantation

Disorders that are treated using Bone Marrow (BM)/Hematopoietic Stem Cell (HSC) transplantation include, without limitation, Acute Myeloid Leukemia (AML), Acute Lymphoblastic Leukemia (ALL), Chronic Lymphocytic Leukemia (CLL), Chronic Myeloid Leukemia (CML), Blastic plasmacytoid dendritic cell neoplasm (BPDCN), peripheral T cell lymphoma, follicular lymphoma, diffuse large B cell lymphoma, Hodgkin lymphoma, non-Hodgkin lymphoma, neuroblastoma, non-malignant inherited and acquired marrow disorders (e.g. sickle cell anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, or congenital thrombocytopenia), multiple myeloma, and Severe Combined Immunodeficiency (SCID).

### Hematopoietic Malignancies

Most hematopoietic malignancies comprise functionally heterogeneous cells, with only a subset, known as cancer stem cells, responsible for tumor maintenance. Cancer stem cells are so named because they possess qualities reminiscent of normal tissue stem cells including self-renewal, prolonged survival, and the ability to give rise to cells with more differentiated characteristics (Jones R J and Armstrong S A, Biol Blood Marrow Transplant. 2008 January; 14 (Supplement 1): 12-16).

A transforming event in hematopoietic stem cells can produce several different malignancies, including, without limitation, chronic myeloid leukemia, myelodysplastic syndrome, acute myeloid leukemia, and probably even acute lymphocytic leukemia, depending on the degree of differentiation associated with the oncogenic hit (Jones R J and Armstrong S A, Biol Blood Marrow Transplant. 2008 January; 14 (Supplement 1): 12-16).

The cancer stem cell concept is based on the idea that tumors of a specific tissue often appear to “attempt” to recapitulate the cellular heterogeneity found in the tissues of origin, and thus there are cells in the tumor that are stem-cell like giving rise to the varied cell types. A fundamental test for this hypothesis is whether tumor cells can be separated into those that have the ability to regenerate the tumor, and those that do not possess this ability. This cellular hierarchy has been most clearly demonstrated in acute myelogenous leukemias where some AMLs possess cells with a unique immunophenotype that are able to initiate leukemias in immunodeficient mice whereas most cells are unable to initiate leukemia development. Furthermore, the cells that initiate leukemias also give rise to cells that have lost tumor-initiating activity and thus recapitulate the cellular heterogeneity found in the original tumor (Lapidot T et al., Nature. 1994; 367: 645-648; Bonnet D et al., Nat Med. 1997; 3: 730-737).

### Acute Myeloid Leukemia

Acute myeloid leukemia (AML) is a clonal disorder characterized by arrest of differentiation in the myeloid lineage coupled with an accumulation of immature progenitors in the bone marrow, resulting in hematopoietic failure (Poll yea D A et al., British Journal of Haematology (2011) 152(5): 523-542). There is wide patient-to-patient heterogeneity in the appearance of the leukemic blasts. The discovery of leukemia-initiating cells in acute myeloid leukemias (AMLs) started with the discovery that the large majority of AML blasts do not proliferate and only a small minority is capable of forming new colonies (Testa U, Annals of Hematology (2011) 90(3): 245-271). A common feature to all AML cases is the arrested aberrant differentiation leading to an accumulation of more than 20% blast cells in the bone marrow (Gilliland, D G and Tallman M S, Cancer Cell (2002) 1(5): 417-420).

More than 80% of myeloid leukemias are associated with at least one chromosomal rearrangement (Pandolfi P P, Oncogene (2001) 20(40): 5726-5735), and over 100 different chromosomal translocations have been cloned (Gilliland, D G and Tallman M S, Cancer Cell (2002) 1 (5): 417-420). These translocations frequently involve genes encoding transcription factors that have been shown to play an important role in hematopoietic lineage development. Thus, alteration of the transcriptional machinery appears to be a common mechanism leading to arrested differentiation (Pandolfi P P, Oncogene (2001) 20(40): 5726-5735; Tenen D G, Nature Reviews of Cancer (2003) 3(2): 89-101).

Clinical investigation and experimental animal models suggest that at least two genetic alterations are required for the clinical manifestation of acute leukemia. According to the model proposed by Gilliland & Tallman (Cancer Cell (2002) 1(5): 417-420), cooperation between class I activating mutations and class II mutations that induce termination of differentiation give rise to AML. The class I mutations, such as mutations in the receptor tyrosine kinase genes FLT3 and KIT, RAS family members, and loss of function of neurofibromin 1, confer proliferative and/or survival advantage to hematopoietic progenitors, typically as a consequence of aberrant activation of signal transduction pathways. The class II mutations lead to a halt in differentiation via interference with transcription factors or co-activators (Frankfurt O et al., Current Opinion in Oncology (2007) 19(6): 635-649). While the leukemia stem cell (LSC) appears to share many of the cell surface markers previously identified for HSC such as CD34, CD38, HLA-DR, and CD71, several groups have reported surface markers that are differentially expressed in the two populations. For example, CD90 or Thy-1 has been described as potentially specific of the LSC compartment. Thy-1 is downregulated in normal hematopoiesis as the most primitive stem cells progress toward the progenitor stage. (Hope K J et al., Archives of Medical Research (2003) 34(6): 507-514).

The interaction between CXCL12 (stromal cell-derived factor-1 alpha) and its receptor CXCR4 on leukemic progenitor cells contributes to their homing to the bone marrow microenvironment. CXCR4 levels are significantly elevated in leukemic cells from patients with AML, and CXCR4 expression is associated with poor outcome (Konopleva M Y and Jordan C T, Biology and Therapeutic Targeting (2011) 29(5): 591-599).

Constitutive activation of the nuclear factor kappa f3 (NF-kβ) pathway I primary human AML stem cells provided evidence that NF-κβ plays a significant role in the overall survival of LSCs as well as AML cell types in general. (Konopleva M Y and Jordan C T, Biology and Therapeutic Targeting (2011) 29(5): 591-599).

AML patients have poor clinical prognosis and limited therapeutic options, with myeloablation followed by hematopoietic stem cell transplantation (HSCT) as the only curative treatment. The commonly used conditioning regimens indiscriminately kill all highly proliferative cell types, leading to life threatening side effects, and are also potentially ineffective against quiescent AML subpopulations.

### Lymphoid Malignancies

Self-renewal capacity in most tissues is lost as cells progress through their normal stages of differentiation; for example, myeloid lineage blood cells beyond the level of hematopoietic stem cells no longer possess self-renewal capacity. A notable exception to differentiation-associated loss of self-renewal is the lymphoid system, where self-renewal capacity is preserved until the memory lymphocyte stage in order to maintain life-long immune memory (Fearon D T et al., Science. 2001; 293: 248-250; Luckey C J et al., Proc Natl Acad Sci USA. 2006; 103: 3304-3309). Somatic hypermutation serves as a marker for the stage of differentiation at which B cell malignancies arise. In general, the presence of somatic hypermutation identifies a tumor as having arisen in germinal center or post-germinal center B cells, while the absence of mutation identifies pre-germinal center B cells. In contrast to myeloid malignancies but consonant with the lineage's preserved self-renewal capacity, immunoglobulin (lg) mutation patterns suggest that B cell malignancies can arise from cells throughout the stages of B cell differentiation (Lapidot T et al., Nature. 1994; 367: 645-648; Bonnet D and Dick J E, Nat Med. 1997; 3: 730-737; Jones R J et al., J Natl Cancer Inst. 2004; 96: 583-585).

### Multiple Myeloma

Multiple myeloma (MM) has generally been considered a disease of malignant plasma cells with many of the clinical consequences of the disease resulting from the plasma cell bulk. However, normal plasma cells are terminally differentiated and lack self-renewal capacity and it has been clear for over 30 years that only a minority of cells from mouse and human MM were clonogenic. These rare clonogenic cells have been termed “tumor stem cells” (Park C H et al., J Natl Cancer Inst. 1971; 46: 411-422; Hamburger A W and Salmon S E, Science. 1977; 197: 461-463). MM plasma cells arise from a small population of self-renewing cancer stem cells that resemble memory B cells. Not only do these clonotypic B cells circulate in most patients but they also are resistant to many standard anti-MM agents, and thus appear to be responsible for most disease relapses (Matsui W H et al., Blood. 2004; 103: 2332-2336; Kukreja A et al., J Exp Med. 2006; 203: 1859-1865; Jones R J and Armstrong S A, Biol Blood Marrow Transplant. 2008 January; 14 (Supplement 1): 12-16).

### Hodgkin's Lymphoma

Reed-Sternberg (RS) cells, the hallmark of Hodgkin's lymphoma (HL), are the only blood cells other than plasma cells to occasionally express CD138 (Carbone A et al., Blood. 1998; 92: 2220-2228). It has been shown that HL cell lines include a small population of cells that lack the RS markers CD15 and CD30 present on the rest of the cells, while expressing markers consistent with a memory B cell phenotype (Newcom S R et al., Int J Cell Cloning. 1988; 6: 417-431; Jones R J et al., Blood. 2006; 108: 470). This small subpopulation of phenotypic memory B cells possessed all of the clonogenic capacity within the HL cell lines. Most HL patients, including those with early stage disease, harbor circulating memory B cells with the same clonal lg gene rearrangement as the patients' RS cells (Jones R J et al., Blood. 2006; 108: 470; Jones R J and Armstrong S A, Biol Blood Marrow Transplant. 2008 January; 14 (Supplement 1): 12-16). These data suggest that these clonotypic memory B cells likely represent the HL stem cells.

### Treatment of Hematological Malignancies

Hematopoietic stem cells (HSCs) are used in bone marrow transplantation for treatment of hematological malignancies as well as nonmalignant disorders (Warner et al, Oncogene (2004) 23(43): 7164-7177). Until researchers discovered which cellular components were responsible for the engraftment of the donor hematopoietic and immune systems in marrow-ablated patients, bone marrow (BM) had been transplanted as an unfractionated cell pool for many years (See, e.g., Sergio Paulo Bydlowski and Felipe de Lara Janz (2012). Hematopoietic Stem Cell in Acute Myeloid Leukemia Development, Advances in Hematopoietic Stem Cell Research, Dr. Rosana Pelayo (Ed.), ISBN: 978-953-307-930-1). Preparation or conditioning of a patient for bone marrow/hematopoietic stem cell (BM/HSC) transplant is a critical element of the procedure. It serves two main purposes: (1) it provides adequate immunosuppression of the patient and clears sufficient niche space in the bone marrow for the transplanted HSC, which allows transplanted cells to engraft in the recipient; and (2) it often helps to eradicate the source of the malignancy.

Conditioning of patients has traditionally been achieved by administering maximally tolerated doses of a cocktail of chemotherapeutic agents with or without radiation. Components of the cocktail are often chosen to have non-overlapping toxicities. All preparative regimens currently in use are toxic and have severe side effects that can be life threatening. Among these side effects are mucositis, nausea and vomiting, alopecia, diarrhea, rash, peripheral neuropathies, infertility, pulmonary toxicities and hepatic toxicities. Many of these side effects are especially dangerous for older and sick patients, and often become a decisive component in deciding whether a patient will receive a transplant.

Thus, a need exists to prepare or condition patients eligible for bone marrow/hematopoietic stem cell (BM/HSC) transplant without these toxicities.

A need also exists to treat hematologic malignancies, such as AML, without these toxicities.

### SUMMARY OF THE INVENTION

In some aspects, the disclosure provides a humanized antibody or antigen binding fragment thereof that binds (such as specifically binds) to human and rhesus monkey FLT3. In some aspects, the disclosure provides a humanized antibody or antigen binding fragment thereof that binds (such as specifically binds) to human FLT3.

In some embodiments, the disclosure provides an anti-FLT3 humanized antibody or antigen binding fragment thereof, wherein the antibody or fragment comprises a light chain variable region (VL) comprising an amino acid sequence with at least 95% identity to any one of the sequences selected from the group consisting of SEQ ID NO:1, SEQ ID NO:2, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38.

In some embodiments, the disclosure provides an anti-FLT3 humanized antibody or antigen binding fragment thereof, wherein the antibody or fragment comprises a heavy chain variable region (VH) comprising an amino acid sequence with at least 95% identity to any one of the sequences selected from the group consisting of: SEQ ID NO:3, SEQ ID NO:18, SEQ ID NO:19, SEQ ID NO:20, SEQ ID NO:21, SEQ ID NO:22, SEQ ID NO:23, SEQ ID NO;24, SEQ ID NO:25, SEQ ID NO:26, and SEQ ID NO:27.

In some embodiments, the disclosure provides an anti-FLT3 humanized antibody or antigen binding fragment thereof, wherein the antibody or fragment comprises:
(i) a light chain variable region (VL) comprising an amino acid sequence with at least 95% identity to any one of the sequences selected from the group consisting of: SEQ ID NO:1, SEQ ID NO:2, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38; and/or
(ii) a heavy chain variable region (VH) comprising an amino acid sequence with at least 95% identity to any one of the sequences selected from the group consisting of: SEQ ID NO:3, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, and SEQ ID NO:27.

In any of the foregoing or related aspects and embodiments, the VL comprises the amino acid sequence of SEQ ID NO:1, and the VH comprises the amino acid sequence of SEQ ID NO:3. In any of the foregoing or related aspects and embodiments, the VL comprises the amino acid sequence of SEQ ID NO:2, and the VH comprises the amino acid sequence of SEQ ID NO:3.

In any of the foregoing or related aspects and embodiments, the disclosure provides anti-FLT3 humanized antibodies or fragments thereof, wherein the VL comprises complementarity determining regions (CDRs) having at least 97%, 98%, 99% or 100% identity to the amino acid sequences of CDR-L1 of SEQ ID NO:86, CDR-L2 of SEQ ID NO: 87, and CDR-L3 of SEQ ID NO: 88. In some of these embodiments, the CDRs are as determined by Kabat.

In any of the foregoing or related aspects and embodiments, the disclosure provides anti-FLT3 humanized antibodies or fragments thereof, wherein the VH comprises CDRs having at least 97%, 98%, 99% or 100% identity to the amino acid sequences of CDR-H1 of SEQ ID NO: 89, CDR-H2 of SEQ ID NO: 90, and CDR-L3 of SEQ ID NO:91. In some of these embodiments, the CDRs are as determined by Kabat.

In any of the foregoing or related aspects and embodiments, the disclosure provides anti-FLT3 humanized antibodies or fragments thereof, wherein (i) the VL comprises complementarity determining regions (CDRs) having at least 97%, 98%, 99% or 100% identity to the amino acid sequences of CDR-L1 of SEQ ID NO:86, CDR-L2 of SEQ ID NO: 87, and CDR-L3 of SEQ ID NO: 88, and (ii) the VH comprises CDRs having at least 97%, 98%, 99% or 100% identity to the amino acid sequences of CDR-H1 of SEQ ID NO: 89, CDR-H2 of SEQ ID NO: 90, and CDR-L3 of SEQ ID NO:91. In some of these embodiments, the CDRs are as determined by Kabat.

In any of the foregoing or related aspects and embodiments, the antigen binding fragment of the humanized anti-FLT3 antibodies described herein is a single chain variable domain (scFv) (such as scFv comprising any VH and any VL described herein or referenced in the foregoing aspects and embodiments). In some embodiments, the scFv comprises (or substantially consists or consists of) the amino acid sequence selected from the group comprising: SEQ ID NO:4, SEQ ID NO:5, SEQ ID NO:44, SEQ ID NO:45, SEQ ID NO:46, SEQ ID NO:47, and SEQ ID NO:49. In some embodiments, the scFv comprises (or substantially consists of or consists of) the amino acid sequence selected from the group consisting of: SEQ ID NO:4, SEQ ID NO:5, SEQ ID NO:44, SEQ ID NO:45, SEQ ID NO:46, SEQ ID NO:47, and SEQ ID NO:49. In some embodiments, the scFv comprises the amino acid sequence of SEQ ID NO:4. In some embodiments, the scFv comprises the amino acid sequence of SEQ ID NO:5. In some embodiments, the scFv comprises a linker between the VL and the VH, wherein the linker has the formula (Gly₃₋₄-Ser)₁₋₄. In some embodiments, the scFv comprises a linker between the VL and the VH, wherein the linker is GGGGSGGGGSGGGSGGGGS (SEQ ID NO:53).

In any of the foregoing or related aspects and embodiments, the anti-FLT3 antibodies and fragments thereof described herein (e.g., scFv) do not compete (or do not substantially compete) with FLT3 ligand for binding to FLT3.

In some aspects, the disclosure provides nucleic acids encoding any of the anti-FLT3 antibodies and antigen binding fragments described herein (e.g., scFv). In some aspects, the disclosure provides a vector comprising a nucleic acid encoding any of the anti-FLT3 antibodies and antigen binding fragments described herein (e.g., scFv). In some aspects, the disclosure provides a recombinant receptor (e.g., a chimeric antigen receptor) comprising any of the anti-FLT3 antigen binding fragments described herein (e.g., scFv). In some aspects, the disclosure provides nucleic acids encoding recombinant receptors (e.g., chimeric antigen receptors) comprising any of the anti-FLT3 antigen binding fragments described herein (e.g., scFv). In some aspects, the disclosure provides a vector comprising a nucleic acid encoding a recombinant receptor comprising any of the anti-FLT3 antigen binding fragments described herein (e.g., scFv).

In some aspects, the disclosure provides a chimeric antigen receptor (CAR), wherein the CAR comprises: (i) an extracellular domain comprising (a) an antibody or fragment of any of the foregoing or related aspects, or (b) an anti-FLT3 scFv of any of the foregoing or related aspects and embodiments; (ii) a transmembrane domain; and (iii) an intracellular domain.

In any of the foregoing or related aspects and embodiments of the CAR, the transmembrane domain is a CD3 transmembrane domain, a CD4 transmembrane domain, a CD8 transmembrane domain, or a CD28 transmembrane domain. In some embodiments of the CAR, the transmembrane domain is a CD8 transmembrane domain (e.g., CD8 alpha transmembrane domain).

In any of the foregoing or related aspects and embodiments of the CAR, the intracellular domain comprises an activation domain (e.g., wherein, when the CAR is expressed in a T cell, the activation domain transmits an activation signal after the extracellular domain binds FLT3). In some embodiments, the disclosure provides a CARs wherein the activation domain (in the intracellular domain) comprises an intracellular signaling domain of CD3zeta, CD3epsilon, or FcRgamma. In some embodiments, the disclosure provides a CAR comprising CD3zeta activation domain/intracellular signaling domain. In some embodiments of the CAR, the intracellular domain further comprises one or more co-stimulatory domains. In some embodiments, the one or more co-stimulatory domains are from one or more of: CD28, 4-1BB, CD27, OX40 or ICOS. In some embodiments, the one or more co-stimulatory domains are from CD28 and/or 4-1BB.

In any of the foregoing or related aspects and embodiments of the CAR, the CAR comprises a spacer or hinge region between the extracellular domain and the transmembrane domain. In some embodiments, the spacer or hinge region is from the extracellular domain of CD8 (e.g., CD8 alpha).

In any of the foregoing or related aspects and embodiments of the CAR, the extracellular domain further comprises a cleavable signal peptide.

In any of the foregoing or related aspects and embodiments of the CAR, the extracellular domain comprises an scFv comprising the amino acid sequence of SEQ ID NO:4; the transmembrane domain comprises a CD8 transmembrane domain; and the intracellular domain comprises an intracellular signaling domain of CD3zeta and a co-stimulatory domain of CD28 and/or 4-1BB.

In some embodiments, the CAR described herein comprises (or substantially consists of or consists of) the amino acid sequence selected from the group comprising: SEQ ID NO:6, SEQ ID NO:9, SEQ ID NO:10, SEQ ID NO:11, SEQ ID NO:12, SEQ ID NO:13, SEQ ID NO:14, and SEQ ID NO:15. In some embodiments, the CAR described herein comprises (or substantially consists of or consists of) the amino acid sequence of SEQ ID NO:6. In some embodiments, the CAR described herein comprises (or substantially consists of or consists of) the amino acid sequence of SEQ ID NO:9. In some embodiments, the CAR described herein comprises (or substantially consists of or consists of) the amino acid sequence of SEQ ID NO:10. In some embodiments, the CAR described herein comprises (or substantially consists of or consists of) the amino acid sequence of SEQ ID NO:11. In some embodiments, the CAR described herein comprises (or substantially consists of or consists of) the amino acid sequence of SEQ ID NO:12. In some embodiments, the CAR described herein comprises (or substantially consists of or consists of) the amino acid sequence of SEQ ID NO:13. In some embodiments, the CAR described herein comprises (or substantially consists of or consists of) the amino acid sequence of SEQ ID NO:14. In some embodiments, the CAR described herein comprises (or substantially consists of or consists of) the amino acid sequence of SEQ ID NO:15.

In any of the foregoing or related aspects and embodiments of the CAR, the CAR further comprises a safety switch polypeptide (e.g., wherein the safety switch polypeptide is bound to the CAR by a self-cleaving peptide). In some embodiments, the safety switch polypeptide is iCasp9 or EGFRt. In some embodiments, the self-cleaving peptide is T2A, P2A, E2A, F2A or IRES. In some embodiments, the self-cleaving peptide is T2A.

In any of the foregoing or related aspects and embodiments of the CAR, an immune cell (e.g., a T cell) expressing the CAR is activated or stimulated to proliferate when the extracellular domain binds to FLT3 (e.g., on the surface of a cancer cell, hematopoietic stem cell, hematopoietic progenitor cell or dendritic cell). In some embodiments, the CAR, when expressed on the surface of an immune cells (e.g., a T cell), directs the immune cell to kill a cell expressing FLT3.

In some aspects, the disclosure provides an immune cell (e.g., a T cell) or a population of immune cells (e.g., T cells) expressing a CAR of any of the foregoing or related aspects and embodiments. In some aspects, the disclosure provides an immune cell (e.g., a T cell) a population of immune cells (e.g., T cells) comprising a nucleic acid encoding a CAR of any of the foregoing or related aspects and embodiments. In any of the foregoing or related aspects and embodiments, the immune cell is a T cell, a NK cell, a macrophage or a monocyte. In some embodiments, the immune cell is a T cell.

In any of the foregoing or related aspects and embodiments, the immune cell (e.g., T cell) comprises a nucleic acid, wherein the nucleic acid comprises a sequence selected from the group comprising: SEQ ID NO:60, SEQ ID NO:63, SEQ ID NO:64, SEQ ID NO:65, SEQ ID NO:66, SEQ ID NO:67, SEQ ID NO:68, and SEQ ID NO:69.

In any of the foregoing or related aspects and embodiments, the immune cell (e.g., a T cell) has been derived from a subject (e.g., a human) before introducing the CAR or the nucleic acid. In some embodiments, the immune cell expressing the CAR or comprising the nucleic acid is further expanded to generate a population of immune cells.

In some embodiments, any of the anti-FLT3 CARs described herein are cytotoxic against AML cells in vitro.

In some embodiments, any of the immune cells described herein are characterized by stable expression of any of the anti-FLT3 CARs described herein.

In some embodiments, any of the immune cells expressing an anti-FLT3 CAR described herein are characterized by high proliferative potential.

In some aspects, the disclosure provides a pharmaceutical composition comprising (i) a humanized anti-FLT3 antibody or fragment of any one of the foregoing or related aspects and embodiments, and (ii) a pharmaceutically acceptable carrier. In some embodiments, the disclosure provides a pharmaceutical composition comprising (i) an scFv of any one of the foregoing or related aspects and embodiments, and (ii) a pharmaceutically acceptable carrier.

In some aspects, the disclosure provides a pharmaceutical composition comprising (i) an immune cell (e.g., a T cell) of any one of the foregoing or related aspects and embodiments, and (ii) a pharmaceutically acceptable carrier.

In some aspects, the disclosure provides a pharmaceutical composition comprising (i) a population of immune cells (e.g., T cells) of any one of the foregoing or related aspects and embodiments, and (ii) a pharmaceutically acceptable carrier.

In some aspects, the disclosure provides a method of treating a hematologic cancer in a subject in need thereof, wherein the method comprises administering to the subject (e.g., a therapeutically effective amount of): (i) a humanized anti-FLT3 antibody or fragment (e.g., scFv) of any one of the foregoing or related aspects and embodiments, or (ii) a pharmaceutical composition comprising such humanized anti-FLT3 antibody or fragment (e.g., scFv).

In some aspects, the disclosure provides a method of treating a hematologic cancer in a subject in need thereof, wherein the method comprises administering to the subject (e.g., a therapeutically effective amount of): (i) an immune cell (e.g., a T cell) of any of the foregoing or related aspects and embodiments (such as the cell expressing any of the CARs described herein), (ii) a population of immune cells (e.g., T cells) of any of the foregoing or related aspects and embodiments (such as the cells expressing any of the CARs described herein), or (ii) a pharmaceutical composition of such immune cells or population of immune cells.

In some aspects, the disclosure provides methods for preparing or conditioning a subject in need thereof for hematopoietic cell transplantation, wherein the method comprises administering to the subject (e.g., a therapeutically effective amount of): (i) a humanized anti-FLT3 antibody or fragment (e.g., scFv) of any one of the foregoing or related aspects and embodiments, or (ii) a pharmaceutical composition comprising such humanized anti-FLT3 antibody or fragment (e.g., scFv).

In some aspects, the disclosure provides methods for preparing or conditioning a subject in need thereof for hematopoietic cell transplantation, wherein the method comprises administering to the subject (e.g., a therapeutically effective amount of): (i) an immune cell (e.g. a T cell) of any of the foregoing or related aspects and embodiments (such as the cell expressing any of the CARs described herein), (ii) a population of immune cells of any of the foregoing or related aspects and embodiments (such as the cell expressing any of the CARs described herein), or (ii) a pharmaceutical composition of such immune cells or population of immune cells.

In any of the foregoing or related aspects and embodiments of the conditioning method, the method further comprises performing hematopoietic cell transplantation to the subject after the administering. In some embodiments, the hematopoietic cell transplantation comprises transplantation to the subject of hematopoietic stem cells and/or hematopoietic progenitor cells. In some embodiments, the performing of the hematopoietic cell transplantation occurs 5 days to 6 weeks after the administering. In some embodiments, the performing of the hematopoietic cell transplantation occurs about 2 to 3 weeks after the administering.

In any of the foregoing or related aspects and embodiments, the hematologic cancer is acute myeloid leukemia (AML), acute lymphoblastic leukemia (ALL), chronic myeloid leukemia (CML), chronic lymphocytic leukemia (CLL), blastic plasmacytoid dendritic cell neoplasm (BPDCN), peripheral T cell lymphoma, follicular lymphoma, diffuse large B cell lymphoma, Hodgkin lymphoma, non-Hodgkin lymphoma, neuroblastoma, a non-malignant inherited or acquired marrow disorder, multiple myeloma, or a dendritic cell neoplasm. In some embodiments, the hematologic cancer is AML. In some embodiments, the hematologic cancer is ALL. In some embodiments, the hematologic cancer is a dendritic cell neoplasm. In some embodiments, the hematologic cancer is blastic plasmacytoid dendritic cell neoplasm (BPDCN). In some embodiments, the hematologic cancer is a B-lineage leukemia.

In any of the foregoing or related aspects and embodiments, the subject in need thereof has a hematologic cancer (such as any cancer described herein).

In some embodiments, the administering described herein (e.g., in a therapeutically effective amount) reduces the cell population expressing FLT3 by at least 60% (e.g., at least 70%, or at least by 75%) in the subject. In some embodiments, the administering described herein (e.g., in a therapeutically effective amount) reduces the cell population expressing FLT3 by at least 80% (e.g., at least by 90%, at least by 95%) in the subject. The reductions can be in any one or more of blood, bone marrow cells and/or cancer cells of the subject relative to baseline.

In some embodiments, the administering described herein (e.g., in a therapeutically effective amount) reduces HSCs and/or HSPCs (e.g., HSCs and early progenitors) by at least 60% (e.g., at least by 70%, at least by 75%) in the subject. In some embodiments, the administering described herein (e.g., in a therapeutically effective amount) reduces HSCs and/or HSPCs (e.g., HSCs and early progenitors) by at least 80% (e.g., at least by 90%, at least by 95%) in the subject. The reduction can be in blood and/or bone marrow cells of the subject relative to baseline.

In any of the foregoing or related aspects and embodiments, the administering specifically targets human CD34⁺ hematopoietic stem cells and/or hematopoietic progenitor cells. In some embodiments, the administering described herein (e.g., in a therapeutically effective amount) reduces CD34+ HSPCs (e.g., HSCs and early progenitors) by at least 60% (e.g., at least by 70%, at least by 75%) in the subject. In some embodiments, the administering described herein (e.g., in a therapeutically effective amount) reduces CD34+ HSPCs (e.g., HSCs and early progenitors) by at least 80% (e.g., at least by 90%, at least by 95%) in the subject. The reduction can be in blood and/or bone marrow cells of the subject relative to baseline.

In some embodiments, the administering described herein (e.g., in a therapeutically effective amount) reduces dendritic cells by at least 60% (e.g., at least by 70%, at least by 75%) in the subject. In some embodiments, the administering described herein (e.g., in a therapeutically effective amount) reduces dendritic cells by at least 80% (e.g., at least by 90%, at least by 95%) in the subject. The reduction can be in blood and/or bone marrow cells of the subject relative to baseline.

In some embodiments, the administering reduces bone marrow lineage frequencies and numbers in the subject (e.g., reduces bone marrow frequencies and/or numbers by at least 50%, at least 55%, at least 600%, at least 65%, at least 70%, at least 75%, at least 80% or at least 85% relative to baseline levels). In some embodiments, the administering described herein reduces circulating myeloid lineages in the subject (e.g., reduces circulating myeloid lineages by at least 60%, at least 65%, at least 70%, at least 75%, at least 80% or at least 85% relative to baseline levels).

In some embodiments, the administering reduces human CD34⁺CD38⁺ cell population in bone marrow mononuclear cells of the subject (e.g., by at least 50%, at least 55%, at least 60% or at least 65% relative to baseline levels), and/or reduces human CD34⁺CD38⁻ cell population in bone marrow mononuclear cells of the subject (e.g., by at least 60%, at least 65%, at least 70%, at least 75%, at least 80% or at least 85% relative to baseline levels).

In some embodiments of the methods described herein, the methods described herein are effective to treat the cancers described herein (e.g., AML) and/or condition the patient for HSCT. In some embodiments of the methods described herein, the methods described herein are effective to slow progression of the cancers described herein (e.g., AML). In some embodiments of the methods described herein, the methods described herein are effective to reduce tumor burden of the cancers described herein (e.g., AML). In some embodiments of the methods described herein, the methods described herein are effective to increase survival of a subject having a cancer described herein (e.g., AML).

In any of the foregoing or related aspects and embodiments, the therapeutically effective amount of the anti-FLT3 CAR-expressing immune cells or the population of immune cells is a dose from about 50,000,000 to 10,000,000,000 cells. In some embodiments, the therapeutically effective amount of the anti-FLT3 CAR-expressing immune cells or the population of immune cells is a dose from about 100,000,000 to 2,000,000,000 cells. In some embodiments, the therapeutically effective amount of the anti-FLT3 CAR-expressing immune cells or the population of immune cells is a dose from about 200,000,000 to 1,000,000,000 cells. In some embodiments, the therapeutically effective amount of the anti-FLT3 CAR-expressing immune cells or the population of immune cells is a dose from about 300,000,000 to 700,000,000 cells.

In any of the foregoing or related aspects and embodiments of the methods described herein, the administration is intravenous. In some embodiments, the intravenous administration is by infusion into the subject. In some embodiments, the intravenous administration is by a bolus injection into the subject.

In some embodiments of the methods described herein, the administering occurs once. In some embodiments of the methods described herein, the administering is every 3-7 days for 2 to 3 weeks.

In any of the foregoing or related aspects and embodiments of the methods described herein, the method further comprises the following steps prior to the administering step:
(i) collecting of blood from the subject;
(ii) isolating immune cells (e.g., T cells) from the blood;
(iii) introducing a nucleic acid encoding a CAR of any of the foregoing or related aspects and embodiments into the isolated immune cells; and
(iv) expanding the isolated immune cells obtained in step (iii), wherein the expanding yields the immune cells or the population of immune cells administered during the administering step.

In any of the foregoing or related aspects and embodiments, the pharmaceutical compositions described herein further comprise a checkpoint inhibitor. In any of the foregoing or related aspects and embodiments, the methods described herein further comprise administering a checkpoint inhibitor. In some embodiments, the checkpoint inhibitor is an antagonist of PD1, PD-L1 or CTLA4 (e.g., any such antagonist known in the art, e.g., an antagonistic antibody such as an antagonistic anti-PD1 antibody).

In any of the foregoing or related aspects and embodiments, the subject is a human (for example, the subject being treated using any of the methods described herein).

### Definitions

As used herein, the term “about,” when used to modify a numeric value, indicate that deviations of up to 10% above and below the numeric value remain within the intended meaning of the recited value.

As used herein, the term “VL” refers to the light chain variable region of an antibody.

As used herein, the term “VH” refers to the heavy chain variable region of an antibody.

As used herein, the term “percent (%) amino acid sequence identity” or “percent sequence identity” with respect to a reference polypeptide sequence is defined as the percentage of amino acid residues in a candidate sequence that are identical with the amino acid residues in the reference polypeptide sequence. Percent sequence identity is determined after aligning the sequences and introducing gaps, if necessary, to achieve the maximum percent sequence identity. Alignment for purposes of determining percent amino acid sequence identity can be achieved in various ways that are known in the art. Example alignment tools include but are not limited to BLASTp, BLAST-2, ALIGN (e.g., ALIGN-2) or Megalign (DNASTAR) software.

### BRIEF DESCRIPTION OF THE DRAWINGS

FIG. 1A shows binding competition of chimeric antibody 1-18BA (comprising mouse VL (SEQ ID NO: 25) and mouse VH (SEQ ID NO:27) and human IgG) with and without FLT3 ligand in REH cells.

FIGS. 1B and 1C show binding affinities of humanized anti-FLT3 IgG (having a VL of SEQ ID NO: 1 and VH of SEQ ID NO: 3) and humanized anti-FLT3 scFv (SEQ ID NO: 4 further comprising a His Tag on the C terminus) to REH cells, respectively.

FIGS. 2A-2C: FIG. 2A is a schematic showing the production of autologous CAR-T cells and its use as an autologous CAR T therapy. FIG. 2B is a schematic showing the CAR structure of an anti-FLT3 scFV CAR which targets FLT3 expressing cells (e.g. HSPCs, Dendritic cells, and acute myeloid leukemia (AML)). FIG. 2C is a schematic demonstrating the mechanism of cell killing of target FLT3 cells by anti-FLT3 CAR T cells; activation of the CAR by FLT3 on a target cell induces expression of cell perforin and granzyme to induce apoptosis in the target cell.

FIGS. 3A-3D: FIG. 3A is an outline of the methods for generating anti-FLT3 CAR T cells and assessing transduction efficiency and cell cytotoxicity. FIG. 3B is a bar graph showing transduction efficiency, reported as % GFP⁺ T cells, of different viral MOIs over time in T-cells transduced with anti-FLT3 CAR comprising anti-FLT3 scFv (CAR encoded by SEQ ID NO: 16 (encoding domains in the following orientation: signal peptide-linker-scFV of SEQ ID NO: 4-linker-CD8α hinge-CD8α transmembrane domain-CD28-41BB-CD3ζ-T2A-GFP)). FIG. 3C is a plot showing fold-expansion of the CAR T cells (from FIG. 2A) transduced at MOI 10. FIG. 3D are flow plots displaying percent (%) GFP expression at different MOIs on day 10 of the CAR T cell culture (top) and anti-FLT3 scFv expression with an anti-Fab APC antibody (Jackson ImmunoResearch, no. 109-607-003) versus GFP expression (bottom), using the CAR T cells from FIG. 2A.

FIGS. 4A-4C show that anti-FLT3-CAR T cells (as described in FIG. 3B) are cytotoxic against MOLM-13 AML cells. FIG. 4A shows representative experimental methods. FIG. 4B shows representative flow plots showing frequencies of dead MOLM13 target cells (7-AAD⁺ CellTrace⁺) at E:T ratios of 1:1 at 24 (top) and 48 hours (bottom) of co-culture with untransduced or anti-FLT3 CAR-T cells. FIG. 4C shows bar graphs representing mean and s.e.m. of target cell (MOLM13) killing at indicated E:T ratio at 24 (top) and 48 hours (bottom).

FIGS. 5A-5E show in vivo efficacy of anti-FLT3 CAR T against MOLM-13 AML cells: FIG. 5A shows a timeline of engraftment of anti-FLT3 CAR3a T cells (as described in FIG. 3B) in mice harboring MOLM-13 cells (an AML cell line). FIG. 5B is a survival curve of mice treated with control T cells or the anti-FLT3 CAR-T cells 73 d. FIG. 5C shows overall frequency of human CD45+ MOLM-13 cells in in peripheral blood mononuclear fraction before and after treatment with control or CAR-T cells shown for individual mice. FIG. 5D shows frequency of total T cells and the GFP+ anti-FLT3 CAR3a-T cells in peripheral blood mononuclear fraction after treatment with control or GFP+ CAR T cells. FIG. 5E shows frequency of MOLM-13 cells after treatment with control T cells or the anti-FLT3 CAR3a-T cells in peripheral blood mononuclear fraction. In FIGS. 5C and 5D, “X” indicates no mice were measured in the control group as all mice were dead by day 28.

FIGS. 6A-6D show successful conditioning with autologous CAR T cells (of mice “humanized” by engrafting with the human bank cells): FIG. 6A shows a timeline of mice transplanted with CD123 (CD34+) cells and administered either control T cells or anti-FLT3 CAR T cells (as described in FIG. 3B). FIG. 6B shows overall frequency of human CD45 cells in MNC fraction before and after treatment with control T cells or the anti-FLT3 CAR-T cells shown for individual mice in the two cohorts. FIG. 6C shows lineage frequencies (T cells (CD3⁺), B cells (CD19⁺), and myeloid cells (CD33⁺)) before and after treatment with control T cells or the anti-FLT3 CAR-T cells shown as averages of all mice in the two cohorts. FIG. 6D shows fold change in lineage frequencies relative to pre-treatment frequencies shown for individual mice in control T cells and the anti-FLT3 CAR T cohorts. Myeloid compartment shows significant decline in the anti-FLT3 CAR-T cell treated mice compared to those treated with control T cells.

FIGS. 7A-7D: FIG. 7A shows femurs and tibias from mice transplanted with anti-FLT3-CAR T cells (as described in FIG. 3B) and control T cells (no gross anatomical differences were observed). FIG. 7B shows total cell count and flow cytometry analysis of MNCs from BM (BM-MNCs) and frequency of human CD45+ cells in control T cell and the anti-FLT3-CAR T cell transplants. FIG. 7C shows lineage frequencies (T cells (CD3⁺), B cells (CD19⁺), and myeloid cells (CD33⁺)) in the BM-MNCs shown as an average of all mice in the two cohorts. FIG. 7D shows lineage cell counts (T cells (CD3⁺), B cells (CD19⁺), and myeloid cells (CD33⁺)) in BM before and after treatment with control or the anti-FLT3 CAR-T cells shown for individual mice in the two cohorts.

FIG. 8A-8B: FIG. 8A shows representative contour plots gated on mCD45−hCD45+Lin− in control T cells and anti-FLT3 CAR T (as described in FIG. 3B) treated mice (showing significant depletion of HSPC CD38+CD34+ and CD38−CD34+ populations in the anti-FLT3 CAR T treated mice compared to controls), and shows summary graphs of CD38+CD34+ and CD38−CD34+ HSPCs as a percentage of total bone marrow mononuclear cells (BM-MNCs) shown for individual mice in control T cell and the anti-FLT3 CAR T treated mice. Anti-FLT3 CAR T cell treated mice have significantly fewer progenitors in the bone marrow compared to control mice. FIG. 8B shows frequencies of hematopoietic stem cells (HSC, CD90+CD45RA−) and multi-potent progenitors (MPP, CD90−CD45RA−) cells as a percentage of total BM-MNCs shown for individual mice treated with control T cells or the anti-FLT3 CAR T cells. Anti-FLT3 CAR T cell treated mice have significantly fewer progenitors in the bone marrow compared to control mice.

FIGS. 9A-9D: FIG. 9A shows flow cytometry plots measuring transduction efficiency of suicide CAR vectors based on surface expression of anti-FLT3 scFv in human T cells, showing frequencies of anti-FLT3 CAR3a-T cells, anti-FLT3-CAR3a-EGFRt (the resulting CAR has an amino acid sequence of SEQ ID NO: 7 and encodes domains in the following orientation: signal peptide-linker-scFV of SEQ ID NO: 4-linker-CD8α hinge-CD8α transmembrane domain-CD28-41BB-CD3ζ-T2A-EGFRt), and anti-FLT3-CAR-icasp9 (the resulting CAR has amino acid sequence of SEQ ID NO: 8 and encodes domains in the following orientation: signal peptide-linker-scFV of SEQ ID NO: 4-linker-CD8α hinge-CD8α transmembrane domain-CD28-41BB-CD3ζ-T2A-iCasp9) cells (35.3%, 27.5% and 16.9/6, respectively). FIG. 9B is a schematic of the in vitro cytotoxicity test of anti-CAR T cells with suicide switches CAR3a-EGFRt or CAR3a-icasp9 compared to the original construct CAR3a against target AML NOMO-1 cells (expressing FLT3) at various effector:target (E:T) cell ratios (10:1, 5:1, 2:1, 1:1, 1:2 and 1:5). FIG. 9C shows representative dot plots showing the flow data after excluding debris after 24 hours of co-culture of effector and target cells. Target cells were identified as CellTraceViolet+ and effector cells as CellTraceViolet−. The figure shows frequencies of dead (7AAD+) target cells after gating on the CellTraceViolet+ cells. FIG. 9D is a bar graph demonstrating the frequency of dead (7AAD+) cells at various T cell effector: NOMO-1 target cell ratios for the anti-FLT3 CARs co-cultured with NOMO-1 cells for 24 hours. All FLT3 CAR T cells show significantly more cytotoxic effect against FLT3+ NOMO-1 cells compared to control T cells. There is no significant difference in cytotoxic effect between either of the two suicide CAR T cells and the original CAR construct.

FIGS. 10A-10C: FIG. 10A shows flow plots demonstrating surface expression of the anti-FLT3 CAR3a (detecting scFv) and EGFRt (using cetuximab) in T cells transduced with the CAR3a-T2A-EGFRt lentiviral vector (as described in FIG. 9A). FIG. 10B is a schematic of the in vitro antibody dependent cellular cytotoxicity (ADCC) test for CAR3a-T2A-EGFRt T cells. FIG. 10C is a graph demonstrating the percent (%) remaining anti-FLT3 CAR T cells after treatment with various doses of cetuximab, where T cells were cultured alone, with total allogenic MNC cells or with allogenic MNCs depleted of T cells. Transduced T cells cultured alone show no significant decrease in CAR3a expressing cells after treatment with cetuximab, whereas transduced cells cultured with total allogenic MNCs or such MNCs depleted of T cells show dose dependent depletion of CAR3a expressing cells with cetuximab. Results support function of ADCC against EGFRt expressing anti-FLT CAR T cells in vitro.

FIGS. 11A-11D: FIG. 11A shows a timeline of an in vivo experiment measuring survival and frequency of CAR-T cells in peripheral blood of mice harboring EGFP-MOLM-13 cells after treatment with CAR3a-T2A-EGFRt-T (as described in FIG. 9A) cells or control T cells. FIG. 11B shows a survival curve of mice treated with control T cells or anti-FLT3 CAR3a EFGRt-T cells (with and without cetuximab), generated up to 65 days post AML injection. FIG. 11C shows frequency of MOLM-13 (mCD45−hCD45+EGFP+) cells and T cells (mCD45-hCD45+CD3+) in peripheral blood (PB) at 2, 4, and 6 weeks post treatment with control T cells or anti-FLT3 CAR3a-T cells (with or without cetuximab). FIG. 11D shows relative amount of circulating anti-FLT3 CAR3a EFGRt-T cells (with and without cetuximab) at 4 and 6 weeks post administration of the CAR T-cells as measured by CAR DNA levels (normalized to human actin DNA).

FIGS. 12A-12K show plasmid constructs for CARs. FIG. 12A shows plasmid expressing the CAR of SEQ ID NO: 16. FIG. 12B shows the plasmid expressing the CAR of SEQ ID NO: 7. FIG. 12C shows the plasmid expressing the CAR of SEQ ID NO: 8. FIG. 12D shows the plasmid expressing the CAR of SEQ ID NO: 6. FIG. 12E shows the plasmid expressing the CAR of SEQ ID NO: 12. FIG. 12F shows the plasmid expressing the CAR of SEQ ID NO: 11. FIG. 12G shows the plasmid expressing the CAR of SEQ ID NO: 10. FIG. 12H shows the plasmid expressing the CAR of SEQ ID NO: 9. FIG. 12I shows the plasmid expressing the CAR of SEQ ID NO: 13. FIG. 12J shows the plasmid expressing the CAR of SEQ ID NO: 14. FIG. 12K shows the plasmid expressing the CAR of SEQ ID NO: 15.

### DETAILED DESCRIPTION

In certain aspects, and without being bound to any specific mechanism of action, to address the challenges of efficacy and resistance in targeting cancers, described herein are antibodies, antigen-binding fragments and CAR T cells which can specifically and efficaciously target and kill Fms-like Tyrosine Kinase 3 (FLT3) expressing cells. The antibodies, fragments and CAR T cells described herein can target FLT3 expressed on the surface of cancer cells (e.g., leukemic cells, such as AIL blasts), as well as HSCs/HSPCs, and specifically eliminate such cells, allowing for subsequent cancer therapy and/or hematopoietic stem cell transplantation. The antibodies and antigen-binding fragments (e.g., scFvs) described herein can bind an extracellular, membrane proximal FLT3 domain, outside the regions commonly mutated in cancer, and do not compete for binding to FLT3 with FLT3 ligand. Unlike other known therapies, the antibodies, fragments, CAR T cells, compositions and methods described herein can target FLT3-expressing cells regardless of commonly known mutations in the FLT3 receptor.

In one aspect, provided herein are humanized antibodies specifically binding FLT3, or antigen binding fragments thereof (such as heavy chain variable regions (VH), light chain variable regions (VL) and single chain fragments (such as scFVs)). In certain embodiments, the humanized anti-FLT3 antibodies and antigen binding fragments thereof provided herein specifically bind human and monkey (e.g. Rhesus macaque) FLT3. In certain embodiments, the humanized anti-FLT3 antibodies and antigen binding fragments provided herein specifically bind human FLT3.

In another aspect, provided herein are nucleic acids encoding the humanized anti-FLT3 antibodies and antigen binding fragments provided herein. Also provided herein are vectors comprising nucleic acids encoding the humanized anti-FLT3 antibodies and antigen binding fragments provided herein. Also provided are cells expressing such nucleic acids for producing such antibodies and fragments, and methods of making such antibodies and fragments.

In one aspect, provided herein are chimeric antibodies specifically binding FLT3. In certain embodiments, the chimeric anti-FLT3 antibodies provided herein specifically bind human and monkey (e.g. Rhesus macaque) FLT3. In certain embodiments, the chimeric anti-FLT3 antibodies provided herein specifically bind human FLT3. Also provided herein are nucleic acids encoding the chimeric anti-FLT3 antibodies provided herein. Also provided herein are vectors comprising nucleic acids encoding the chimeric anti-FLT3 antibodies provided herein. Also provided are cells expressing such nucleic acids for producing such antibodies, and methods of making such antibodies and fragments.

In another aspect, provided herein are recombinant receptors comprising the anti-FLT3 antibodies or antigen binding fragments thereof described herein. In certain embodiment, provided herein are chimeric antigen receptors (CARs) comprising the anti-FLT3 antibodies or antigen binding fragments thereof described herein.

In another aspect, provided herein are immune cells comprising the CARs described herein (e.g., CAR T cells).

In yet another aspect, provided herein are methods of use of the humanized anti-FLT3 antibodies, antigen binding fragments thereof, recombinant receptors such as CARs, and immune cells (e.g., CAR T cells) described herein. In certain embodiment, provided herein are methods of treatment of hematological malignancies (e.g., AML) using anti-FLT3 CAR immune cells (e.g., by administering anti-FLT3 CAR T cells to a human). In certain embodiment, provided herein are methods of HSC transplant conditioning using anti-FLT3 CAR T cells (e.g., by administering anti-FLT3 CAR T cells to a human). In some embodiments, the methods of HSC transplant conditioning can be followed by hematopoietic cell transplantation. In certain embodiment, provided herein are methods of treatment of hematological malignancies (e.g., AML) using anti-FLT3 antibodies or antigen binding fragments thereof (e.g., by administering anti-FLT3 antibody or fragment to a human). In certain embodiment, provided herein are methods of HSC transplant conditioning using anti-FLT3 antibodies or antigen binding fragments thereof (e.g., by administering anti-FLT3 antibody or fragment to a human). In some embodiments, the methods of HSC transplant conditioning can be followed by hematopoietic cell transplantation.

### Anti-FLT3 Antibodies

Provided herein are antibodies and antigen-binding fragments thereof that bind to FLT3. References to antibody fragments made herein refer to antigen-binding fragments of the described antibodies. In certain embodiments, provided herein are antibodies and fragments thereof that specifically bind human and rhesus monkey FLT3. In certain embodiments, provided herein are antibodies and fragments thereof that specifically bind human FLT3. The antibodies and fragments described herein may display cross-reactivity with a FLT3 from one or more other species (in addition to human and rhesus monkey). In some embodiments, also contemplated are antibodies and fragments thereof that specifically bind human and/or monkey (e.g., rhesus monkey) FLT3, and do not display cross-reactivity with FLT3 from other species. In certain embodiments, provided herein are humanized antibodies and antigen-binding fragments thereof that bind to FLT3. In certain embodiments, provided herein are chimeric antibodies and antigen-binding fragments thereof that bind to FLT3.

In some embodiments, the contemplated anti-FLT3 antibodies and fragments comprise any CDRs described herein. In some embodiments, provided herein are single-chain variable fragments (scFV) comprising any CDRs described herein. In some embodiments, the contemplated anti-FLT3 antibodies and fragments comprise any light chain variable region described herein and/or any heavy chain variable region described herein. In some embodiments, provided herein are single-chain variable fragments (scFV) comprising any light chain variable region described herein and/or any heavy chain variable region described herein.

In some embodiments, the contemplated humanized anti-FLT3 antibodies and fragments comprise any CDRs described herein. In some embodiments, the contemplated humanized anti-FLT3 antibodies and fragments comprise any light chain variable region described herein and/or any heavy chain variable region described herein.

In some embodiments, the described anti-FLT3 antibodies and fragments comprise a light chain variable region having a sequence with at least 95% identity to any light chain variable region described herein and/or a heavy chain variable region having a sequence with at least 95% identity to any heavy chain variable region described herein. In some embodiments, provided herein are single-chain variable fragments (scFv) comprising a sequence with at least 95% identity to any light chain variable region described herein and/or a heavy chain variable region having a sequence with at least 95% identity to any heavy chain variable region described herein.

In some embodiments, the described anti-FLT3 antibodies and fragments comprise a light chain variable region having a sequence with at least 95% identity to any light chain variable region described herein (with at least 97% identity in the CDR region) and/or a heavy chain variable region having a sequence with at least 95% identity to any heavy chain variable region described herein (with at least 97% identity in the CDR region). In some embodiments, provided herein are single-chain variable fragments (scFV) comprising a sequence with at least 95% identity to any light chain variable region described herein (with at least 97% identity in the CDR region) and/or a heavy chain variable region having a sequence with at least 95% identity to any heavy chain variable region described herein (with at least 97% identity in the CDR region).

### Complementarity-Determining Regions

Complementarity-determining regions (CDRs) are defined in various ways in the art, including the Kabat, Chothia, AbM, Contact, and IMGT. In some embodiments, the CDRs of an antibody are defined according to the Kabat system. The Kabat system is based on sequence variability (see, e.g., Kabat E A et al, (1991) Sequences of Proteins of Immunological Interest, Fifth Edition, U.S. Department of Health and Human Services, NIH Publication No. 91-3242; Kabat E A & Wu T T (1971) Ann NY Acad Sci 190: 382-391). In some embodiments, the CDRs of the antibodies described herein are determined using the Kabat system.

In some embodiments, the CDRs of an antibody are defined according to the Chothia System. The Chothia system is based on the location of immunoglobulin structural loop regions (see, e.g., Tramontano A et al, (1990) J Mol Biol 215(1): 175-82; Chothia C & Lesk A M, (1987), J Mol Biol 196: 901-917; U.S. Pat. No. 7,709,226; Al-Lazikani B et al., (1997) J Mol Biol 273. 927-948; and Chothia C et al, (1992) J Mol Biol 227: 799-817). The term “Chothia CDRs,” and like terms are recognized in the art and refer to antibody CDR sequences as determined according to the method of Chothia and Lesk, 1987, J. Mol. Biol., 196:901-917 (see also, e.g., U.S. Pat. No. 7,709,226 and Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001)). In some embodiments, the CDRs of the antibodies described herein are determined using the Chothia system.

In some embodiments, the CDRs of an antibody are defined according to the AbM System. The AbM system is based on hypervariable regions that represent a compromise between the Kabat CDRs and Chothia structural loops, and where CDRs are determined using Oxford Molecular's AbM antibody modeling software (Oxford Molecular Group, Inc.). In some embodiments, the CDRs of the antibodies described herein are determined using the AbM numbering system.

In some embodiments, the CDRs of an antibody are defined according to the IMGT system (see “IMGT®, the international ImMunoGeneTics information System® website imgt.org, founder and director: Marie-Paule Lefranc, Montpellier, France; see, e.g., Lefranc, M.-P. et al., 1999, Nucleic Acids Res., 27:209-212 and Lefranc, M.-P., 1999, The Immunologist, 7:132-136 and Lefranc, M.-P. et al., 1999, Nucleic Acids Res., 27:209-212). In some embodiments, the CDRs of the antibodies described herein are determined using the IMGT system.

In some embodiments, the CDRs of an antibody are defined according to the Contact system. The Contact definition is based on an analysis of the available complex crystal structures (bioinf.org.uk/abs) (see e.g., Martin A. “Protein Sequence and Structure Analysis of Antibody Variable Domains,” in Antibody Engineering, Kontermann and Diibel, eds., Chapter 31, pp. 422-439, Springer-Verlag, Berlin (2001), and MacCallum R M et al., (1996) J Mol Biol 5: 732-745). In some embodiments, the CDRs of the antibodies described herein are determined using the Contact system.

The Kabat, Chothia, AbM, IMGT and/or Contact CDR positions may vary depending on the antibody, and may be determined according to methods known in the art.

In some embodiments, provided herein are anti-FLT3 antibodies or fragments thereof having a light chain variable region comprising a complementarity determining region 1 (CDR-L1) having the amino acid sequence of SEQ ID NO: 86. In some embodiments, provided herein are humanized anti-FLT3 antibodies or fragments thereof having a light chain variable region comprising a complementarity determining region 2 (CDR-L2) having the amino acid sequence of SEQ ID NO: 87. In some embodiments, provided herein are anti-FLT3 antibodies or fragments thereof having a light chain variable region comprising a complementarity determining region 3 (CDR-L3) comprising the amino acid sequence of SEQ ID NO: 88. In some embodiments, provided herein are anti-FLT3 antibodies or fragments thereof having a light chain variable region comprising CDR-L1, CDR-L2 and CDR-L3 having SEQ ID NOs: 86, 87, and 88, respectively. In certain embodiments, the anti-FLT3 antibodies or fragments are humanized.

In some embodiments, provided herein are anti-FLT3 antibodies or fragments thereof having a heavy chain variable region comprising a complementarity determining region 1 (CDR-H1) having the amino acid sequence of SEQ ID NO: 89. In some embodiments, provided herein are anti-FLT3 antibodies or fragments thereof having a heavy chain variable region comprising a complementarity determining region 2 (CDR-H2) having the amino acid sequence of SEQ ID NO: 90. In some embodiments, provided herein are anti-FLT3 antibodies or fragments thereof having a heavy chain variable region comprising a complementarity determining region 3 (CDR-H3) having the amino acid sequence of SEQ ID NO: 91. In some embodiments, provided herein are anti-FLT3 antibodies or fragments thereof having a heavy chain variable region comprising CDR-H1, CDR-H2 and CDR-H3 having SEQ ID NOs: 89, 90, and 91, respectively. In certain embodiments, the anti-FLT3 antibodies or fragments are humanized.

In some embodiments, provided herein are anti-FLT3 antibodies or fragments thereof (e.g., scFv) comprising (i) a light chain variable region comprising CDR-L1 of SEQ ID NO:86, CDR-L2 of SEQ ID NO: 87, and/or CDR-L3 of SEQ ID NO: 88, and/or (ii) a heavy chain variable region comprising CDR-H1 of SEQ ID NO: 89, CDR-H2 of SEQ ID NO: 90, and/or CDR-L3 of SEQ ID NO:91. In certain embodiments, the anti-FLT3 antibodies or fragments are humanized.

In some embodiments, provided herein are anti-FLT3 antibodies or fragments thereof (e.g., scFv) comprising (i) a light chain variable region comprising CDR-L1 of SEQ ID NO:86, CDR-L2 of SEQ ID NO: 87, and CDR-L3 of SEQ ID NO: 88, and (ii) a heavy chain variable region comprising CDR-H1 of SEQ ID NO: 89, CDR-H2 of SEQ ID NO: 90, and CDR-L3 of SEQ ID NO:91. In certain embodiments, the anti-FLT3 antibodies or fragments are humanized.

In certain embodiments, the CDRs of the antibodies described herein are determined using the Kabat system.

In some embodiments, provided herein are anti-FLT3 antibodies or fragments thereof (e.g., scFv) comprising CDRs of any of the antibodies described herein, which are defined according to any of the above-described CDR defining systems (e.g., Kabat). In certain embodiments, the anti-FLT3 antibodies or fragments are humanized.

In some embodiments, provided herein are anti-FLT3 antibodies or fragments thereof (e.g., scFv) comprising (i) one, two or all three CDRs of the variable region of SEQ ID NO: 28, and/or (ii) one, two or all three CDRs of the variable region of SEQ ID NO: 17. In some embodiments, provided herein are anti-FLT3 antibodies or fragments thereof (e.g., scFv) comprising three CDRs of the variable region of SEQ ID NO: 28 and three CDRs of the variable region of SEQ ID NO. 17. In certain embodiments, the anti-FLT3 antibodies or fragments are humanized (e.g., a humanized antibody or fragment of an anti-FLT3 antibody having a heavy chain variable region comprising SEQ ID NO:17 and/or a light chain variable region comprising SEQ ID NO:28). In specific embodiments, the CDRs are as determined by Kabat.

In some embodiments, provided herein are anti-FLT3 antibodies or fragments thereof (e.g., scFv) comprising one, two, three, four, five or all six CDRs of any mouse anti-FLT3 antibody described in US Patent Pub. No. 20190127464. In some embodiments, provided herein are anti-FLT3 antibodies or fragments thereof (e.g., scFv) comprising one, two, three, four, five or all six CDRs as of a mouse anti-FLT3 antibody described in US Patent Pub. No. 20190389955 as having a VL of SEQ ID NO:25 and a VH of SEQ ID NO:27 (based on SEQ ID NOs in US Patent Pub. No. 20190389955). In some embodiments, provided herein are anti-FLT3 antibodies or fragments thereof (e.g., scFv) comprising all six CDRs of any mouse anti-FLT3 antibody described in US Patent Pub. No. 20190127464 (e.g., an antibody described in US Patent Pub. No. 20190127464a as having a VL of SEQ ID NO:5 and a VH of SEQ ID NO:7). In certain embodiments, the anti-FLT3 antibodies or fragments are humanized. In specific embodiments, the CDRs are as determined by Kabat.

Also contemplated herein are anti-FLT3 antibodies or fragments thereof (e.g., scFv) with a substitution, deletion or insertion in the CDR sequences described above. In certain embodiments, provided herein are anti-FLT3 antibodies or fragments thereof (e.g., scFv) having at least 97% CDR sequence identity to the CDRs described herein. In some embodiments, provided herein are anti-FLT3 antibodies or fragments thereof (e.g., scFv) having at least 98% CDR sequence identity to the CDRs described herein. In some embodiments, provided herein are anti-FLT3 antibodies or fragments thereof (e.g., scFv) having at least 99% CDR sequence identity to the CDRs described herein. In some embodiments, provided herein are anti-FLT3 antibodies or fragments thereof (e.g., scFv) having one, two or up to three substitutions, deletions or insertions in the CDR sequences described herein. In some embodiments, provided herein are anti-FLT3 antibodies or fragments thereof (e.g., scFv) having one, two or up to two substitutions, deletions or insertions in any one CDR sequence described herein. In some embodiments, provided herein are anti-FLT3 antibodies or fragments thereof (e.g., scFv) having one, two, three, four, five, six, seven, eight, nine or up to ten total number of substitutions, deletions or insertions in the six CDRs of the antibodies and fragments described herein. In some embodiments, provided herein are anti-FLT3 antibodies or fragments thereof (e.g., scFv) having one, two or up to three total number of substitutions, deletions or insertions in the six CDRs of the antibodies and fragments described herein. In certain embodiments, the anti-FLT3 antibodies or fragments are humanized.

As is known in the art, the CDRs are surrounded by framework regions. In certain embodiments, the anti-FLT3 antibodies or fragments described herein have human or human derived framework regions. In some embodiments of the anti-FLT3 antibodies and fragments described herein, the framework regions are human framework regions. In some embodiments of the anti-FLT3 antibodies and fragments described herein, the framework regions are human-derived framework regions.

Human framework regions that may be used and are known in the art include, without limitation: (i) human germline framework regions, (ii) human mature (somatically mutated) framework regions, (iii) framework regions selected using the “best-fit” method, (iv) framework regions derived from the consensus sequence of human antibodies of a particular subgroup of light and heavy chain variable regions, and (v) framework regions derived from screening FR libraries. See, e.g., Baca et al., J. Biol. Chem. 272: 10678-10684 (1997); Chothia et al., J. Mol. Biol. 278:457-479 (1998), Carter et al. Proc Natl. Acad. Sci. USA, 89:4285 (1992); Presta et al. J. Immunol., 151:2623 (1993), Sims et al. J. Immunol. 151:2296 (1993), Rosok et al., J. Biol. Chem. 271:22611-22618 (1996); and Almagro and Fransson, Front. Biosci. 13:1619-1633 (2008).

Any CDRs described herein can be inserted into any framework regions described herein using known DNA recombinant techniques.

### Exemplary Anti-FLT3 Antibodies: VL and VH

In certain embodiments, provided herein are anti-FLT3 antibodies or fragments thereof (e.g., scFv) comprising a heavy chain variable region comprising an amino acid sequence selected from any one of SEQ ID NOs: 3 and 17-27. In some embodiments, provided herein are anti-FLT3 antibodies or fragments thereof (e.g., scFv) comprising a heavy chain variable region comprising an amino acid sequence having at least 80%, at least 85%, at least 90%, at least 91%, at least 92%, at least 93%, at least 94%, at least 95%, at least 96%, at least 97%, at least 98%, or at least 99% identity to an amino acid sequence selected from any one of SEQ ID NOs: 3 and 17-27. In some embodiments, provided herein are anti-FLT3 antibodies or fragments thereof (e.g., scFv) comprising a heavy chain variable region comprising an amino acid sequence having at least 95%, at least 96%, at least 97%, at least 98%, or at least 99% identity to an amino acid sequence selected from any one of SEQ ID NOs: 3 and 17-27. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising a heavy chain variable region comprising an amino acid sequence having at least 95%, at least 96%, at least 97%, at least 98%, or at least 99% identity to an amino acid sequence selected from any one of SEQ ID NOs: 3 and 17-27, with at least 95% (or at least 96%, 97%, 98%, 99% or 100%) identity in the framework regions and at least 97% (or at least 98%, 99% or 100% identity) in the CDR regions.

In certain embodiments, provided herein are anti-FLT3 antibodies or fragments thereof (e.g., scFv) comprising a light chain variable region comprising an amino acid sequence selected from any one of SEQ ID NOs: 1, 2 and 28-38. In some embodiments, provided herein are anti-FLT3 antibodies or fragments thereof (e.g., scFv) comprising a light chain variable region comprising an amino acid sequence having at least 80%, at least 85%, at least 90%, at least 91%, at least 92%, at least 93%, at least 94%, at least 95%, at least 96%, at least 97%, at least 98%, or at least 99% identity to an amino acid sequence selected from any one of SEQ ID NOs: 1, 2 and 28-38. In some embodiments, provided herein are anti-FLT3 antibodies or fragments thereof (e.g., scFv) comprising a light chain variable region comprising an amino acid sequence having at least 95%, at least 96%, at least 97%, at least 98%, or at least 99% identity to an amino acid sequence selected from any one of SEQ ID NOs: 1, 2 and 28-38. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising a light chain variable region comprising an amino acid sequence having at least 95%, at least 96%, at least 97%, at least 98%, or at least 99% identity to an amino acid sequence selected from any one of SEQ ID NOs: 1, 2 and 28-38, with at least 95% (or at least 96%, 97%, 98%, 99% or 100%) identity in the framework regions and at least 97% (or at least 98%, 99% or 100% identity) in the CDR regions.

In certain embodiments, provided herein are anti-FLT3 antibodies or fragments thereof (e.g., scFv) comprising (i) a heavy chain variable region comprising an amino acid sequence selected from any one of SEQ ID NOs: 3 and 17-27, and (ii) a light chain variable region comprising an amino acid sequence selected from any one of SEQ ID NOs: 1, 2 and 28-38. In some embodiments, provided herein are anti-FLT3 antibodies or fragments thereof (e.g., scFv) comprising a heavy chain variable region comprising (i) an amino acid sequence having at least 80%, at least 85%, at least 90%, at least 91%, at least 92%, at least 93%, at least 94%, at least 95%, at least 96%, at least 97%, at least 98%, or at least 99% identity to an amino acid sequence selected from any one of SEQ ID NOs: 3 and 17-27, and (ii) a light chain variable region comprising an amino acid sequence having at least 80%, at least 85%, at least 90%, at least 91%, at least 92%, at least 93%, at least 94%, at least 95%, at least 96%, at least 97%, at least 98%, or at least 99% identity to an amino acid sequence selected from any one of SEQ ID NOs: 1, 2 and 28-38. In some embodiments, provided herein are anti-FLT3 antibodies or fragments thereof (e.g., scFv) comprising (i) a heavy chain variable region comprising an amino acid sequence having at least 95%, at least 96%, at least 97%, at least 98%, or at least 99% identity to an amino acid sequence selected from any one of SEQ ID NOs: 3 and 17-27, and (ii) a light chain variable region comprising an amino acid sequence having at least 95%, at least 96%, at least 97%, at least 98%, or at least 99% identity to an amino acid sequence selected from any one of SEQ ID NOs: 1, 2 and 28-38. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a heavy chain variable region comprising an amino acid sequence having at least 95%, at least 96%, at least 97%, at least 98%, or at least 99% identity to an amino acid sequence selected from any one of SEQ ID NOs: 3 and 17-27, with at least 95% (or at least 96%, 97%, 98%, 99% or 100%) identity in the framework regions and at least 97% (or at least 98%, 99% or 100% identity) in the CDR regions, and (ii) a light chain variable region comprising an amino acid sequence having at least 95%, at least 96%, at least 97%, at least 98%, or at least 99% identity to an amino acid sequence selected from any one of SEQ ID NOs: 1, 2 and 28-38, with at least 95% (or at least 96%, 97%, 98%, 99% or 100%) identity in the framework regions and at least 97% (or at least 98%, 99% or 100% identity) in the CDR regions.

In some embodiments, contemplated herein are anti-FLT3 antibodies or fragments thereof (e.g., scFv) comprising any of the described light chain variable regions and any of the above described heavy chain variable regions.

In certain embodiments, provided herein are humanized anti-FLT3 antibodies or fragments thereof (e.g., scFv) comprising (i) a heavy chain variable region (VH) comprising an amino acid sequence of SEQ ID NO: 3, and/or (ii) a light chain variable region (VL) comprising an amino acid sequence of SEQ ID NO: 1. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 3, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 1. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 3, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 1, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 3, and/or (ii) a VL comprising an amino acid sequence of SEQ ID NO: 2. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 3, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 2. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 3, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 2, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 18, and/or (ii) a VL comprising an amino acid sequence of SEQ ID NO: 29. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 18, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 29. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 18, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 29, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 19, and/or (ii) a VL comprising an amino acid sequence of SEQ ID NO: 30. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 19, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 30. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 19, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 30, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 20, and/or (ii) a VL comprising an amino acid sequence of SEQ ID NO: 31. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 20, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 31. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 20, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 31, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 21, and/or (ii) a VL comprising an amino acid sequence of SEQ ID NO: 32. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 21, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 32. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 21, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 32, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 22, and/or (ii) a VL comprising an amino acid sequence of SEQ ID NO: 33. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 22, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 33. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 22, with at least 95% (or at least 96%, 97%, 98%, 99% or 100%/6) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 33, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 23, and/or (ii) a VL comprising an amino acid sequence of SEQ ID NO: 34. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 23, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 34. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 23, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 34, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 24, and/or (ii) a VL comprising an amino acid sequence of SEQ ID NO: 35. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 24, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 35. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 24, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 35, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 25, and/or (ii) a VL comprising an amino acid sequence of SEQ ID NO: 36. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 25, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 36. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 25, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 36, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 26, and/or (ii) a VL comprising an amino acid sequence of SEQ ID NO: 37. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 26, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 37. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 26, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 37, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are humanized anti-FLT3 antibodies or fragments thereof (e.g., scFv) comprising (i) a VH comprising an amino acid sequence of SEQ ID NO: 27, and/or (ii) a VL comprising an amino acid sequence of SEQ ID NO: 38. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 27, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 38. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 27, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 38, with at least 95% (or at least 96%, 97%, 98%, 99% or 100%) identity in the framework regions and at least 97% (or at least 98%, 99% or 100% identity) in the CDR regions. In some embodiments, provided herein are humanized anti-FLT3 antibodies or fragments thereof (e.g., scFv) comprising both the VH and the VL comprising the sequences specified in this paragraph.

In certain embodiments, provided herein are chimeric anti-FLT3 antibodies or fragments thereof (e.g., scFv) comprising (i) a VH comprising an amino acid sequence of SEQ ID NO: 17, and/or (ii) a VL comprising an amino acid sequence of SEQ ID NO: 28. In some embodiments, provided herein are anti-FLT3 antibodies or fragments thereof (e.g., scFv) comprising (i) a VH comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 17, and/or (ii) a VL comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 28. In certain embodiments, substitutions, insertions or deletions in these sequences occur in regions outside the CDRs (i.e., in the framework regions). In certain embodiments, provided herein are anti-FLT3 antibodies or fragments thereof (e.g., scFv) comprising (i) a VH comprising an amino acid sequence having at least 95%, at least 96%, at least 97%, at least 98%, or at least 99% identity to an amino acid sequence of SEQ ID NO: 17, with at least 95% (or at least 96%, 97%, 98%, 99% or 100%) identity in the framework regions and at least 97% (or at least 98%, 99% or 100% identity) in the CDR regions, and/or (ii) a VL comprising an amino acid sequence having at least 95%, at least 96%, at least 97%, at least 98%, or at least 99% identity to an amino acid sequence of SEQ ID NO: 28, with at least 95% (or at least 96%, 97%, 98%, 99% or 100%) identity in the framework regions and at least 97% (or at least 98%, 99% or 100% identity) in the CDR regions. In some embodiments, provided herein are chimeric anti-FLT3 antibodies or fragments thereof (e.g., scFv) comprising both the VH and the VL comprising the sequences specified in this paragraph.

scFvs

In certain embodiments, provided herein are scFv fragments of the humanized anti-FLT3 antibodies described herein. In certain embodiments, provided herein are scFv fragments comprising any VH and/or VL described herein, including any VH and VL pairs described herein. Methods of making single chain variable fragment antibodies are known in the art. For example, an scFv antibody can be made by fusing a heavy chain variable region (VH) with a light chain variable region via a short peptide linker. Suitable short peptide linkers are known in the art, and exemplary linkers are described herein.

In certain embodiments, provided herein is an anti-FLT3 scFv fragment comprising an amino acid sequence selected from any one of SEQ ID NOs: 4, 5, and 40-49. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence selected from any one of SEQ ID NOs: 4, 5, and 40-49. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence selected from any one of SEQ ID NOs: 4, 5, and 40-49, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising an amino acid sequence selected from any one of SEQ ID NOs: 4, 5, 44-47 and 49. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence selected from any one of SEQ ID NOs: 4, 5, 44-47 and 49. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence selected from any one of SEQ ID NOs: 4, 5, 44-47 and 49, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising the amino acid sequence of SEQ ID NO: 4. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 4. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence of SEQ ID NO: 4, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising the amino acid sequence of SEQ ID NO: 5. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 5. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence of SEQ ID NO: 5, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising the amino acid sequence of SEQ ID NO: 44. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 44. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence of SEQ ID NO: 44, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising the amino acid sequence of SEQ ID NO: 45. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 45. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence of SEQ ID NO: 45, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising the amino acid sequence of SEQ ID NO: 46. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 46. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence of SEQ ID NO: 46, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising the amino acid sequence of SEQ ID NO: 47. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 47. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence of SEQ ID NO: 47, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising the amino acid sequence of SEQ ID NO: 49. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 49. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence of SEQ ID NO: 49, with at least 95% (or at least 96%, 97%, 98%, 99% or 100%) identity in the framework regions and at least 97% (or at least 98%, 99% or 100% identity) in the CDR regions.

In certain embodiments, provided herein is an anti-FLT3 scFv fragment comprising the amino acid sequence of SEQ ID NO: 39. In some embodiments, provided herein are anti-FLT3 scFv fragments comprising an amino acid sequence having at least 85%, at least 90%, at least 91%, at least 92%, at least 93%, at least 94%, at least 95%, at least 96%, at least 97%, at least 98%, or at least 99% identity to an amino acid sequence of SEQ ID NO: 39. In certain embodiments, substitutions, insertions or deletions in these sequences occur in regions outside the CDRs (i.e., in the framework regions). In certain embodiments, provided herein are anti-FLT3 scFv fragments comprising an amino acid sequence having at least 95%, at least 96%, at least 97%, at least 98%, or at least 99% identity to an amino acid sequence of SEQ ID NO: 39, with at least 95% (or at least 96%, 97%, 98%, 99% or 100%) identity in the framework regions and at least 97% (or at least 98%, 99% or 100% identity) in the CDR regions.

Linkers that can be Used in scFvs

In some embodiments, the disclosure provides anti-FLT3 single-chain variable fragments (scFv) comprising one or more linkers linking a VH and a VL. A “linker” is a functional group which covalently attaches two or more polypeptides or nucleic acids so that they are connected to one another. The linker can be any linker known in the art. In some embodiments, the linker comprises hydrophilic amino acids. In some embodiments, the linker comprises glycine and serine.

In some embodiments, the linker has the formula (Gly₃₋₄-Ser)₁₋₄. In some embodiments, the linker is a Gly₄Ser linker, repeated from 1 to 4 times. In some embodiments, the linker is a Gly₃Ser linker, repeated from 1 to 4 times. In some embodiments, the linker comprises Gly₄Ser and Gly₃Ser, each repeated from 1 to 4 times.

In certain embodiments, the linker is 4 to 25 amino acids in length. In certain embodiments, the linker is 4 to 21 amino acids in length. In some embodiments, the linker is 4, 5, 6, 7, 8, 9, 10, 11, 12, 13, 14, 15, 16, 17, 18, 19, 20, or 21 amino acids in length. In some embodiments, the linker is 5 amino acids in length. In some embodiments, the linker is 10 amino acids in length. In some embodiments, the linker is 15 amino acids in length. In some embodiments, the linker is 19 amino acids in length. In some embodiments, the linker is 20 amino acids in length.

In some embodiments, the linker comprises the amino acid sequence of SEQ ID NO:50. In some embodiments, the linker comprises the amino acid sequence of SEQ ID NO: 51. In some embodiments, the linker comprises the amino acid sequence of SEQ ID NO: 52. In some embodiments, the linker comprises the amino acid sequence of SEQ ID NO: 53. In some embodiments, the linker comprises the amino acid sequence of SEQ ID NO: 54.

In some embodiments, the linker comprises the nucleotide sequence of SEQ ID NO: 55. In some embodiments, the linker comprises the nucleotide sequence of SEQ ID NO: 56. In some embodiments, the linker comprises the nucleotide sequence of SEQ ID NO: 57. In some embodiments, the linker comprises the nucleotide sequence of SEQ ID NO: 58. In some embodiments, the linker comprises the nucleotide sequence of SEQ ID NO: 59.

In some embodiments, provided herein are anti-FLT3 scFv fragments comprising any linker described herein linking any light chain variable region (VL) described herein to any heavy chain variable region (VH) described herein (or any VL/VH pair described herein). In some embodiments, provided herein are anti-FLT3 scFv fragments comprising any linker described herein linking a VL comprising an amino acid sequence selected from the group consisting of SEQ ID NO: 1, SEQ ID NO: 2, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38 to a VH comprising an amino acid sequence selected from the group consisting of SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

In some embodiments, provided herein are anti-FLT3 scFv fragments comprising a linker of SEQ ID NO:50 linking a VL comprising an amino acid sequence selected from the group consisting of SEQ ID NO: 1, SEQ ID NO: 2, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38 to a VH comprising an amino acid sequence selected from the group consisting of SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

In some embodiments, provided herein are anti-FLT3 scFv fragments comprising a linker of SEQ ID NO:51 linking a VL comprising an amino acid sequence selected from the group consisting of SEQ ID NO: 1, SEQ ID NO: 2, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38 to a VH comprising an amino acid sequence selected from the group consisting of SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

In some embodiments, provided herein are anti-FLT3 scFv fragments comprising a linker of SEQ ID NO:52 linking a VL comprising an amino acid sequence selected from the group consisting of SEQ ID NO: 1, SEQ ID NO: 2, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38 to a VH comprising an amino acid sequence selected from the group consisting of SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

In some embodiments, provided herein are anti-FLT3 scFv fragments comprising a linker of SEQ ID NO:53 linking a VL comprising an amino acid sequence selected from the group consisting of SEQ ID NO: 1, SEQ ID NO: 2, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38 to a VH comprising an amino acid sequence selected from the group consisting of SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

In some embodiments, provided herein are anti-FLT3 scFv fragments comprising a linker of SEQ ID NO:54 linking a VL comprising an amino acid sequence selected from the group consisting of SEQ ID NO: 1, SEQ ID NO: 2, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38 to a VH comprising an amino acid sequence selected from the group consisting of SEQ ID NO: 3, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

### Additional Anti-FLT3 Antibodies, Fragments and Characteristics

In some embodiments, described herein are anti-FLT3 antibodies, wherein the antibody is an immunoglobulin comprising any VH and VL regions described herein. The immunoglobulin molecules that can be used are of any type (e.g., IgG, IgE, IgM, IgD, IgY, IgA). The immunoglobulin molecules that can be used are of any class (e.g., IgG1, IgG2, IgG3, IgG4, IgA1, IgA2). The immunoglobulin molecules that can be used are of any subclass. In some embodiments, the immunoglobulin is IgG.

In some embodiments, described herein are single domain anti-FLT3 antibodies, having only the heavy chain or only the light chain (comprising any VH or VL described herein). Is some embodiments, described herein are single domain anti-FLT3 antibodies having only the heavy chain (comprising any VH described herein).

In some embodiments, described herein are antigen-binding fragments of anti-FLT3 antibodies, which include, without limitation, an Fv fragment, a Fab fragment, a F(ab′) fragment, a F(ab′)₂ fragment or a disulfide-linked Fv (sdFv).

In some embodiments, described herein are chimeric anti-FLT3 antibodies or antigen-binding fragments thereof, where the chimeric antibody has murine variable region and a constant region of another species (e.g., human).

In some embodiments, described herein are multi-specific anti-FLT3 antibodies and fragments (e.g., bi-specific antibodies and fragments), which in addition to specifically binding FLT3 (using the antigen-binding fragments described herein) specifically bind one or more additional antigens (e.g., a second additional antigen). The one or several additional antigens can be antigens exposed on a surface of target cells (e.g., AML cells).

In some embodiments, described herein are anti-FLT3 antibodies and fragments thereof which have a binding affinity for a FLT3 protein with an EC50 from about 0.1 nM to 100 nM, 0.5 nM to 50 nM or 1 nM to 10 nM. In some embodiments, described herein are anti-FLT3 antibodies and fragments thereof which have a binding affinity for a FLT3 protein with an EC50 that is less than about 100 nM, less than about 75 nM, less than about 50 nM, less than about 25 nM, less than about 10 nM, less than about 5 nM, less than about 3 nM, less than about 2 nM, or less than about 1 nM. In some embodiments, described herein are anti-FLT3 antibodies and fragments thereof which have a binding affinity for a FLT3 protein with an EC50 that is less than 15 nM, less than 10 nM, less than 5 nM or less than 2.5 nM.

In some embodiments, described herein are anti-FLT3 antibodies and fragments thereof which mediate antibody-dependent cell-mediated cytotoxicity (ADCC). As known in the art, the ADCC is triggered when antibody bound to the surface of a cell interacts with Fc receptors on a natural killer (NK) cells; NK cells express the receptor Fc.gamma.RIII (CD16), which recognizes the IgG1 and IgG3 subclasses. The ADCC killing mechanism involves the release of cytoplasmic granules containing perforin and granzymes.

In some embodiments, the anti-FLT3 antibodies and fragments thereof described herein (e.g. scFv) do not compete with FLT3 ligand for binding to FLT3. In some embodiments, the anti-FLT3 antibodies and fragments described herein bind to FLT3 in the presence of FLT3 ligand (or after pre-treatment of cells with FLT3 ligand) approximately the same as in the absence of FLT3 ligand (or without pre-treatment of cells with FLT3 ligand), e.g., in vitro (using any methodology to assess competitive binding known in the art or described herein, see, e.g., Example 1). In some embodiments, the binding of anti-FLT3 antibodies and fragments described herein to FLT3 is reduced by less than 25%, less than 20%, less than 15%, less than 10%, less than 5%, less than 3% or less than 1% in the presence of FLT3 ligand (or after pre-treatment of cells with FLT3 ligand), relative to in the absence of FLT3 ligand (or without pre-treatment of cells with FLT3 ligand), e.g., in vitro (using any methodology to assess competitive binding known in the art or described herein, see, e.g., Example 1). In some embodiments, the binding of anti-FLT3 antibodies and fragments described herein to FLT3 is reduced by less than 5%, less than 3% or less than 1% in the presence of FLT3 ligand (or after pre-treatment of cells with FLT3 ligand), relative to in the absence of FLT3 ligand (or without pre-treatment of cells with FLT3 ligand), e.g., in vitro (using any methodology to assess competitive binding known in the art or described herein, see, e.g., Example 1).

Provided herein are anti-FLT3 antibodies and fragments thereof described herein (e.g., scFvs) that can target and eliminate or kill FLT3-expressing cells. Provided herein are anti-FLT3 antibodies and fragments thereof (e.g., scFvs) that can target FLT3 expressed on the surface of cells. For example, provided herein are the anti-FLT3 antibodies and fragments thereof described herein that can target FLT3 expressed on the surface of cancer cells (e.g., leukemic cells, such as AML blasts). Also provided herein are the anti-FLT3 antibodies and fragments thereof described herein that can target FLT3 expressed on the surface of HSCs and/or HSPCs. Also provided herein are the anti-FLT3 antibodies and fragments thereof described herein that can target FLT3 expressed on the surface of any hematopoietic cell lineages expressing FLT3 described herein or known in the art. Without being bound by any theory or mechanism of action, the anti-FLT3 antibodies and fragments thereof described herein (e.g., scFvs) can bind an extracellular, membrane proximal FLT3 domain.

In some embodiments, the anti-FLT3 antibodies and fragments thereof described herein bind both wild type and mutant FLT3 (e.g., FLT3 known or determined to be mutated in cancer, such as the cancer treated using such antibodies/fragments). In some embodiments, the anti-FLT3 antibodies and fragments thereof described herein bind a region of FLT3 not mutated in cancer (e.g., not known to be mutated in cancer or determined not to be mutated in cancer, such as the cancer being treated using the described antibodies/fragments). In some embodiments, the anti-FLT3 antibodies and fragments thereof described herein bind to or target (e.g., kill) FLT3-expressing cells irrespective whether the cells express wild type or mutant FLT3. In some embodiments, the anti-FLT3 antibodies and fragments thereof described herein bind to or target (e.g., kill) FLT3-expressing cancer cells expressing wild type and mutant FLT3. In some embodiments, the anti-FLT3 antibodies and fragments thereof described herein bind to or target (e.g., kill) FLT3-expressing cancer cells expressing mutant FLT3 (e.g., known to express mutant FLT3 or determined to express mutant FLT3, such as having any mutation in FLT3 known in the art).

### Making of Antibodies

The anti-FLT3 antibodies and antigen-binding fragments thereof described herein can be made by any method known in the art and/or described herein.

Methods of making monoclonal antibodies are known in the art, e.g., using hybridoma technology. See e.g, Harlow E and Lane D, Antibodies: A Laboratory Manual (Cold Spring Harbor Press, 2^{nd} ed. 1988); Hammerling G J et al., in: Monoclonal Antibodies and T-Cell Hybridomas 563 (Elsevier, N Y, 1981); Kohler G and Milstein C, 1975, Nature 256.495, Goding J W (Ed), Monoclonal Antibodies: Principles and Practice, pp. 59-103 (Academic Press, 1986). In using hybridoma technology, a mouse or another appropriate host animal can be immunized with the target protein (e.g., FLT3) to elicit lymphocytes to produce antibodies that will specifically bind to the target protein, and then the lymphocytes are fused with myeloma cells to form a hybridoma. The hybridoma cells are then grown in a culture medium and assayed for production of antibodies. The binding specificity of antibodies produced by this method can be determined by methods known in the art, e.g., enzyme-linked immunoabsorbent assay (ELISA), immunoprecipitation or radioimmunoassay (RIA). The monoclonal antibodies can be further purified.

Monoclonal antibodies can also be made using recombinant and phage display technologies and using humanized mice. See, e.g., Brinkman U et al., 1995, J. Immunol. Methods 182:41-50; Ames R S et al., 1995, J Immunol. Methods 184:177-186; Laffleur et al, 2012, Methods Mol. Biol. 901:149-59; Persic L. et al., 1997, Gene 187:9-18.

Methods of making chimeric antibodies are known in the art. See, e.g., Morrison S L, 1985, Science 229:1202-7; Gillies S D et al., 1989, J. Immunol. Methods 125:191-202; Oi V T & Morrison S L, 1986, BioTechniques 4.214-221 When making a chimeric antibody, a variable region of one species (e.g., murine) is joined with a constant region of another species (e.g., human).

Methods of making humanized antibodies are known in the art, including without limitation by CDR grafting. See, e.g., Padlan E A (1991) Mol Immunol 28(4/5): 489-498; Studnicka G M et al, (1994) Prot Engineering 7(6): 805-814; and Roguska M A et al, (1994) PNAS 91: 969-973; Tan P et al, (2002) J Immunol 169: 1119-25; Caldas C et al, (2000) Protein Eng. 13(5): 353-60; Morea V et al, (2000), Methods 20(3) 267-79; Baca M et al, (1997) J Biol Chem 272(16): 10678-84; Roguska M A et al, (1996) Protein Eng 9(10): 895 904; Couto J R et al, (1995) Cancer Res. 55 (23 Supp): 5973s-5977s; Couto J R et al, (1995) Cancer Res 55(8) 1717-22; Sandhu J S (1994) Gene 150(2): 409-10; Pedersen J T et al, (1994) J Mol Biol 235(3): 959-73).

Methods of making human antibodies are known in the art and include phage display methods using antibody libraries derived from human immunoglobulin sequences. See, e.g., International Publication Nos. WO 98/46645, WO 98/50433, WO 98/24893, WO 98/16654, WO 96/34096, WO 96/33735, and WO 91/10741.

Methods of making antibody fragments, including single chain Fv (scFv), are also known in the art. See, e.g., Ahmad et al., 2012, Clinical and Developmental Immunology, doi: 10.1155/2012/980250; Wang et al., 2006, Anal. Chem 78, 997-1004, Pansri et al., 2009, BMC Biotechnology 9:6. For example, scFvs can be constructed by fusing heavy and light chain variable regions via short polypeptide linkers (using recombinant expression techniques), and scFv antibodies having desired antigen-binding properties can be selected by methods known in the art. Further, Fab and F(ab′)₂ fragments can be produced by proteolytic cleavage of immunoglobulin molecules using papain and pepsin, respectively.

Methods of making single domain antibodies (e.g., without light chains) are also known in the art. See, e.g, Riechmann L & Muyldermans S, 1999, J Immunol 231.25-38; Nuttall S D et al., 2000, Curr Pharm Biotechnol. 1(3):253-263; Muyldernans S, 2001, J Biotecnol 74(4):277-302.

Methods of making bispecific antibodies are well-known in the art. See, e.g., Konternan, 2012, MAbs 4.182-197; Gramer et al., 2013, MAbs 5.962-973.

Methods of making mouse anti-FLT3 antibodies are described in US Patent Pub. No. 20190137464 and US Patent Pub. No. 20190389955, each of which is incorporated by reference herein in its entirety and specifically as describing the making of mouse anti-FLT3 antibodies. Methods of making humanized anti-FLT3 antibodies and chimeric anti-FLT3 antibodies are described in this application (see, e.g., the Examples).

Methods of recombinant production of antibodies are also known in the art. In some embodiments, for recombinant production of an anti-FLT3 antibody (or an antigen binding fragment thereof), a nucleic acid encoding the antibody (or an antigen binding fragment thereof) is isolated and inserted into one or more vectors for expression in a host cell. In some embodiments, a method of making the anti-FLT3 antibody is provided, wherein the method comprises culturing a host cell comprising a nucleic acid encoding the antibody under conditions suitable for expression of the antibody, and recovering the antibody from the host cell (or host cell culture medium) and, optionally further purifying the antibody. In some embodiments, a method of making an antigen binding fragment of the anti-FLT3 antibody is provided, wherein the method comprises culturing a host cell comprising a nucleic acid encoding said fragment under conditions suitable for expression of the fragment, and recovering the fragment from the host cell (or host cell culture medium) and, optionally further purifying the fragment.

### Recombinant Receptors Such as Chimeric Antigen Receptors

In one aspect, provided herein are recombinant receptors comprising any anti-FLT3 antibody or antigen-binding fragment thereof described herein. In some embodiments, provided herein are recombinant receptors comprising any antigen binding fragment of any anti-FLT3 antibody described herein. In some embodiments, provided herein are recombinant receptors comprising any anti-FLT3 VH and/or VL described herein. In some embodiments, provided herein are recombinant receptors comprising any anti-FLT3 scFv described herein. Among the contemplated recombinant receptors are functional non-TCR antigen receptors. In some embodiments, provided herein is a chimera of a signaling domain of the T cell receptor (TCR) complex and an FLT3 antigen recognizing domain (e.g., an anti-FLT3 scFv, such as any one described herein). In some embodiments, the recombinant receptors provided here are chimeric antigen receptors (CARs). Also provided herein are cells (e.g., immune cells) expressing the recombinant receptors (e.g., CARs) described herein. A T cell expressing a CAR is referred to herein as a CAR T cell. Also provided herein are uses of cells (e.g., immune cells) expressing the recombinant receptors (e.g., CARs) described herein in therapy, such as treatment of diseases associated with FLT3 expression. In some embodiments, provided herein are uses of cells (e.g., immune cells) expressing the recombinant receptors (e.g., CARs) described herein in the treatment of cancer (e.g., AML, ALL or dendritic cell neoplasm). In some embodiments, provided herein are uses of cells (e.g., immune cells) expressing the recombinant receptors (e.g., CARs) described herein in condition a subject before hematopoietic cell transplantation.

Examples of antigen receptors, including CARs, are well known in the art. Methods of their making are also well known in the art. See, e.g., Sadelain et al., Cancer Discov. 2013 April; 3 (4): 388-398; Davila et al., 2013, PLOS ONE 8 (4): e61338; Turtle et al., Curr. Opin. Immunol., 2012, 24 (5):633-39; Wu et al., Cancer, 2012, 18(2): 160-75.

The CARs provided herein generally include an extracellular domain comprising any anti-FLT3 antibody or fragment described herein (e.g., any anti-FLT3 antigen binding fragment described herein). In certain embodiments, the CARs provided herein further include a transmembrane domain (such as any transmembrane domain described herein) and an intracellular domain (such as any intracellular domain described herein). In some embodiments, the CARs provided herein further include linkers between the extracellular domain and the transmembrane domain, and/or between the transmembrane domain and the intracellular domain. Exemplary linkers that can be used in the CARs provided herein are described herein. In some embodiments, the linker comprises hydrophilic amino acids. In some embodiments, the linker comprises glycine and serine.

Four generations of chimeric antigen receptors (CARs) are known in the art. First generation CARs join an antibody-derived scFv to the CD3zeta ((or z) intracellular signaling domain of the T-cell receptor through hinge and transmembrane domains. Second generation CARs incorporate an additional domain into the intracellular signaling domain, e.g., CD28, 4-1BB (41BB), or ICOS, to supply a costimulatory signal. Third-generation CARs contain two costimulatory domains (fused with the TCR CD3zeta chain). Third-generation costimulatory domains may include, e.g., any combination of at least two of: CD27, CD28, 4-1BB, ICOS, and 0X40. Fourth generation CARs may comprise one or more stimulatory cytokines. Examples of CARs include CARs comprising an extracellular antigen-binding domain (e.g., comprising an antigen-binding scFv), a linker or hinge region, a transmembrane domain, and an intracellular domain comprising one (first generation), two (second generation), or three (third generation) signaling domains derived from CD3z and/or co-stimulatory molecules (Maude et al, Blood. 2015; 125(26):4017-4023; Kakarla and Gottschalk, Cancer J. 2014; 20(2): 151-1 55). Functionally, the CD3z signaling domain of the T-cell receptor, when engaged, will activate and induce proliferation of T-cells but can lead to anergy (a lack of reaction by the body's defense mechanisms, resulting in direct induction of peripheral lymphocyte tolerance). Lymphocytes are considered anergic when they fail to respond to a specific antigen. The addition of a costimulatory domain in second-generation CARs may improve replicative capacity and persistence of modified T-cells. Third generation CARs combine multiple signaling domains (costimulatory) which may augment potency. Fourth generation CARs express stimulatory cytokines which may improve expansion and persistence after transplantation. Any such CARs are provided herein, where the extracellular domain comprises an anti-FLT3 antigen-binding fragment (e.g., any anti-FLT3 antigen binding fragment, e.g., scFv, described herein).

In some embodiments, provided herein is a first generation CAR. In some embodiments, provided herein is a second generation CAR. In some embodiments, provided herein is a third generation CAR. In some embodiments, provided herein is a fourth generation CAR.

In some embodiments, the CAR comprises an extracellular (ecto) domain comprising an anti-FLT3 antigen binding domain (e.g., scFv), a transmembrane domain, and an intracellular (endo) domain. In some embodiments, the CAR comprises an extracellular (ecto) domain comprising an anti-FLT3 antigen binding domain (e.g., scFv), a transmembrane domain, and an intracellular (endo) domain comprising an activation domain and a co-stimulatory domain.

### Extracellular Domain/Ectodomain

In certain embodiments, the extracellular domain comprises any anti-FLT3 antibody or antigen-binding fragment thereof described herein (see, e.g., disclosure in the “Anti-FLT3 Antibodies” section above describing contemplated anti-FLT3 antibodies and fragments thereof, including subsections describing anti-FLT3 VH and/or VL regions that can be used). In certain embodiments, the extracellular domain comprises any anti-FLT3 single-chain variable fragment (scFv) described herein (see, e.g., disclosure in the “Anti-FLT3 Antibodies” section above, including subsections describing anti-FLT3 scFvs, VH and/or VL regions that can be used in the scFvs, and linkers that can be used to join the described VH and VL regions). Because the anti-FLT3 fragments, such as scFv fragments, that can be used in the extracellular domain of the anti-FLT3 CARs are described elsewhere in this application, only specific, non-limiting examples of anti-FLT3 scFvs are specifically discussed in this section. The scFv in the extracellular domain of the CAR enables binding of the CAR to the target cell expressing FLT3 on its surface (i.e., enabling the CAR to bind its target antigen).

In a specific embodiment, an anti-FLT3 scFv comprises an amino acid sequence of SEQ ID NO:4 (or an amino acid sequence that has at least 95% identity to SEQ ID NO:4). In a specific embodiment, an anti-FLT3 scFv comprises an amino acid sequence of SEQ ID NO:5 (or an amino acid sequence that has at least 95% identity to SEQ ID NO:5). In a specific embodiment, an anti-FLT3 scFv comprises an amino acid sequence of SEQ ID NO:44 (or an amino acid sequence that has at least 95% identity to SEQ ID NO:44). In a specific embodiment, an anti-FLT3 scFv comprises an amino acid sequence of SEQ ID NO:45 (or an amino acid sequence that has at least 95% identity to SEQ ID NO:45). In a specific embodiment, an anti-FLT3 scFv comprises an amino acid sequence of SEQ ID NO:46 (or an amino acid sequence that has at least 95% identity to SEQ ID NO:46). In a specific embodiment, an anti-FLT3 scFv comprises an amino acid sequence of SEQ ID NO:47 (or an amino acid sequence that has at least 95% identity to SEQ ID NO:47). In a specific embodiment, an anti-FLT3 scFv comprises an amino acid sequence of SEQ ID NO:49 (or an amino acid sequence that has at least 95% identity to SEQ ID NO:49).

In some embodiments, the extracellular domain of the CAR comprises a signal peptide or a leader sequence. In some embodiments, the extracellular domain of the CAR comprises a cleavable signal peptide. In some embodiments, the extracellular domain of the CAR comprises a signal peptide before the anti-FLT3 antigen-binding domain (e.g., N-terminal to the antigen-binding domain). Signal peptides are known in the art for use in CAR constructs. Some signal peptides help direct the nascent protein of the CAR to the endoplasmic reticulum. In some embodiments, the signal peptide is a GM-CSF signal peptide or an Igk-chain signal peptide In some embodiments, the signal peptide comprises the amino acid sequence of SEQ ID NO: 71. In some embodiments, the signal peptide comprises an amino acid sequence having at least 80%, at least 85%, at least 90%, at least 95%, or at least 99% identity to SEQ ID NO: 71. In some embodiments, the signal peptide comprises the nucleotide sequence of SEQ ID NO: 77. In some embodiments, the signal peptide comprises an amino acid sequence having at least 80%, at least 85%, at least 90%, at least 95%, or at least 99% identity to SEQ ID NO: 77.

In some embodiments, a linker connects a signal peptide to the anti-FLT3 antigen-binding domain (such as any anti-FLT3 antigen binding fragment, e.g., scFv, described herein). In some embodiments, a linker connects a signal peptide to the anti-FLT3 light chain variable region (such as any anti-FT3 VL described herein). In some embodiments, a linker connects a signal peptide to the anti-FLT3 heavy chain variable region (such as any anti-FT3 VH described herein). In some embodiments, the linker connecting a signal peptide to the antigen-binding domain comprises 1-25 amino acids (e.g., 1, 2, 3, 4, or 5 amino acids), optionally comprising glycine and/or serine. In some embodiments, the linker connecting a signal peptide to the antigen-binding domain is a two amino acid linker. In some embodiments, the linker connecting a signal peptide to the antigen-binding domain is a glycine serine (e.g., GS) linker.

In some embodiments, a linker connects an extracellular domain to a spacer or hinge region. In some embodiments, the linker connecting an extracellular domain to a spacer or hinge region comprises 1-25 amino acids (e.g., 1, 2, 3, 4, or 5 amino acids), optionally comprising glycine and/or serine. In some embodiments, the linker connecting an extracellular domain to a spacer or hinge region is a two amino acid linker. In some embodiments, the linker connecting an extracellular domain to a spacer or hinge region is a glycine serine (e.g., GS) linker.

### A Spacer or Hinge Region

In some embodiments, the extracellular domain is connected to the transmembrane domain by a hinge or spacer region. The hinge region can be any hinge region known in the art. Examples of hinge regions include, but are not limited to, those from CD8. CD28, or derived from IgG1, IgG2, or IgG4 In some embodiments, the hinge region is from the CD8 extracellular domain. In some embodiments, the hinge region is a CD8 (e.g., CD8α) hinge. In some embodiments, the hinge region is a CD28 hinge. In some embodiments, the CD8α hinge comprises the amino acid sequence of SEQ ID NO: 72. In some embodiments, the CD8α hinge comprises an amino acid sequence having at least 80%, at least 85%, at least 90%, at least 95%, or at least 99% identity to SEQ ID NO: 72 In some embodiments, the CD8α hinge comprises the nucleotide sequence of SEQ ID NO: 78. In some embodiments, the CD8α hinge comprises a nucleotide sequence having at least 80%, at least 85%, at least 90%, at least 95%, or at least 99% identity to SEQ ID NO: 78.

### Transmembrane Domain

A transmembrane domain is a hydrophobic alpha helix that spans the membrane of a cell. For a chimeric antigen receptor (CAR), the transmembrane domain enables insertion of the CAR into the cell membrane.

In some embodiments, the transmembrane domain is a transmembrane domain of any one or more of the following. 4-1BB/CD137, an activating NK cell receptor, an immunoglobulin protein, B7-H3, BALER, BLAME (SLAMF8), BTLA, CD100 (SEMA4D), CD103, CD160 (BY55), CD18, CD19, CD19a, CD22, CD247, CD27, CD276 (B7-13), CD28, CD29, CD3, CD3 delta, CD3 epsilon, CD3 gamma, CD3 zeta, CD30, CD4, CD40, CD49a, CD49D, CD49f, CD69, CD7, CD84, CD8, CD 8 alpha, CI) 8 beta, CD96 (Tactile), CD11a, CD11b, CD11c, CD11d, CD5, CD9, CD 16, CD33, CD37, CD64, CD80, CD86, CD134, CD137 or CD154 CEACAM1, CRT AM, CTLA4, PD-1, cytokine receptor, DAP-10, DNAM1 (CD226), Fc gamma receptor, GADS, GITR, HVEM (LIGHTR), 1A4, ICAM-1, Ig alpha (CD79a), IL-2R beta, IL-2R gamma, IL-7R alpha, inducible T cell costimulator (ICOS), an integrin, ITGA4, ITGA6, ITGAD, ITGAE, ITGAL, ITGAMJTGAX, ITGB2, ITGB7, ITGB1, KIRDS2, LAT, LFA-1, a ligand that specifically binds with CD83, LIGHT, LTBR, Ly9 (CD229), lymphocyte function-associated antigen-1 (LFA-1), an MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), a Signaling Lymphocytic Activation Molecule (a SLAM protein), SLAM (SLAMF1), SLAMF4 (CD244), SLAMF6 (NTB-A), SLAMF7, SLP-76, a TNF receptor protein, TNFR2, TNFSF14, a Toll ligand receptor, TRANCE/RANKL, VLA1, and VLA-6. In some embodiments, the transmembrane domain of a CAR provided herein is from a CD3 transmembrane domain, a CD4 transmembrane domain, a CD8 transmembrane domain, a CD28 transmembrane domain or a 4-1-BB transmembrane domain.

In some embodiments, the transmembrane domain of a CAR provided herein is a CD8 (e.g., CD8α) transmembrane domain. In some embodiments, the transmembrane domain of a CAR provided herein is a CD3 transmembrane domain. In some embodiments, the transmembrane domain of a CAR provided herein is a CD4 transmembrane domain. In some embodiments, the transmembrane domain of a CAR provided herein is a CD28 transmembrane domain. In some embodiments, the transmembrane domain of a CAR provided herein is a 4-1-BB transmembrane domain. In some embodiments, the CD8α transmembrane domain comprises the amino acid sequence of SEQ ID NO: 73. In some embodiments, the CD8α transmembrane domain comprises an amino acid sequence having at least 80%, at least 85%, at least 90%, at least 95%, or at least 99% identity to SEQ ID NO: 73. In some embodiments, the CD8α transmembrane domain comprises the nucleic acid sequence of SEQ ID NO: 79. In some embodiments, the CD8α transmembrane domain comprises a nucleic acid sequence having at least 80%, at least 85%, at least 90%, at least 95%, or at least 99% identity to SEQ ID NO: 79.

### Intracellular Domain/Endodomain

The intracellular domain (i.e., endodomain) of a CAR is the functional end of the receptor. Following antigen recognition, receptors cluster and a signal is transmitted to the cell. The intracellular domain includes a signaling domain which relays an internal signal to active the immune cell expressing the CAR. In some embodiments, the intracellular domain of a CAR provided herein comprises CD3 intracellular signaling (or activation) domain. A CD3ζ signaling domain contains three immunoreceptor tyrosine-based activation motifs (ITAMS). The ITAMs transmit an activation signal to the cell comprising the CAR after an antigen binds to the CAR.

In some embodiments, the CD3ζ signaling domain comprises the amino acid sequence of SEQ ID NO: 76. In some embodiments, the CD3 signaling domain comprises an amino acid sequence having at least 80%, at least 85%, at least 90%, at least 95%, or at least 99% identity to SEQ ID NO: 76. In some embodiments, the CD3ζ signaling domain comprises the nucleic acid sequence of SEQ ID NO: 82. In some embodiments, the CD3ζ signaling domain comprises a nucleic acid sequence having at least 80%, at least 85%, at least 90%, at least 95%, or at least 99% identity to SEQ ID NO: 82.

In some embodiments, the intracellular domain of a CAR provided herein comprises a CD3ε (epsilon) signaling domain. In some embodiments, the intracellular domain of a CAR provided herein comprises an FcγR (FcRgamma) signaling domain. Other activation domains known in the art can also be used.

In some embodiments, the intracellular domain/endodomain comprises a co-stimulatory domain. In some embodiments, the intracellular domain comprises a stimulatory domain (e.g. CD3ζ signaling domain or another activation domain) and a co-stimulatory domain. In some embodiments, the co-stimulatory domain is a co-stimulatory domain of any one or more of: CD28, ICOS, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD11a/CD18), CD3 gamma, CD3 delta, CD3 epsilon, CD247, CD276 (B7-1-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, SLAM proteins, activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, 1A4, CD49), ITGA6, VLA-6, CD49f, ITGAD, CD1 Id, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2134), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

In some embodiments, the intracellular domain comprises a CD28 co-stimulatory domain. In some embodiments, the CD28 co-stimulatory domain comprises the amino acid sequence of SEQ ID NO:74. In some embodiments, the CD28 co-stimulatory domain comprises an amino acid sequence having at least 80%, at least 85%, at least 90%, at least 95%, or at least 99% identity to SEQ ID NO: 74. In some embodiments, the CD28 co-stimulatory domain comprises the nucleic acid sequence of SEQ ID NO:80. In some embodiments, the CD28 co-stimulatory domain comprises a nucleic acid sequence having at least 80%, at least 85%, at least 90%, at least 95%, or at least 99% identity to SEQ ID NO: 80.

In some embodiments, the intracellular domain comprises a 4-1BB co-stimulatory domain. In some embodiments, the 4-1BB co-stimulatory domain comprises the amino acid sequence of SEQ ID NO:75. In some embodiments, the 4-1BB co-stimulatory domain comprises an amino acid sequence having at least 80%, at least 85%, at least 90%, at least 95%, or at least 99% identity to SEQ ID NO: 75. In some embodiments, the 4-1BB co-stimulatory domain comprises the nucleic acid sequence of SEQ ID NO:81. In some embodiments, the 4-1BB co-stimulatory domain comprises a nucleic acid sequence having at least 80%, at least 85%, at least 90%, at least 95%, or at least 99% identity to SEQ ID NO: 81.

In some embodiments, the intracellular domain comprises both CD28 and 4-1BB co-stimulatory domains. In some embodiments, the intracellular domain comprises the amino acid sequence of SEQ ID NO: 74 and SEQ ID NO: 75.

In some embodiments, the intracellular domain comprises a co-stimulatory domain of CD27. In some embodiments, the intracellular domain comprises a co-stimulatory domain of OX40. In some embodiments, the intracellular domain comprises a co-stimulatory domain of ICOS. In some embodiments, the intracellular domain comprises a CD3ζ signaling domain and a 4-1BB co-stimulatory domain. In some embodiments, the intracellular domain comprises a CD3ζ signaling domain and a CD28 co-stimulatory domain. In some embodiments, the intracellular domain comprises a CD3ζ signaling domain, a 4-1BB co-stimulatory domain, and a CD28 co-stimulatory domain.

In some embodiments, the intracellular domain comprises a CD3ζ signaling domain having the amino acid sequence of SEQ ID NO: 76, a 4-1BB co-stimulatory domain having the amino acid sequence of SEQ ID NO: 75, and a CD28 co-stimulatory domain having the amino acid sequence of SEQ ID NO: 74.

### Safely Switch

In some embodiments, the CAR comprises a safety switch. In some embodiments, the safety switch is selected from, but not limited to, herpes simplex virus thymidine kinase (hsv-tk), inducible Caspase 9 (icasp9), and a truncated human epidermal growth factor receptor (EGFRt) polypeptide. In some embodiments, a suicide gene is included within the vector comprising nucleic acids encoding any of the CARs described herein. In this way, administration of a prodrug designed to activate the safety switch (e.g., AP1903 that activates iCasp9) triggers apoptosis in the safety switch and activated CAR-expressing cells.

In some embodiments, the suicide gene/safety switch is icasp9. In some embodiments, the icasp9 enables immune cell elimination (e.g., a T cell) after a chemical inducer of dimerization (e.g., AP1903 or AP20187) is administered. In some embodiments, the icasp9 is encoded by the nucleic acid sequence of SEQ ID NO:85. In some embodiments, the icasp9 gene comprises a nucleic acid sequence having at least 80%, at least 85%, at least 90%, at least 95%, or at least 99% identity to SEQ ID NO: 85. In some embodiments, the icasp9 safety switch comprises the amino acid sequence of SEQ ID NO:105. In some embodiments, the icasp9 safety switch comprises an amino acid sequence having at least 80%, at least 85%, at least 90%, at least 95%, or at least 99% identity to SEQ ID NO:105.

In some embodiments, the suicide gene/safety switch is EGFRt. In some embodiments, the EGFRt enables immune cell elimination (e.g., a T cell) after an anti-EGFR monoclonal antibody (e.g. cetuximab) is administered. In some embodiments, the EGFRt is encoded by the nucleic acid sequence of SEQ ID NO: 84. In some embodiments, the EGFRt gene comprises a nucleic acid sequence having at least 80%, at least 85%, at least 90%, at least 95%, or at least 99% identity to SEQ ID NO: 84. In some embodiments, the EGFRt safety switch comprises the amino acid sequence of SEQ ID NO:104. In some embodiments, the EGFRt safety switch comprises an amino acid sequence having at least 80%, at least 85%, at least 90%, at least 95%, or at least 99% identity to SEQ ID NO:104.

In some embodiments, the safety-switch-containing CARs described herein include a self-cleaving peptide connecting the safety switch to the CAR. Exemplary self-cleaving peptides include the 2A family of peptides (e.g., T2A, E2A, F2A, and P2A peptides) and IRES. In some embodiments, the self-cleaving peptide is a T2A peptide. In some embodiments, the T2A peptide is encoded by the nucleic acid sequence of SEQ ID NO: 83. In some embodiments, the T2A is encoded by a nucleic acid sequence having at least 80%, at least 85%, at least 90%, at least 95%, or at least 99% identity to SEQ ID NO: 83. In some embodiments, the T2A peptide comprises the amino acid sequence of SEQ ID NO:103. In some embodiments, the T2A peptide comprises an amino acid sequence having at least 80%, at least 85%, at least 90%, at least 95%, or at least 99% identity to SEQ ID NO:103.

In some embodiments, the self-cleaving peptide is an E2A peptide. In some embodiments, the self-cleaving peptide is an F2A peptide. In some embodiments, the self-cleaving peptide is an P2A peptide. In some embodiments, the self-cleaving peptide is an IRES peptide.

### Isolated Nucleic Acids Expressing CARs

Provided herein are nucleic acid sequences (polynucleotides) that encode one or more of the CARs provided herein. In some embodiments, the polynucleotides are contained within any vector suitable for the transformation of immune cells (e.g., T cells). In some embodiments, immune cells are transformed using synthetic vectors, lentiviral vectors, retroviral vectors, autonomously replicating plasmids, a virus (e.g., a retrovirus, lentivirus, adenovirus, or herpes virus).

Lentiviral vectors suitable for transformation of T lymphocytes include, but are not limited to, e.g., the lentiviral vectors described in U.S. Pat. Nos. 5,994,136; 6,165,782; 6,428,953; 7,083,981; and 7,250,299, the disclosures of which are hereby incorporated by reference in their entireties. HIV vectors suitable for transformation of T lymphocytes include, but are not limited to, e.g., the vectors described in U.S. Pat. No. 5,665,577, the disclosure of which is hereby incorporated by reference in its entirety.

In some embodiments, the CAR comprises the nucleic acid of SEQ ID NO: 60. In some embodiments, the CAR comprises the nucleic acid of SEQ ID NO: 61. In some embodiments, the CAR comprises the nucleic acid of SEQ ID NO: 62. In some embodiments, the CAR comprises the nucleic acid of SEQ ID NO: 63. In some embodiments, the CAR comprises the nucleic acid of SEQ ID NO: 64. In some embodiments, the CAR comprises the nucleic acid of SEQ ID NO: 65. In some embodiments, the CAR comprises the nucleic acid of SEQ ID NO: 66. In some embodiments, the CAR comprises the nucleic acid of SEQ ID NO: 67. In some embodiments, the CAR comprises the nucleic acid of SEQ ID NO: 68. In some embodiments, the CAR comprises the nucleic acid of SEQ ID NO: 69. In some embodiments, the CAR comprises the nucleic acid of SEQ ID NO: 70.

In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 60. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 61. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 62. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 63. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 64. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 65. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 66. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 67. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 68. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 69. In some embodiments, the CAR comprises a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99% identity of SEQ ID NO: 70.

In some embodiments, the CAR is expressed by the plasmid of SEQ ID NO: 92. In some embodiments, the CAR is expressed by the plasmid of SEQ ID NO: 93. In some embodiments, the CAR is expressed by the plasmid of SEQ ID NO: 94. In some embodiments, the CAR is expressed by the plasmid of SEQ ID NO: 95. In some embodiments, the CAR is expressed by the plasmid of SEQ ID NO: 96. In some embodiments, the CAR is expressed by the plasmid of SEQ ID NO: 97. In some embodiments, the CAR is expressed by the plasmid of SEQ ID NO: 98. In some embodiments, the CAR is expressed by the plasmid of SEQ ID NO: 99. In some embodiments, the CAR is expressed by the plasmid of SEQ ID NO: 100. In some embodiments, the CAR is expressed by the plasmid of SEQ ID NO: 101. In some embodiments, the CAR is expressed by the plasmid of SEQ ID NO: 102.

### Methods of Making CARs

Methods of making CARs are generally known in the art and are described, for example, in U.S. Pat. Nos. 6,410,319; 7,446,191; U.S. Pat. Publication No. 2010/065818, U.S. Pat. No. 8,822,647, PCT Publication No. WO 2014/031687; U.S. Pat. No. 7,514,537; and Brentjens et al., 2007, Clin. Cancer Res. 13:5426, each of which is hereby incorporated by reference in its entirety.

Binding of the extracellular antigen-binding domain (e.g., an anti-FLT3 antigen binding fragment or scFv described herein) of a presently disclosed CAR to FLT3 can be confirmed using methods known in the art. For example, enzyme-linked immunosorbent assay (ELISA), radioimmunoassay (RIA), FACS analysis, bioassay (e.g., growth inhibition), or Western Blot assay can be used. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA). The radioactive isotope can be detected by such means as the use of a y counter or a scintillation counter or by autoradiography. In some embodiments, the extracellular antigen binding domain is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In some embodiments, the CAR is labeled with GFP. In some embodiments, the GFP labeled CAR comprises the nucleic acid sequence of SEQ ID NO: 70. In some embodiments, the GFP labeled CAR comprises the amino acid sequence of SEQ ID NO: 16.

### Exemplary CAR Constructs

In some embodiments, a CAR provided herein comprises the following domains:

Signal Peptide-linker1-VL-linker2-VH-linker3-hinge-TM domain-one or two co-stimulatory domains-signaling/activation domain. In some embodiments, the order of the domains is as specified here. In some embodiments, any one or more of the linker domains are absent.

In some embodiments, a CAR provided herein comprises the following domains: Signal Peptide-linker1-VL-linker2-VH-linker3-hinge-TM domain-one or two co-stimulatory domains-signaling/activation domain—self-cleaving peptide—safety switch. In some embodiments, the order of the domains is as specified here. In some embodiments, any one or more of the linker domains are absent.

In some embodiments, a CAR provided herein comprises the following domains: Signal Peptide-linker1-VL-linker2-VH-linker3-CD8hinge-CD8TM-CD28 co-stimulatory domain and/or 4-1BB co-stimulatory domain-CD3ζ signaling domain. In some embodiments, the order of the domains is as specified here (but, e.g., where the co-stimulatory domains if both are present appear in any order).

In some embodiments, a CAR provided herein comprises the following domains: Signal Peptide-linker1-VL-linker2-VH-linker3-CD8αhinge-CD8αTM-CD28 co-stimulatory domain-4-1BB co-stimulatory domain-CD3ζ signaling domain. In some embodiments, the order of the domains is as specified here.

In some embodiments, of the CARs exemplified in this section: the VL is selected from an amino acid sequence comprising one of SEQ ID NOs: 1, 2, and 28-38, and the VH is selected from an amino acid sequence comprising one of SEQ ID NOs: 3, and 17-27.

In some embodiments, a CAR provided herein comprises the following domains: Signal Peptide-linker1-VL-linker2-VH-linker3-CD8αhinge-CD8αTM-CD28 co-stimulatory domain-4-1BB co-stimulatory domain-CD3ζ signaling domain; wherein
(i) the VL is selected from an amino acid sequence comprising one of SEQ ID NOs: 1, 2, and 28-38,
(ii) the VH is selected from an amino acid sequence comprising one of SEQ ID NOs: 3, and 17-27,
(iii) linker 2 comprises SEQ ID NO: 53,
(iv) the signal peptide comprises SEQ ID NO: 71,
(v) the CD8αhinge comprises SEQ ID NO: 72,
(vi) the CD8αTM comprises SEQ ID NO: 73,
(vii) the CD28 co-stimulatory domain comprises SEQ ID NO: 74,
(viii) the 4-1BB co-stimulatory domain comprises SEQ ID NO: 75, and
(ix) the CD3ζ signaling domain comprises SEQ ID NO: 76.

In some embodiments, the CARs provided herein comprise: (i) an extracellular domain comprising any one of SEQ ID NOs: 4, 5, 44, 45, 46, 47 and 49; (ii) a transmembrane domain; and (iii) an intracellular domain.

In some embodiments, the CARs provided herein comprise: (i) an extracellular domain comprising an scFv comprising the amino acid sequence of SEQ ID NOs: 4; (ii) a transmembrane domain; and (iii) an intracellular domain.

In some embodiments, the CARs provided herein comprise: (i) an extracellular domain comprising an scFv comprising the amino acid sequence of SEQ ID NOs: 5; (ii) a transmembrane domain; and (iii) an intracellular domain.

In some embodiments, the CAR comprises (i) an extracellular domain comprising an scFv comprising the amino acid sequence of SEQ ID NO: 4, (ii) a transmembrane domain comprising a CD8α transmembrane domain, and (iii) an intracellular domain comprising an intracellular signaling domain of CD3ζ and a co-stimulatory domain of CD28 and/or 4-1BB.

In some embodiments, the CAR comprises (i) an extracellular domain comprising an scFv comprising the amino acid sequence of SEQ ID NO: 5, (ii) a transmembrane domain comprising a CD8α transmembrane domain, and (iii) an intracellular domain comprising an intracellular signaling domain of CD3ζ and a co-stimulatory domain of CD28 and/or 4-1BB.

In some embodiments, the CAR comprises (i) an extracellular domain comprising an scFv comprising the amino acid sequence of SEQ ID NO: 4, (ii) a transmembrane domain comprising a CD8α transmembrane domain, (iii) an intracellular domain comprising an intracellular signaling domain of CD3ζ and a co-stimulatory domain of CD28 and/or 4-1BB, (iv) and a safety switch polypeptide.

In some embodiments, the CAR comprises (i) an extracellular domain comprising an scFv comprising the amino acid sequence of SEQ ID NO: 5, (ii) a transmembrane domain comprising a CD8α transmembrane domain, (iii) an intracellular domain comprising an intracellular signaling domain of CD3ζ and a co-stimulatory domain of CD28 and/or 4-1BB, (iv) and a safety switch polypeptide.

Provided herein are exemplary CAR constructs. In some embodiments, the CAR comprises the nucleic acid sequence of any one of SEQ ID NOs: 60-70. In some embodiments, the CAR comprises the nucleic acid sequence of SEQ ID NO: 60. In some embodiments, the CAR comprises the nucleic acid sequence of SEQ ID NO: 61. In some embodiments, the CAR comprises the nucleic acid sequence of SEQ ID NO: 62. In some embodiments, the CAR comprises the nucleic acid sequence of SEQ ID NO: 63. In some embodiments, the CAR comprises the nucleic acid sequence of SEQ ID NO: 64. In some embodiments, the CAR comprises the nucleic acid sequence of SEQ ID NO: 65. In some embodiments, the CAR comprises the nucleic acid sequence of SEQ ID NO: 66. In some embodiments, the CAR comprises the nucleic acid sequence of SEQ ID NO: 67. In some embodiments, the CAR comprises the nucleic acid sequence of SEQ ID NO: 68. In some embodiments, the CAR comprises the nucleic acid sequence of SEQ ID NO: 69. In some embodiments, the CAR comprises the nucleic acid sequence of SEQ ID NO: 70.

In some embodiments, the CAR comprises the amino acid sequence of any one of SEQ ID NOs: 6-16. In some embodiments, the CAR comprises the amino acid sequence of SEQ ID NO: 6. In some embodiments, the CAR comprises the amino acid sequence of SEQ ID NO: 7. In some embodiments, the CAR comprises the amino acid sequence of SEQ ID NO: 8. In some embodiments, the CAR comprises the amino acid sequence of SEQ ID NO: 9. In some embodiments, the CAR comprises the amino acid sequence of SEQ ID NO: 10. In some embodiments, the CAR comprises the amino acid sequence of SEQ ID NO: 11. In some embodiments, the CAR comprises the amino acid sequence of SEQ ID NO: 12. In some embodiments, the CAR comprises the amino acid sequence of SEQ ID NO: 13. In some embodiments, the CAR comprises the amino acid sequence of SEQ ID NO: 14. In some embodiments, the CAR comprises the amino acid sequence of SEQ ID NO: 15. In some embodiments, the CAR comprises the amino acid sequence of SEQ ID NO: 16.

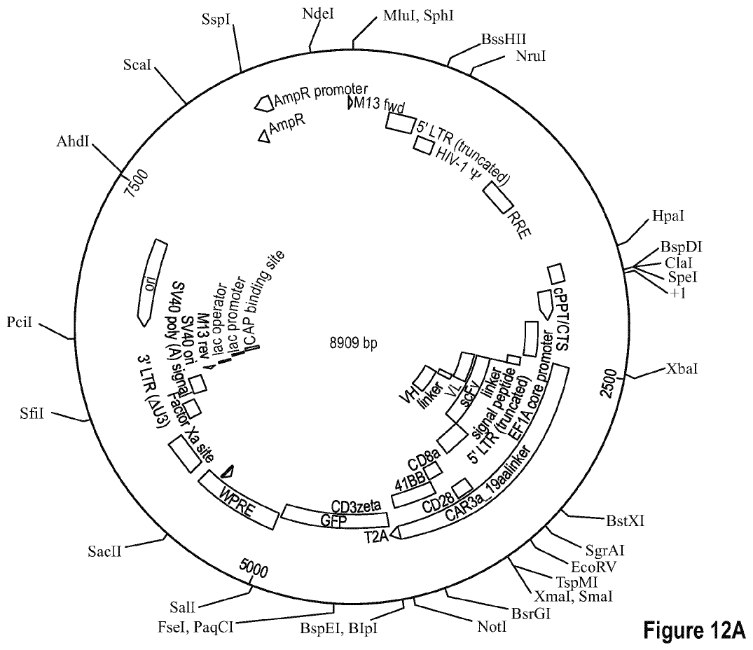

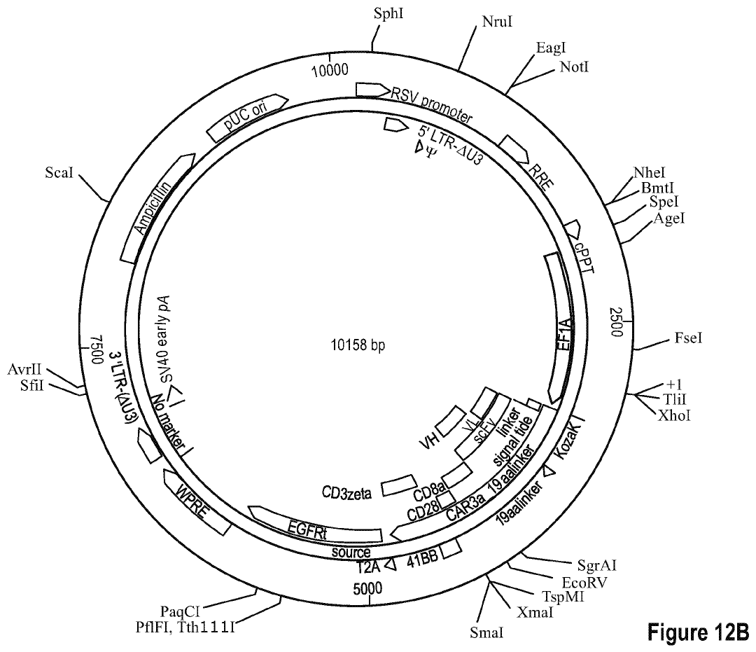

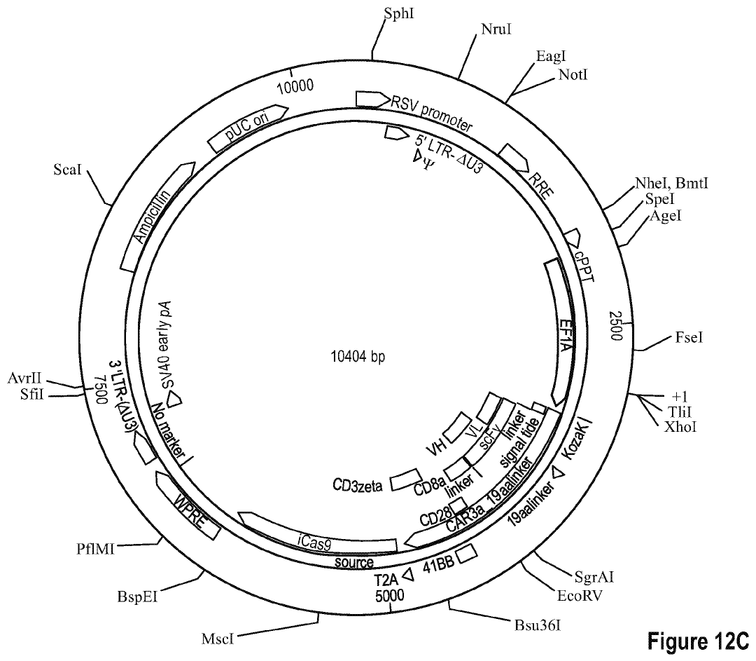

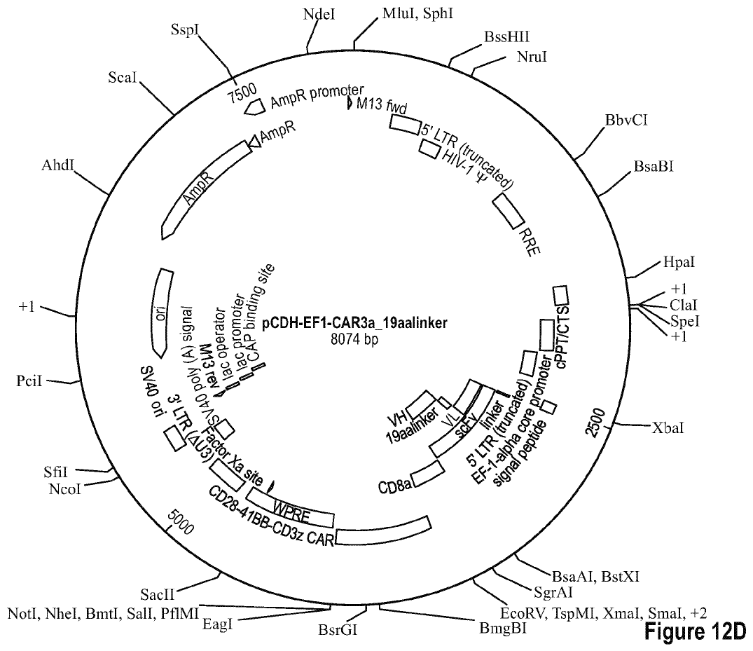

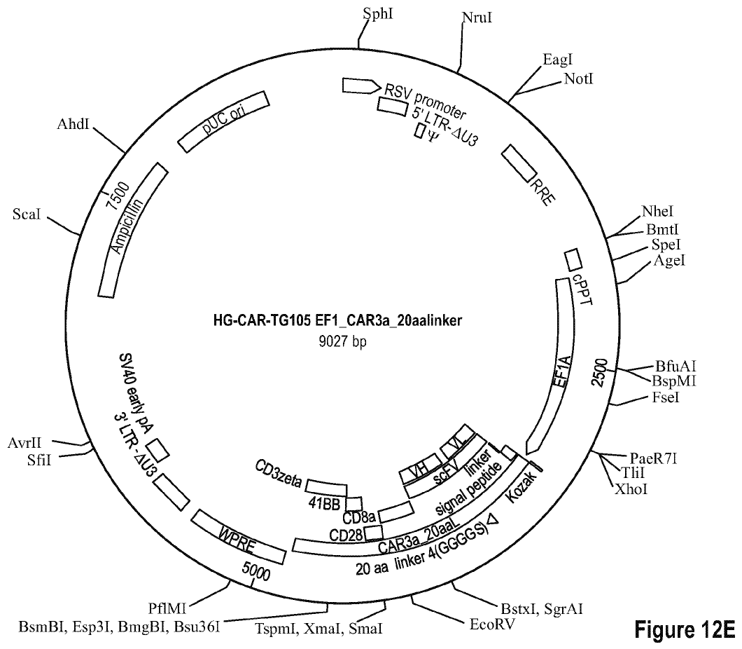

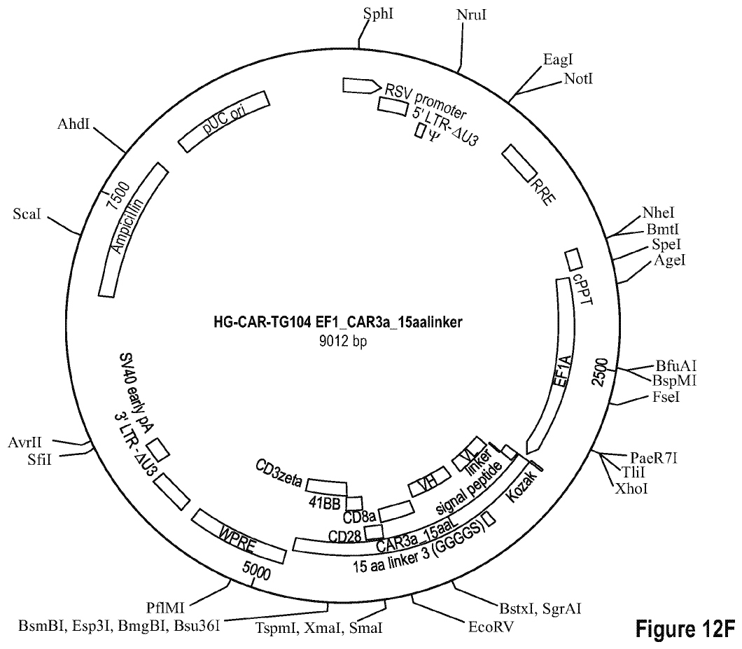

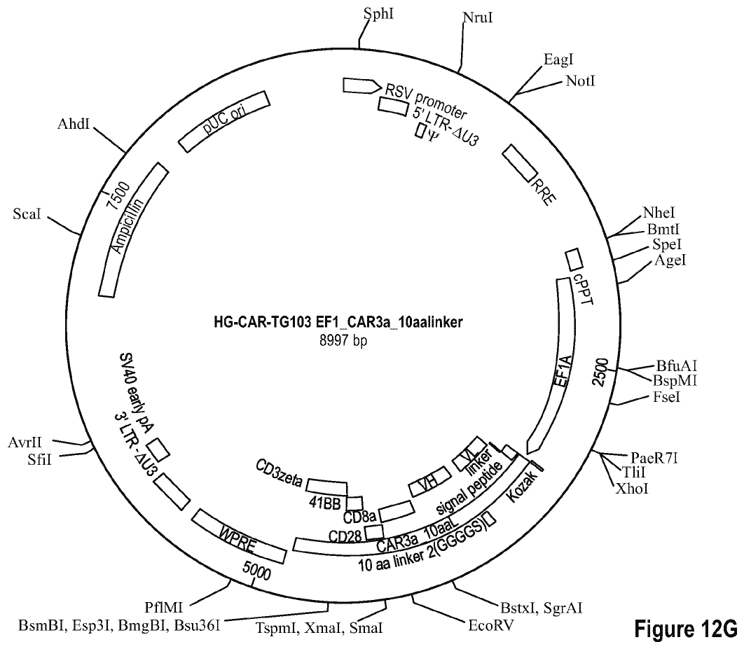

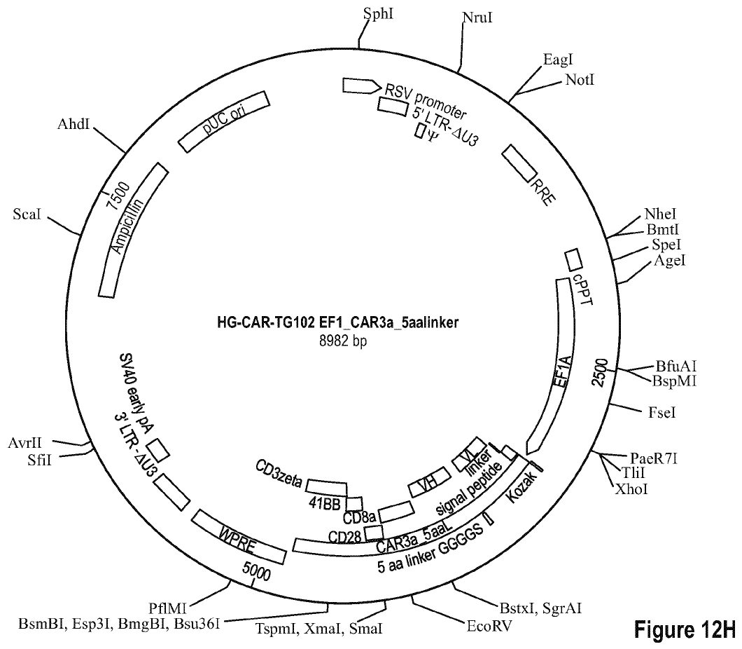

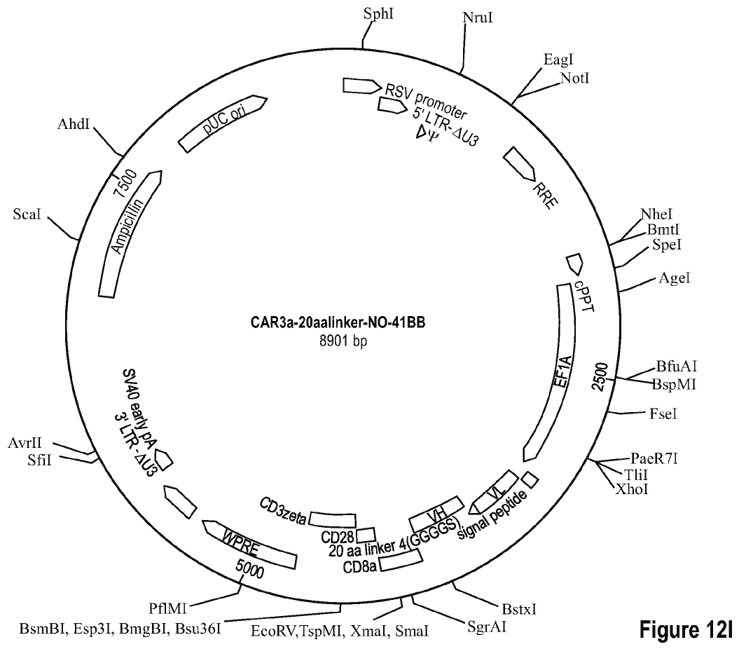

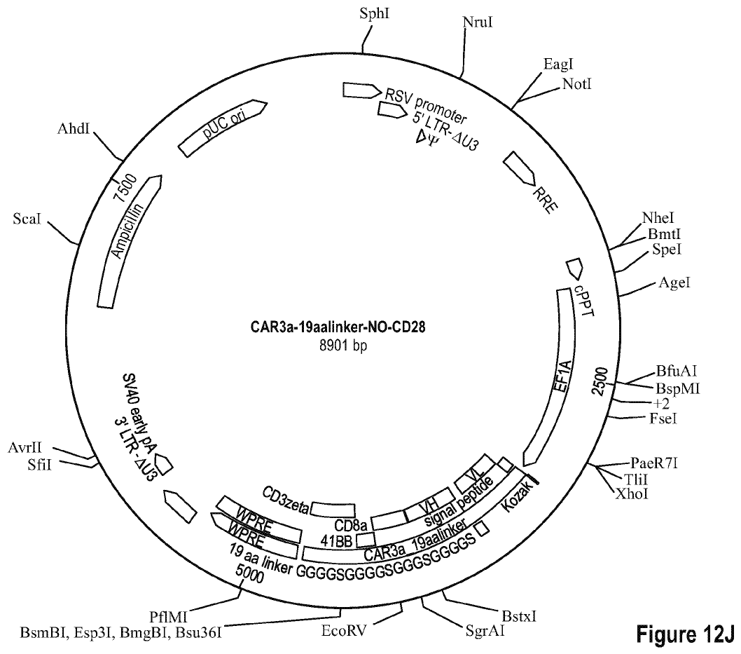

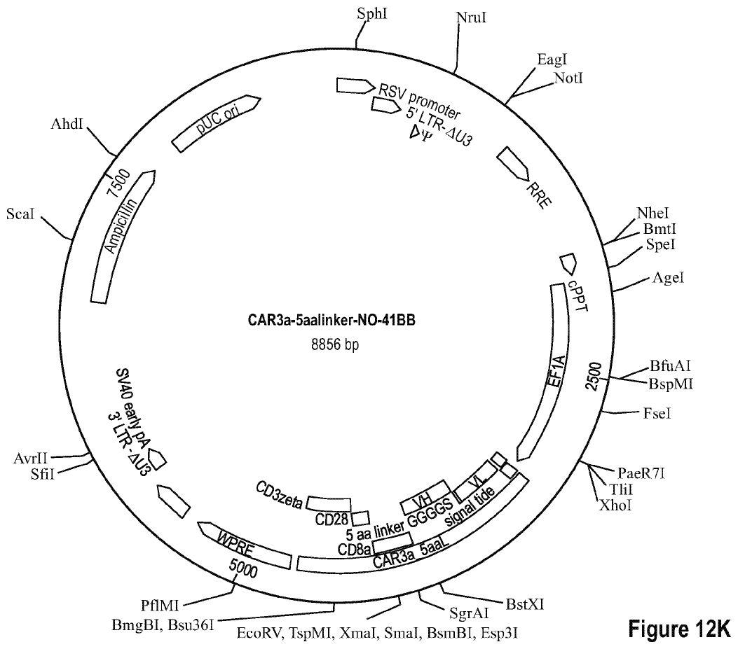

In some embodiments, the CAR (such as the CAR of SEQ ID NO: 16) is expressed by the plasmid depicted in FIG. 12A. In some embodiments, the CAR (such as the CAR of SEQ ID NO: 7) is expressed by the plasmid depicted in FIG. 12B. In some embodiments, the CAR (such as the CAR of SEQ ID NO: 8) is expressed by the plasmid depicted in FIG. 12C. In some embodiments, the CAR (such as the CAR of SEQ ID NO: 6) is expressed by the plasmid depicted in FIG. 12D. In some embodiments, the CAR (such as the CAR of SEQ ID NO: 12) is expressed by the plasmid depicted in FIG. 12E. In some embodiments, the CAR (such as the CAR of SEQ ID NO: 11) is expressed by the plasmid depicted in FIG. 12F. In some embodiments, the CAR (such as the CAR of SEQ ID NO: 10) is expressed by the plasmid depicted in FIG. 12G. In some embodiments, the CAR (such as the CAR of SEQ ID NO: 9) is expressed by the plasmid depicted in FIG. 12H. In some embodiments, the CAR (such as the CAR of SEQ ID NO: 13) is expressed by the plasmid depicted in FIG. 12I. In some embodiments, the CAR (such as the CAR of SEQ ID NO: 14) is expressed by the plasmid depicted in FIG. 12J. In some embodiments, the CAR (such as the CAR of SEQ ID NO: 15) is expressed by the plasmid depicted in FIG. 12K.

In some embodiments, the extracellular antigen-binding domain (e.g., scFv) of a CAR described herein comprises (a) a light chain variable region comprising the amino acid sequence set forth in SEQ ID NO: 1, and (b) a heavy chain variable region comprising the amino acid sequence set forth in SEQ ID NO: 3.

In some embodiments, the extracellular antigen-binding domain (e.g., scFv) of a CAR described herein comprises (a) a light chain variable region comprising the amino acid sequence set forth in SEQ ID NO: 2, and (b) a heavy chain variable region comprising the amino acid sequence set forth in SEQ ID NO: 3.

In some embodiments, the extracellular antigen-binding domain (e.g., scFv) of a CAR described herein comprises (a) a light chain variable region comprising the amino acid sequence set forth in SEQ ID NO: 28, and (b) a heavy chain variable region comprising the amino acid sequence set forth in SEQ ID NO: 17.

In some embodiments, the extracellular antigen-binding domain (e.g., scFv) of a CAR described herein comprises (a) a light chain variable region comprising the amino acid sequence set forth in SEQ ID NO: 29, and (b) a heavy chain variable region comprising the amino acid sequence set forth in SEQ ID NO: 18.

In some embodiments, the extracellular antigen-binding domain (e.g., scFv) of a CAR described herein comprises (a) a light chain variable region comprising the amino acid sequence set forth in SEQ ID NO: 30, and (b) a heavy chain variable region comprising the amino acid sequence set forth in SEQ ID NO: 19.

In some embodiments, the extracellular antigen-binding domain (e.g., scFv) of a CAR described herein comprises (a) a light chain variable region comprising the amino acid sequence set forth in SEQ ID NO: 31, and (b) a heavy chain variable region comprising the amino acid sequence set forth in SEQ ID NO: 20.

In some embodiments, the extracellular antigen-binding domain (e.g., scFv) of a CAR described herein comprises (a) a light chain variable region comprising the amino acid sequence set forth in SEQ ID NO: 32, and (b) a heavy chain variable region comprising the amino acid sequence set forth in SEQ ID NO: 21.

In some embodiments, the extracellular antigen-binding domain (e.g., scFv) of a CAR described herein comprises (a) a light chain variable region comprising the amino acid sequence set forth in SEQ ID NO: 33, and (b) a heavy chain variable region comprising the amino acid sequence set forth in SEQ ID NO: 22.

In some embodiments, the extracellular antigen-binding domain (e.g., scFv) of a CAR described herein comprises (a) a light chain variable region comprising the amino acid sequence set forth in SEQ ID NO: 34, and (b) a heavy chain variable region comprising the amino acid sequence set forth in SEQ ID NO: 23.

In some embodiments, the extracellular antigen-binding domain (e.g., scFv) of a CAR described herein comprises (a) a light chain variable region comprising the amino acid sequence set forth in SEQ ID NO: 35, and (b) a heavy chain variable region comprising the amino acid sequence set forth in SEQ ID NO: 24.

In some embodiments, the extracellular antigen-binding domain (e.g., scFv) of a CAR described herein comprises (a) a light chain variable region comprising the amino acid sequence set forth in SEQ ID NO: 36, and (b) a heavy chain variable region comprising the amino acid sequence set forth in SEQ ID NO: 25.

In some embodiments, the extracellular antigen-binding domain (e.g., scFv) of a CAR described herein comprises (a) a light chain variable region comprising the amino acid sequence set forth in SEQ ID NO: 37, and (b) a heavy chain variable region comprising the amino acid sequence set forth in SEQ ID NO: 26.

In some embodiments, the extracellular antigen-binding domain (e.g., scFv) of a CAR described herein comprises (a) a light chain variable region comprising the amino acid sequence set forth in SEQ ID NO: 38, and (b) a heavy chain variable region comprising the amino acid sequence set forth in SEQ ID NO: 27.

In some embodiments, the extracellular antigen-binding domain (e.g., scFv) comprises the amino acid sequence set forth in SEQ ID NO: 4. In some embodiments, the extracellular antigen-binding domain (e.g., scFv) comprises the amino acid sequence set forth in SEQ ID NO: 5. In some embodiments, the extracellular antigen-binding domain (e.g., scFv) comprises the amino acid sequence set forth in SEQ ID NO: 44. In some embodiments, the extracellular antigen-binding domain (e.g., scFv) comprises the amino acid sequence set forth in SEQ ID NO: 45. In some embodiments, the extracellular antigen-binding domain (e.g., scFv) comprises the amino acid sequence set forth in SEQ ID NO: 46. In some embodiments, the extracellular antigen-binding domain (e.g., scFv) comprises the amino acid sequence set forth in SEQ ID NO: 47. In some embodiments, the extracellular antigen-binding domain (e.g., scFv) comprises the amino acid sequence set forth in SEQ ID NO: 49.

In some embodiments, the extracellular antigen-binding domain (e.g. scFV) of a CAR described herein comprises (a) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from the group consisting of SEQ ID NO: 1, SEQ ID NO: 2, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38; and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from the group consisting of SEQ ID NO: 3, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, and SEQ ID NO: 27.

In some embodiments, the extracellular antigen-binding domain (e.g. scFV) of a CAR described herein comprises (a) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 1; and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 3.

In some embodiments, the extracellular antigen-binding domain (e.g. scFv) comprises (a) a light chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 2; and (b) a heavy chain variable region comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO: 3.

In some embodiments, the extracellular antigen-binding domain (e.g. scFv) comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence selected from the group consisting of SEQ ID NO:4, SEQ ID NO: 5, SEQ ID NO: 44, SEQ ID NO: 45, SEQ ID NO: 46, SEQ ID NO: 47, and SEQ ID NO: 49.

In some embodiments, the extracellular antigen-binding domain (e.g. scFv) comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO:4.

In some embodiments, the extracellular antigen-binding domain (e.g. scFv) comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence of SEQ ID NO:5.

### CAR-Expressing Immune Cells

Provided herein are immune cells comprising (expressing) the CARs described herein. Provided herein are immune effector cells (e.g, T lymphocytes) comprising (expressing) the CARs described herein. Any immune cell with one or more effector functions (e.g., cytotoxic cell killing activity, secretion of cytokines, induction of antibody directed cell cytotoxicity (ADCC), and/or complement-dependent cytotoxicity (CDC)) can be used. In some embodiments, the CARs described herein are transduced, transfected, or infected into an immune cell (e.g., a T cell).

In some embodiments, the immune cell is a T lymphocyte. T lymphocytes or “T cells” include, but are not limited to thymocytes, immature T lymphocytes, mature T lymphocytes, resting T lymphocytes, or activated T lymphocytes. In some embodiments, a T cell is a T helper (Th) cell, for example a T helper 1 (Th1) or a T helper 2 (Th2) cell. The T cell can be a helper T cell (HTL; CD4⁺ T cell) CD4⁺ T cell, a cytotoxic T cell (CTL; CD8⁺ T cell), CD4⁺CD8⁺ T cell, CD4⁻CD8⁻ T cell, or any other subset of T cells. Other illustrative populations of T cells suitable for use in some embodiments include naive T cells and memory T cells. In some embodiments, the T lymphocyte is a naive T lymphocyte or MHC restricted T lymphocyte. In some embodiments, the T lymphocytes provided herein are tumor infiltrating lymphocytes (TILs).

In some embodiments, the immune cell is a natural killer cell (NK cell). In some embodiments, the immune cell is an NKT cell.

In some embodiments, the immune cell is a monocyte.

In some embodiments, the immune cell is a macrophage.

As would be understood by the skilled person, other cells may also be used as immune effector cells with the CARs as described herein.

In some embodiments, the immune cells are allogeneic. In some embodiments, the immune cells are autologous. In some embodiments, the immune cells are allogeneic T cells. In some embodiments, the immune cells are autologous T cells. In some embodiments, the immune cells are obtained from a subject that is not the subject to be treated with the CAR expressing immune cells.

In some embodiments, the immune cells are obtained from a healthy donor. In some embodiments, the immune cells are obtained from a patient afflicted with a cancer or a tumor. In some embodiments, the immune cells are isolated from a tumor biopsy, or are expanded from immune cells isolated from a tumor biopsy. In some embodiments, the immune cells are isolated from, but not limited to, bone marrow, fetal, neonate or adult or other hematopoietic cell source, e.g., fetal liver, peripheral blood, lymph node tissue, thymus tissue, spleen tissue, or umbilical cord blood.

In some embodiments, T lymphocytes are obtained from a healthy donor. In some embodiments, T lymphocytes are obtained from a patient afflicted with a cancer or a tumor. In some embodiments, T lymphocytes are obtained from a patient afflicted with a cancer or a tumor. In some embodiments, T lymphocytes are isolated from a tumor biopsy, or are expanded from T lymphocytes isolated from a tumor biopsy. In some embodiments, T cells are isolated from, but not limited to, bone marrow, fetal, neonate or adult or other hematopoietic cell source, e.g., fetal liver, peripheral blood, lymph node tissue, thymus tissue, spleen tissue, or umbilical cord blood.

Various techniques known in the art can be employed to separate the cells. Monoclonal antibodies are particularly useful for identifying markers associated with particular cell lineages and/or stages of differentiation for both positive and negative selections. A large proportion of terminally differentiated cells can be initially removed by a relatively crude separation. For example, magnetic bead separations can be used initially to remove large numbers of irrelevant cells. Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, chip, elutriation or any other convenient technique. Additional techniques for separation and analysis include, but are not limited to, flow cytometry, which can have varying degrees of sophistication, e.g., a plurality of color channels, low angle and obtuse light scattering detecting channels, impedance channels. The cells can be selected against dead cells, by employing dyes associated with dead cells such as propidium iodide (PI).

In some embodiments, the disclosure provides a population of immune cells comprising (e.g., expressing) a CAR as described herein (e.g., for the treatment of cancer or conditioning before hematopoietic transplant). For example, a population of immune cells can be obtained from peripheral blood mononuclear cells (PBMCs) of a patient (e.g., diagnosed with any cancer described herein) and modified to express a CAR described herein. The PBMCs can be CD4⁺, CD8⁺, or CD4⁺ and CD8⁺.

The disclosure provides methods for making immune cells which express any of the CARs described herein. In some embodiments, the method comprises transfecting or transducing immune cells isolated from an individual such that the immune cells express one or more CAR as described herein. Methods for transfection, transduction, and infection are well known in the art. In some embodiments, an immune cell described herein is transformed with a polynucleotide encoding a CAR described herein. In some embodiments, a T cell is transformed with a polynucleotide encoding a CAR described herein. In some embodiments, an immune cell described herein is expanded (i.e. proliferated) prior to and/or subsequent to transformation with a nucleic acid encoding a CAR described herein.

In some embodiments, immune cells are isolated from an individual and genetically modified to express a CAR without further manipulation in vitro, and then re-administered into the individual. In some embodiments, immune cells are first activated and stimulated to proliferate in vitro prior to being genetically modified to express a CAR. Immune cells may be cultured or expanded before and/or after being genetically modified (i.e., transduced or transfected to express a CAR contemplated herein).

In some embodiments, immune cells for an autologous CAR therapy are prepared by collecting white blood cells of a subject, isolating T cells from the white blood cells (e.g., using CD3/CD28 beads), transducing the T cells with an anti-FLT3 CAR (such as any CAR described herein), expanding the anti-FLT3 CAR T cells, thus producing a population of anti-FLT3 CAR T cells that can be used in autologous CAR T therapy. Such cells can be infused into the same subject from whom the original white blood cells were obtained. In some embodiments, instead of T cells other immune cells are isolated from the subject, transduced with an anti-FLT3 CAR, and expanded for use in autologous therapy.

In some embodiments, an immune cell (e.g., T cell) expresses from about 1 to about 4, from about 2 to about 4, from about 3 to about 4, from about 1 to about 2, from about 1 to about 3, or from about 2 to about 3 vector copy numbers/cell of a CAR described herein.

In some embodiments, an immune cell (e.g., T cell) expresses a CAR comprising the nucleic acid sequence of any one of SEQ ID NOs: 60-70. In some embodiments, an immune cell (e.g., T cell) expresses a CAR comprising the amino acid sequence of any one of SEQ ID NOs: 6-16. In some embodiments, an immune cell (e.g., T cell) expresses a CAR comprising a nucleic acid sequence having at least 75%, at least 80%, at least 85%, at least 90%, at least 95%, at least 98% or at least 99% identity to any one of SEQ ID NOs: 60-70. In some embodiments, an immune cell (e.g., T cell) expresses a CAR comprising an amino acid sequence having at least 75%, at least 80%, at least 85%, at least 90%, at least 95%, at least 98% or at least 99% identity to any one of SEQ ID NOs: 6-16.

In some embodiments, a T cell expresses a CAR comprising the nucleic acid sequence of SEQ ID NO: 60. In some embodiments, a T cell expresses a CAR comprising the nucleic acid sequence of SEQ ID NO: 61. In some embodiments, a T cell expresses a CAR comprising the nucleic acid sequence of SEQ ID NO: 62. In some embodiments, a T cell expresses a CAR comprising the nucleic acid sequence of SEQ ID NO: 63. In some embodiments, a T cell expresses a CAR comprising the nucleic acid sequence of SEQ ID NO: 64. In some embodiments, a T cell expresses a CAR comprising the nucleic acid sequence of SEQ ID NO: 65. In some embodiments, a T cell expresses a CAR comprising the nucleic acid sequence of SEQ ID NO: 66. In some embodiments, a T cell expresses a CAR comprising the nucleic acid sequence of SEQ ID NO: 67. In some embodiments, a T cell expresses a CAR comprising the nucleic acid sequence of SEQ ID NO: 68. In some embodiments, a T cell expresses a CAR comprising the nucleic acid sequence of SEQ ID NO: 69. In some embodiments, a T cell expresses a CAR comprising the nucleic acid sequence of SEQ ID NO: 70. In any of these embodiments, instead of a T cell another immune cell can be used, e.g., a NK cell, a macrophage or a monocyte.

In some embodiments, a T cell expresses a CAR comprising the amino acid sequence of SEQ ID NO: 6. In some embodiments, a T cell expresses a CAR comprising the amino acid sequence of SEQ ID NO: 7. In some embodiments, a T cell expresses a CAR comprising the amino acid sequence of SEQ ID NO: 8. In some embodiments, a T cell expresses a CAR comprising the amino acid sequence of SEQ ID NO: 9. In some embodiments, a T cell expresses a CAR comprising the amino acid sequence of SEQ ID NO: 10. In some embodiments, a T cell expresses a CAR comprising the amino acid sequence of SEQ ID NO: 11. In some embodiments, a T cell expresses a CAR comprising the amino acid sequence of SEQ ID NO: 12. In some embodiments, a T cell expresses a CAR comprising the amino acid sequence of SEQ ID NO: 13. In some embodiments, a T cell expresses a CAR comprising the amino acid sequence of SEQ ID NO: 14. In some embodiments, a T cell expresses a CAR comprising the amino acid sequence of SEQ ID NO: 15. In some embodiments, a T cell expresses a CAR comprising the amino acid sequence of SEQ ID NO:16. In any of these embodiments, instead of a T cell another immune cell can be used, e.g., a NK cell, a macrophage or a monocyte.

In some embodiments, an immune cell (e.g., T cell) expresses any CAR described herein comprising an scFv of the amino acid sequence of any one of SEQ ID NOs: 4, 5, 44, 45, 46, 48 or 49. In some embodiments, an immune cell (e.g., T cell) expresses any CAR described herein comprising an scFv of SEQ ID NO: 4. In some embodiments, an immune cell (e.g., T cell) expresses any CAR described herein comprising an scFv of SEQ ID NO: 5. In some embodiments, an immune cell (e.g., T cell) expresses any CAR described herein comprising an scFv of SEQ ID NO: 44. In some embodiments, an immune cell (e.g., T cell) expresses any CAR described herein comprising an scFv of SEQ ID NO: 45. In some embodiments, an immune cell (e.g., T cell) expresses any CAR described herein comprising an scFv of SEQ ID NO: 46. In some embodiments, an immune cell (e.g., T cell) expresses any CAR described herein comprising an scFv of SEQ ID NO: 47. In some embodiments, an immune cell (e.g., T cell) expresses any CAR described herein comprising an scFv of SEQ ID NO: 49.

### Pharmaceutical Compositions

Provided herein are pharmaceutical compositions comprising (i) any anti-FLT3 antibody or fragment described herein (e.g, any scFv described herein) or any anti-FLT3 CAR expressing immune cell described herein (including a population of immune cells), and (ii) a pharmaceutically acceptable carrier. Appropriate pharmaceutically acceptable carriers including, but not limited to, excipients and stabilizers are known in the art (see, e.g. Remington's Pharmaceutical Sciences (1990) Mack Publishing Co., Easton, PA).

In some embodiments, pharmaceutically acceptable carriers include but are not limited to an isotonic agent, a buffer, a suspending agent, a dispersing agent, an emulsifying agent, a wetting agent, a sequestering agent, a chelating agent, a pH buffering agent, a solubility enhancer, an antioxidant, an anesthetic, and/or an antimicrobial agent. In some embodiments, the carriers are selected from, but not limited to, one or more of water, saline, phosphate buffered saline, dextrose, glycerol, ethanol and the like, starch, lactose, sucrose, gelatin, malt, propylene, silica gel, sodium stearate, and dextrose as well as combinations thereof. In some embodiments, the pharmaceutically acceptable carriers further comprise auxiliary substances such as wetting or emulsifying agents, preservatives or buffers, which enhance the shelf life or effectiveness of the binding proteins.

In some embodiments, when administered parenterally, the pharmaceutical acceptable carriers include, but are not limited to, physiological saline or phosphate buffered saline (PBS), solutions containing agents such as glucose, polyethylene glycol, polypropylene glycol, or other agents.

In some embodiments, the pharmaceutical composition is formulated to provide rapid, sustained, or delayed release of the active ingredient after administration. Formulations for providing rapid, sustained, or delayed release of the active ingredient after administration are known in the art (Mishra, M. K. (2016). Handbook of encapsulation and controlled release. Boca Raton, CRC Press, Taylor & Francis Group, CRC Press is an imprint of the Taylor & Francis Group, an Informa business, incorporated herein by reference in its entirety).

In some embodiments, a pharmaceutical composition provided herein comprises any anti-FLT3 antibody or fragment described herein (e.g, any scFv described herein) or any anti-FLT3 CAR expressing immune cell described herein (including a population of immune cells) and one or more other therapeutic agents (e.g., an anti-cancer agent) in a pharmaceutically acceptable carrier.

In some embodiments, a pharmaceutical composition is formulated for any route of administration to a subject. In some embodiments, the pharmaceutical composition is formulated for injection and prepared as a liquid solution, suspension, emulsion, or solid form suitable for making into a solution or suspension prior to injection.

In some embodiments, the anti-FLT3 antibody or fragment described herein (e.g, any scFv described herein) or the anti-FLT3 CAR expressing immune cell described herein (including a population of immune cells) in the pharmaceutical composition is present in a therapeutically effective amount. Therapeutically effective amounts are determined by methods known in the art.

### Therapeutic Methods

### Cancer Treatment

In some embodiments, the disclosure provides methods for treating cancer comprising administering any anti-FLT3 antibody or fragment described herein, any pharmaceutical composition described herein, or any anti-FLT3 CAR expressing immune cells described herein.

In some embodiments, the method of treating cancer comprises administering to a subject any anti-FLT3 antibody or fragment described herein or any anti-FLT3 CAR expressing immune cells described herein that binds to a FLT3 epitope of a cell (e.g., of a target cell). In some embodiments, the method of treating cancer comprises administering to a subject any anti-FLT3 antibody or fragment described herein or any anti-FLT3 CAR expressing immune cells described herein that binds to a FLT3 epitope of a cancer cell (e.g., AML cell).

In some embodiments, the disclosure provides a method of treating cancer that is resistant to other cancer therapy or therapies (e.g., vaccine, chemotherapy, radiotherapy, small molecule therapy, or immunotherapy (such as treatment with another antibody). In some embodiments, the cancer is resistant to chemotherapy. In some embodiments, the cancer is resistant to radiotherapy. In some embodiments, the cancer is resistant to small molecule therapy. In some embodiments, the cancer is resistant to immunotherapy.

The methods described herein are suitable for treating cancers that are expected, known, or determined to express FLT3 on the surface of their cells.

In some embodiments, the administration of any anti-FLT3 antibody or fragment described herein, any pharmaceutical composition described herein, or any anti-FLT3 CAR expressing immune cells described herein, in accordance with the methods described herein is carried out to achieve or result in one or more of the following: (i) a decrease in cancer cell frequency or number, (ii) a reduction in the growth of the cancer or increase in the number of cancer cells, (iii) inhibition of the progression of cancer cell growth, (iv) the regression of cancer, (v) inhibition of a recurrence of the cancer, (vi) eradication of the cancer, (vii) reduction or amelioration of the severity or duration of one or more symptoms of the cancer, (viii) the inhibition of the development or onset of one or more symptoms associated with cancer, (ix) the enhancement or improvement of the therapeutic effect of another anti-cancer therapy, (x) increase in life expectancy or survival of a subject, (xi) reduction in hospitalization (e.g. length of hospitalization) in a subject, (xii) improvement in a subject's quality of life, (xiii) a reduction in mortality, (xiv) an increase in a relapse free survival or length of remission in a subject. In some embodiments, the administration of any anti-FLT3 antibody or fragment described herein, any pharmaceutical composition described herein, or any anti-FLT3 CAR expressing immune cells described herein, in accordance with the methods described herein is carried out to achieve or result in reduction of tumor burden in a subject (e.g., effective to reduce tumor burden relative to tumor burden in the subject prior to treatment).

In some embodiments, the administration of any anti-FLT3 antibody or fragment described herein, any pharmaceutical composition described herein, or any anti-FLT3 CAR expressing immune cells described herein is effective to treat cancer in a subject (e.g., decreases cancer cell frequency or number, reduces cancer cell growth or proliferation, increases life expectancy or survival, eradicates cancer, or improves one or more symptoms of cancer), when used alone or in combination with another therapy.

In some embodiments, administration to a subject of an anti-FLT3 antibody or fragment described herein, a pharmaceutical composition described herein, or an anti-FLT3 CAR expressing immune cells described herein is effective to reduce cell frequency or number of cancer cells, or eliminate cancer cells. In some embodiments, administration to a subject of an anti-FLT3 antibody or fragment described herein, a pharmaceutical composition described herein, or an anti-FLT3 CAR expressing immune cells described herein is effective to reduce the number or frequency of cancer cells by at least 30%, at least 40%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of cancer cells in the subject before administration of this therapy). In some embodiments, administration to a subject of an anti-FLT3 antibody or fragment described herein, a pharmaceutical composition described herein, or an anti-FLT3 CAR expressing immune cells described herein is effective to reduce the number or frequency of cancer cells by at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of cancer cells in the subject before administration of this therapy). In some embodiments, administration to a subject of an anti-FLT3 antibody or fragment described herein, a pharmaceutical composition described herein, or an anti-FLT3 CAR expressing immune cells described herein is effective to reduce the number or frequency of cancer cells by at least 75%, at least 80%, at least 85%, at least 90%, at least 95%, at least 97%, at least 98%, at least 99% or about 100%, relative to control or baseline (e.g., relative to the level of cancer cells in the subject before administration of this therapy). In some embodiments, administration to a subject of an anti-FLT3 antibody or fragment described herein, a pharmaceutical composition described herein, or an anti-FLT3 CAR expressing immune cells described herein is effective to reduce the number or frequency of cancer cells by at least 90%, at least 95%, at least 97%, at least 98%, at least 99% or about 100%, relative to control or baseline (e.g., relative to the level of cancer cells in the subject before administration of this therapy).

In some embodiments, administration to a subject of an anti-FLT3 antibody or fragment described herein, a pharmaceutical composition described herein, or an anti-FLT3 CAR expressing immune cells described herein is effective to treat any of the cancers described herein (e.g., AML). In some embodiments, administration to a subject of an anti-FLT3 antibody or fragment described herein, a pharmaceutical composition described herein, or an anti-FLT3 CAR expressing immune cells described herein is effective to slow progression of any of the cancers described herein (e.g., AML). In some embodiments, administration to a subject of an anti-FLT3 antibody or fragment described herein, a pharmaceutical composition described herein, or an anti-FLT3 CAR expressing immune cells described herein is effective to reduce tumor burden of any the cancers described herein (e.g., AML). In some embodiments, administration to a subject of an anti-FLT3 antibody or fragment described herein, a pharmaceutical composition described herein, or an anti-FLT3 CAR expressing immune cells described herein is effective to increase survival of the subject having any cancer described herein. In some embodiments, administration of an anti-FLT3 antibody or fragment described herein, a pharmaceutical composition described herein, or an anti-FLT3 CAR expressing immune cells described herein is effective to increase median survival of subjects relative to subjects not treated or treated with a placebo. In some embodiments, administration of an anti-FLT3 antibody or fragment described herein, a pharmaceutical composition described herein, or an anti-FLT3 CAR expressing immune cells described herein is effective to increase median survival of subjects relative to subjects treated with a standard of care therapy.

Examples of the cancer cells that can be reduced in number or eliminated using the methods described herein include, without limitation, blast cells of acute myeloid leukemia (AML), lymphoblasts or leukemic blasts of acute lymphocytic leukemia (ALL), myeloblasts of chronic myeloid leukemia (CML), Blastic plasmacytoid dendritic cell neoplasm (BPDCN), and blasts of chronic lymphocytic leukemia (CLL).

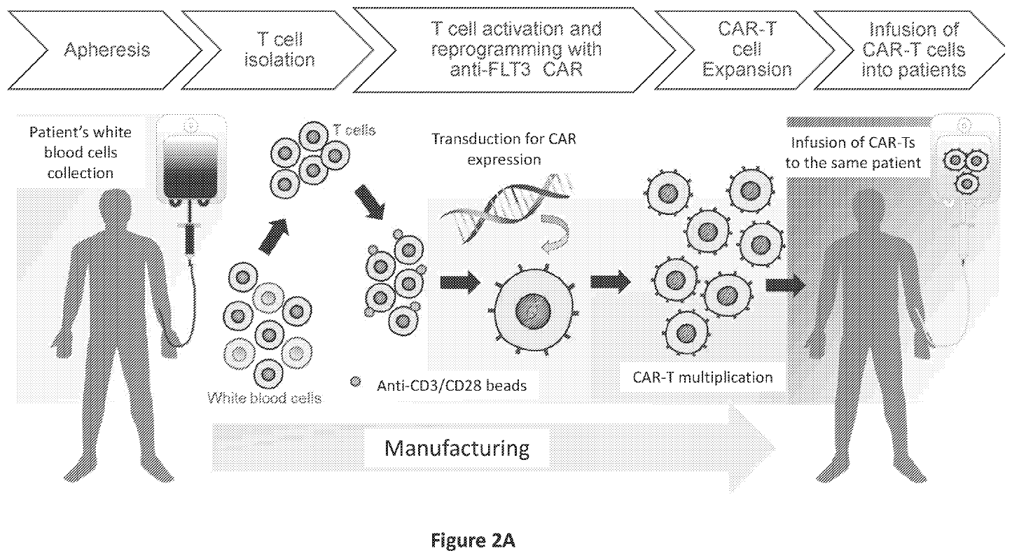

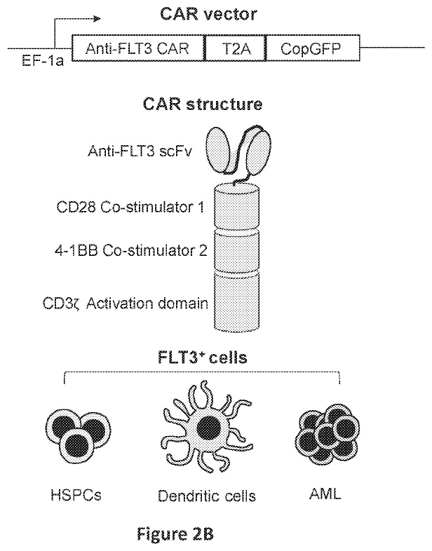

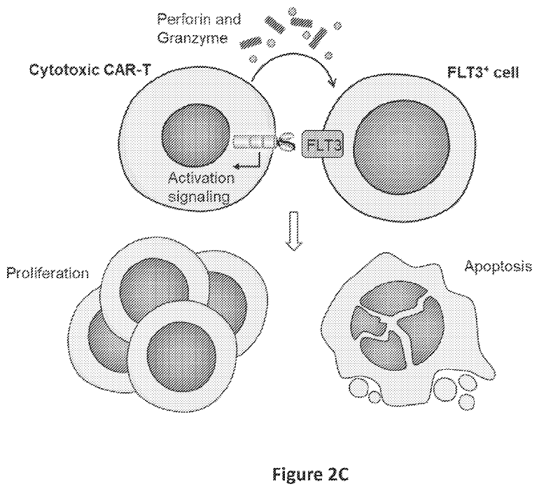

In certain embodiments, the immune cells expressing any anti-FLT3 CAR described herein are used in the methods of treatment a subject described herein. In some embodiments, the anti-FLT3 CAR expressing immune cells are autologous to the subject being treated. In some embodiments, blood (e.g., white blood cells) is collected from a subject (e.g., by apheresis), followed by isolating immune cells (e.g., T cells) from the blood (e.g., using anti-CD3/CD28 beads), followed by introducing a nucleic acid encoding an anti-FLT3 CAR into the isolated immune cells (which may optionally be followed by expanding the isolated immune cells comprising an anti-FLT3 CAR), and then followed by administering (e.g., by infusion) thus obtained immune cells comprising an anti-FLT3 CAR to the subject (i.e., the same subject from which the immune cells were isolated). This autologous CAR T therapy is depicted in FIG. 2A. In other embodiments, the anti-FLT3 CAR expressing immune cells are not autologous to the subject being treated.

### Hematopoietic Cell Conditioning

In some embodiments, the disclosure provides methods for preparing or conditioning a subject in need thereof for hematopoietic cell transplantation. In some embodiments, a subject in need thereof is a patient that qualifies for, will be receiving or is receiving bone marrow (BM) hematopoietic stem cell and/or hematopoietic progenitor cell transplantation. In some embodiments, the subject in need of a hematopoietic cell transplantation has cancer (such as any cancer described herein).

In some embodiments, the disclosure provides methods for preparing or conditioning a subject in need thereof for hematopoietic cell transplantation wherein the subject is administered any anti-FLT3 antibody or fragment described herein, any pharmaceutical composition described herein, or any anti-FLT3 CAR expressing immune cells described herein.

In some embodiments, the method of preparing or conditioning a subject comprises administering to a subject any anti-FLT3 antibody or fragment described herein or any anti-FLT3 CAR expressing immune cells described herein that binds to a FLT3 epitope on a hematopoietic stem cell. In some embodiments, the method of preparing or conditioning a subject comprises administering to a subject any anti-FLT3 antibody or fragment described herein or any anti-FLT3 CAR expressing immune cells described herein that binds to a FLT3 epitope on a hematopoietic progenitor cell. In some embodiments, the method of preparing or conditioning a subject comprises administering to a subject any anti-FLT3 antibody or fragment described herein or any anti-FLT3 CAR expressing immune cells described herein that binds to a FLT3 epitope on a dendritic cell. In some embodiments, the method of preparing or conditioning a subject comprises administering to a subject any anti-FLT3 antibody or fragment described herein or any anti-FLT3 CAR expressing immune cells described herein that binds to a FLT3 epitope on a myeloid cell. In some embodiments, the method of preparing or conditioning a subject comprises administering to a subject any anti-FLT3 antibody or fragment described herein or any anti-FLT3 CAR expressing immune cells described herein that binds to a FLT3 epitope on a lymphoid cell.

In some embodiments, administration to a subject of an anti-FLT3 antibody or fragment described herein, a pharmaceutical composition described herein, or an anti-FLT3 CAR expressing immune cells described herein is effective to condition a subject prior to hematopoietic cell transplantation.

In certain embodiments, the immune cells expressing any anti-FLT3 CAR described herein are used in the methods of treatment a subject described herein. In some embodiments, the anti-FLT3 CAR expressing immune cells are autologous to the subject being treated. In some embodiments, blood is collected from a subject, followed by isolating immune cells (e.g, T cells) from the blood, followed by introducing a nucleic acid encoding an anti-FLT3 CAR into the isolated immune cells (which may optionally be followed by expanding the isolated immune cells comprising an anti-FLT3 CAR), and then followed by administering thus obtained autologous immune cells comprising an anti-FLT3 CAR to the subject. In other embodiments, the anti-FLT3 CAR expressing immune cells are not autologous to the subject being treated.

In some embodiments, an anti-FLT3 antibody or fragment described herein, a pharmaceutical composition described herein or an anti-FLT3 CAR expressing immune cells described herein is effective to significantly reduce cell frequency or number, or eliminate, hematopoietic stem cells (HSC) and/or hematopoietic progenitor cells (HPCs) (e.g., early hematopoietic progenitors). In some embodiments, administration to a subject of an anti-FLT3 antibody or fragment described herein, a pharmaceutical composition described herein, or an anti-FLT3 CAR expressing immune cells described herein is effective to reduce the number or frequency of HSCs and/or HPCs (e.g., early HPCs) by at least 30%, at least 40%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or an anti-FLT3 CAR expressing immune cells described herein is effective to reduce the number or frequency of HSCs and/or HPCs (e.g., early HPCs) by at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or an anti-FLT3 CAR expressing immune cells described herein is effective to reduce the number or frequency of HSCs and/or HPCs (e.g., early HPCs) by at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or an anti-FLT3 CAR expressing immune cells described herein is effective to reduce the number or frequency of HSCs and/or HPCs (e.g., early HPCs) by at least 90%, at least 95%, at least 97%, at least 98%, at least 99% or about 100%, relative to control or baseline (e.g., relative to the level of the cells in the subject before administration of this therapy). In some of these embodiments, the reduction of HSCs and/or HPCs (e.g., early HPCs) is in bone marrow of the subject being treated (e.g., in bone marrow mononuclear cells).

In some embodiments, an anti-FLT3 antibody or fragment described herein, a pharmaceutical composition described herein or an anti-FLT3 CAR expressing immune cells described herein is effective to significantly reduce cell frequency or number, or eliminate, multi-potent progenitor cells (MPPs) and/or common progenitor cells (CPs). In some embodiments, administration to a subject of an anti-FLT3 antibody or fragment described herein, a pharmaceutical composition described herein, or an anti-FLT3 CAR expressing immune cells described herein is effective to reduce the number or frequency of MPPs and/or CPs by at least 30%, at least 40%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or an anti-FLT3 CAR expressing immune cells described herein is effective to reduce the number or frequency of MPPs and/or CPs by at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or an anti-FLT3 CAR expressing immune cells described herein is effective to reduce the number or frequency of MPPs and/or CPs by at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or an anti-FLT3 CAR expressing immune cells described herein is effective to reduce the number or frequency of MPPs and/or CPs by at least 90%, at least 95%, at least 97%, at least 98%, at least 99% or about 100%, relative to control or baseline (e.g., relative to the level of the cells in the subject before administration of this therapy). In some of these embodiments, the reduction of MPPs or CPs is in bone marrow of the subject being treated (e.g., in bone marrow mononuclear cells).

According to some embodiments, administration to a subject of an anti-FLT3 antibody or fragment described herein, a pharmaceutical composition described herein, or an anti-FLT3 CAR expressing immune cells described herein is effective to condition patients undergoing bone marrow (BM) HSC and/or HPC (e.g., early HPC) transplantation. In some embodiments, the subject receives HSC transplantation. In some embodiments, the subject receives HPC transplantation. In some embodiments, the subject receives both HSC and HPC (e.g., early HPC) transplantation. In some embodiments, the subject receives MPPs and/or CPs. In some embodiments, the HSC/HPC transplantation is for treating any hematologic cancer described herein, e.g., Acute Myeloid Leukemia (AML), Acute Lymphoblastic Leukemia (ALL), dendritic cell neoplasm, among others.

In some embodiments, administration to a subject of an anti-FLT3 antibody or fragment described herein, a pharmaceutical composition described herein, or an anti-FLT3 CAR expressing immune cells described herein reduces the number or frequency of myeloid cell lineages (e.g., circulating myeloid lineage cells or monocytes). In some embodiments, administration to a subject of an anti-FLT3 antibody or fragment described herein, a pharmaceutical composition described herein, or an anti-FLT3 CAR expressing immune cells described herein reduces the number or frequency of myeloid lineage cells (e.g., circulating myeloid lineage cells or monocytes) by at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or an anti-FLT3 CAR expressing immune cells described herein reduces the number or frequency of myeloid lineage cells (e.g., circulating myeloid lineage cells or monocytes) by at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy).

In some embodiments, administration to a subject of an anti-FLT3 antibody or fragment described herein, a pharmaceutical composition described herein, or an anti-FLT3 CAR expressing immune cells described herein does not significantly reduce the number or frequency of bone marrow lineage cells. In some embodiments, administration to a subject of an anti-FLT3 antibody or fragment described herein, a pharmaceutical composition described herein, or an anti-FLT3 CAR expressing immune cells described herein reduces the number or frequency of bone marrow lineage cells by less than 60%, less than 55%, less than 50%, less than 40%, less than 30% or less than 20% relative to control or baseline (e.g., relative to the level of the cells in the subject before administration of this therapy).

In some embodiments, administration to a subject of an anti-FLT3 antibody or fragment described herein, a pharmaceutical composition described herein, or an anti-FLT3 CAR expressing immune cells described herein reduces the number or frequency of bone marrow lineage cells. In some embodiments, administration to a subject of an anti-FLT3 antibody or fragment described herein, a pharmaceutical composition described herein, or an anti-FLT3 CAR expressing immune cells described herein reduces the number or frequency of bone marrow lineage cells by at least 40%, at least 45%, at least 50%, at least 55%, or at least 60%6 relative to control or baseline (e.g., relative to the level of the cells in the subject before administration of this therapy).

In some embodiments, administration to a subject of an anti-FLT3 antibody or fragment described herein, a pharmaceutical composition described herein, or an anti-FLT3 CAR expressing immune cells described herein reduces a cell population expressing one or more of (e.g., one, two, three, four, five of six of) CD45, FLT3, CD19, CD38, CD33 and CD34. In some of these embodiments, the reduction of a cell population expressing one or more of (e.g., one, two, three, four, five or six of) CD45, FLT3, CD19, CD38, CD33 and CD34 is in bone marrow of the subject being treated (e.g., in bone marrow mononuclear cells). In some of these embodiments, the reduction of a cell population expressing one or more of (e.g., one, two, three, four, five or six of) CD45, FLT3, CD19, CD38, CD33 and CD34 is in circulating blood cells of the subject being treated (e.g., in bone marrow mononuclear cells).

In some embodiments, administration to a subject of an anti-FLT3 antibody or fragment described herein, a pharmaceutical composition described herein, or an anti-FLT3 CAR expressing immune cells described herein reduces a cell population expressing one or more of (e.g., one, two, three, or four of) FLT3, CD38, CD33 and CD34. In some of these embodiments, the reduction of a cell population expressing one or more of (e.g., one, two, three, or four of) CD45, FLT3, CD19, CD38, CD33 and CD34 is in bone marrow of the subject being treated (e.g., in bone marrow mononuclear cells). In some of these embodiments, the reduction of a cell population expressing one or more of (e.g., one, two, three, or four of) CD45, FLT3, CD19, CD38, CD33 and CD34 is in circulating blood cells of the subject being treated (e.g., in bone marrow mononuclear cells).

In some embodiments, administration to a subject of an anti-FLT3 antibody or fragment described herein, a pharmaceutical composition described herein, or an anti-FLT3 CAR expressing immune cells described herein reduces the number or frequency of FLT3 expressing cells. In some embodiments, administration to a subject of an anti-FLT3 antibody or fragment described herein, a pharmaceutical composition described herein, or an anti-FLT3 CAR expressing immune cells described herein reduces the number or frequency of FLT3 expressing cells by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy. In some embodiments, administration to a subject of an anti-FLT3 antibody or fragment described herein, a pharmaceutical composition described herein, or an anti-FLT3 CAR expressing immune cells described herein reduces the number or frequency of FLT3 expressing cells by at least 60% relative to control or baseline (e.g., relative to the level of the cells in the subject before administration of this therapy. In some embodiments, administration to a subject of an anti-FLT3 antibody or fragment described herein, a pharmaceutical composition described herein, or an anti-FLT3 CAR expressing immune cells described herein reduces the number or frequency of FLT3 expressing cells by at least 70% relative to control or baseline (e.g., relative to the level of the cells in the subject before administration of this therapy. In some embodiments, administration to a subject of an anti-FLT3 antibody or fragment described herein, a pharmaceutical composition described herein, or an anti-FLT3 CAR expressing immune cells described herein reduces the number or frequency of FLT3 expressing cells by at least 80% relative to control or baseline (e.g., relative to the level of the cells in the subject before administration of this therapy. In some embodiments, administration to a subject of an anti-FLT3 antibody or fragment described herein, a pharmaceutical composition described herein, or an anti-FLT3 CAR expressing immune cells described herein reduces the number or frequency of FLT3 expressing cells by at least 90% relative to control or baseline (e.g., relative to the level of the cells in the subject before administration of this therapy. In some embodiments, administration to a subject of an anti-FLT3 antibody or fragment described herein, a pharmaceutical composition described herein, or an anti-FLT3 CAR expressing immune cells described herein reduces the number or frequency of FLT3 expressing cells by at least 95% relative to control or baseline (e.g., relative to the level of the cells in the subject before administration of this therapy. In some of these embodiments, the reduction of FLT3 expressing cells is in bone marrow of the subject being treated (e.g., in bone marrow mononuclear cells). In some of these embodiments, the reduction of FLT3 expressing cells is in circulating blood cells of the subject being treated. In some of these embodiments, the reduction of FLT3 expressing cells is reduction of cancer cells in the subject being treated.

In some embodiments, administration to a subject of an anti-FLT3 antibody or fragment described herein, a pharmaceutical composition described herein, or an anti-FLT3 CAR expressing immune cells described herein reduces the number or frequency of CD34+ hematopoietic stem cells. In some embodiments, administration to a subject of an anti-FLT3 antibody or fragment described herein, a pharmaceutical composition described herein, or an anti-FLT3 CAR expressing immune cells described herein reduces the number or frequency of CD34+ hematopoietic stem cells by at least 40%, at least 45%, at least 50%, at least 55%, at least 60%6, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some of these embodiments, the reduction of CD34+ hematopoietic stem cells is in bone marrow of the subject being treated (e.g., in bone marrow mononuclear cells). In some of these embodiments, the reduction of CD34+ hematopoietic stem cells is in circulating blood cells of the subject being treated.

In some embodiments, administration to a subject of an anti-FLT3 antibody or fragment described herein, a pharmaceutical composition described herein, or an anti-FLT3 CAR expressing immune cells described herein reduces the number or frequency of early hematopoietic progenitors. In some embodiments, administration to a subject of an anti-FLT3 antibody or fragment described herein, a pharmaceutical composition described herein, or an anti-FLT3 CAR expressing immune cells described herein reduces the number or frequency of early hematopoietic progenitors by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some of these embodiments, the reduction of early hematopoietic progenitors is in bone marrow of the subject being treated (e.g., in bone marrow mononuclear cells). In some of these embodiments, the reduction of early hematopoietic progenitors is in circulating blood cells of the subject being treated.

In some embodiments, administration to a subject of an anti-FLT3 antibody or fragment described herein, a pharmaceutical composition described herein, or an anti-FLT3 CAR expressing immune cells described herein reduces the number or frequency of dendritic cells. In some embodiments, administration to a subject of an anti-FLT3 antibody or fragment described herein, a pharmaceutical composition described herein, or an anti-FLT3 CAR expressing immune cells described herein reduces the number or frequency of dendritic cells by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some of these embodiments, the reduction of dendritic cells is in bone marrow of the subject being treated (e.g., in bone marrow mononuclear cells). In some of these embodiments, the reduction of dendritic cells is in circulating blood cells of the subject being treated.

In some embodiments, administration to a subject of an anti-FLT3 antibody or fragment described herein, a pharmaceutical composition described herein, or an anti-FLT3 CAR expressing immune cells described herein reduces the number or frequency of CD45+CD19+ cells. In some embodiments, administration to a subject of an anti-FLT3 antibody or fragment described herein, a pharmaceutical composition described herein, or an anti-FLT3 CAR expressing immune cells described herein reduces the number or frequency of CD45+CD19+ cells by at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or an anti-FLT3 CAR expressing immune cells described herein reduces the number or frequency of CD45+CD19+ cells by about 55%, about 50%, about 45%, about 40%, or about 35% (or between about 30% and 55%) relative to control or baseline (e.g., relative to the level of the cells in the subject before administration of this therapy). In some of these embodiments, the reduction of CD45+CD19+ cells is in bone marrow of the subject being treated (e.g., in bone marrow mononuclear cells). In some of these embodiments, the reduction of CD45+CD19+ is in circulating blood cells of the subject being treated.

In some embodiments, administration to a subject of an anti-FLT3 antibody or fragment described herein, a pharmaceutical composition described herein, or an anti-FLT3 CAR expressing immune cells described herein reduces the number or frequency of CD34+CD38+ cells. In some embodiments, administration to a subject of an anti-FLT3 antibody or fragment described herein, a pharmaceutical composition described herein, or an anti-FLT3 CAR expressing immune cells described herein reduces the number or frequency of CD34+CD38+ cells by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some of these embodiments, the reduction of CD34+CD38+ cells is in bone marrow of the subject being treated (e.g., in bone marrow mononuclear cells). In some of these embodiments, the reduction of CD34+CD38+ is in circulating blood cells of the subject being treated.

In some embodiments, administration to a subject of an anti-FLT3 antibody or fragment described herein, a pharmaceutical composition described herein, or an anti-FLT3 CAR expressing immune cells described herein reduces the number or frequency of CD34+CD38− cells. In some embodiments, administration to a subject of an anti-FLT3 antibody or fragment described herein, a pharmaceutical composition described herein, or an anti-FLT3 CAR expressing immune cells described herein reduces the number or frequency of CD34+CD38− cells by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or an anti-FLT3 CAR expressing immune cells described herein reduces the number or frequency of CD34+CD38− cells by at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some of these embodiments, the reduction of CD34+CD38− cells is in bone marrow of the subject being treated (e.g., in bone marrow mononuclear cells). In some of these embodiments, the reduction of CD34+CD38− is in circulating blood cells of the subject being treated.

In some embodiments, administration to a subject of an anti-FLT3 antibody or fragment described herein, a pharmaceutical composition described herein, or an anti-FLT3 CAR expressing immune cells described herein reduces a cell population expressing CD34 by at least 60%, at least 70%, at least 75%, at least 80%, at least 90%, at least 95%, at least 97%, or at least 99%.

In some embodiments, administration to a subject of an anti-FLT3 antibody or fragment described herein, a pharmaceutical composition described herein, or an anti-FLT3 CAR expressing immune cells described herein reduces a cell population expressing FLT3 by at least 60%, at least 70%, at least 75%, at least 80%, at least 90%, at least 95%, at least 97%, or at least 99%.

In some embodiments, administration to a subject of an anti-FLT3 antibody or fragment described herein, a pharmaceutical composition described herein, or an anti-FLT3 CAR expressing immune cells described herein reduces the number or frequency of CD33+ cells. In some embodiments, administration to a subject of an anti-FLT3 antibody or fragment described herein, a pharmaceutical composition described herein, or an anti-FLT3 CAR expressing immune cells described herein reduces the number or frequency of CD33+ cells by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some embodiments, administration to a subject of an anti-FLT3 antibody or fragment described herein, a pharmaceutical composition described herein, or anti-FLT3 CAR expressing immune cells described herein reduces a cell population expressing CD33 by at least 60%, at least 70%, at least 75%, at least 80%, at least 90%, at least 95%, at least 97%, or at least 99%. In some of these embodiments, the reduction of CD33 expressing cells is in bone marrow of the subject being treated (e.g., in bone marrow mononuclear cells). In some of these embodiments, the reduction of CD33 expressing cells is in circulating blood cells of the subject being treated.

In some embodiments, administration to a subject of an anti-FLT3 antibody or fragment described herein, a pharmaceutical composition described herein, or an anti-FLT3 CAR expressing immune cells described herein reduces the number or frequency of CD1c+ myeloid dendritic cells. In some embodiments, administration to a subject of an anti-FLT3 antibody or fragment described herein, a pharmaceutical composition described herein, or an anti-FLT3 CAR expressing immune cells described herein reduces the number or frequency of CD1c+ myeloid dendritic cells by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some of these embodiments, the reduction of CD1c+ myeloid dendritic cells is in bone marrow of the subject being treated (e.g., in bone marrow mononuclear cells). In some of these embodiments, the reduction of CD1c+ myeloid dendritic cells is in circulating blood cells of the subject being treated.

In some embodiments, administration to a subject of an anti-FLT3 antibody or fragment described herein, a pharmaceutical composition described herein, or an anti-FLT3 CAR expressing immune cells described herein reduces the number or frequency of CD141+ myeloid dendritic cells. In some embodiments, administration to a subject of an anti-FLT3 antibody or fragment described herein, a pharmaceutical composition described herein, or an anti-FLT3 CAR expressing immune cells described herein reduces the number or frequency of CD141+ myeloid dendritic cells by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy). In some of these embodiments, the reduction of CD141+ myeloid dendritic cells is in bone marrow of the subject being treated (e.g., in bone marrow mononuclear cells). In some of these embodiments, the reduction of CD141+ myeloid dendritic cells is in circulating blood cells of the subject being treated.

In some embodiments, administration to a subject of an anti-FLT3 antibody or fragment described herein, a pharmaceutical composition described herein, or an anti-FLT3 CAR expressing immune cells described herein reduces the number or frequency of CD303⁺ plasmacytoid dendritic cells. In some embodiments, administration to a subject of an anti-FLT3 antibody or fragment described herein, a pharmaceutical composition described herein, or an anti-FLT3 CAR expressing immune cells described herein reduces the number or frequency of CD303⁺ plasmacytoid dendritic cells by at least 40%, at least 45%, at least 50%, at least 55%, at least 60%, at least 65%, at least 70%, at least 75%, at least 80%, at least 85%, at least 90%, at least 95%, at least 97%, at least 98%, at least 99% or about 100%, relative to control or baseline (e.g., relative to the level of the cells in the subject before administration of this therapy. In some of these embodiments, the reduction of CD303⁺ plasmacytoid dendritic cells is in bone marrow of the subject being treated (e.g., in bone marrow mononuclear cells). In some of these embodiments, the reduction of CD303⁺ plasmacytoid dendritic cells is in circulating blood cells of the subject being treated.

In some embodiments, the method of HSC/HPC transplantation comprises transplantation of donor HSC/HPC cells. In some embodiments, donor cells are from a healthy subject. In other embodiments, HSC/HPC transplantation comprises transplantation of autologous cells (e.g., obtained before the onset of disease being treated).

In some embodiments, the disclosure provides methods of hematopoietic stem cell/hematopoietic progenitor cell transplantation in a subject comprising:
(i) reducing the number of hematopoietic stem cells (HSCs) and/or hematopoietic progenitor cells (HPCs) by administering an anti-FLT3 antibody or fragment described herein, a pharmaceutical composition described herein, or anti-FLT3 CAR expressing immune cells described herein to the subject,
(ii) transplanting HSCs/HPCs (e.g., donor HSCs/HPCs) to the subject.

In some embodiments, the disclosure provides methods of hematopoietic stem cell/hematopoietic progenitor cell transplantation in a subject comprising:
(i) reducing the number of hematopoietic stem cells (HSCs) and/or hematopoietic progenitor cells (HPCs) by administering a population of immune cells expressing a CAR having the amino acid sequence selected from the group consisting of: SEQ ID NOs: 6 and 9-15 to the subject,
(ii) transplanting HSCs/HPCs (e.g., donor HSCs/HPCs) to the subject.

### Cancers to be Treated

Cancers can be treated in accordance with the methods described herein. In some embodiments, the cancer to be treated is a hematopoietic or hematologic cancer. Examples of hematologic cancers that are treated in accordance with the methods described herein include, but are not limited to, acute myeloid leukemia (AML), acute lymphoblastic leukemia (ALL), chronic myeloid leukemia (CML), chronic lymphocytic leukemia (CLL), blastic plasmacytoid dendritic cell neoplasm (BPDCN), peripheral T cell lymphoma, follicular lymphoma, diffuse large B cell lymphoma, Hodgkin lymphoma, non-Hodgkin lymphoma, neuroblastoma, a non-malignant inherited or acquired marrow disorder, multiple myeloma, and a dendritic cell neoplasm. In some embodiments, the cancer is a hematologic cancer. In some embodiments, the cancer is acute myeloid leukemia (AML). In some embodiments, the cancer is acute lymphoblastic leukemia (ALL). In some embodiments, the cancer is chronic myeloid leukemia (CML). In some embodiments, the cancer is chronic lymphocytic leukemia (CLL). In some embodiments, the cancer is blastic plasmacytoid dendritic cell neoplasm (BPDCN). In some embodiments, the cancer is peripheral T cell lymphoma. In some embodiments, the cancer is follicular lymphoma. In some embodiments, the cancer is diffuse large B cell lymphoma. In some embodiments, the cancer is Hodgkin lymphoma. In some embodiments, the cancer is non-Hodgkin lymphoma. In some embodiments, the cancer is neuroblastoma. In some embodiments, the cancer is a non-malignant inherited or acquired marrow disorder. In some embodiments, the cancer is multiple myeloma. In some embodiments, the cancer is a dendritic cell neoplasm.

In some embodiments, the cancer is the result of a non-malignant inherited or acquired marrow disorder. Examples of non-malignant inherited or acquired marrow disorders that are treated in accordance with the methods described herein include, but are not limited to, sickle anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, and congenital thrombocytopenia. In some embodiments, the non-malignant inherited or acquired marrow disorder is sickle cell anemia. In some embodiments, the non-malignant inherited or acquired marrow disorder is beta-thalassemia major. In some embodiments, the non-malignant inherited or acquired marrow disorder is refractory Diamond-Blackfan anemia. In some embodiments, the non-malignant inherited or acquired marrow disorder is myelodysplastic syndrome. In some embodiments, the non-malignant inherited or acquired marrow disorder is idiopathic severe aplastic anemia. In some embodiments, the non-malignant inherited or acquired marrow disorder is paroxysmal nocturnal hemoglobinuria. In some embodiments, the non-malignant inherited or acquired marrow disorder is pure red cell aplasia. In some embodiments, the non-malignant inherited or acquired marrow disorder is Fanconi anemia. In some embodiments, the non-malignant inherited or acquired marrow disorder is amegakaryocytosis. In some embodiments, the non-malignant inherited or acquired marrow disorder is congenital thrombocytopenia.

### Methods of Administration

Any anti-FLT3 antibody or fragment described herein, any pharmaceutical composition described herein, or any anti-FLT3 CAR expressing immune cells described herein can be administered to a subject by any suitable means which include, but are not limited to, parenteral (e.g., intravenous, intraarterial, intramuscular, intraosseous, intracerebral, intracerebroventricular, intrathecal, subcutaneous), intraperitoneal, intratumoral, intrapulmonary, intradermal, transdermal, conjunctival, intraocular, intranasal, intratracheal, oral and local intralesional routes of administration. In some embodiments, any anti-FLT3 antibody or fragment described herein, any pharmaceutical composition described herein, or any anti-FLT3 CAR expressing immune cell described herein (where reference to the immune cell also includes population of such immune cells) is administered intravenously, intraarterially, intraperitoneally, or intratumorally.

In some embodiments, any anti-FLT3 antibody or fragment described herein, any pharmaceutical composition described herein, or any anti-FLT3 CAR expressing immune cell described herein is administered intravenously (e.g., by a bolus or continuous infusion). In some embodiments, any anti-FLT3 antibody or fragment described herein, any pharmaceutical composition described herein, or any anti-FLT3 CAR expressing immune cell described herein is administered intraperitoneally. In some embodiments, any anti-FLT3 antibody or fragment described herein, any pharmaceutical composition described herein, or any anti-FLT3 CAR expressing immune cell described herein is administered intramuscularly. In some embodiments, any anti-FLT3 antibody or fragment described herein, any pharmaceutical composition described herein, or any anti-FLT3 CAR expressing immune cell described herein is administered subcutaneously. In some embodiments, any anti-FLT3 antibody or fragment described herein, any pharmaceutical composition described herein, or any anti-FLT3 CAR expressing immune cell described herein is administered intratumorally (such as by an injection into the tumor of the cancer being treated). In some embodiments, any anti-FLT3 antibody or fragment described herein, any pharmaceutical composition described herein, or any anti-FLT3 CAR expressing immune cell described herein is administered intravenously, intraperitoneally, or intratumorally.

Various dosing schedules of the anti-FLT3 antibodies and fragments described herein, pharmaceutical compositions described herein, and anti-FLT3 CAR expressing immune cells described herein are contemplated including single administration or multiple administrations over a period of time. The methods of administration include, without limitation, bolus administration, pulse infusions, and continuous infusions.

In some embodiments, any anti-FLT3 antibody or fragment described herein, any pharmaceutical composition described herein, or any anti-FLT3 CAR expressing immune cell described herein is administered one, two, three, four, five, six, seven, eight, nine, ten, eleven, twelve, thirteen, fourteen, or more times. In some embodiments, any anti-FLT3 antibody or fragment described herein, any pharmaceutical composition described herein, or any anti-FLT3 CAR expressing immune cell described herein is administered once. In some embodiments, any anti-FLT3 antibody or fragment described herein, any pharmaceutical composition described herein, or any anti-FLT3 CAR expressing immune cell described herein is effective in methods described herein when administered intravenously once (e.g., without further repeat administrations).

In some embodiments, any anti-FLT3 antibody or fragment described herein, any pharmaceutical composition described herein, or any anti-FLT3 CAR expressing immune cell described herein is administered every about 1 to 7 days for about 1 to 8 weeks. In some embodiments, any anti-FLT3 antibody or fragment described herein, any pharmaceutical composition described herein, or any anti-FLT3 CAR expressing immune cell described herein is administered every about 1 to 7 days for about 1 to 4 weeks. In some embodiments, any anti-FLT3 antibody or fragment described herein, any pharmaceutical composition described herein, or any anti-FLT3 CAR expressing immune cell described herein is administered every about 3 to 7 days for about 2 to 3 weeks. In some embodiments, any anti-FLT3 antibody or fragment described herein, any pharmaceutical composition described herein, or any anti-FLT3 CAR expressing immune cell described herein is administered from every about 3 days for about 2 weeks to every about 7 days for about 3 weeks. In some embodiments, any anti-FLT3 antibody or fragment described herein, any pharmaceutical composition described herein, or any anti-FLT3 CAR expressing immune cell described herein is administered every about 2 to 4 days for about 2 to 3 weeks (e.g., 2 weeks or 3 weeks).

In some embodiments, any anti-FLT3 antibody or fragment described herein, any pharmaceutical composition described herein, or any anti-FLT3 CAR expressing immune cell described herein is administered 1 day, 2 days, 3 days, 4 days, 5 days, 6 days, or 7 days a week (e.g., once a week, twice a week, every other day or every day). In some embodiments, any anti-FLT3 antibody or fragment described herein, any pharmaceutical composition described herein, or any anti-FLT3 CAR expressing immune cell described herein is administered for 1 week, 2 weeks, 3 weeks, 4 weeks, 5 weeks, 6 weeks, 7 weeks or 8 weeks. In some embodiments, any anti-FLT3 antibody or fragment described herein, any pharmaceutical composition described herein, or any anti-FLT3 CAR expressing immune cell described herein is administered for less than 6 weeks, less than 5 weeks, less than 4 weeks, less than 3 weeks or less than 2 weeks. In some embodiments, any anti-FLT3 antibody or fragment described herein, any pharmaceutical composition described herein, or any anti-FLT3 CAR expressing immune cell described herein is administered once in every two days or less frequently (e.g., for 1 to 3 weeks). In some embodiments, any anti-FLT3 antibody or fragment described herein, any pharmaceutical composition described herein, or any anti-FLT3 CAR expressing immune cell described herein is administered once in every three days or less frequently (e.g., for 1 to 3 weeks). In some embodiments, any anti-FLT3 antibody or fragment described herein, any pharmaceutical composition described herein, or any anti-FLT3 CAR expressing immune cell described herein is administered once in every four days or less frequently (e.g., for 1 to 3 weeks). In some embodiments, any anti-FLT3 antibody or fragment described herein, any pharmaceutical composition described herein, or any anti-FLT3 CAR expressing immune cell described herein is administered once in every five days or less frequently (e.g., for 1 to 3 weeks). In some embodiments, any anti-FLT3 antibody or fragment described herein, any pharmaceutical composition described herein, or any anti-FLT3 CAR expressing immune cell described herein is administered once a week or less frequently (e.g., for 1 to 3 weeks).

In some embodiments, the administration (of the antibodies, fragments, compositions or immune cells described herein) is every 3 days for about 2 weeks. In some embodiments, the administration is every 4 days for about 2 weeks. In some embodiments, the administration is every 5 days for about 2 weeks. In some embodiments, the administration is every 7 days for about 2 weeks. In some embodiments, the administration is every 3 days for about 3 weeks. In some embodiments, the administration is every 4 days for about 3 weeks. In some embodiments, the administration is every 5 days for about 3 weeks. In some embodiments, the administration is every 7 days for about 3 weeks.

In some embodiments, the administration is once a week for 1 week, 2 weeks, 3 weeks, 4 weeks, 5 weeks, or 6 weeks. In some embodiments, the administration is twice a week for 1 week, 2 weeks, 3 weeks, 4 weeks, 5 weeks, or 6 weeks. In some embodiments, the administration is three times a week for 1 week, 2 weeks, 3 weeks, 4 weeks, 5 weeks, or 6 weeks. In some embodiments, the administration is four times a week for 1 week, 2 weeks, 3 weeks, 4 weeks, 5 weeks, or 6 weeks. In some embodiments, the administration is five times a week for 1 week, 2 weeks, 3 weeks, 4 weeks, 5 weeks, or 6 weeks. In some embodiments, the administration is six times a week for 1 week, 2 weeks, 3 weeks, 4 weeks, 5 weeks, or 6 weeks. In some embodiments, the administration is seven times a week for 1 week, 2 weeks, 3 weeks, 4 weeks, 5 weeks, or 6 weeks.

In some embodiments, the administration is once every week, once every two weeks, once every three weeks, once every four weeks, once every five weeks, or once every 6 weeks. In some embodiments, the administration is once, two, three, four, five, six, seven, eight, nine ten, eleven, twelve, thirteen, fourteen, fifteen, sixteen, seventeen, eighteen, nineteen, or twenty times (e.g., in the course of treatment).

The administrations described herein include regimens wherein the initial dose of any therapy described herein is followed by one or more lower doses, or wherein the initial dose is followed by one or more higher doses. In some embodiments, the initial dose is followed by one or more lower doses. In some embodiments, the initial dose is followed by one or more higher doses.

In some embodiments, the initial treatment period (where any therapy described herein is administered, e.g., once a month, once in two weeks, once a week, twice a week or three times a week) is followed by a withdrawal period in which the therapy is not administered (for, e.g., a week, two weeks, three weeks, four weeks, six weeks, two months, three months, four months, six months or one year), and then followed by a second treatment period (where the therapy is administered, e.g., once a month, once in two weeks, once a week, twice a week or three times a week). Such initial treatment and such second treatment periods can last, for example, two weeks, three weeks, four weeks, six weeks (where the initial treatment period can be the same or different from the second treatment period). This course of treatment (having the initial treatment period, a withdrawal period and a second treatment period) can be repeated twice, three times, four times, five times, six times, ten times or more than ten times.

In some embodiments, a therapeutically effective amount of any anti-FLT3 antibody or fragment described herein, any pharmaceutical composition described herein, or any anti-FLT3 CAR expressing immune cells described herein is administered to a subject or patient. A therapeutically effective amount depends on the method used, the cancer being treated, the severity of cancer being treated, the route of administration, the target site, the condition of the patient (e.g., age, body weight, health), the responsiveness of the patient, other medications used by the patient, and other factors to be considered at the discretion of the medical practitioner performing the treatment.

In some embodiments, the anti-FLT3 CAR expressing immune cells (e.g., T cells) are administered in an amount of about 1×10⁶, about 5×10⁶, about 1×10⁷, about 2×10⁷, about 3×10⁷, about 4×10⁷, about 5×10⁷, about 6×10⁷, is about 7×10⁷, about 8×10⁷, about 9×10⁷, about 1×10⁸, about 2×10⁸, about 3×10⁸, is about 4×10⁸, about 5×10⁸, about 6×10⁸, is about 7×10⁸, about 8×10⁸, about 9×10⁸, about 1×10⁹, about 2×10⁹, about 3×10⁹, is about 4×10⁹, about 5×10⁹, about 6×10⁹, is about 7×10⁹, about 8×10⁹, about 9×10⁹, about 1×10¹⁰, about 2×10¹⁰, about 3×10¹⁰, is about 4×10¹⁰, or about 5×10¹⁰ cells.

In some embodiments, the anti-FLT3 CAR expressing immune cells (e.g., T cells) are administered in an amount of about 5×10⁷, about 6×10⁷, is about 7×10⁷, about 8×10⁷, about 9×10⁷, about 1×10⁸, about 2×10⁸, about 3×10⁸, is about 4×10⁸, about 5×10⁸, about 6×10⁸, is about 7×10⁸, about 8×10⁸, about 9×10⁸, about 1×10⁹, about 2×10⁹, about 3×10⁹, is about 4×10⁹, about 5×10⁹, about 6×10⁹, is about 7×10⁹, about 8×10⁹, about 9×10⁹, or 1×10¹⁰ cells.

In some embodiments, the anti-FLT3 CAR expressing immune cells (e.g., T cells) are administered in an amount of about 1×10⁸, about 2×10⁸, about 3×10⁸, is about 4×10⁸, about 5×10⁸, about 6×10⁸, is about 7×10⁸, about 8×10⁸, about 9×10⁸, about 1×10⁹, about 2×10⁹ cells.

In some embodiments, the anti-FLT3 CAR expressing immune cells (e.g., T cells) are administered in an amount from about 5×10⁷ to about 1×10¹⁰ cells. In some embodiments, the anti-FLT3 CAR expressing immune cells (e.g., T cells) are administered in an amount from about 1×10⁸ to about 2×10⁹ cells.

In some embodiments, the dosage of any anti-FLT3 antibody or fragment described herein is from about 0.01 mg/kg to about 10 mg/kg of the patient's body weight. In some embodiments, the dosage of any anti-FLT3 antibody or fragment described herein is from about 0.01 mg/kg to about 2 mg/kg of the patient's body weight. In some embodiments, the dosage of any anti-FLT3 antibody or fragment described herein is from about 0.05 mg/kg to about 1 mg/kg of the patient's body weight. In some embodiments, the dosage of any anti-FLT3 antibody or fragment described herein is from about 0.1 mg/kg to about 0.5 mg/kg of the patient's body weight. In some embodiments, the dosage of any anti-FLT3 antibody or fragment described herein is from about 0.1 mg/kg to about 0.3 mg/kg of the patient's body weight. In some embodiments, the dosage of any anti-FLT3 antibody or fragment described herein is about 0.01 mg/kg, about 0.1 mg/kg, about 0.5 mg/kg, about 1 mg/kg, about 1.5 mg/kg, or about 2 mg/kg of the patient's body weight. In some embodiments, the dosage of any anti-FLT3 antibody or fragment described herein is about 0.1 mg/kg of the patient's body weight. In some embodiments, the dosage of any anti-FLT3 antibody or fragment described herein is about 0.2 mg/kg of the patient's body weight. In some embodiments, the dosage of any anti-FLT3 antibody or fragment described herein is about 0.3 mg/kg of the patient's body weight.

In some embodiments, the hematopoietic cell transplantation occurs 5 days to 5 weeks after the administering of any anti-FLT3 antibody or fragment described herein, any pharmaceutical composition described herein, or any anti-FLT3 CAR expressing immune cells described herein. In some embodiments, the performing of the hematopoietic cell transplantation occurs about 2 to 3 weeks after the administering of any anti-FLT3 antibody or fragment described herein, any pharmaceutical composition described herein, or any anti-FLT3 CAR expressing immune cells described herein. In some embodiments, the performing of the hematopoietic cell transplantation occurs about 1 week to 4 weeks after the administering. In some embodiments, the performing of the hematopoietic cell transplantation occurs about 10 days to 25 days after the administering. In some embodiments, the performing of the hematopoietic cell transplantation occurs about 10 days to 20 days after the administering. In some embodiments, the performing of the hematopoietic cell transplantation occurs about 2 weeks after the administering. In some embodiments, the performing of the hematopoietic cell transplantation occurs about 3 weeks after the administering. In some embodiments, the performing of the hematopoietic cell transplantation occurs at least 5 days or 1 week after the administering. In some embodiments, the performing of the hematopoietic cell transplantation occurs at least 2 weeks after the administering. In some embodiments, the performing of the hematopoietic cell transplantation occurs less than 3 weeks after the administering. In some embodiments, the performing of the hematopoietic cell transplantation occurs less than 4 weeks after the administering. In some embodiments, the performing of the hematopoietic cell transplantation occurs less than 5 weeks after the administering.

### Patient Populations

In some embodiments, a patient or subject is treated with any anti-FLT3 antibody or fragment described herein, any pharmaceutical composition described herein, or any anti-FLT3 CAR expressing immune cell described herein. In some embodiments, the patient or subject is a mammal, e.g. a human, a non-human primate, a dog, a cat, a rabbit, a cow, a horse, a goat, a sheep, or a pig. In some embodiments, the subject is a human.

In some embodiments, the patient or subject being treated in accordance with the methods described herein has (e.g., has been diagnosed with) cancer. Methods for cancer diagnosis are known in the art. In some embodiments, the cancer is early stage cancer. In some embodiments, the cancer is advanced stage cancer.

In some embodiments, the patient or subject being treated in accordance with the methods described herein has (e.g., has been diagnosed with) a hematopoietic or hematologic cancer. In some embodiments, the hematologic cancer is Acute Myeloid Leukemia (AML), Acute Lymphoblastic Leukemia (ALL), Chronic Lymphocytic Leukemia (CLL), Chronic Myeloid Leukemia (CML), blastic plasmacytoid dendritic cell neoplasm (BPDCN), peripheral T cell lymphoma, follicular lymphoma, diffuse large B cell lymphoma, Hodgkin lymphoma, non-Hodgkin lymphoma, neuroblastoma, multiple myeloma, a non-malignant inherited or acquired marrow disorder, or a dendritic cell neoplasm.

In some embodiments, the patient or subject being treated in accordance with the methods described herein has been diagnosed with Acute Myeloid Leukemia (AML). In some embodiments, the patient or subject being treated in accordance with the methods described herein has been diagnosed with Acute Lymphoblastic Leukemia (ALL). In some embodiments, the patient or subject being treated in accordance with the methods described herein has been diagnosed with Chronic Lymphocytic Leukemia (CLL). In some embodiments, the patient or subject being treated in accordance with the methods described herein has been diagnosed with Chronic Myeloid Leukemia (CML). In some embodiments, the patient or subject being treated in accordance with the methods described herein has been diagnosed with blastic plasmacytoid dendritic cell neoplasm (BPDCN). In some embodiments, the patient or subject being treated in accordance with the methods described herein has been diagnosed with peripheral T cell lymphoma. In some embodiments, the patient or subject being treated in accordance with the methods described herein has been diagnosed with follicular lymphoma. In some embodiments, the patient or subject being treated in accordance with the methods described herein has been diagnosed with diffuse large B cell lymphoma. In some embodiments, the patient or subject being treated in accordance with the methods described herein has been diagnosed with Hodgkin lymphoma. In some embodiments, the patient or subject being treated in accordance with the methods described herein has been diagnosed with non-Hodgkin lymphoma. In some embodiments, the patient or subject being treated in accordance with the methods described herein has been diagnosed with neuroblastoma. In some embodiments, the patient or subject being treated in accordance with the methods described herein has been diagnosed with multiple myeloma. In some embodiments, the patient or subject being treated in accordance with the methods described herein has been diagnosed with a dendritic cell neoplasm.

In some embodiments, the patient or subject being treated in accordance with the methods described herein has (e.g., has been diagnosed with) a non-malignant inherited acquired marrow disorder. In some embodiments, the non-malignant inherited acquired marrow disorder is sickle cell anemia, beta-thalassemia major, refractory Diamond-Blackfan anemia, myelodysplastic syndrome, idiopathic severe aplastic anemia, paroxysmal nocturnal hemoglobinuria, pure red cell aplasia, Fanconi anemia, amegakaryocytosis, congenital thrombocytopenia, or Severe Combined Immunodeficiency (SCID).

In some embodiments, the patient or subject being treated in accordance with the methods described herein has been diagnosed with sickle cell anemia. In some embodiments, the patient or subject being treated in accordance with the methods described herein has been diagnosed with beta-thalassemia major. In some embodiments, the patient or subject being treated in accordance with the methods described herein has been diagnosed with refractory Diamond-Blackfan anemia. In some embodiments, the patient or subject being treated in accordance with the methods described herein has been diagnosed with myelodysplastic syndrome. In some embodiments, the patient or subject being treated in accordance with the methods described herein has been diagnosed with idiopathic severe aplastic anemia. In some embodiments, the patient or subject being treated in accordance with the methods described herein has been diagnosed with paroxysmal nocturnal hemoglobinuria. In some embodiments, the patient or subject being treated in accordance with the methods described herein has been diagnosed with pure red cell aplasia. In some embodiments, the patient or subject being treated in accordance with the methods described herein has been diagnosed with Fanconi anemia. In some embodiments, the patient or subject being treated in accordance with the methods described herein has been diagnosed with amegakaryocytosis. In some embodiments, the patient or subject being treated in accordance with the methods described herein has been diagnosed with congenital thrombocytopenia. In some embodiments, the patient or subject being treated in accordance with the methods described herein has been diagnosed with Severe Combined Immunodeficiency (SCID).

In some embodiments, the patient or subject being treated has previously undergone one or more cancer therapies (e.g. vaccine, small molecule targeted therapy, chemotherapy, radiotherapy, or immunotherapy), and has developed resistance to one or more of the previous cancer therapies. In some embodiments, the patient or subject being treated is resistant to chemotherapy. In some embodiments, the patient or subject being treated is resistant to small molecule targeted therapy. In some embodiments, the patient or subject being treated is resistant to another immunotherapy.

In some embodiments, the patient or subject has a type of cancer that is known or expected to express FLT3 on the surface of its cells.

In some embodiments, the patient or subject being treated has a cancer that has been determined, using methods known in the art, to express FLT3 on the surface of its cells that can be targeted by any anti-FLT3 antibody or fragment described herein or any anti-FLT3 CAR expressing immune cell described herein.

In some embodiments, the patient or subject being treated in accordance with the methods described herein is in need of hematopoietic cell transplantation. In some embodiments, the patient or subject being treated in accordance with the methods described herein is in need of bone marrow transplantation with hematopoietic stem cells and/or hematopoietic progenitor cells.

### Combination Therapies and Kits

In some embodiments, any anti-FLT3 antibody or fragment described herein, any pharmaceutical composition described herein, or any anti-FLT3 CAR expressing immune cell described herein is administered to a subject in combination with one or more anti-cancer therapies. In some embodiments, the anti-cancer therapy is a chemotherapy, surgery, radiation therapy, an antibody therapy, a small molecule therapy, or another anti-cancer therapy known in the art.

In some embodiments, any anti-FLT3 antibody or fragment described herein, any pharmaceutical composition described herein, or any anti-FLT3 CAR expressing immune cell described herein is administered to a subject in combination with chemotherapy. Examples of types of chemotherapeutic agents that can be used in the methods described herein include, without limitation, an alkylating agent, a nitrosourea agent, an antimetabolite, a topoisomerase inhibitor, an aromatase inhibitor, an antitumor antibiotic, an alkaloid derived from a plant, a hormone antagonist, a P-glycoprotein inhibitor, and a platinum complex derivative. Specific examples of chemotherapeutic drugs that can be used in the methods described herein include, without limitation, taxol, paclitaxel, nab-paclitaxel, 5-fluorouracil (5-FU), gemcitabine, daunorubicin, colchicin, mitoxantrone, tamoxifen, cyclophosphamide, mechlorethamine, busulfan, uramustine, mustargen, ifosamide, bendamustine, carmustine, lomustine, semustine, fotemustine, streptozocin, thiotepa, mitomycin, diaziquone, tetrazine, altretamine, mitozolomide, temozolomide, procarbazine, hexamethylmelamine, altretamine, hexalen, trofosfamide, estramustine, treosulfan, mannosulfan, triaziquone, carboquone, nimustine, ranimustine, azathioprine, sulfanilamide, fluoropyrimidine, thiopurine, thioguanine, mercaptopurine, cladribine, capecitabine, pemetrexed, fludarabine, hydroxyurea, nelarabine or clofarabine, cytarabine, decitabine, pralatrexate, floxuridine, thioquanine, azacitidine, cladribine, pentostatin, mercaptopurine, imatinib, dactinomycin, cerubidine, actinomycin, luteomycin, epirubicin, idarubicin, plicamycin, vincristin, vinorelbine, vinflunine, paclitaxel, docetaxel, etoposide, teniposide, periwinkle, vinca, taxane, irinotecan, topotecan, camptothecin, teniposide, pirarubicin, novobiocin, merbarone, aclarubicin, amsacrine, antiandrogen, anti-estrogen, bicalutamide, medroxyprogesterone, fluoxymesterone, diethylstilbestrol, estrace, octreotide, megestrol, raloxifene, toremifene, fulvestrant, prednisone, flutamide, leuprolide, goserelin, aminoglutethimide, testolactone, anastrozole, letrozole, exemestane, vorozole, formestane, fadrozole, androstene, resveratrol, myosmine, catechin, apigenin eriodictyol isoliquiritigenin, mangostin, amiodarone, azithromycin, captopril, clarithromycin, cyclosporine, piperine, quercetine, quinidine, quinine, reserpine, ritonavir, tariquidar, verapamil, cisplatin, carboplatin, oxaliplatin, transplatin, nedaplatin, satraplatin, triplatin and carboplatin.

In some embodiments, any anti-FLT3 antibody or fragment described herein, any pharmaceutical composition described herein, or any anti-FLT3 CAR expressing immune cell described herein is administered to a subject in combination with one or more antitumor agents selected from the following group: anthracyclines (e.g. daunomycin and doxorubicin), auristatin, methotrexate (MTX), vindesine, neocarzinostatin, cis-platinum, chlorambucil, cytosine arabinoside, 5-fluorouridine, melphalan, ricin and calicheamicin including combination chemotherapy such with doxorubicin, bleomycin, vinblastine, and dacarbazine (ABVD), BEACOPP or escalated BEACOPP (bleomycin, etoposide, doxorubicin, cyclophosphamide, vincristine, procarbazine, and prednisone) and Stanford V (doxorubicin, vinblastine, mechlorethamine, vincristine, bleomycin, etoposide, and prednisone). In some embodiments, any anti-FLT3 antibody or fragment described herein, any pharmaceutical composition described herein, or any anti-FLT3 CAR expressing immune cell described herein is administered to a subject in combination with one or more of: immunotherapy (e.g. anti-CD20 antibody rituximab), immunotoxin (e.g., Brentuximab vedotin (SGN-35), which is an immunotoxin comprised of a CD-30 directed antibody linked to the antitubulin agent monomethyl auristatin E (MMAE)), adoptive immunotherapy (cytotoxic T lymphocytes), programmed death 1 (PD-1) blockade (e.g., nivolumab, pembrolizumab).

In some embodiments, any anti-FLT3 antibody or fragment described herein, any pharmaceutical composition described herein, or any anti-FLT3 CAR expressing immune cell described herein is administered to a subject with a cancer in combination with the chemotherapy drug(s) indicated for said cancer, which chemotherapy drug(s) can be optionally administered in the dosage and/or regime of administration indicated for said cancer (e.g., AML or ALL).

In some embodiments, any anti-FLT3 antibody or fragment described herein, any pharmaceutical composition described herein, or any anti-FLT3 CAR expressing immune cell described herein is administered to a subject in combination with immunotherapy. In some embodiments, the immunotherapy comprises administering a checkpoint inhibitor. In some embodiments, the checkpoint inhibitor is an anti-PD1 antagonist, an anti-PD-L1 antagonist, and an anti-CTLA4 antagonist. In some embodiments, the checkpoint inhibitor is an anti-PD1 antagonist. In some embodiments, the checkpoint inhibitor is an anti-PD-1 antibody (such as an antagonistic anti-PD-1 antibody). In some embodiments, the checkpoint inhibitor is an anti-PD-L1 antagonist. In some embodiments, the checkpoint inhibitor is an anti-PD-L1 antibody (such as an antagonistic anti-PD-L1 antibody). In some embodiments, the checkpoint inhibitor is an anti-CTLA4 antagonist (e.g., an antagonistic anti-CTLA4 antibody). In some embodiments, the checkpoint inhibitor is a Lag3 antagonist. In some embodiments, the checkpoint inhibitor is Tim3 antagonist. In some embodiments, the checkpoint inhibitor is a TIGIT antagonist. In some embodiments, the checkpoint inhibitor is an OX40 antagonist.

In some embodiments, the anti-PD1 antagonist is selected from the group consisting of, but not limited to, nivolumab, pembrolizumab, PDR001, Pembrolimumab (Bio X Cell), Bio X Cell Clone J116 (Cat. #BE0188), cemiplimab, and pidilizumab. In some embodiments, the anti-PD-L1 antagonist is selected from the group consisting of, but not limited to, atezolizumab, avelumab, durvalumab, YW243.55.S70, MPDL3280A, MDX-1105, and BMS-936559 In some embodiments, the anti-CTLA4 antagonist is selected from, but not limited to, ipilimumab and tremelimumab.

In some embodiments, an immune cell comprising an anti-FLT3 CAR described herein, such as an anti-FLT3 CAR having the amino acid sequence of any one of the following SEQ ID NOs: 6 and 9-15, or an immune cell comprising any one of the following nucleic acid sequences. SEQ ID Nos: 60 and 63-69, is administered to a subject in combination with a chemotherapy. In some embodiments, any anti-FLT3 antibody or fragment having the VH and/or VL described herein or any scFv described herein is administered to a subject in combination with a chemotherapy.

In some embodiments, an immune cell comprising an anti-FLT3 CAR described herein, such as an anti-FLT3 CAR having the amino acid sequence of any one of the following SEQ ID NOs: 6 and 9-15, or an immune cell comprising any one of the following nucleic acid sequences: SEQ ID Nos: 60 and 63-69, is administered to a subject in combination with an immunotherapy (e.g., a checkpoint inhibitor). In some embodiments, any anti-FLT3 antibody or fragment having the VH and/or VL described herein or any scFv described herein is administered to a subject in combination with an immunotherapy (e.g., a checkpoint inhibitor).

In some embodiments, an immune cell comprising an anti-FLT3 CAR described herein, such as an anti-FLT3 CAR having the amino acid sequence of any one of the following SEQ ID NOs: 6 and 9-15, or an immune cell comprising any one of the following nucleic acid sequences: SEQ ID Nos: 60 and 63-69, is administered to a subject in combination with an anti-PD1 antagonist (e.g., an anti-PD1 antibody). In some embodiments, an immune cell comprising an anti-FLT3 CAR described herein, such as an anti-FLT3 CAR having the amino acid sequence of any one of the following SEQ ID NOs: 6 and 9-15, or an immune cell comprising any one of the following nucleic acid sequences: SEQ ID Nos: 60 and 63-69, is administered to a subject in combination with an anti-PD-L1 antagonist (e.g., an anti-PDL1 antibody). In some embodiments, an immune cell comprising an anti-FLT3 CAR described herein, such as an anti-FLT3 CAR having the amino acid sequence of any one of the following SEQ ID NOs: 6 and 9-15, or an immune cell comprising any one of the following nucleic acid sequences: SEQ ID Nos: 60 and 63-69, is administered to a subject in combination with an anti-CTLA4 antagonist (e.g., anti-CTLA4 antibody). In some embodiments, any anti-FLT3 antibody or fragment having the VH and/or VL described herein or any scFv described herein is administered to a subject in combination with any anti-PD-1 antagonist, any anti-PD-L1 antagonist or any anti-CTLA4 antagonist described herein.

In some embodiments, any anti-FLT3 antibody or fragment described herein, any pharmaceutical composition described herein, or any anti-FLT3 CAR expressing immune cell described herein is administered to a subject in combination with radiation therapy (e.g., x-ray, gamma ray, electron beams).

In some embodiments, the checkpoint inhibitor is administered prior to administration of any anti-FLT3 antibody or fragment described herein, any pharmaceutical composition described herein, or any anti-FLT3 CAR expressing immune cell described herein. In some embodiments, the checkpoint inhibitor is administered concomitantly with any anti-FLT3 antibody or fragment described herein, any pharmaceutical composition described herein, or any anti-FLT3 CAR expressing immune cell described herein. In some embodiments, the checkpoint inhibitor is administered after administration of any anti-FLT3 antibody or fragment described herein, any pharmaceutical composition described herein, or any anti-FLT3 CAR expressing immune cell described herein.

In some embodiments, any anti-FLT3 antibody or fragment described herein, any pharmaceutical composition described herein, or any anti-FLT3 CAR expressing immune cell described herein is administered to a subject before, during, or after a second therapy.

In some embodiments, the subject being treated in accordance with the methods described herein has not received an anti-cancer therapy prior to the administration of any anti-FLT3 antibody or fragment described herein, any pharmaceutical composition described herein, or any anti-FLT3 CAR expressing immune cell described herein. In some embodiments, any anti-FLT3 antibody or fragment described herein, any pharmaceutical composition described herein, or any anti-FLT3 CAR expressing immune cell described herein is administered to a subject that has received an anti-cancer therapy prior to administration of the antibody or fragment. In some, embodiments, any anti-FLT3 antibody or fragment described herein, any pharmaceutical composition described herein, or any anti-FLT3 CAR expressing immune cell described herein is administered to a subject recovering from or receiving an immunosuppressive therapy.

In some embodiments, provided herein are kits comprising any anti-FLT3 antibody or fragment described herein, any pharmaceutical composition described herein, or any anti-FLT3 CAR expressing immune cells described herein and one or more additional anti-cancer agents. In some embodiments, provided herein are kits comprising (i) any anti-FLT3 antibody or fragment described herein, any pharmaceutical composition described herein, or any anti-FLT3 CAR expressing immune cells described herein (e.g., in a therapeutically effective amount), and (ii) one or more chemotherapeutic drugs (e.g., in a therapeutically effective amount, which may be less than the therapeutic amount of the drug or drugs when used without the antibody, fragment or immune cell). In some embodiments, provided herein are kits comprising (i) any anti-FLT3 antibody or fragment described herein, any pharmaceutical composition described herein, or any anti-FLT3 CAR expressing immune cells described herein (e.g., in a therapeutically effective amount), and (ii) one or more checkpoint inhibitors described herein (e.g., in a therapeutically effective amount, which may be less than the therapeutic amount of the drug or drugs when used without the antibody, fragment or immune cell). In some embodiments, provided herein are kits comprising (i) any anti-FLT3 antibody or fragment described herein, any pharmaceutical composition described herein, or any anti-FLT3 CAR expressing immune cells described herein (e.g., in a therapeutically effective amount), and (ii) one or more anti-PD1 antibody, anti-PD-li antibody or anti-CTLA4 antibody (e.g., in a therapeutically effective amount, which may be less than the therapeutic amount of the drug or drugs when used without the antibody, fragment or immune cell).

The following examples are offered by way of illustration and not by way of limitation. Various other embodiments of the invention may be practiced, given the general description provided herein.

### EXAMPLES

### Example 1: Generation of Humanized Antibodies and Single Chain Variable Fragments

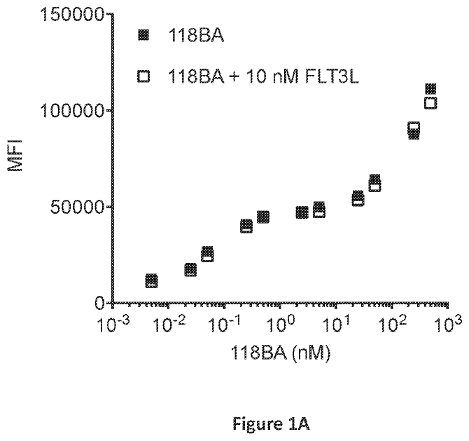

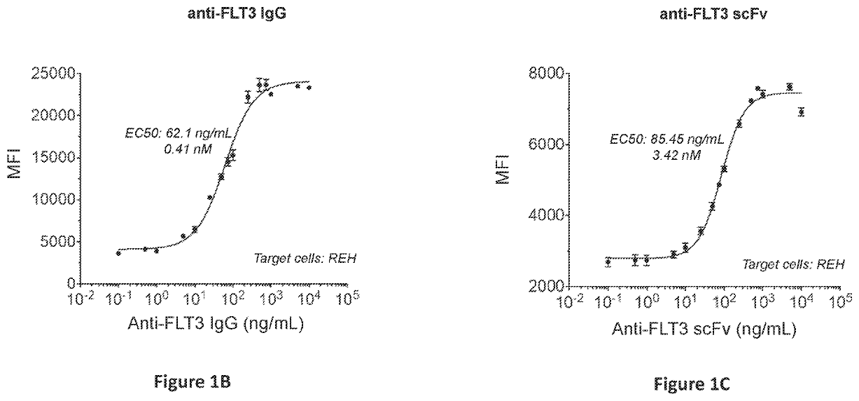

Prior to humanization, a chimeric anti-FLT3 antibody was evaluated for competitive binding with FLT3 ligand (FLT3L). Specifically, REH cells were incubated with 10 nM of recombinant human FLT3L (R&D systems) for 20 minutes and washed with PBS+2% BCS+2 mM EDTA (flow buffer). Cells were then stained with various concentrations of the chimeric monoclonal antibody prepared in flow buffer. Cells were washed five times with flow buffer and stained with anti-human IgG Fc antibody conjugated to Alexa Fluor 488 (Jackson Immunoresearch Laboratories, 109-545-008). Cells were stained with 7-AAD (7-AAD Viability Staining Solution, Biolegend 420404) followed by flow cytometry analysis. The binding of the chimeric antibody 1-18BA (comprising a mouse VL (SEQ ID NO: 28) and mouse VH (SEQ ID NO:17) and human IgG) was not reduced with FLT3L pre-treatment, and instead was about the same as without the FLT3L pre-treatment, as shown in FIG. 1A. This suggests that 118BA does not compete with FLT3L for binding to FLT3.

To generate the humanized antibodies and single-chain variable fragments described herein, the following methods were used.

### Materials and Methods

### Variable Domain Analysis and CDR Identification

For the purpose of identifying CDRs and analyzing the closest matching germline sequences the IMGT Domain Gap align tool was used: http_www_imgt.org/3Dstructure-DB/cgi/DomainGapAlign.cgi.

### Molecular Modelling

Molecular models were built for VH and VL domains based on homology to previously published antibody crystal structures using software.

### Gene Synthesis and Cloning

Variable heavy and variable light domains (for FLT3) were designed with appropriate restriction sites at the 5′ and 3′ ends to enable cloning into Absolute Antibody cloning and expression vectors. Variable domains sequences were codon optimized for expression in human cells. Following gene synthesis the variable domains were cloned into Absolute Antibody vectors of the appropriate species and type. The correct sequence was verified by Sanger sequencing with raw data analyzed using DNASTAR Lasergene software. Once confirmed plasmid DNA preps of the appropriate size were performed to generate a sufficient quantity of high quality DNA for transfection.

### Expression and Purification

Once the plasmids were generated, HEK 293 (human embryonic kidney 293) mammalian cells were passaged to the optimum stage for transient transfection. Cells were transiently transfected with heavy and light chain expression vectors and cultured for a further 6 days. Cultures were harvested by centrifugation at 4000 rpm and filtered through a 0.22 μM filter. A first step of purification was performed by Protein A affinity chromatography with elution using citrate pH3.0 buffer followed by neutralization with 0.5M Tris, pH 9.0. Eluted protein was then buffer exchanged into PBS using a desalting column. Antibody concentration was determined by UV spectroscopy and the antibodies concentrated as necessary.

### Antibody Analytics

Antibody purity was determined by SDS-PAGE (sodium dodecyl sulphate polyacrylamide gel electrophoresis) and HPLC (high performance liquid chromatography). SEC-HPLC was performed on an Agilent 1100 series instrument using an appropriate size exclusion column (SEC). Antibody expression titre was determined by Protein A HPLC.

### Humanization

### Sequence Analysis

The VH and VL sequences for 1-18BA (SEQ ID NO: 17 and SEQ ID NO: 28, respectively), which were generated using methods described in US Patent Pub. No. 20190389955 (the entirety of which is incorporated herein), were run through the IGMT Gap Align tool to analyze against all known antibody germline sequences. CDR regions were assigned using the IMGT definition. The sequence is most clearly aligned to muse, specifically the IGHV8-8 family for the VH and IGKV9-124 for the VL.

### Molecular Modelling

To enable structure guided humanization, models were built for the 1-18BA murine VH and VL sequences.

### Germline Selection

The VH and VL sequences were aligned with an Absolute Antibody database of human germline sequences. Table 1 shows the germline sequences that were selected as frameworks for humanization.

TABLE 1
Heavy and light chain germline sequences selected
as humanization frameworks and their percent identity
to the original murine VH and VL sequences.

| | Germline ID | % Identity |
|---|---|---|
| Heavy Chain | IGHV2-5*05 | 68.4 |
| | IGHV4-38-2*02 | 61.6 |
| | IGHV6-1*01 | 50 |
| Light Chain | IGKV1-16*01 | 71.6 |
| | IGKV3-20*01 | 59.4 |
| | IGKV4-1*01 | 58.4 |

### CDR Grafting

To humanize the antibodies, the VH and VL sequences were run through a CDR grafting algorithm to transfer the CDRs from the murine antibody 1-18BA (having VH of SEQ ID NO: 17 and VL of SEQ ID NO: 28) onto the selected human germline sequences. Although CDRs are defined as being primarily responsible for binding to an antigen it is possible for amino acids outside of these regions, in what are known as framework regions, to either be involved directly in binding or to play a role in correctly orientating the CDRs. A structure guided approach was used to determine which of the framework amino acids to retain in the as the original mouse amino acid for the sake of retaining binding integrity. Table 2 summarizes the sequences that were generated.

TABLE 2
Original mouse and humanized sequences generated for 1-18BA

| | | | | % | |
|---|---|---|---|---|---|
| | | | | Identity | SEQ |
| | | Human | | to | ID |
| ID | Format | Germline | Sequence | human | NO |
| cAb1977 | Mouse | n/a | QVTLKESGPGILQPSQTLSLTCSFSG | | 17 |
| -VH | | | FSLSTSTMGVGWIRQPSGKGLEWL | | |
| | | | LHILWNDSKYYNPALKSRLTISKDT | | |
| | | | YNKQVFLKIANVDTADTATYYCAR | | |
| | | | IVYYSTYVGYFDVWGAGTTVTVSS | | |
| cAb1978 | Humanized | IGHV2 | QVTLKESGPTLVKPTQTLTLTCTFS | 91.8 | 3 |
| -VH | | -5*05 | GFSLSTSTMGVGWIRQPPGKALEW | | |
| | | | LAHILWNDSKRYNPSLKSRLTITKD | | |
| | | | TSKKQVVLTMTNMDPVDTATYYC | | |
| | | | ARIVYYSTYVGYFDVWGQGTTVTV | | |
| | | | SS | | |
| cAb1981 | Humanized | IGHV2 | QITLKESGPTLVKPTQTLTLTCTFSG | 93.9 | 18 |
| -VH | | -5*05 | FSLSTSTMGVGWIRQPPGKALEWL | | |
| | | | AHILWNDDKRYGPSLKSRLTITKDT | | |
| | | | SKKQVVLTMTNMDPVDTATYYCA | | |
| | | | RIVYYSTYVGYFDVWGQGTTVTVS | | |
| | | | S | | |
| cAb1984 | Humanized | IGHV4 | QVQLQESGPGLVKPSETLSLTCTFS | 83.8 | 22 |
| -VH | | -38- | GFSLSTSTMGVGWIRQPPGKGLEWI | | |
| | | 2*02 | GHILWNDSKYYNPSLKSRVTISKDT | | |
| | | | SKKQFSLKLSSVTAADTAVYYCARI | | |
| | | | VYYSTYVGYFDVWGQGTTVTVSS | | |
| cAb1987 | Humanized | IGHV6 | QVQLQQSGPGLVKPSQTLSLTCAFS | 76.2 | 25 |
| -VH | | -1*01 | GFSLSTSTMGVGWIRQSPSRGLEWL | | |
| | | | GHILWNDSKDYNPSVKSRITINKDT | | |
| | | | SKKQFSLQLNSVTPEDTAVYYCARI | | |
| | | | VYYSTYVGYFDVWGQGTTVTVSS | | |
| cAb1977 | Mouse | n/a | DIQMTQSPSSLSASLGERVSLTCRA | | 28 |
| -VL | | | SQEISGYLSWLQQKPDGTIKRLIYA | | |
| | | | ASTLHSGVPKRFSGSRSGSDYSLTIS | | |
| | | | RLESEDVADYYCLQYASYPFTFGS | | |
| | | | GTKLEIK | | |
| cAb1978 | Humanized | IGKV1 | DIQMTQSPSSLSASVGDRVTITCRA | 87.4 | 1 |
| -VL | | -16*01 | SQEISGYLSWLQQKPGKAIKRLIYA | | |
| | | | ASTLQSGVPSRFSGSRSGSDYTLTIS | | |
| | | | SLQPEDFATYYCLQYASYPFTFGQG | | |
| | | | TKLEIK | | |
| cAb1979 | Humanized | IGKV3 | EIVMTQSPGTLSLSPGERATLSCRAS | 82.3 | 29 |
| -VL | | -20*01 | QEISGYLSWLQQKPGQAIRRLIYAA | | |
| | | | STRATGIPDRFSGSRSGSDYTLTISR | | |
| | | | LEPEDFAVYYCLQYASYPFTFGQGT | | |
| | | | KLEIK | | |
| cAb1980 | Humanized | IGKV4 | DIVMTQSPDSLAVSLGERATINCKA | 78.2 | 2 |
| -VL | | -1*01 | SQEISGYLSWLQQKPGQPIKRLIYA | | |
| | | | ASTRESGVPDRFSGSRSGSDYTLTIS | | |
| | | | SLQAEDVAVYYCLQYASYPFTFGQ | | |
| | | | GTKLEIK | | |

### Sequence Liability Analysis

To ensure that no highly undesirable sequence liabilities had been introduced into the humanized sequences the original mouse and humanized sequences were run through an Absolute Antibody sequence liability tool. Sequence liabilities of most concern are glycosylation motifs and free Cysteins. The original parental VH sequence contains an N-linked glycosylation motif. The motif is within CDR-H2 and may have an impact on binding. This motif was left in most of the humanized VH sequences apart from cAb1981.

### Antibody Production and Analytics

### Antibody Cloning

As described above, a total of 4 humanized heavy chains and 3 humanized light chains were designed. Each of these were synthesized separately and cloned as both human IgG1s and scFvs. At the point of transfection all possible combinations of the humanized sequences were made to create a total of 12 different humanized IgGs and 12 humanized scFvs.

### Antibody Expression and Purification

All antibodies were expressed at small scale and the proteins then purified by either Protein A or Nickel chromatography. All the purified products looked as expected under non-reducing and reducing SDS-PAGE. With the exception of cAB1984-10.0, all IgGs expressed well. The scFvs showed mixed expression levels with 6 of them failing to express complete (cAb1977, cAb1081, cAb1982, cAb1983 and cAb1988). IgG and scFV yields are shown in Tables 3 and 4.

TABLE 3
Table showing chimeric human IgG and humanized IgGs.
VH and VL protein sequences are shown along with
the percentage identity to human germline sequences,
titre amount of final antibody purified and
the monomer content.

| | | VH | VL | | Titre | Purified | Monomer |
|---|---|---|---|---|---|---|---|
| ID | Type | sequence | sequence | | (mg/l) | (mg) | (%) |
| cAb1977-10.0 | Chimeric | [symbol] | [symbol] | n/a | 59 | 4.7 | 100 |
| | human IgG1 | | | | | | |
| cAb1978-10.0 | Humanized | [symbol] | [symbol] | VH: 91.8 | 150 | 12.0 | 100 |
| | human IgG1 | | | VL: 87.4 | | | |
| cAb1979-10.0 | Humanized | [symbol] | [symbol] | VH: 91.8 | 106 | 8.5 | 100 |
| | human IgG1 | | | VL: 82.3 | | | |
| cAb1980-10.0 | Humanized | [symbol] | [symbol] | VH: 91.8 | 113 | 9.0 | 99.2 |
| | human IgG1 | | | VL: 78.2 | | | |
| cAb1981-10.0 | Humanized | [symbol] | [symbol] | VH: 3.9 | 148 | 11.8 | 99.1 |
| | haman IgG1 | | | VL: 87.4 | | | |
| cAb1982-10.0 | Humanized | [symbol] | [symbol] | VH: 93.9 | 100 | 8.0 | 100 |
| | human IgG1 | | | VL: 82.3 | | | |
| cAb1983-10.0 | Humanized | [symbol] | [symbol] | VH: 93.9 | 60 | 4.8 | 98.3 |
| | honnan IgG1 | | | VL: 78.2 | | | |
| cAb1984-10.0 | Humanized | [symbol] | [symbol] | VH: 83.8 | 6 | 0.5 | 94.7 |
| | human IgG1 | | | VL: 874 | | | |
| c461985-10.0 | Humanized | [symbol] | [symbol] | VH: 83.8 | 73 | 5.8 | 97.9 |
| | human | | | VL: 82.3 | | | |
| | IgG1 | | | | | | |
| CA51986-10.0 | Humanized | [symbol] | [symbol] | VH: 83.8 | 110 | 8.8 | 96.6 |
| | human | | | VL: 78.2 | | | |
| | IgG1 | | | | | | |
| CA61987-10.0 | Humanized | [symbol] | [symbol] | VH: 76.2 | 59 | 4.7 | 97.0 |
| | human | | | VL: 87.4 | | | |
| | IgG1 | | | | | | |
| cAb1988-10.0 | Humanized | [symbol] | [symbol] | VH: 76.2 | 88 | 7.0 | 98.5 |
| | human | | | VL: 82.3 | | | |
| | IgG1 | | | | | | |
| cAb1989-10.0 | Humanized | [symbol] | [symbol] | VH: 76.2 | 88 | 7.0 | 98.3 |
| | human | | | VL: 78.2 | | | |
| IgG1 | | | | | | | |

[symbol]

indicates data missing or illegible when filed

TABLE 4
Table showing mouse scFv and humanized scFvs.
Protein sequences are shown along with the percentage
identity to human germline sequences, titre and
amount of final scFv purified.

| | | ScFv | | Titre | Purified |
|---|---|---|---|---|---|
| ID | Type | sequence | % | (mg/l) | (mg) |
| cAb1977-30.11 | Chimeric | [symbol] | n/a | 0 | 0 |
| | ScFv | | | | |
| cAb1978-30.11 | Humanized | [symbol] | VH: 91.8 | 8 | 0.6 |
| | ScFv | | VL: 87.4 | | |
| CAD1979-30.11 | Humanized | [symbol] | VH: 91.8 | 0 | 0 |
| | ScFv | | VL: 82.3 | | |
| cAb1980-30.11 | Humanized | [symbol] | VH: 91.8 | 44 | 3.5 |
| | ScFv | | VL: 78.2 | | |
| CAB1981-30.11 | Humanized | [symbol] | VH: 3.9 | 0 | 0 |
| | ScFv | | VL: 87.4 | | |
| cAb1982-30.11 | Humanized | [symbol] | VH: 93.9 | 0 | 0 |
| | ScFv | | VI: 82.3 | | |
| cAb1983-30.11 | Humanized | [symbol] | VH: 93.9 | 0 | 0 |
| | ScFv | | VI: 78.2 | | |
| cAb1984-30.11 | Humanized | [symbol] | VH: 83.8 | 33 | 2.6 |
| | ScFv | | VL: 87.4 | | |
| CAB1985-30.11 | Humanized | [symbol] | VH: 83.8 | 6 | 0.5 |
| | ScFv | | VL: 82.3 | | |
| CAb1986-30.11 | Humanized | [symbol] | VH: 83.8 | 13 | 1 |
| | ScFv | | VL: 78.2 | | |
| cAb1987-30.11 | Humanized | [symbol] | VH: 762 | 50 | 4 |
| | ScFv | | VL: 87.4 | | |
| cAb1988-30.11 | Humanized | [symbol] | VH: 76.2 | 0 | 0 |
| | ScFv | | VL: 82.3 | | |
| cAb1989-30.11 | Humanized | [symbol] | VH: 76.2 | 38 | 3 |
| | ScFv | | VL: 78.2 | | |

[symbol]

indicates data missing or illegible when filed

### Aggregation Analytics

Purified IgGs were analyzed for aggregation and fragmentation by SEC-HPLC. Monomer content is reported in Table 3. All antibodies show more than 95% monomer purity and the majority show more than 98% monomer purity.

### Example 2: Binding Affinities of Newly Discovered Fully Humanized Anti-FLT3 IgG Clone and its scFv

This example shows that a humanized anti-FLT3 antibody, such as the antibodies described herein, have high binding affinity for FLT3. Specifically, this example shows that the humanized anti-FLT3 IgG and anti-FLT3 scFv have high binding affinities to cells expressing FLT3.

A fully humanized anti-human FLT3 monoclonal IgG1, clone hum 1-18BA-v1, clone ID cAb1978-30.11, antibody (having VL of SEQ ID NO: 1 and VH of SEQ ID NO: 3) and his-tagged scFv, clone hum 1-18BA-v1, antibody with the same VH and VL as the IgG1 (comprising SEQ ID NO: 4 and a His tag on the C terminus) were developed as described above. Binding affinity of the scFv molecule was compared to the IgG1 monoclonal molecule using REH cells (acute lymphocytic leukemia cell line, ATCC, no. CRL-8286), which highly express FLT3. REH cells were incubated with varying concentrations (10⁻¹ to 10⁴ ng/mL) of the IgG1 or scFv molecules diluted in FACS buffer (hereinafter referred to simply as “buffer,” phosphate buffered saline (PBS) (Caisson Labs, no. PBL06)+2% BCS (GE Healthcare, no. SH30073.04)+1 mM EDTA (Invitrogen, no. 15575020)) in a final volume of 100 μL, in triplicate, for 30 minutes at 4° C. Cells were washed three times with buffer and incubated with secondary antibodies. IgG1 was detected with 1:200 goat anti-human Fc FITC (Jackson) secondary antibody and scFv was detected with anti-His FITC (1:200) secondary antibody. Secondary antibodies were incubated in 100 μL final volume in triplicate for 30 minutes at 4° C. Cells were washed once with buffer and resuspended in 200 uL buffer+1:100 7-AAD Viability Staining Solution (Biolegend, no. 420404), and analyzed by flow cytometry. Flow cytometry acquisition was performed with a Beckman Coulter CytoFLEX (Beckman Coulter), and analysis was performed with FlowJo (Treestar Inc, Ashland, OR). Plots show median fluorescence intensity of REH cells versus concentration of the primary antibody used. Variable slope (four parameters) curve was fit to the data and EC50 was used to compare binding affinities. The IgG1 molecule has an EC50 of 62.1 ng/mL (0.41 nM) and the scFv molecule has an EC50 of 85.45 ng/mL (3.42 nM) (FIGS. 1B-1C).

### Example 3: Generation of Third Generation Anti-FLT3 Chimeric Antigen Receptor

To generate a CAR, a lentiviral vector that encodes a third generation CAR under the transcriptional control of the EF-1α promoter was used (FIG. 2B). The CAR encodes a polypeptide comprising the anti-FLT3 scFv sequence described in Example 1 (SEQ ID NO:4), followed by CD28 and 4-1BB co-stimulatory domains, followed by the CD3ζ activation domain. The CAR sequence is followed by CopGFP sequence linked by a self-cleaving T2A sequence (SEQ ID NO. 16). This vector permits dual expression of the CAR and CopGFP from a single RNA transcript. All constructs were verified by sequencing.

FLT3 is expressed on hematopoietic stem and progenitor cells (HSPCs) and dendritic cells, and in acute myeloid leukemia. The expression of the CAR in T-cells allows for MHC-independent primary activation via the anti-FLT3 scFv when T cells are exposed to FLT3⁺ target cells. Through the co-stimulatory and activation domains in the CAR, this activation leads to expression of perforin and granzyme which ultimately are cytotoxic to target cells (FIG. 2C).

### Example 4: Isolation and Transduction of T Cells

To generate anti-FLT3 CAR T cells, the following protocol was carried out. This example shows that anti-FLT3 CAR T cells were successfully generated, that expression of the anti-FLT3 CAR T cells peaked at 4 days after transduction, and that T cells expressing anti-FLT3 CAR expanded about 125 fold in 18 days.

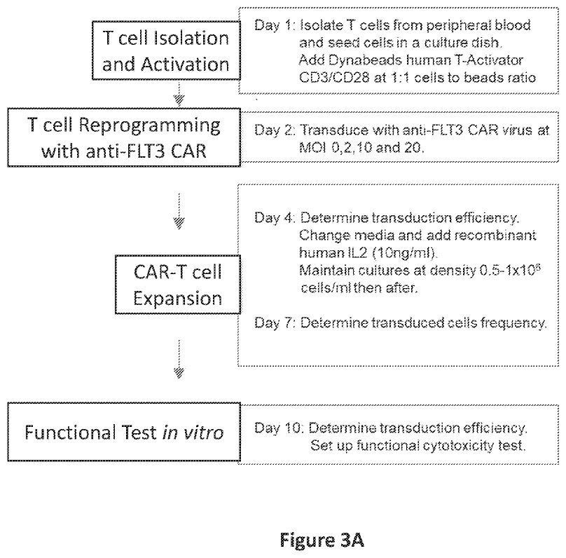

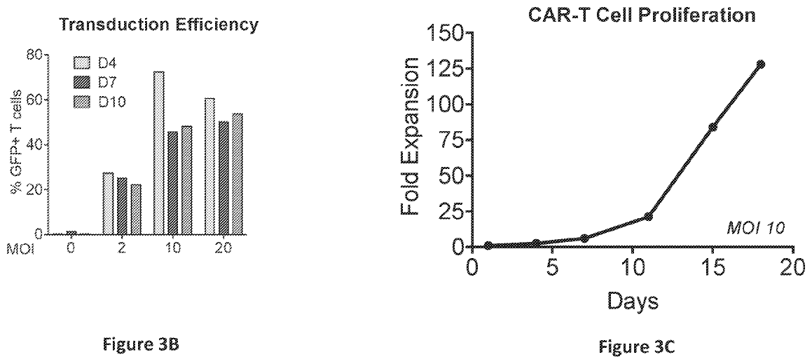

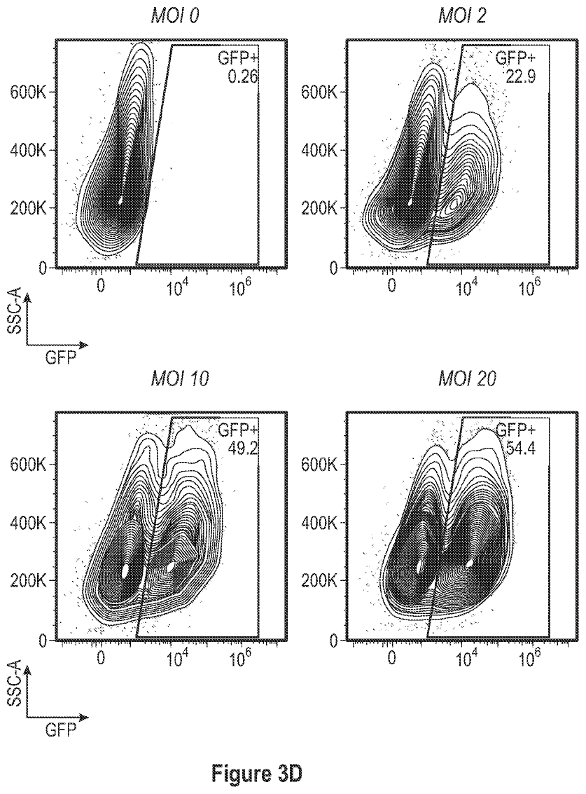

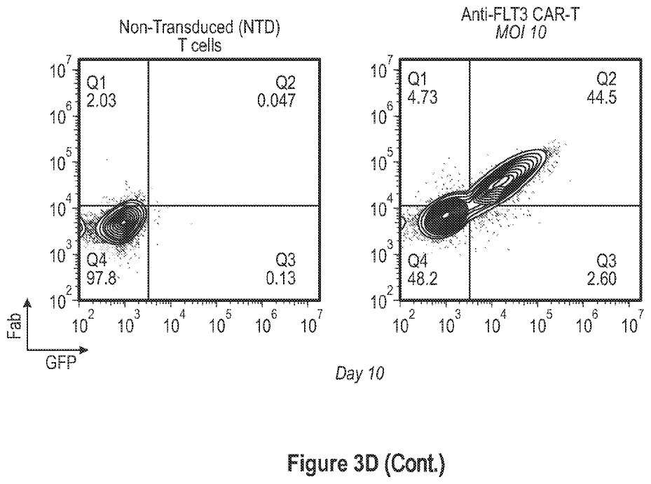

On day 1, T cells were isolated from adult peripheral blood (purchased from New York Blood Center, NYBC) using a negative magnetic isolation kit (StemCELL Technologies, no. 17951). T cells were mixed with Dynabeads (Human T-Activator CD3/CD28, Gibco, no. 111.61D) in a 1:1 cell to bead ratio and seeded in a non-treated 96-well Flat bottom cell culture plate at density 8×10⁴ cells/well or 1.6×10⁵ cells/well in 200 ul culture medium (RPMI 1640 Medium (ATCC, no. 30-2001)+10% heat inactivated fetal bovine serum (FBS) (Biowest, no. S1620)+1% penicillin/streptomycin (Gibco, no. 15140122) or 10⁶ cells/well in 24 well plate in 1 ml medium. On day 2, T cells were transduced with CAR T lentivirus vector at several MOI (0, 2, 5, 10 and 20) using 1:1000 polybrene (5 mg/mL) (VectorBuilder). On day 4, transduction efficiency was determined by flow cytometry. GFP positive cells were successfully transduced. Surface expression of scFv was confirmed using a polyclonal anti-Fab APC antibody (Jackson ImmunoResearch, no. 109-607-003). T cells were stained with the scFv detection antibody for 30 minutes at 4° C. Cells were washed once with buffer and resuspended in 200 μL buffer and analyzed by flow cytometry. Flow cytometry acquisition was performed with a Beckman Coulter CytoFLEX (Beckman Coulter), and analysis was performed with FlowJo (Treestar Inc, Ashland, OR). T cell medium was changed and supplemented with recombinant human IL-2 at a final concentration of 10 ng/ml (Miltenyi Biotec, no. 130097745). T cells were split as needed to maintain cell density between 0.5 and 1×10⁶ cells/ml. On day 7, transduction efficiency was assessed by flow cytometry, as described above. On day 10, transduction efficiency was assessed by flow cytometry, as described above, and T cells are ready for functional cytotoxicity test (FIG. 3A).

CAR T-cells were generated and expanded as described above. Specifically, cells were transduced with a CAR having an amino acid sequence of SEQ ID NO: 16 (with domains in the following orientation: signal peptide-linker-scFV of SEQ ID NO: 4-linker-CD8α hinge-CD8α transmembrane domain-CD28-41BB-CD3ζ-T2A-GFP). Transduction efficiency was determined over a 10 day period at different MOIs. Specifically, GFP expression was measured in cells transduced with the anti-FLT3 CAR3a (SEQ ID NO: 16). GFP expression peaked at day 4 and decreased until day 7 and appeared to stabilize at day 10 for all MOIs (FIG. 3B). When transduced with anti-FLT3 CAR3a, T cells expanded roughly 125-fold in 18 days (FIG. 3C). Expression of the GFP (i.e. expression of the CAR construct) demonstrated linear correlation with expression of scFv as expected (FIG. 3D).

### Example 5: In Vivo and In Vitro Anti-FLT3 CAR-T Cytotoxicity Against AML Cell Line MOLM-13

This example shows that the anti-FLT3 CAR described in Example 4 is effective against an AML cell line in vitro and effective to increase survival in an animal model of leukemia in vivo.

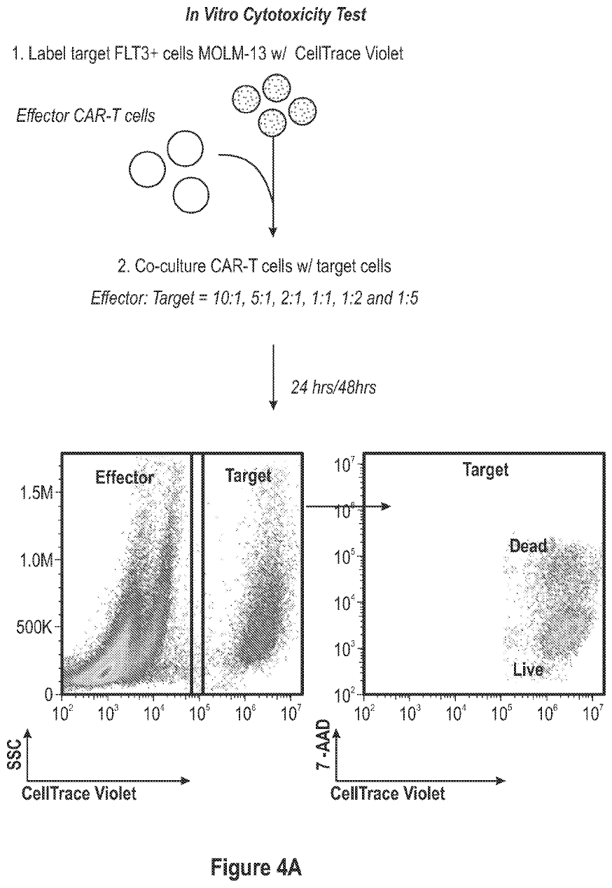

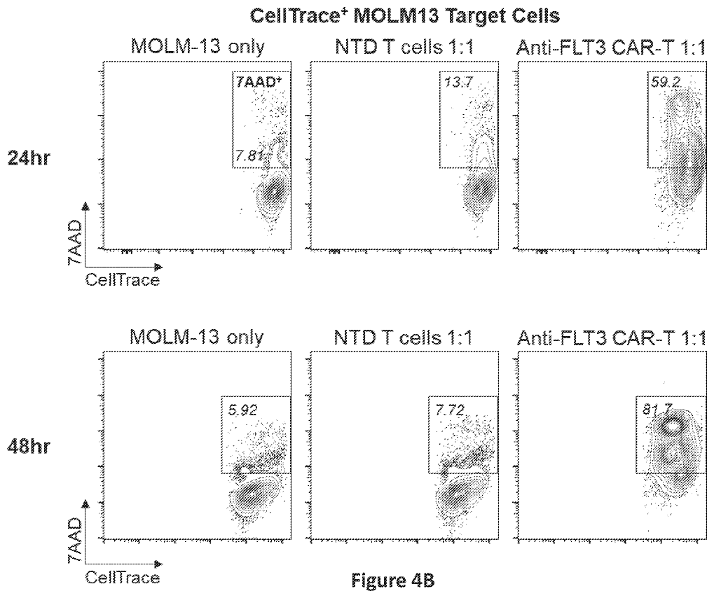

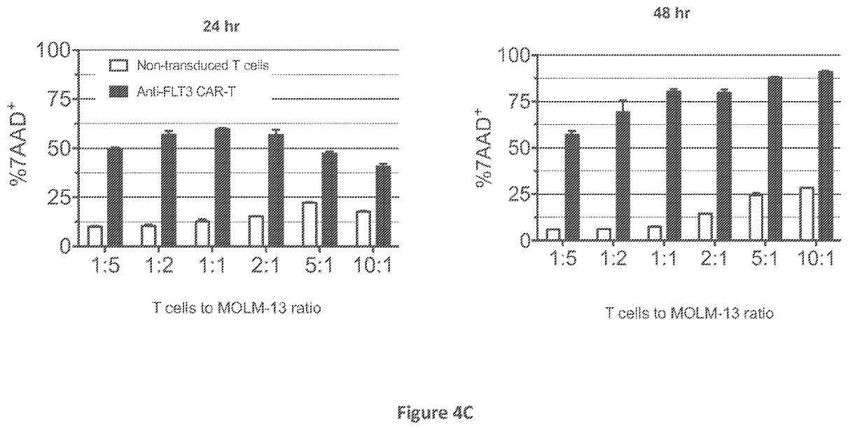

CAR T-cells were generated and expanded as described above and then used in a functional cytotoxicity assay. Specifically, cytotoxicity of cells was measured using AML cell lines that express FLT3, such as MOLM-13 (DSMZ, no. ACC 554) labeled with CellTrace™ Violet Cell Proliferation Kit (Thermo Fisher Scientific, no. C34571) (FIG. 4A). CAR T-cells (effector cells) expressing the CAR encoded by SEQ ID NO: 16 (encoding domains in the following orientation: signal peptide-linker-scFV of SEQ ID NO: 4-linker-CD8α hinge-CD8α transmembrane domain-CD28-41BB-CD3ζ-T2A-GFP) were co-cultured with labeled AML cells (target cells) at various effector to target cell ratios (10:1, 5:1, 2:1, 1:1, 1:2 and 1:5) for 24 and 48 hours. Cells were harvested, washed with FACS buffer, and resuspended in 200 μL of 11:100 7-AAD Viability Staining Solution (Biolegend, no. 420404), and analyzed by flow cytometry. Flow cytometry acquisition was performed with a Beckman Coulter CytoFLEX (Beckman Coulter), and analysis was performed with FlowJo (Treestar Inc, Ashland, OR). Representative dot plots show the flow data after excluding debris. Target cells are identified as CellTraceViolet⁺ and effector cells as CellTraceViolet⁻. Gating on the CellTraceViolet⁺ cells, frequencies of dead (7AAD⁺) and live (7AAD⁻) target cells were determined. Cytotoxicity to MOLM-13 was significantly higher when co-cultured in vitro with anti-FLT3 CAR3a-T cells compared to control T cells at 24 and 48 hours (FIGS. 4B and 4C).

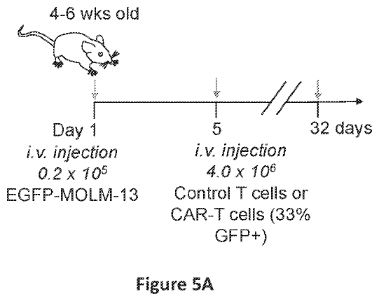

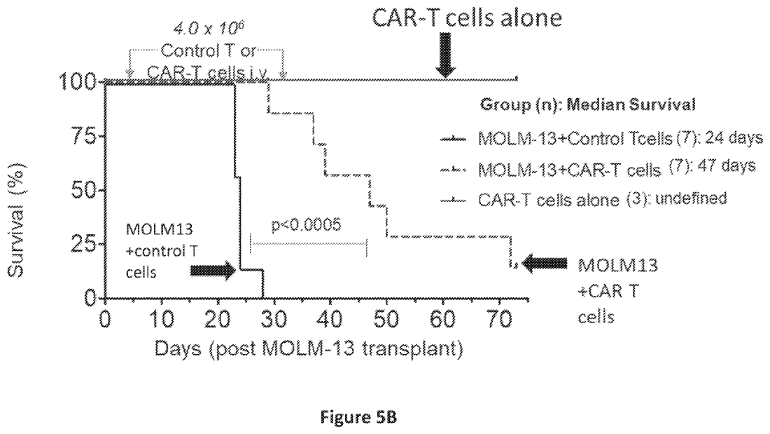

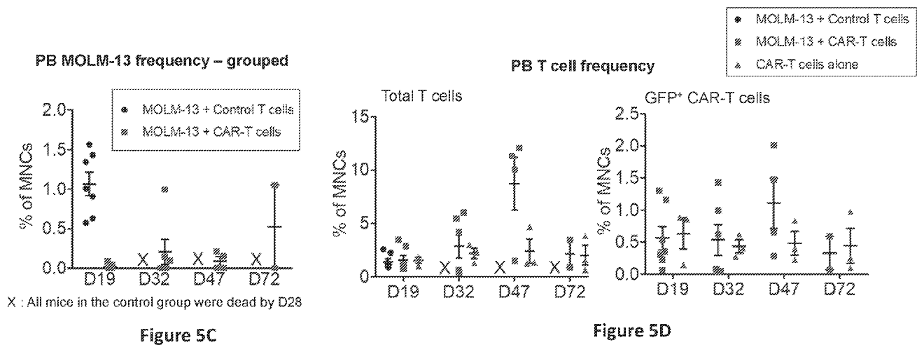

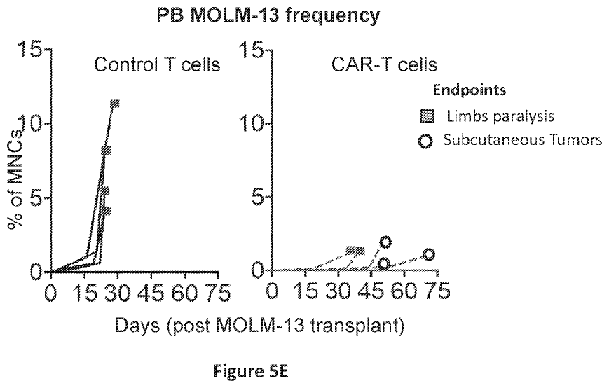

In vivo efficacy of anti-FLT3 CAR3a-T cells against leukemia was evaluated in female NOD.Cg-Prkdc^{scid} Il2rg^{tm1Sug}/JicTac (hereinafter, abbreviated as NOG mice, Taconic, no. NOG-F) into which AML cells, MOLM-13 cell line (DSMZ, no. ACC 554) transduced to express EGFP, were transplanted. For each NOG mouse (n=14), 2×10⁵ EGFP-MOLM-13 cells were transplanted intravenously on day 1. On day 5 and day 32 each mouse received either 4×10⁶ control T cells (n=7) or 4×10⁶ anti-FLT3 CAR3a T cells (33% CAR⁺) (n=7). Some mice received 4×10⁶ CAR-T cells (33% CAR⁺) (n=4) to assess effects of CAR-T alone on mice (i.e. the anti-FLT3 CAR3a T cells without MOLM-13 cells) (FIG. 5A). Mice showing symptoms of physiological distress, cachexia, or hind-leg paralysis were sacrificed. Anti-FLT3 CAR3a-T cell treatment extended the median survival to 47 days compared to 24 days in control T cell mice (FIG. 5B). Mice were bled every two weeks after T cell administration. Peripheral blood mononuclear cells were stained with anti-mouse CD45 APC (BioLegend, no. 103112), anti-human CD45 APC-eFluor780 (Invitrogen, no. 47045942), anti-human CD33 BV510 (BioLegend, no. 366610), and anti-human CD3 PE-Cy7 (BioLegend, no. 300420) and analyzed by flow cytometry to determine frequency of MOLM-13 cells (mCD45⁻hCD45⁺CD33⁺EGFP⁺) and T cells (mCD45⁻hCD45⁺CD3⁺) or CAR-T cells (mCD45⁻hCD45⁺CD3⁺EGFP⁺) in circulation. Flow cytometry acquisition was performed with a Beckman Coulter CytoFLEX (Beckman Coulter), and analysis was performed with FlowJo (Treestar Inc, Ashland, OR). The frequency of T cells (to that of total mononuclear cells) was maintained in anti-FLT3 CAR3a-T cells to day 47 and in some instances to day 72 in treated animals whereas control T cell treated mice were dead by day 28 (FIGS. 5C-5E).

### Example 6: Targeting CD34⁺ Bone Marrow HSPCs with FLT3-CAR-T Cells for Hematopoietic Stem Cell Transplant (HSCT) Conditioning

This example shows that anti-FLT3 CAR T cells described herein are effective to achieve depletion of CD34+ HSPCs. This demonstrates the possibility of use of the anti-FLT3 CAR T cells for HSCT.

### Preparation of Human HSCs and Transplantation

Mononuclear cells (MNCs) from fresh umbilical cord blood (CB) units (Carolina Cord Blood Bank) were isolated by density centrifugation using Ficoll separation medium (StemCELL Technologies, no. 07861). To further purify MNCs, red blood cells were lysed using lysis buffer (Alfa Aesar, no. J62150-AP). CB MNCs were then enriched for human CD34⁺ cells using anti-human CD34 microbeads (Miltenyi Biotec, no. 130-046-703). The CD34⁻ fraction of MNCs was enriched for T cells using negative magnetic isolation (StemCELL Technologies, no. 17951).

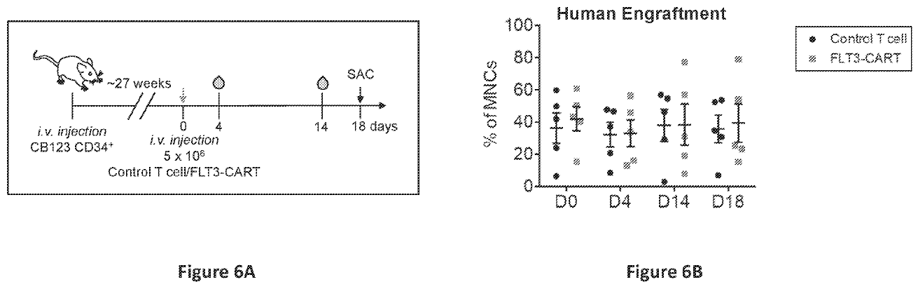

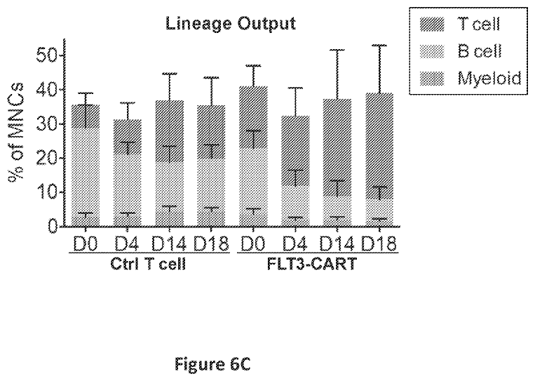

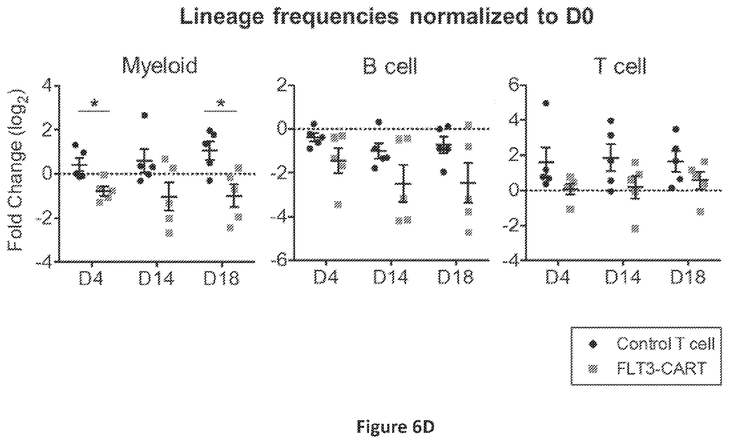

3-4 week old NOG female mice were injected with 2.4×10⁵ CD34⁺ cells and 10⁵ T cells. Mice were bled from the submandibular vein (˜100 μL) every 4 weeks to evaluate human chimerism (FIG. 6A). PBMCs were stained with the following mAb panel to determine level of humanization and lineage development: anti-mouse CD45 APC (BioLegend, no. 103112), anti-human CD45 APC-eFluor780 (Invitrogen, no. 47045942), anti-human CD3 PE-Cy7 (BioLegend, no. 300420), anti-human CD19 PE (BioLegend, no. 302208), anti-human CD33 FITC (BioLegend, no. 303304), anti-human CD4 BV605 (BioLegend, 317438), anti-human CD8 BV510 (BioLegend, no. 344732), anti-human CD45RA BV650 (BioLegend, no. 304136), anti-human CD45RO Pacific Blue (BioLegend, no. 304216).

Conditioning with Autologous CAR-T Cells

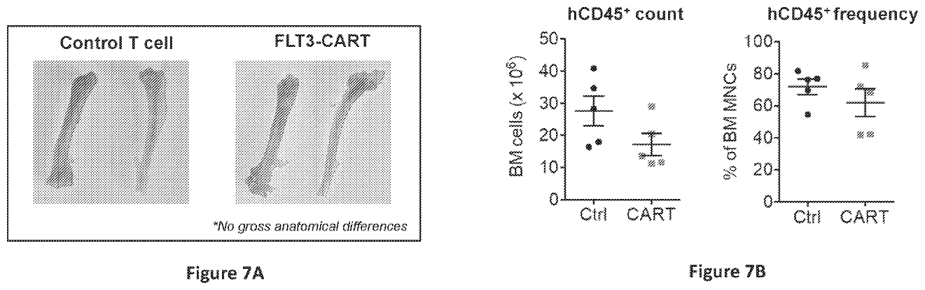

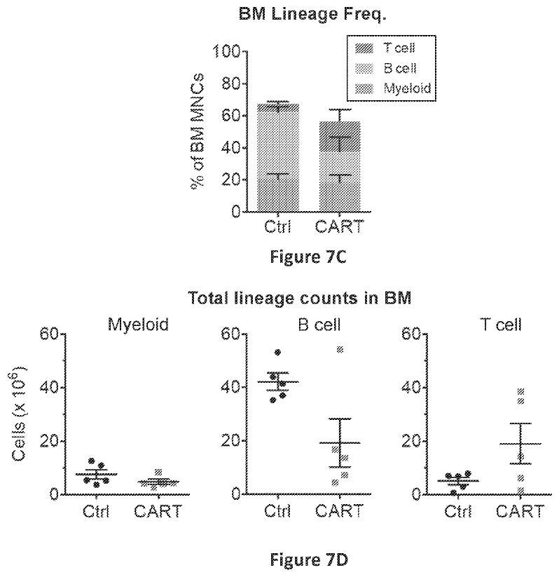

When the mice described above (FIG. 6A) showed robust human engraftment (>1% human CD45⁺), 27 weeks post transplantation, mice received either 5×10⁶ autologous control T cells (expanded in same way as CAR-T cells) or 5×10⁶ autologous CAR-T cells (expressing the CAR described in Example 3). Mice were bled on day 4, 14 and 18 post treatment with T cells. Mice were euthanized on day 18 post treatment with T cells and peripheral blood and bone marrow (BM) was isolated. Overall frequency of human CD45 cells in MNC fraction of peripheral blood before and after treatment with control or CAR-T cells was measured and showed similar engraftment between control T cells and anti-FLT3 CAR T cells (FIG. 6B). Lineage frequencies (T cells (CD3⁺), B cells (CD19⁺), and myeloid cells (CD33⁺)) before and after treatment with control or CAR-T cells were measured in peripheral blood (FIG. 6C) and changes in the frequencies over time was measured (FIG. 6D). Myeloid cells showed significant decline in anti-FLT3 CAR-T cell treated mice compared to those treated with control T cells. Changes in the cell frequency were then measured in isolated bone marrow (BM). Femurs and tibias from mice were visually assessed for gross anatomical differences and showed no difference between control T cell and anti-FLT3 CAR T cell treated animals (FIG. 7A). Total cell counts of MNCs from BM (BM-MNCs) were recorded. BM-MNCs were also analyzed by flow cytometry as described above and frequencies of human CD45⁺ cells were determined (FIG. 7B). Lineage frequencies (T cells (CD3⁺), B cells (CD19⁺), and myeloid cells (CD33⁺)) in the BM-MNCs were measured (FIG. 7C). Lineage cell counts (T cells (CD3⁺), B cells (CD19⁺), and myeloid cells (CD33⁺)) before and after treatment with control or CAR-T cells shown for individual mice in the two cohorts (FIG. 7D). CD45+CD19+ B cell counts were trending lower (by 54.4% compared to control) in mice treated with CAR-T. Without being bound by theory, it is possible B cell counts were reduced due to a reduction in the number of HSCs, MPPs, and CPs.

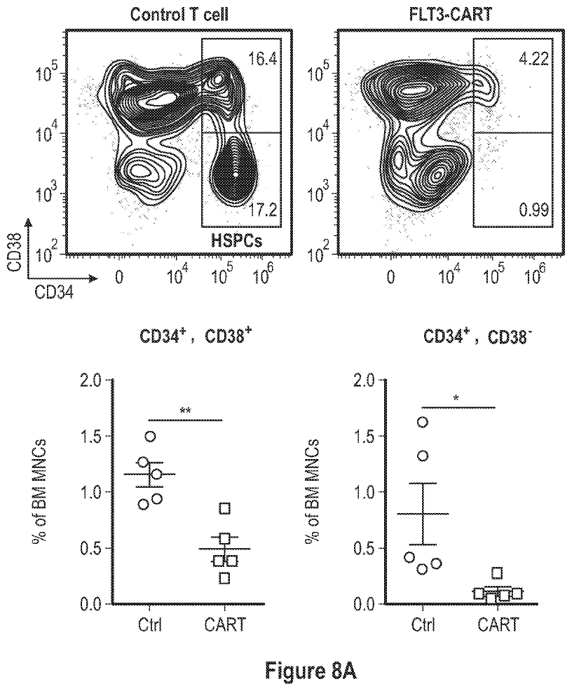

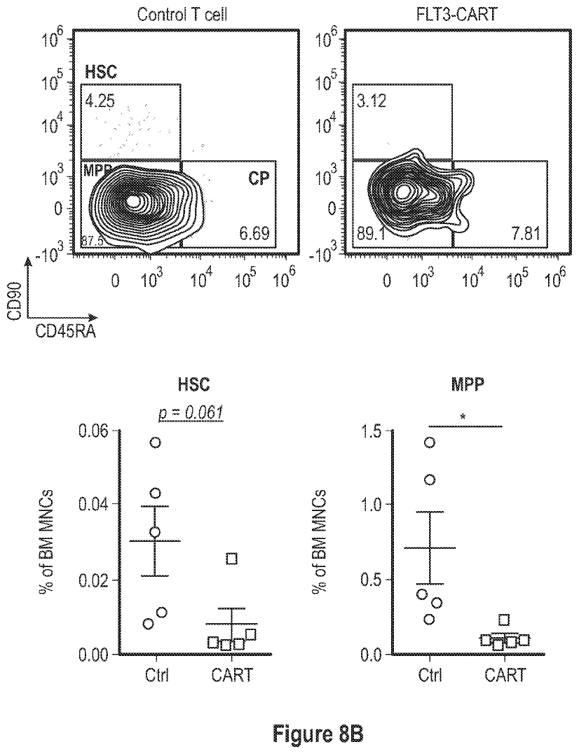

BM-MNCs were stained with the following mAbs panel to determine frequencies of HSPCs: anti-mouse CD45 APC (BioLegend, no. 103112), anti-human CD45 APC-eFluor780 (Invitrogen, no. 47045942), anti-human Lineage cocktail BV510 (BioLegend, no. 348807), anti-human CD34 PE-Cy7 (BioLegend, no. 343516), anti-human CD38 FITC (BioLegend, no. 356610), anti-human CD90 PE (Invitrogen, no. 12090942), and anti-human CD45RA BV650 (BioLegend, no. 304136). Significant depletion of hematopoietic stem and progenitor cells (CD38⁺CD34⁺ and CD38⁻CD34⁺ populations) was observed in FLT3-CAR T treated mice compared to controls (FIG. 8A). CAR T-cell treated mice have significantly fewer progenitors in the bone marrow compared to control mice. Further gating on the CD38⁻CD34⁺ population revealed significant depletion of “true” hematopoietic stem cells (HSCs) (CD90⁺CD45RA⁻), multi-potent progenitors (MPPs) (CD90⁻CD45RA⁻), and common progenitors (CPs) (CD90⁻CD45RA⁺) (FIGS. 8A and 8B).

### Example 7: Generating a CAR Construct with a Suicide Switch

CAR constructs incorporating a suicide safety switch were generated. Specifically, an epidermal growth factor receptor (EGFR) based safety switch, EGFRt (truncated EGFR), was co-expressed on the CAR plasmid after a T2A peptide sequence (the resulting CAR has an amino acid sequence of SEQ ID NO: 7 and comprises domains in the following orientation: signal peptide-linker-scFV of SEQ ID NO: 4-linker-CD8α hinge-CD8α transmembrane domain-CD28-41BB-CD3ζ-T2A-EGFRt). Self-cleavage of peptide by T2A allows EGRFt to be expressed on the surface. “Suicide” is achieved by treatment with cetuximab (anti-EGFR mAb) that will target T cells for opsonization in vivo.

A second construct, an inducible Caspase9 (iCasp9) based safety suicide switch, was generated. iCasp9 molecule was co-expressed on the CAR plasmid after a T2A peptide sequence (the resulting CAR has amino acid sequence of SEQ ID NO: 8 and has domains in the following orientation: signal peptide-linker-scFV of SEQ ID NO: 4-linker-CD8α hinge-CD8α transmembrane domain-CD28-41BB-CD3ζ-T2A-iCasp9). Self-cleavage of peptide by T2A allows iCasp9 to be expressed on the surface. “Suicide” is achieved by treatment with AP1903 (rimiducid) which leads to dimerization of iCasp9 and triggers apoptotic pathways.

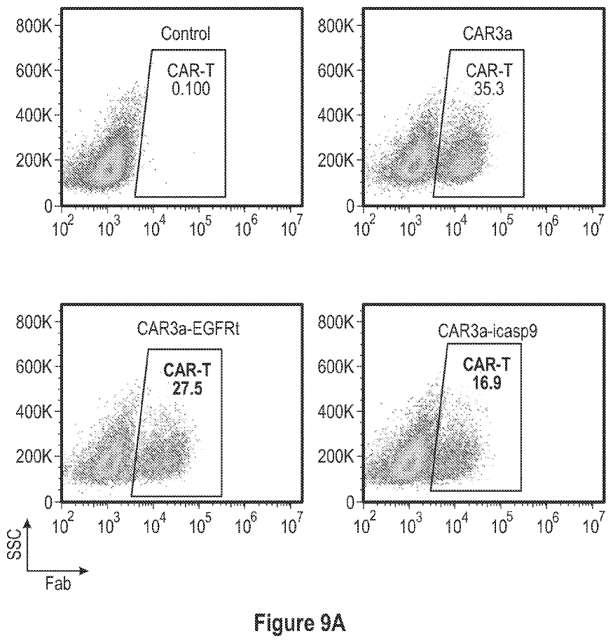

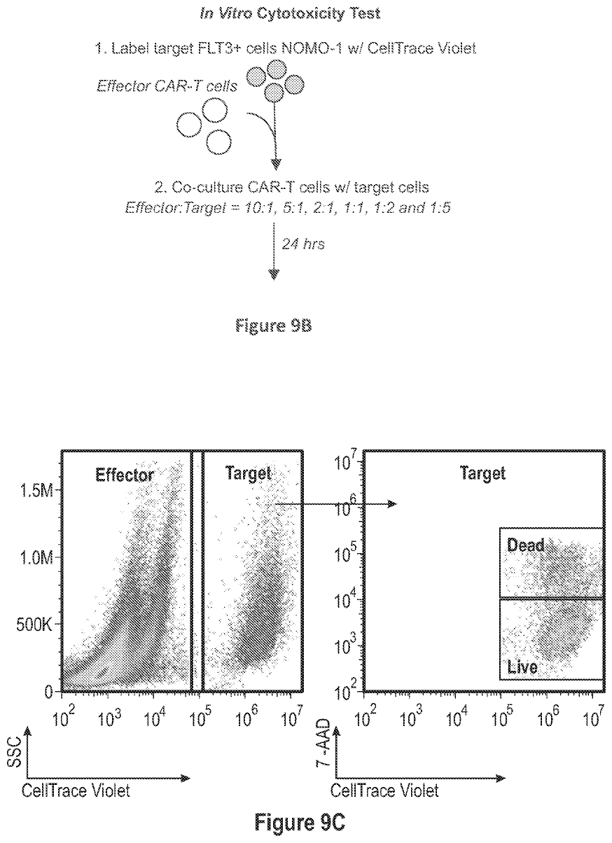

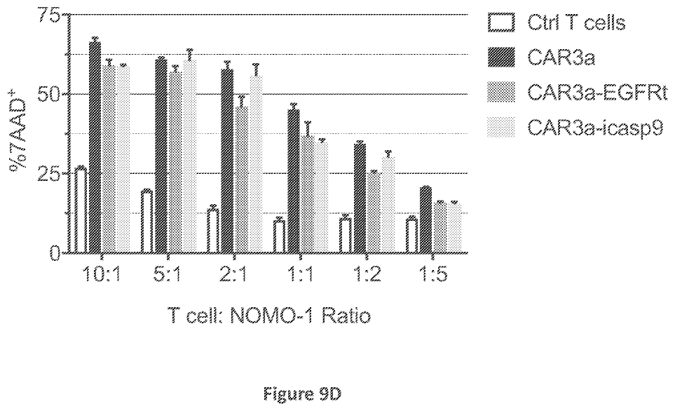

Transduction efficiency of suicide-CAR vectors based on surface expression of anti-FLT3 scFv in human T cells was measured. Expression of scFv was detected using a polyclonal anti-Fab APC antibody (Jackson ImmunoResearch, no. 109-607-003). T cells were stained with the scFv detection antibody for 30 minutes at 4° C. Cells were washed once with buffer and resuspended in 200 μL buffer+1:100 7-AAD Viability Staining Solution (Biolegend, no. 420404), and analyzed by flow cytometry. Flow cytometry acquisition was performed with a Beckman Coulter CytoFLEX (Beckman Coulter), and analysis was performed with FlowJo (Treestar Inc, Ashland, OR). CAR-T frequencies were determined as 35.3%, 27.5% and 16.9% in anti-FLT3 CAR (SEQ ID NO: 16), CAR3a-EGFRt (SEQ ID NO: 7) and CAR3a-icasp9 (SEQ ID NO: 8) samples respectively (FIG. 9A).

Evaluation of Cytotoxicity with Suicide CAR3a Constructs CAR3a-EGFRt and CAR3a-icasp9

In vitro cytotoxicity of CAR-T cells with suicide switches CAR3a-EGFRt and CAR3a-icasp9 compared to the original construct anti-FLT3 CAR3a against an AML cell line was measured. NOMO-1 cells, AML cells that express FLT3, were labeled with CellTrace™ Violet Cell Proliferation Kit (Thermo Fisher Scientific, no. C34571). CART-cells (effector cells) were combined with labeled NOMO-1 (target cells) at various effector to target cell ratios (10:1, 5:1, 2:1, 1:1, 1:2 and 1:5). The amount of total T cells used was kept the same without accounting for differences in CAR-T frequencies (FIG. 9B). After 24 hours of co-culture, all cells were collected, labeled with 7-AAD Viability Staining Solution (Biolegend, no. 420404), and analyzed by flow cytometry. Flow cytometry acquisition was performed with a Beckman Coulter CytoFLEX (Beckman Coulter), and analysis was performed with FlowJo (Treestar Inc, Ashland, OR). Representative dot plots show the flow data after excluding debris. Target cells were identified as CellTraceViolet⁺ and effector cells as CellTraceViolet⁻. Frequencies of 7AAD⁺ dead target cells when incubated with T cells expressing CAR-3a, CAR3a-EGFRt and CAR3a-icasp9 at various effector:target ratios were measured. All CAR-T cells show significantly more cytotoxic effect against FLT3⁺ NOMO-1 cells compared to control T cells. There was no significant difference in cytotoxic effect between either of the two suicide CAR-T cells (i.e. anti-FLT3 CAR-EGFRt and anti-FLT3 CAR-icasp9) and the original CAR construct (anti-FLT3 CAR3a) (FIG. 9C).

### Example 8: Cetuximab Mediated Depletion of CAR-Ts Via ADCC In Vitro as a Functional Test of EGFRt-CART Cell Suicide Switch

This example demonstrates the successful depletion of CAR T cells expressing the EGFRt suicide gene via antibody dependent cell cytotoxicity.

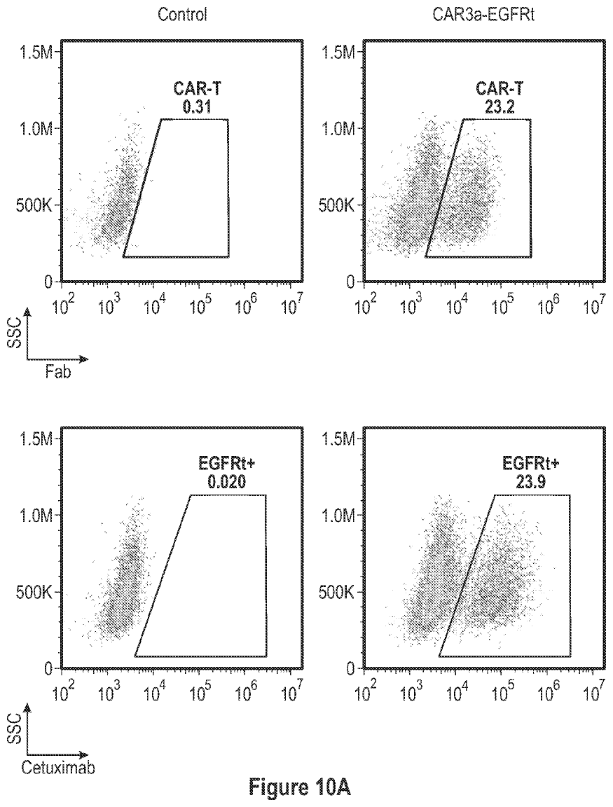

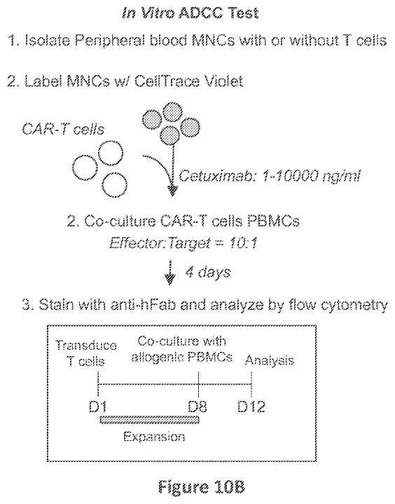

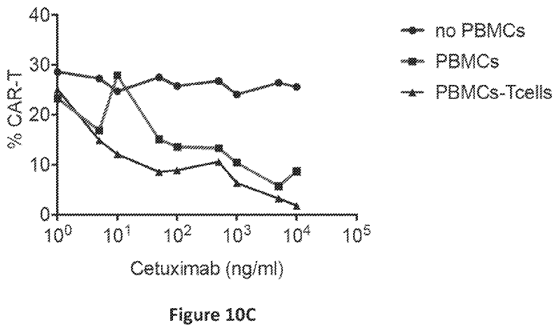

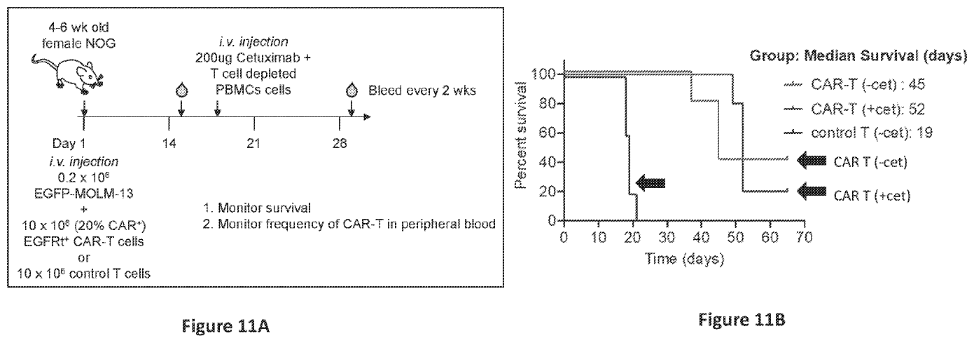

To validate expression of the EGFRt suicide switch, human T cells were transduced with the plasmid depicted in FIG. 12B which expressed CAR3a-T2A-EGFRt (SEQ ID NO: 7 and encodes domains in the following orientation: signal peptide-linker-scFV of SEQ ID NO: 4-linker-CD8α hinge-CD8α transmembrane domain-CD28-41BB-CD3ζ-T2A-EGFRt) lentiviral vector. Surface expression of the anti-FLT3 CAR3a was detected using a polyclonal anti-Fab APC antibody (Jackson ImmunoResearch, no. 109-607-003). Surface expression of the EGFRt was confirmed using cetuximab (Selleckchem A2000) and goat anti-human IgG Fc FITC antibody (Jackson) as a secondary antibody (FIG. 10A).

An antibody dependent cellular cytotoxicity (ADCC) test was performed to measure cetuximab mediated depletion of CAR3a-T2A-EGFRt T cells via ADCC. Specifically, CAR3a-T2A-EGFRt-transduced T cells were expanded as described above. On day 8, allogenic mononuclear cells (MNCs) (from New York Blood Center (NYBC)) (effector cells) either total or depleted of T cells using negative magnetic isolation (StemCELL Technologies, no. 17951) were labeled with CellTrace as previously described and added to culture with transduced T cells (target cells) at an effector to target ratio of 10:1. Cetuximab was added to co-culture at concentrations ranging from 1-10000 ng/mL. On day 12, all cells from co-cultures were collected and stained to detect scFv as previously described and analyzed by flow cytometry (FIG. 10B). Transduced T cells cultured alone (no PBMCs) show no significant decrease in CAR3a expressing cells after treatment with Cetuximab (FIG. 10C). Transduced T cells cultured with total allogenic MNCs (PBMCs) show dose dependent depletion of CAR3a expressing cells with Cetuximab. Transduced T cells cultured with allogenic MNCs depleted of T cells (PBMCs-Tcells) show dose dependent depletion of CAR3a expressing cells with Cetuximab. Results support function of antibody-dependent cellular cytotoxicity (ADCC) against EGFRt expressing CART-cells in vitro.

### Example 9: Efficacy of EGFRt-CAR T Against AML In Vivo and Cetuximab Mediated Depletion of CAR-Ts Via ADCC In Vivo

This example demonstrates that anti-FLT3 CAR T cells expressing an EGFRt suicide gene are able to improve survival in mice with AML (MOLM-13 cells) and are depleted upon treatment with cetuximab via antibody dependent cell cytotoxicity.

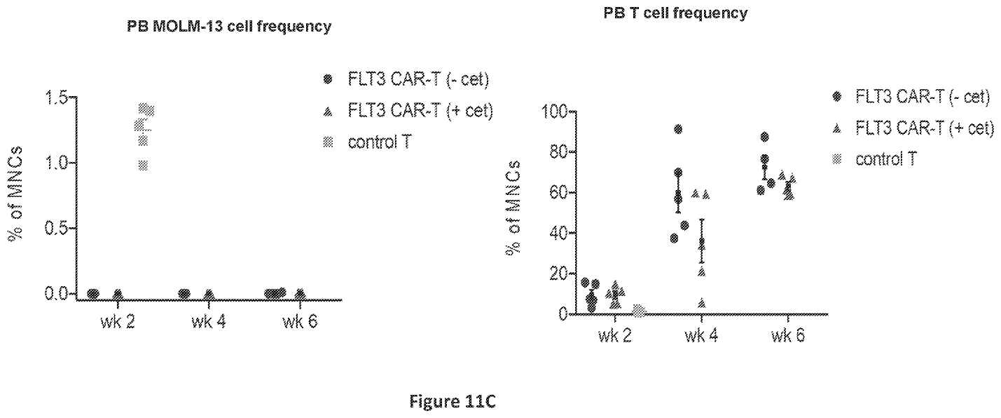

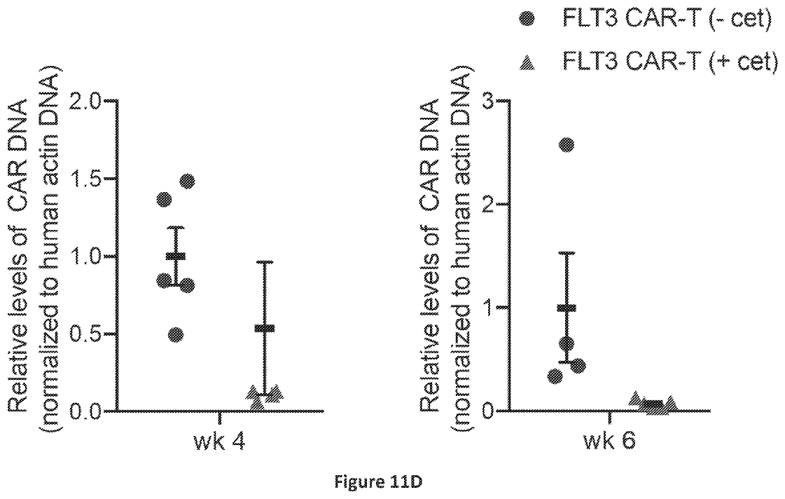

In vivo efficacy of anti-FLT3 EGFRt-CAR-T cells (as described in Example 8) against leukemia was evaluated in female NOD.Cg-Prkdc^{scid} Il2rg^{tm1Sug}/JicTac (hereinafter, abbreviated as NOG mice, Taconic, no. NOG-F) into which an AML cell line, MOLM-13 (DSMZ, no. ACC 554) transduced to express EGFP, was transplanted. For each NOG mouse (n=15), 2×10⁵ EGFP-MOLM-13 cells were transplanted intravenously on day 1. Also on day 1, each mouse received either 10×10⁶ control T cells (n=5) or 10×10⁶ EGFRt-CAR-T cells (20% CAR⁺) (n=10). On day 18, all mice (n=15) received 9×10⁶ effector cells (MNCs depleted of T cells) and one group of EGFRt-CART mice (n=5) also received an i.p. injection of 200 ug of Cetuximab. Three groups of mice are CAR T (−) cetuximab (n=5) (no cetuximab), CAR T (+) cetuximab (n=5), and control T (−) cetuximab (n=5) (FIG. 11A). Survival curves were generated up to 65 days post AML injection. Mice showing symptoms of physiological distress, cachexia, or hind-leg paralysis were sacrificed. FLT3 CAR-T treatment (without induction of CART suicide) extended the median survival to 45 days compared to 19 days in control T cell mice. The median survival of CAR T+ cetuximab mice was 52 days (FIG. 11B). Mice were bled every two weeks to determine engraftment of AML and T cells. PBMCs were stained with anti-mouse CD45 APC (BioLegend, no. 103112), anti-human CD45 APC-eFluor780 (Invitrogen, no. 47045942), and anti-human CD3 PE-Cy7 (BioLegend, no. 300420) and analyzed by flow cytometry to determine frequency of MOLM-13 cells (mCD45-hCD45⁺ EGFP⁺) and T cells (mCD45⁻hCD45⁺CD3⁺). (Left) Mice treated with CAR-T cells show much less proliferation of MOLM-13 compared to control T cell treated mice at week 2. By week 4 and week 6, all control T cell mice were found dead or were euthanized. (Right) T cell frequencies in peripheral blood (mCD45⁻hCD45⁺CD3⁺) were determined for each group various timepoints (FIG. 11C). The relative amount of circulating CAR-T cells in mice were also determined at week 4 and week 6 by quantifying relative levels of CAR specific DNA by PCR Cetuximab treatment effectively reduced the frequency of circulating CAR-T cells (FIG. 11D).

SEQUENCE LISTING

| SEQ | | |
|---|---|---|
| ID | | |
| NO | Description | Sequence |
| 1 | Light chain variable | DIQMTQSPSSLSASVGDRVTITCRASQEIS |
| | region of cAb1978 | GYLSWLQQKPGKAIKRLIYAASTLQSGVPS |
| | | RFSGSRSGSDYTLTISSLQPEDFATYYCLQ |
| | | YASYPFTFGQGTKLEIK |
| 2 | Light chain variable | DIVMTQSPDSLAVSLGERATINCKASQEIS |
| | region of cAb1980 | GYLSWLQQKPGQPIKRLIYAASTRESGVPD |
| | | RFSGSRSGSDYTLTISSLQAEDVAVYYCLQ |
| | | YASYPFTFGQGTKLEIK |
| 3 | Heavy chain | QVTLKESGPTLVKPTQTLTLTCTFSGFSLS |
| | variable region of | TSTMGVGWIRQPPGKALEWLAHILWNDSKR |
| | cAb1978 and 1980 | YNPSLKSRLTITKDTSKKQVVLTMTNMDPV |
| | | DTATYYCARIVYYSTYVGYFDVWGQGTTVT |
| | | VSS |
| 4 | scFv of cAb1978 | DIQMTQSPSSLSASVGDRVTITCRASQEIS |
| | | GYLSWLQQKPGKAIKRLIYAASTLQSGVPS |
| | | RFSGSRSGSDYTLTISSLQPEDFATYYCLQ |
| | | YASYPFTFGQGTKLEIKGGGGSGGGGSGGG |
| | | SGGGGSQVTLKESGPTLVKPTQTLTLTCTF |
| | | SGFSLSTSTMGVGWIRQPPGKALEWLAHIL |
| | | WNDSKRYNPSLKSRLTITKDTSKKQVVLTM |
| | | TNMDPVDTATYYCARIVYYSTYVGYFDVWG |
| | | QGTTVTVSS |
| 5 | scFv of cAb1980 | DIVMTQSPDSLAVSLGERATINCKASQEIS |
| | | GYLSWLQQKPGQPIKRLIYAASTRESGVPD |
| | | RFSGSRSGSDYTLTISSLQAEDVAVYYCLQ |
| | | YASYPFTFGQGTKLEIKGGGGGGGGSGGGS |
| | | GGGGSQVTLKESGPTLVKPTQTLTLTCTFS |
| | | GFSLSTSTMGVGWIRQPPGKALEWLAHILW |
| | | NDSKRYNPSLKSRLTITKDTSKKQVVLTMT |
| | | NMDPVDTATYYCARIVYYSTYVGYFDVWGQ |
| | | GTTVTVSS |
| 6 | Amino acid | MALPVTALLLPLALLLHAARPgsDIQMTQSPSS |
| | sequence of anti- | LSASVGDRVTITCRASQEISGYLSWLQQKPGKA |
| | FLT3 CAR with 19 | IKRLIVAASTLQSGVPSRFSGSRSGSDYTLTIS |
| | amino acid linker | SLQPEDFATYYCLQYASYPFTFGQGTKLEIKGG |
| | (SIGNAL | GGSGGGGSGGGSGGGGSQVTLKESGPTLVKPTQ |
| | PEPTIDE-linker- | TLTLTCTESGESLSTSTMGVGWIRQPPGKALEW |
| | SAB1978-30.11 | LAHILWNDSKRYNPSLKSRLTITKDTSKKQVVL |
| | VL-19AA | TMTNMDPVDTATYYCARIVYYSTYVGYFDVWGQ |
| | LINKER- | GTTVTVSSgsFVPVELPAKPTTTPAPRPPTPAP |
| | SAB1978-30.11 | TIASQPLSLRPEACRPAAGGAVHTRGLDFACDI |
| | VH-linker- | YIWAPLAGTCGVLLLSLVITLYCNHRNRSKRSR |
| | CD8αHINGE- | LLHSDYMNMTPRRPGPTRKHYQPYAPPRDFAAY |
| | CD8αTM-CD28- | RSKRGRKKLLYIFKQPEMRPVQTTQEEDGCSCR |
| | 41BB-cd3ζ) | FPEEEEGGCEL |
| | encoded by pCDH- | rvkfsrsadapayqqgqnqlynelnlgrreeyd |
| | EF1-CAR3a_19aa | vldkrrgrdpemggkprrknpqeglynelqkdk |
| | linker plasmid | maeayseigmkgerrrgkghdglyqglstatkd |
| | Where each domain | tydalhmqalppr |
| | matches the case | |
| | (i.e. capital or | |
| | lowercase), bold, | |
| | italic, and/or | |
| | underlined | |
| | formatting between | |
| | the sequence | |
| | description and the | |
| | sequence. | |
| 7 | Amino acid | MALPVTALLLPLALLLHAARPgsDIQMTQSPSSLS |
| | sequence of anti- | ASVGDRVTITCRASQEISGYLSWLQQKPGKAIKRL |
| | FLT3 CAR with 19 | IYAASTLQSGVPSRFSGSRSGSDYTLTISSLQPED |
| | amino acid linker | FATYYCLQYASYPFTFGQGTKLEIKGGGGSGGGGS |
| | and EGFRt | GGGSGGGGSQVTLKESGPTLVKPTQTLTLTCTFSG |
| | (SIGNAL | FSLSTSTMGVGWIRQPPGKALEWLAHILWNDSKRY |
| | PEPTIDE-linker- | NPSLKSRLTITKDTSKKQVVLTMTNMDPVDTATYY |
| | SAB1978-30.11 | CARIVYYSTYVGYFDVWGQGTTVTVSSgsFVPVFL |
| | VL-19AA | PAKPTTTPAPRPPTPAPTIASQPLSLRPEACRPAA |
| | LINKER- | GGAVHTRGLDFACDIYIWAPLAGTCGVLLLSLVIT |
| | SAB1978-30.11 | LYCNHRNRSKRSRLLHSDYMNMTPRRPGPTRKHYQ |
| | VH-linker- | PYAPPRDFAAYRSKRGRKKLLYIFKQPFMRPVQTT |
| | CD8αHINGE- | QEEDGCSCRFPEEEEGGCELrvkfsrsadapayqq |
| | CD8αTM-CD28- | gqnqlynelnlgrreeydvldkrrgrdpemggkpr |
| | 41BB-cd3ζ- | rknpqeglynelqkdkmaeayseigmkgerrrgkg |
| | LINKER-t2a- | hdglyqglstatkdtydalhmqalpprGSGegres |
| | egfrt) encoded by | lltcgdveenpgpmlllvtslIlcelphpafllip |
| | EF1_CAR3a_19aa | rkvcngigigefkdslsinatnikhfknctsisgd |
| | linkerT2Aegfrt_VB | lhilpvafrgdsfthtppldpqeldilktvkeitg |
| | 200623 plasmid | flliqawpenrtdlhafenleiirgrtkqhgqfsl |
| | Where each domain | avvslnitsigIrslkeisdgdviisgnknlcyan |
| | matches the case, | tinwkklfgtsgqktkiisnrgensckatgqvcha |
| | bold, italic, and/or | lcspegewgpeprdcvscrnvsrgrecvdkccll |
| | underlined | egeprefvenseciqchpeclpqamnitctgrgp |
| | formatting between | dnciqcahyidgphcvktcpagvmgenntlvwky |
| | the description and | adaghvchlchpnctygetgpglegcptngpkip |
| | the sequence. | siatgmvgalllllvvalgiglfm |
| 8 | Amino acid | MALPVTALLLPLALLLHAARPgsDIQMTQSPSSLS |
| | sequence of anti- | ASVGDRVTITCRASQEISGYLSWLQQKPGKAIKRL |
| | FLT3 CAR with 19 | IYAASTLQSGVPSRFSGSRSGSDYTLTISSLQPED |
| | amino acid linker | FATYYCLQYASYPFTFGQGTKLEIKGGGGSGGGGS |
| | and iCasp9 | GGGSGGGGSQVTLKESGPTLVKPTQTLTLTCTESG |
| | (SIGNAL | FSLSTSTMGVGWIRQPPGKALEWLAHILWNDSKRY |
| | PEPTIDE-linker- | NPSLKSRLTITKDTSKKQVVLTMTNMDPVDTATYY |
| | SAB1978-30.11 | CARIVYYSTYVGYFDVWGQGTTVTVSSgsFVPVEL |
| | VL-19AA | PAKPTTTPAPRPPTPAPTIASQPLSLRPEACRPAA |
| | LINKER- | GGAVHTRGLDFACDIYIWAPLAGTCGVLLLSLVIT |
| | SAB1978-30.11 | LYCNHRNRSKRSRLLHSDYMNMTPRRPGPTRKHYQ |
| | VH-linker- | PYAPPRDFAAYRSKRGRKKLLYIFKQPFMRPVQTT |
| | CD8αHINGE- | QEEDGCSCRFPEEEEGGCELrvkfsrsadapayqq |
| | CD8αTM-CD28- | gqnqlynelnlgrreeydvldkrrgrdpemggkpr |
| | 41BB-cd3ζ- | rknpqeglynelqkdkmaeayseigmkgerrrgkg |
| | LINKER-t2a- | hdglyqglstatkdtydalhmqalpprGSGegrgs |
| | iCasp9) encoded | lltcgdveenpgp |
| | by | mgvqvetispgdgrtfpkrgqtcvvhytgm |
| | pCDH-EF1- | ledgkkvdssrdrnkpfkfmlgkqevirgw |
| | CAR3a_19aa | eegvaqmsvgqrakltispdyaygatghpg |
| | linker T2A_icasp9 | iipphatlvfdvellklesgggstarqaak |
| | plasmid | lskptlenltpvvIrpeirkpevlrpetpr |
| | Where each domain | pvdigsggfgdvgalesIrgnadlayilsm |
| | itches the case, | epcghcliinnvnfcresgIrtrtgsnidc |
| | bold, italic, and/or | ekIrrrfsslhfmvevkgdltakkmvlall |
| | underlined | elaqqdhgaldcevvvilshgcqashlqfp |
| | formatting between | gavygtdgepvsvekivnifngtscpslgg |
| | the description and | kpklffiqacggeqkdhgfevastspedes |
| | the sequence. | pgsnpepdatpfqeglrtfdqldaisslpt |
| | | psdifvsystfpgfvswrdpksgswyvetl |
| | | ddifeqwahsedlqsllIrvanavsvkgiy |
| | | kqmpgefnflrkklffkts |
| 9 | Amino acid | MALPVTALLLPLALLLHAARPGSDIQMTQS |
| | sequence of anti- | PSSLSASVGDRVTITCRASQEISGYLSWLQ |
| | FLT3 CAR with 5 | QKPGKAIKRLIYAASTLQSGVPSRFSGSRS |
| | amino acid linker | GSDYTLTISSLQPEDFATYYCLQYASYPFT |
| | (Signal peptide- | FGQGTKLEIKGGGGSQVTLKESGPTLVKPT |
| | linker-sAb 1978- | QTLTLTCTFSGFSLSTSTMGVGWIRQPPGK |
| | 30.11 VL-19aa | ALEWLAHILWNDSKRYNPSLKSRLTITKDT |
| | linker-sAb1978- | SKKQVVLTMTNMDPVDTATYYCARIVYYST |
| | 30.11 VH-linker- | YVGYFDVWGQGTTVTVSSGSFVPVFLPAKP |
| | CD8ahinge- | TTTPAPRPPTPAPTIASQPLSLRPEACRPA |
| | CD8aTM-CD28- | AGGAVHTRGLDFACDIYIWAPLAGTCGVLL |
| | 41BB-CD3zeta) | LSLVITLYCNHRNRSKRSRLLHSDYMNMTP |
| | encoded by | RRPGPTRKHYQPYAPPRDFAAYRSKRGRKK |
| | HG-CAR-TG105- | LLYIFKQPFMRPVQTTQEEDGCSCRFPEEE |
| | EF1_CAR3a_5aa | EGGCELRVKFSRSADAPAYQQGQNQLYNEL |
| | linker plasmid | NLGRREEYDVLDKRRGRDPEMGGKPRRKNP |
| | | QEGLYNELQKDKMAEAYSEIGMKGERRRGK |
| | | GHDGLYQGLSTATKDTYDALHMQALPPR |
| 10 | Amino acid | MALPVTALLLPLALLLHAARPGSDIQMTQS |
| | sequence of anti- | PSSLSASVGDRVTITCRASQEISGYLSWLQ |
| | FLT3 CAR with 19 | QKPGKAIKRLIYAASTLQSGVPSRFSGSRS |
| | amino acid linker | GSDYTLTISSLQPEDFATYYCLQYASYPFT |
| | (Signal peptide- | FGQGTKLEIKGGGGSGGGGSQVTLKESGPT |
| | linker-sAb 1978- | LVKPTQTLTLTCTFSGFSLSTSTMGVGWIR |
| | 30.11 VL-19aa | QPPGKALEWLAHILWNDSKRYNPSLKSRLT |
| | linker-sAb1978- | ITKDTSKKQVVLTMTNMDPVDTATYYCARI |
| | 30.11 VH-linker- | VYYSTYVGYFDVWGQGTTVTVSSGSFVPVF |
| | CD8ahinge- | LPAKPTTTPAPRPPTPAPTIASQPLSLRPE |
| | CD8aTM-CD28- | ACRPAAGGAVHTRGLDFACDIYIWAPLAGT |
| | 41BB-CD3zeta) | CGVLLLSLVITLYCNHRNRSKRSRLLHSDY |
| | encoded by | MNMTPRRPGPTRKHYQPYAPPRDFAAYRSK |
| | HG-CAR-TG105- | RGRKKLLYIFKQPFMRPVQTTQEEDGCSCR |
| | EF1_CAR3a_10aa | FPEEEEGGCELRVKFSRSADAPAYQQGQNQ |
| | linker | LYNELNLGRREEYDVLDKRRGRDPEMGGKP |
| | | RRKNPQEGLYNELQKDKMAEAYSEIGMKGE |
| | | RRRGKGHDGLYQGLSTATKDTYDALHMQAL |
| | | PPR |
| 11 | Amino acid | MALPVTALLLPLALLLHAARPGSDIQMTQS |
| | sequence of anti- | PSSLSASVGDRVTITCRASQEISGYLSWLQ |
| | FLT3 CAR with 15 | QKPGKAIKRLIYAASTLQSGVPSRFSGSRS |
| | amino acid linker | GSDYTLTISSLQPEDFATYYCLQYASYPFT |
| | (Signal peptide- | FGQGTKLEIKGGGGSGGGGSGGGGSQVTLK |
| | linker-sAb1978- | ESGPTLVKPTQTLTLTCTESGFSLSTSTMG |
| | 30.11 VL-19aa | VGWIRQPPGKALEWLAHILWNDSKRYNPSL |
| | linker-sAb 1978- | KSRLTITKDTSKKQVVLTMTNMDPVDTATY |
| | 30.11 VH-linker- | YCARIVYYSTYVGYFDVWGQGTTVTVSSGS |
| | CD8ahinge- | FVPVFLPAKPTTTPAPRPPTPAPTIASQPL |
| | CD8aTM-CD28- | SLRPEACRPAAGGAVHTRGLDFACDIYIWA |
| | 41BB-CD3zeta) | PLAGTCGVLLLSLVITLYCNHRNRSKRSRL |
| | encoded by | LHSDYMNMTPRRPGPTRKHYQPYAPPRDFA |
| | HG-CAR-TG105- | AYRSKRGRKKLLYIFKQPFMRPVQTTQEED |
| | EF1 CAR3a 15aal | GCSCRFPEEEEGGCELRVKFSRSADAPAYQ |
| | inker plasmid | QGQNQLYNELNLGRREEYDVLDKRRGRDPE |
| | | MGGKPRRKNPQEGLYNELQKDKMAEAYSEI |
| | | GMKGERRRGKGHDGLYQGLSTATKDTYDAL |
| | | HMQALPPR |
| 12 | Amino acid | MALPVTALLLPLALLLHAARPGSDIQMTQS |
| | sequence of anti- | PSSLSASVGDRVTITCRASQEISGYLSWLQ |
| | FLT3 CAR with 20 | QKPGKAIKRLIYAASTLQSGVPSRFSGSRS |
| | amino acid linker | GSDYTLTISSLQPEDFATYYCLQYASYPFT |
| | (Signal peptide- | FGQGTKLEIKGGGGSGGGGSGGGGSGGGGS |
| | linker-sAb1978- | QVTLKESGPTLVKPTQTLTLTCTFSGFSLS |
| | 30.11 VL-19aa | TSTMGVGWIRQPPGKALEWLAHILWNDSKR |
| | linker-sAb1978- | YNPSLKSRLTITKDTSKKQVVLTMTNMDPV |
| | 30.11 VH-linker- | DTATYYCARIVYYSTYVGYFDVWGQGTTVT |
| | CD8ahinge- | VSSGSFVPVFLPAKPTTTPAPRPPTPAPTI |
| | CD8aTM-CD28- | ASQPLSLRPEACRPAAGGAVHTRGLDFACD |
| | 41BB-CD3zeta) | IYIWAPLAGTCGVLLLSLVITLYCNHRNRS |
| | encoded by | KRSRLLHSDYMNMTPRRPGPTRKHYQPYAP |
| | HG-CAR-TG10S- | PRDFAAYRSKRGRKKLLYIFKQPFMRPVQT |
| | EF1_CAR3a_20aa | TQEEDGCSCRFPEEEEGGCELRVKFSRSAD |
| | linker plasmid | APAYQQGQNQLYNELNLGRREEYDVLDKRR |
| | | GRDPEMGGKPRRKNPQEGLYNELQKDKMAE |
| | | AYSEIGMKGERRRGKGHDGLYQGLSTATKD |
| | | TYDALHMQALPPR |
| 13 | Amino acid anti- | MALPVTALLLPLALLLHAARPGSDIQMTQS |
| | FLT3 CAR with 20 | PSSLSASVGDRVTITCRASQEISGYLSWLQ |
| | amino acid linker | QKPGKAIKRLIYAASTLQSGVPSRFSGSRS |
| | (Signal peptide- | GSDYTLTISSLQPEDFATYYCLQYASYPFT |
| | linker-sAb 1978- | FGQGTKLEIKGGGGSGGGGSGGGGSGGGGS |
| | 30.11 VL-19aa | QVTLKESGPTLVKPTQTLTLTCTFSGFSLS |
| | linker-sAb1978- | TSTMGVGWIRQPPGKALEWLAHILWNDSKR |
| | 30.11 VH-linker- | YNPSLKSRLTITKDTSKKQVVLTMTNMDPV |
| | CD8ahinge- | DTATYYCARIVYYSTYVGYFDVWGQGTTVT |
| | CD8aTM-CD28- | VSSGSFVPVFLPAKPTTTPAPRPPTPAPTI |
| | CD3zeta) encoded | ASQPLSLRPEACRPAAGGAVHTRGLDFACD |
| | by | IYIWAPLAGTCGVLLLSLVITLYCNHRNRS |
| | CAR3a-20aalinker- | KRSRLLHSDYMNMTPRRPGPTRKHYQPYAP |
| | NO-41BB plasmid | PRDFAAYRSRVKFSRSADAPAYQQGQNQLY |
| | | NELNLGRREEYDVLDKRRGRDPEMGGKPRR |
| | | KNPQEGLYNELQKDKMAEAYSEIGMKGERR |
| | | RGKGHDGLYQGLSTATKDTYDALHMQALPP |
| | | R |
| 14 | Amino acid | MALPVTALLLPLALLLHAARPGSDIQMTQS |
| | sequence of anti- | PSSLSASVGDRVTITCRASQEISGYLSWLQ |
| | FLT3 CAR with 19 | QKPGKAIKRLIYAASTLQSGVPSRFSGSRS |
| | amino acid linker | GSDYTLTISSLQPEDFATYYCLQYASYPFT |
| | (Signal peptide- | FGQGTKLEIKGGGGSGGGGSGGGSGGGGSQ |
| | linker-sAb1978- | VTLKESGPTLVKPTQTLTLTCTFSGFSLST |
| | 30.11 VL-19aa | STMGVGWIRQPPGKALEWLAHILWNDSKRY |
| | linker-sAb1978- | NPSLKSRLTITKDTSKKQVVLTMTNMDPVD |
| | 30.11 VH-linker- | TATYYCARIVYYSTYVGYFDVWGQGTTVTV |
| | CD8ahinge- | SSGSFVPVFLPAKPTTTPAPRPPTPAPTIA |
| | CD8aTM-41BB- | SQPLSLRPEACRPAAGGAVHTRGLDFACDI |
| | CD3zeta) encoded | YIWAPLAGTCGVLLLSLVITLYCNHRNKRG |
| | by | RKKLLYIFKQPFMRPVQTTQEEDGCSCRFP |
| | CAR3a-19aalinker- | EEEEGGCELRVKFSRSADAPAYQQGQNQLY |
| | NO-CD28 plasmid | NELNLGRREEYDVLDKRRGRDPEMGGKPRR |
| | | KNPQEGLYNELQKDKMAEAYSEIGMKGERR |
| | | RGKGHDGLYQGLSTATKDTYDALHMQALPP |
| | | R |
| 15 | Amino acid | MALPVTALLLPLALLLHAARPGSDIQMTQS |
| | sequence of anti- | PSSLSASVGDRVTITCRASQEISGYLSWLQ |
| | FLT3 CAR with 5 | QKPGKAIKRLIYAASTLQSGVPSRFSGSRS |
| | amino acid linker | GSDYTLTISSLQPEDFATYYCLQYASYPFT |
| | (Signal peptide- | FGQGTKLEIKGGGGSQVTLKESGPTLVKPT |
| | linker-sAb1978- | QTLTLTCTFSGFSLSTSTMGVGWIRQPPGK |
| | 30.11 VL-19aa | ALEWLAHILWNDSKRYNPSLKSRLTITKDT |
| | linker-sAb1978- | SKKQVVLTMTNMDPVDTATYYCARIVYYST |
| | 30.11 VH-linker- | YVGYFDVWGQGTTVTVSSGSFVPVFLPAKP |
| | CD8ahinge- | TTTPAPRPPTPAPTIASQPLSLRPEACRPA |
| | CD8aTM-CD28- | AGGAVHTRGLDFACDIYIWAPLAGTCGVLL |
| | CD3zeta) encoded | LSLVITLYCNHRNRSKRSRLLHSDYMNMTP |
| | by | RRPGPTRKHYQPYAPPRDFAAYRSRVKFSR |
| | CAR3a-5aalinker- | SADAPAYQQGQNQLYNELNLGRREEYDVLD |
| | NO-41BB | KRRGRDPEMGGKPRRKNPQEGLYNELQKDK |
| | | MAEAYSEIGMKGERRRGKGHDGLYQGLSTA |
| | | TKDTYDALHMQALPPR |
| 16 | Amino acid | MALPVTALLLPLALLLHAARPGSDIQMTQS |
| | sequence of anti- | PSSLSASVGDRVTITCRASQEISGYLSWLQ |
| | FLT3 CAR with 19 | QKPGKAIKRLIYAASTLQSGVPSRFSGSRS |
| | amino acid linker | GSDYTLTISSLQPEDFATYYCLQYASYPFT |
| | and GFP (Signal | FGQGTKLEIKGGGGSGGGGSGGGSGGGGSQ |
| | peptide-linker- | VTLKESGPTLVKPTQTLTLTCTFSGFSLST |
| | sAb1978-30.11 | STMGVGWIRQPPGKALEWLAHILWNDSKRY |
| | VL-19aa linker- | NPSLKSRLTITKDTSKKQVVLTMTNMDPVD |
| | sAb1978-30.11 | TATYYCARIVYYSTYVGYFDVWGQGTTVTV |
| | VH-linker- | SSGSFVPVFLPAKPTTTPAPRPPTPAPTIA |
| | CD8ahinge- | SQPLSLRPEACRPAAGGAVHTRGLDFACDI |
| | CD8aTM-CD28- | YIWAPLAGTCGVLLLSLVITLYCNHRNRSK |
| | 41BB-CD3zeta- | RSRLLHSDYMNMTPRRPGPTRKHYQPYAPP |
| | T2A-GFP) encoded | RDFAAYRSKRGRKKLLYIFKQPFMRPVQTT |
| | by pCDH-EF1- | QEEDGCSCRFPEEEEGGCELRVKFSRSADA |
| | CAR3a 19aa | PAYQQGQNQLYNELNLGRREEYDVLDKRRG |
| | linker T2A GFP | RDPEMGGKPRRKNPQEGLYNELQKDKMAEA |
| | | YSEIGMKGERRRGKGHDGLYQGLSTATKDT |
| | | YDALHMQALPPRAAAEGRGSLLTCGDVEEN |
| | | PGPSGMESDESGLPAMEIECRITGTLNGVE |
| | | FELVGGGEGTPKQGRMTNKMKSTKGALTFS |
| | | PYLLSHVMGYGFYHFGTYPSGYENPFLHAI |
| | | NNGGYTNTRIEKYEDGGVLHVSFSYRYEAG |
| | | RVIGDFKVVGTGFPEDSVIFTDKIIRSNAT |
| | | VEHLHPMGDNVLVGSFARTFSLRDGGYYSF |
| | | VVDSHMHFKSAIHPSILQNGGPMFAFRRVE |
| | | ELHSNTELGIVEYQHAFKTPIAFARSRAQS |
| | | SNSAVDGTAGPGSTGSR |
| 17 | Heavy chain | QVTLKESGPGILQPSQTLSLTCSFSGFSLS |
| | variable region of | TSTMGVGWIRQPSGKGLEWLLHILWNDSKY |
| | cAb1977-10.0 | YNPALKSRLTISKDTYNKQVFLKIANVDTA |
| | | DTATYYCARIVYYSTYVGYFDVWGAGTTVT |
| | | VSS |
| 18 | Heavy chain | QVTLKESGPTLVKPTQTLTLTCTFSGFSLS |
| | variable region of | TSTMGVGWIRQPPGKALEWLAHILWNDSKR |
| | cAb1979-10.0 | YNPSLKSRLTITKDTSKKQVVLTMTNMDPV |
| | | DTATYYCARIVYYSTYVGYFDVWGQGTTVT |
| | | VSS |
| 19 | Heavy chain | QITLKESGPTLVKPTQTLTLTCTFSGFSLS |
| | variable region of | TSTMGVGWIRQPPGKALEWLAHILWNDDKR |
| | cAb1981-10.0 | YGPSLKSRLTITKDTSKKQVVLTMTNMDPV |
| | | DTATYYCARIVYYSTYVGYFDVWGQGTTVT |
| | | VSS |
| 20 | Heavy chain | QITLKESGPTLVKPTQTLTLTCTFSGFSLS |
| | variable region of | TSTMGVGWIRQPPGKALEWLAHILWNDDKR |
| | cAb1982-10.0 | YGPSLKSRLTITKDTSKKQVVLTMTNMDPV |
| | | DTATYYCARIVYYSTYVGYFDVWGQGTTVT |
| | | VSS |
| 21 | Heavy chain | QITLKESGPTLVKPTQTLTLTCTFSGFSLS |
| | variable region of | TSTMGVGWIRQPPGKALEWLAHILWNDDKR |
| | cAb1983-10.0 | YGPSLKSRLTITKDTSKKQVVLTMTNMDPV |
| | | DTATYYCARIVYYSTYVGYFDVWGQGTTVT |
| | | VSS |
| 22 | Heavy chain | QVQLQESGPGLVKPSETLSLTCTFSGFSLS |
| | variable region of | TSTMGVGWIRQPPGKGLEWIGHILWNDSKY |
| | cAb1984-10.0 | YNPSLKSRVTISKDTSKKQFSLKLSSVTAA |
| | | DTAVYYCARIVYYSTYVGYFDVWGQGTTVT |
| | | VSS |
| 23 | Heavy chain | QVQLQESGPGLVKPSETLSLTCTFSGFSLS |
| | variable region of | TSTMGVGWIRQPPGKGLEWIGHILWNDSKY |
| | cAb1985-10.0 | YNPSLKSRVTISKDTSKKQFSLKLSSVTAA |
| | | DTAVYYCARIVYYSTYVGYFDVWGQGTTVT |
| | | VSS |
| 24 | Heavy chain | QVQLQESGPGLVKPSETLSLTCTFSGFSLS |
| | variable region of | TSTMGVGWIRQPPGKGLEWIGHILWNDSKY |
| | cAb1986-10.0 | YNPSLKSRVTISKDTSKKQFSLKLSSVTAA |
| | | DTAVYYCARIVYYSTYVGYFDVWGQGTTVT |
| | | VSS |
| 25 | Heavy chain | QVQLQQSGPGLVKPSQTLSLTCAFSGFSLS |
| | variable region of | TSTMGVGWIRQSPSRGLEWLGHILWNDSKD |
| | cAb1987-10.0 | YNPSVKSRITINKDTSKKQFSLQLNSVTPE |
| | | DTAVYYCARIVYYSTYVGYFDVWGQGTTVT |
| | | VSS |
| 26 | Heavy chain | QVQLQQSGPGLVKPSQTLSLTCAFSGFSLS |
| | variable region of | TSTMGVGWIRQSPSRGLEWLGHILWNDSKD |
| | cAb1988-10.0 | YNPSVKSRITINKDTSKKQFSLQLNSVTPE |
| | | DTAVYYCARIVYYSTYVGYFDVWGQGTTVT |
| | | VSS |
| 27 | Heavy chain | QVQLQQSGPGLVKPSQTLSLTCAFSGFSLS |
| | variable region of | TSTMGVGWIRQSPSRGLEWLGHILWNDSKD |
| | cAb1989-10.0 | YNPSVKSRITINKDTSKKQFSLQLNSVTPE |
| | | DTAVYYCARIVYYSTYVGYFDVWGQGTTVT |
| | | VSS |
| 28 | Light chain variable | DIQMTQSPSSLSASLGERVSLTCRASQEIS |
| | region of cAb1977- | GYLSWLQQKPDGTIKRLIYAASTLHSGVPK |
| | 10.0 | RFSGSRSGSDYSLTISRLESEDVADYYCLQ |
| | | YASYPFTFGSGTKLEIK |
| 29 | Light chain variable | EIVMTQSPGTLSLSPGERATLSCRASQEIS |
| | region of cAb1979- | GYLSWLQQKPGQAIRRLIYAASTRATGIPD |
| | 10.0 | RFSGSRSGSDYTLTISRLEPEDFAVYYCLQ |
| | | YASYPFTFGQGTKLEIK |
| 30 | Light chain variable | DIQMTQSPSSLSASVGDRVTITCRASQEIS |
| | region of cAb1981- | GYLSWLQQKPGKAIKRLIYAASTLQSGVPS |
| | 10.0 | RFSGSRSGSDYTLTISSLQPEDFATYYCLQ |
| | | YASYPFTFGQGTKLEIK |
| 31 | Light chain variable | EIVMTQSPGTLSLSPGERATLSCRASQEIS |
| | region of cAb1982- | GYLSWLQQKPGQAIRRLIYAASTRATGIPD |
| | 10.0 | RESGSRSGSDYTLTISRLEPEDFAVYYCLQ |
| | | YASYPFTFGQGTKLEI |
| 32 | Light chain variable | DIVMTQSPDSLAVSLGERATINCKASQEIS |
| | region of cAb 1983- | GYLSWLQQKPGQPIKRLIYAASTRESGVPD |
| | 10.0 | RFSGSRSGSDYTLTISSLQAEDVAVYYCLQ |
| | | YASYPFTFGQGTKLEIK |
| 33 | Light chain variable | DIQMTQSPSSLSASVGDRVTITCRASQEIS |
| | region of cAb 1984- | GYLSWLQQKPGKAIKRLIYAASTLQSGVPS |
| | 10.0 | RFSGSRSGSDYTLTISSLQPEDFATYYCLQ |
| | | YASYPFTFGQGTKLEIK |
| 34 | Light chain variable | EIVMTQSPGTLSLSPGERATLSCRASQEIS |
| | region of cAb1985- | GYLSWLQQKPGQAIRRLIYAASTRATGIPD |
| | 10.0 | RFSGSRSGSDYTLTISRLEPEDFAVYYCLQ |
| | | YASYPFTFGQGTKLEIK |
| 35 | Light chain variable | DIVMTQSPDSLAVSLGERATINCKASQEIS |
| | region of cAb1986- | GYLSWLQQKPGQPIKRLIYAASTRESGVPD |
| | 10.0 | RFSGSRSGSDYTLTISSLQAEDVAVYYCLQ |
| | | YASYPFTFGQGTKLEIK |
| 36 | Light chain variable | DIQMTQSPSSLSASVGDRVTITCRASQEIS |
| | region of cAb 1987- | GYLSWLQQKPGKAIKRLIYAASTLQSGVPS |
| | 10.0 | RFSGSRSGSDYTLTISSLOPEDFATYYCLQ |
| | | YASYPFTFGQGTKLEIK |
| 37 | Light chain variable | EIVMTQSPGTLSLSPGERATLSCRASQEIS |
| | region of cAb 1988- | GYLSWLQQKPGQAIRRLIYAASTRATGIPD |
| | 10.0 | RFSGSRSGSDYTLTISRLEPEDFAVYYCLQ |
| | | YASYPFTFGQGTKLEIK |
| 38 | Light chain variable | DIVMTQSPDSLAVSLGERATINCKASQEIS |
| | region of cAb1989- | GYLSWLQQKPGQPIKRLIYAASTRESGVPD |
| | 10.0 | RFSGSRSGSDYTLTISSLQAEDVAVYYCLQ |
| | | YASYPFTFGQGTKLEIK |
| 39 | Amino acid | DIQMTQSPSSLSASLGERVSLTCRASQEIS |
| | sequence of | GYLSWLQQKPDGTIKRLIYAASTLHSGVPK |
| | cAb1977-10.0 scFv | RFSGSRSGSDYSLTISRLESEDVADYYCLQ |
| | | YASYPFTFGSGTKLEIKGGGGSGGGGSGGG |
| | | SGGGGSQVTLKESGPGILQPSQTLSLTCSF |
| | | SGFSLSTSTMGVGWIRQPSGKGLEWLLHIL |
| | | WNDSKYYNPALKSRLTISKDTYNKQVFLKI |
| | | ANVDTADTATYYCARIVYYSTYVGYFDVWG |
| | | AGTTVTVSS |
| 40 | Amino acid | EIVMTQSPGTLSLSPGERATLSCRASQEIS |
| | sequence of | GYLSWLQQKPGQAIRRLIYAASTRATGIPD |
| | cAb1979-10.0 scFv | RFSGSRSGSDYTLTISRLEPEDFAVYYCLQ |
| | | YASYPFTFGQGTKLEIKGGGGSGGGGSGGG |
| | | SGGGGSQVTLKESGPTLVKPTQTLTLTCTF |
| | | SGFSLSTSTMGVGWIRQPPGKALEWLAHIL |
| | | WNDSKRYNPSLKSRLTITKDTSKKQVVLTM |
| | | TNMDPVDTATYYCARIVYYSTYVGYFDVWG |
| | | QGTTVTVSS |
| 41 | Amino acid | DIQMTQSPSSLSASVGDRVTITCRASQEIS |
| | sequence of | GYLSWLQQKPGKAIKRLIYAASTLQSGVPS |
| | cAb1981-10.0 scFv | RFSGSRSGSDYTLTISSLQPEDFATYYCLQ |
| | | YASYPFTFGQGTKLEIKGGGGSGGGGSGGG |
| | | SGGGGSQITLKESGPTLVKPTQTLTLTCTF |
| | | SGFSLSTSTMGVGWIRQPPGKALEWLAHIL |
| | | WNDDKRYGPSLKSRLTITKDTSKKQVVLTM |
| | | TNMDPVDTATYYCARIVYYSTYVGYFDVWG |
| | | QGTTVTVSS |
| 42 | Amino acid | EIVMTQSPGTLSLSPGERATLSCRASQEIS |
| | sequence of | GYLSWLQQKPGQAIRRLIYAASTRATGIPD |
| | cAb 1982-10,0 scFv | RFSGSRSGSDYTLTISRLEPEDFAVYYCLQ |
| | | YASYPFTFGQGTKLEIKGGGGSGGGGSGGG |
| | | SGGGGSQITLKESGPTLVKPTQTLTLTCTF |
| | | SGFSLSTSTMGVGWIRQPPGKALEWLAHIL |
| | | WNDDKRYGPSLKSRLTITKDTSKKQVVLTM |
| | | TNMDPVDTATYYCARIVYYSTYVGYFDVWG |
| | | QGTTVTVSS |
| 43 | Amino acid | DIVMTQSPDSLAVSLGERATINCKASQEIS |
| | sequence of | GYLSWLQQKPGQPIKRLIYAASTRESGVPD |
| | cAb1983-10.0 scFv | RFSGSRSGSDYTLTISSLQAEDVAVYYCLQ |
| | | YASYPFTFGQGTKLEIKGGGGSGGGGSGGG |
| | | SGGGGSQITLKESGPTLVKPTQTLTLTCTF |
| | | SGFSLSTSTMGVGWIRQPPGKALEWLAHIL |
| | | WNDDKRYGPSLKSRLTITKDTSKKQVVLTM |
| | | TNMDPVDTATYYCARIVYYSTYVGYFDVWG |
| | | QGTTVTVSS |
| 44 | Amino acid | DIQMTQSPSSLSASVGDRVTITCRASQEIS |
| | sequence of | GYLSWLQQKPGKAIKRLIYAASTLQSGVPS |
| | cAb1984-10.0 scFv | RFSGSRSGSDYTLTISSLOPEDFATYYCLQ |
| | | YASYPFTFGQGTKLEIKGGGGSGGGGSGGG |
| | | SGGGGSQVQLQESGPGLVKPSETLSLTCTF |
| | | SGFSLSTSTMGVGWIRQPPGKGLEWIGHIL |
| | | WNDSKYYNPSLKSRVTISKDTSKKQFSLKL |
| | | SSVTAADTAVYYCARIVYYSTYVGYFDVWG |
| | | QGTTVTVSS |
| 45 | Amino acid | EIVMTQSPGTLSLSPGERATLSCRASQEIS |
| | sequence of | GYLSWLQQKPGQAIRRLIYAASTRATGIPD |
| | cAb1985-10.0 scFv | RFSGSRSGSDYTLTISRLEPEDFAVYYCLQ |
| | | YASYPFTFGQGTKLEIKGGGGSGGGGSGGG |
| | | SGGGGSQVQLQESGPGLVKPSETLSLTCTF |
| | | SGFSLSTSTMGVGWIRQPPGKGLEWIGHIL |
| | | WNDSKYYNPSLKSRVTISKDTSKKQFSLKL |
| | | SSVTAADTAVYYCARIVYYSTYVGYFDVWG |
| | | QGTTVTVSS |
| 46 | Amino acid | DIVMTQSPDSLAVSLGERATINCKASQEIS |
| | sequence of | GYLSWLQQKPGQPIKRLIYAASTRESGVPD |
| | cAb1986-10.0 scFv | RFSGSRSGSDYTLTISSLQAEDVAVYYCLQ |
| | | YASYPFTFGQGTKLEIKGGGGSGGGGSGGG |
| | | SGGGGSQVQLQESGPGLVKPSETLSLTCTF |
| | | SGFSLSTSTMGVGWIRQPPGKGLEWIGHIL |
| | | WNDSKYYNPSLKSRVTISKDTSKKQFSLKL |
| | | SSVTAADTAVYYCARIVYYSTYVGYFDVWG |
| | | QGTTVTVSS |
| 47 | Amino acid | DIQMTQSPSSLSASVGDRVTITCRASQEIS |
| | sequence of | GYLSWLQQKPGKAIKRLIYAASTLQSGVPS |
| | cAb1987-10.0 scFv | RFSGSRSGSDYTLTISSLQPEDFATYYCLQ |
| | | YASYPFTFGQGTKLEIKGGGGSGGGGSGGG |
| | | SGGGGSQVQLQQSGPGLVKPSQTLSLTCAF |
| | | SGFSLSTSTMGVGWIRQSPSRGLEWLGHIL |
| | | WNDSKDYNPSVKSRITINKDTSKKQFSLQL |
| | | NSVTPEDTAVYYCARIVYYSTYVGYFDVWG |
| | | OGTTVTVSS |
| 48 | Amino acid | EIVMTQSPGTLSLSPGERATLSCRASQEIS |
| | sequence of | GYLSWLQQKPGQAIRRLIYAASTRATGIPD |
| | cAb1988-10.0 scFv | RFSGSRSGSDYTLTISRLEPEDFAVYYCLQ |
| | | YASYPFTFGQGTKLEIKGGGGSGGGGSGGG |
| | | SGGGGSQVQLQQSGPGLVKPSQTLSLTCAF |
| | | SGFSLSTSTMGVGWIRQSPSRGLEWLGHIL |
| | | WNDSKDYNPSVKSRITINKDTSKKQFSLQL |
| | | NSVTPEDTAVYYCARIVYYSTYVGYFDVWG |
| | | QGTTVTVSS |
| 49 | Amino acid | DIVMTQSPDSLAVSLGERATINCKASQEIS |
| | sequence of | GYLSWLQQKPGQPIKRLIYAASTRESGVPD |
| | cAb1989-10.0 scFv | RFSGSRSGSDYTLTISSLQAEDVAVYYCLQ |
| | | YASYPFTFGQGTKLEIKGGGGSGGGGSGGG |
| | | SGGGGSQVQLQQSGPGLVKPSQTLSLTCAF |
| | | SGFSLSTSTMGVGWIRQSPSRGLEWLGHIL |
| | | WNDSKDYNPSVKSRITINKDTSKKQFSLQL |
| | | NSVTPEDTAVYYCARIVYYSTYVGYFDVWG |
| | | QGTTVTVSS |
| 50 | Amino acid | GGGGS |
| | sequence of | |
| | Gly₄Ser Linker | |
| 51 | Amino acid | GGGGSGGGGS |
| | sequence of | |
| | (Gly₄Ser)₂ Linker | |
| 52 | Amino acid | GGGGSGGGGSGGGGS |
| | sequence of | |
| | (GlyaSer)₃ Linker | |
| 53 | Amino acid | GGGGSGGGGSGGGSGGGGS |
| | sequence of | |
| | (Gly₄Ser)₂ Gly₃Ser | |
| | Gly₄Ser | |
| 54 | Amino acid | GGGGSGGGGSGGGGSGGGGS |
| | sequence of | |
| | (Gly₄Ser)₄ Linker | |
| 55 | Nucleotide | ggtggcggcggctct |
| | sequence of | |
| | Gly₄Ser Linker | |
| 56 | Nucleotide | ggtggcggcggctctggcggcggtggctct |
| | sequence of | |
| | (Gly₄Ser)₂ Linker | |
| 57 | Nucleotide | ggtggcggcggctctggcggcggtggctct |
| | sequence of | ggcggcggtggctcc |
| | (Gly₄Ser)₃ Linker | |
| 58 | Nucleotide | ggtggcggcggctctggcggcggtggctct |
| | sequence of | ggcggtggctccggcggtggtggtagc |
| | (Gly₄Ser)₂ GlysSer | |
| | Gly₄Ser | |
| 59 | Nucleotide | Gtggcggcggctctggcggcggtggctctg |
| | sequence of | gcggcggtggctccggcggtggtggtagC |
| | (Gly₄Ser)₄ Linker | |
| 60 | Nucleic acid | atggccttaccagtgaccgccttgctcctg |
| | sequence of anti- | ccgctggccttgctgctccacgccgccagg |
| | FLT3 CAR with 19 | ccgggatccgacatccagatgacccagagc |
| | amino acid linker | ccctcctctttatccgcctctgtgggcgac |
| | (Signal peptide- | agagtgaccatcacttgtcgtgccagccaa |
| | linker-sAb 1978- | gaaatctccggctatctgtcttggctgcag |
| | 30.11 VL-19aa | cagaagcccggtaaggctatcaagagactc |
| | linker-sAb1978- | atctacgccgccagcactttacagagcgga |
| | 30.11 VH-linker- | gtgcctagcagatttagcggctctcgtagc |
| | CD8ahinge- | ggcagcgattatactttaaccatctcctct |
| | CD8aTM-CD28- | ttacagcccgaagactttgctacctactac |
| | 41BB-CD3zeta) | tgtttacagtacgccagctaccccttcacc |
| | encoded by pCDH- | ttcggtcaaggtaccaagctggagatcaaa |
| | EF1-CAR3a 19aa | ggtggcggcggctctggcggcggtggctct |
| | linker plasmid | ggcggtggctccggcggtggtggtagccaa |
| | | gttaccctcaaggagagcggccccacttta |
| | | gtgaagcctacccagactttaactttaact |
| | | tgtaccttcagcggcttctctttaagcacc |
| | | tccacaatgggcgtgggctggatcagacag |
| | | cctcccggcaaggctctggagtggctggcc |
| | | cacatcctctggaacgacagcaagaggtac |
| | | aacccctctttaaagtctcgtctgaccatc |
| | | accaaggacacctccaagaagcaagtigtg |
| | | ctgaccatgaccaatatggaccccgtggac |
| | | accgccacctattactgcgctcgtatcgtg |
| | | tactactccacctacgtgggctacttcgac |
| | | gtctggggacaaggtaccaccgtgaccgtg |
| | | agctctggatccttcgtgccggtcttcctg |
| | | ccagcgaagcccaccacgacgccagcgccg |
| | | cgaccaccaacaccggcgcccaccatcgcg |
| | | tcgcagcccctgtccctgcgcccagaggcg |
| | | tgccggccagcggcggggggcgcagtgcac |
| | | acgagggggctggacttcgcctgtgatatc |
| | | tacatctgggcgcccttggccgggacttgt |
| | | ggggtccttctcctgtcactggttatcacc |
| | | ctttactgcaaccacaggaacaggagtaag |
| | | aggagcaggctcctgcacagtgactacatg |
| | | aacatgactccccgccgccccgggcccacc |
| | | cgcaagcattaccagccctatgccccacca |
| | | cgcgacttcgcagcctatcgctccaaacgg |
| | | ggcagaaagaaactcctgtatatattcaaa |
| | | caaccatttatgagaccagtacaaactact |
| | | caagaggaagatggctgtagctgccgattt |
| | | ccagaagaagaagaaggaggatgtgaactg |
| | | agagtgaagttcagcaggagcgcagacgcc |
| | | cccgcgtaccagcagggccagaaccagctc |
| | | tataacgagctcaatctaggacgaagagag |
| | | gagtacgatgttttggacaagagacgtggc |
| | | cgggaccctgagatggggggaaagccgaga |
| | | aggaagaaccctcaggaaggcctgtacaat |
| | | gaactgcagaaagataagatggcggaggcc |
| | | tacagtgagattgggatgaaaggcgagcgc |
| | | cggaggggcaaggggcacgatggcctttac |
| | | cagggtctcagtacagccaccaaggacacc |
| | | tacgacgcccttcacatgcaggccctgccc |
| | | cctcgctga |
| 61 | Nucleic acid | atggccttaccagtgaccgccttgctcctg |
| | sequence of anti- | ccgctggccttgctgctccacgccgccagg |
| | FLT3 CAR with 19 | ccgggatccgacatccagatgacccagagc |
| | amino acid linker | ccctcctctttatccgcctctgtgggcgac |
| | and EGFRt (Signal | agagtgaccatcacttgtcgtgccagccaa |
| | peptide-linker- | gaaatctccggctatctgtcttggctgcag |
| | sAb1978-30.11 | cagaagcccggtaaggctatcaagagactc |
| | VL-19aa linker- | atctacgccgccagcactttacagagcgga |
| | sAb1978-30.11 | gtgcctagcagatttagcggctctcgtagc |
| | VH-linker- | ggcagcgattatactttaaccatctcctct |
| | CD8ahinge- | ttacagcccgaagactttgctacctactac |
| | CD8aTM-CD28- | tgtttacagtacgccagctaccccttcacc |
| | 41BB-CD3zeta- | ttcggtcaaggtaccaagctggagatcaaa |
| | T2A-EGFRt) | ggtggcggcggctctggcggcggtggctct |
| | encoded by | ggcggtggctccggcggtggtggtagccaa |
| | EF1_CAR3a_19aa | gttaccctcaaggagagcggccccacttta |
| | linkerT2Aegfit_VB | gtgaagcctacccagactttaactttaact |
| | 200623 plasmid | tgtaccttcagcggcttctctttaagcacc |
| | | tccacaatgggcgtgggctggatcagacag |
| | | cctcccggcaaggctctggagtggctggcc |
| | | cacatcctctggaacgacagcaagaggtac |
| | | aacccctctttaaagtctcgtctgaccatc |
| | | accaaggacacctccaagaagcaagttgtg |
| | | ctgaccatgaccaatatggaccccgtggac |
| | | accgccacctattactgcgctcgtatcgtg |
| | | tactactccacctacgtgggctacttcgac |
| | | gtctggggacaaggtaccaccgtgaccgtg |
| | | agctctggatccttcgtgccggtcttcctg |
| | | ccagcgaagcccaccacgacgccagcgccg |
| | | cgaccaccaacaccggcgcccaccatcgcg |
| | | tcgcagcccctgtccctgcgcccagaggcg |
| | | tgccggccagcggggggggcgcagtgcaca |
| | | cgagggggctggacttcgcctgtgatatct |
| | | acatctgggcgcccttggccgggacttgtg |
| | | gggtccttctcctgtcactggttatcaccc |
| | | tttactgcaaccacaggaacaggagtaaga |
| | | ggagcaggctcctgcacagtgactacatga |
| | | acatgactccccgccgccccgggcccaccc |
| | | gcaagcattaccagccctatgccccaccac |
| | | gcgacttcgcagcctatcgctccaaacggg |
| | | gcagaaagaaactcctgtatatattcaaac |
| | | aaccatttatgagaccagtacaaactactc |
| | | aagaggaagatggctgtagctgccgatttc |
| | | cagaagaagaagaaggaggatgtgaactga |
| | | gagtgaagttcagcaggagcgcagacgccc |
| | | ccgcgtaccagcagggccagaaccagctct |
| | | ataacgagctcaatctaggacgaagagagg |
| | | agtacgatgttttggacaagagacgtggcc |
| | | gggaccctgagatggggggaaagccgagaa |
| | | ggaagaaccctcaggaaggcctgtacaatg |
| | | aactgcagaaagataagatggcggaggcct |
| | | acagtgagattgggatgaaaggcgagcgcc |
| | | ggaggggcaaggggcacgatggcctttacc |
| | | agggtctcagtacagccaccaaggacacct |
| | | acgacgcccttcacatgcaggccctgcccc |
| | | ctcgcggaagcggagagggcaggggaagtc |
| | | ttctaacatgcggggacgtggaggaaaatc |
| | | ccggccccatgcttctcctggtgacaagcc |
| | | ttctgctctgtgagttaccacacccagcat |
| | | tcctcctgatcccacgcaaagtgtgtaacg |
| | | gaataggtattggtgaatttaaagactcac |
| | | tctccataaatgctacgaatattaaacact |
| | | tcaaaaactgcacctccatcagtggcgatc |
| | | tccacatcctgccggtggcatttaggggtg |
| | | actccttcacacatactcctcctctggatc |
| | | cacaggaactggatattctgaaaaccgtaa |
| | | aggaaatcacagggtttttgctgattcagg |
| | | cttggcctgaaaacaggacggacctccatg |
| | | cctttgagaacctagaaatcatacgcggca |
| | | ggaccaagcaacatggtcagttttctcttg |
| | | cagtgtcagcctgaacataacatccttggg |
| | | attacgctccctcaaggagataagtgatgg |
| | | agatgtgataatttcaggaaacaaaaattt |
| | | gtgctatgcaaatacaataaactggaaaaa |
| | | actgtttgggacctccggtcagaaaaccaa |
| | | aattataagcaacagaggtgaaaacagctg |
| | | caaggccacaggccaggtctgccatgcctt |
| | | gtgctcccccgagggctgctggggcccgga |
| | | gcccagggactgcgtctcttgccggaatgt |
| | | cagccgaggcagggaatgcgtggacaagtg |
| | | caaccttctggagggtgagccaagggagtt |
| | | tgtggagaactctgagtgcatacagtgcca |
| | | cccagagtgcctgcctcaggccatgaacat |
| | | cacctgcacaggacggggaccagacaactg |
| | | tatccagtgtgcccactacattgacggccc |
| | | ccactgcgtcaagacctgcccggcaggagt |
| | | catgggagaaaacaacaccctggtctggaa |
| | | gtacgcagacgccggccatgtgtgccacct |
| | | gtgccatccaaactgcacctacggatgcac |
| | | tgggccaggtcttgaaggctgtccaacgaa |
| | | tgggcctaagatcccgtccatcgccactgg |
| | | gatggtgggggccctcctcttgctgctggt |
| | | ggtggccctggggatcggcctcttcatgtg |
| | | a |
| 62 | Nucleic acid | atggccttaccagtgaccgccttgctcctg |
| | sequence of anti- | ccgctggccttgctgctccacgccgccagg |
| | FLT3 CAR with 19 | ccgggatccgacatccagatgacccagagc |
| | amino acid linker | ccctcctctttatccgcctctgtgggcgac |
| | and iCasp9 (Signal | agagtgaccatcacttgtcgtgccagccaa |
| | peptide-linker- | gaaatctccggctatctgtcttggctgcag |
| | sAb1978-30.11 | cagaagcccggtaaggctatcaagagactc |
| | VL-19aa linker- | atctacgccgccagcactttacagagcgga |
| | sAb1978-30.11 | gtgcctagcagatttagcggctctcgtagc |
| | VH-linker- | ggcagcgattatactttaaccatctcctct |
| | CD8ahinge- | ttacagcccgaagactttgctacctactac |
| | CD8aTM-CD28- | tgtttacagtacgccagctaccccttcacc |
| | 41BB-CD3zeta- | ttcggtcaaggtaccaagctggagatcaaa |
| | T2A-iCasp9) | ggtggcggcggctctggcggcggtggctct |
| | encoded by | ggcggtggctccggcggtggtggtagccaa |
| | pCDH-EF1- | gttaccctcaaggagagcggccccacttta |
| | CAR3a_19aa | gtgaagcctacccagactttaactttaact |
| | linker_T2A_ | tgtaccttcagcggcttctctttaagcacc |
| | icasp9 | tccacaatgggcgtgggctggatcagacag |
| | plasmid | cctcccggcaaggctctggagtggctggcc |
| | | cacatcctctggaacgacagcaagaggtac |
| | | aacccctctttaaagtctcgtctgaccatc |
| | | accaaggacacctccaagaagcaagtigtg |
| | | ctgaccatgaccaatatggaccccgtggac |
| | | accgccacctattactgcgctcgtatgtgt |
| | | actactccacctacgtgggctacttcgacg |
| | | tctggggacaaggtaccaccgtgaccgtga |
| | | gctctggatccttcgtgccggtcttcctgc |
| | | cagcgaagcccaccacgacgccagcgccgc |
| | | gaccaccaacaccggcgcccaccatcgcgt |
| | | cgcagcccctgtccctgcgcccagaggcgt |
| | | gccggccagcggcggggggcgcagtgcaca |
| | | cgagggggctggacttcgcctgtgatatct |
| | | acatctgggcgcccttggccgggacttgtg |
| | | gggtccttctcctgtcactggttatcaccc |
| | | tttactgcaaccacaggaacaggagtaaga |
| | | ggagcaggctcctgcacagtgactacatga |
| | | acatgactccccgccgccccgggcccaccc |
| | | gcaagcattaccagccctatgccccaccag |
| | | cgacttcgcagcctatcgctccaaacgggg |
| | | cagaaagaaactcctgtatatattcaaaca |
| | | accatttatgagaccagtacaaactactca |
| | | agaggaagatggctgtagctgccgatttcc |
| | | agaagaagaagaaggaggatgtgaactgag |
| | | agtgaagttcagcaggagcgcagacgcccc |
| | | cgcgtaccagcagggccagaaccagctcta |
| | | taacgagctcaatctaggacgaagagagga |
| | | gtacgatgttttggacaagagacgtggccg |
| | | ggaccctgagatggggggaaagccgagaag |
| | | gaagaaccctcaggaaggcctgtacaatga |
| | | actgcagaaagataagatggcggaggccta |
| | | cagtgagattgggatgaaaggcgagcgccg |
| | | gaggggcaaggggcacgatggcctttacca |
| | | gggtctcagtacagccaccaaggacaccta |
| | | cgacgcccttcacatgcaggccctgccccc |
| | | tcgcggaagcggagagggcaggggaagtct |
| | | tctaacatgcggggacgtggaggaaaatcc |
| | | cggccccatgggagtgcaggtggaaaccat |
| | | ctccccaggagacgggcgcaccttccccaa |
| | | gcgcggccagacctgcgtggtgcactacac |
| | | cgggatgcttgaagatggaaagaaagtgga |
| | | ttcctcccgggacagaaacaagccctttaa |
| | | gtttatgctaggcaagcaggaggtgatccg |
| | | aggctgggaagaaggggttgcccagatgag |
| | | tgtgggtcagagagccaaactgactatatc |
| | | tccagattatgcctatggtgccactgggca |
| | | cccaggcatcatcccaccacatgccactct |
| | | cgtcttcgatgtggagcttctaaaactgga |
| | | atctggaggaggttctactaacaggcaagc |
| | | agcaaagttgtcgaagccaaccctagaaaa |
| | | ccttaccccagtggtgctcagaccagagat |
| | | tcgcaaaccagaggttctcagaccggaaac |
| | | acccagaccagtggacattggttctggagg |
| | | atttggtgatgtcggtgctcttgagagttt |
| | | gaggggaaatgcagatttggcttacatcct |
| | | gagcatggagccctgtggccactgcctcat |
| | | tatcaacaatgtgaacttctgccgtgagtc |
| | | cgggctccgcacccgcactggctccaacat |
| | | cgactgtgagaagttgcggcgtcgcttctc |
| | | ctcgctgcatttcatggtggaggtgaaggg |
| | | cgacctgactgccaagaaaatggtgctggc |
| | | tttgctggagctggcgcagcaggaccacgg |
| | | tgctctggactgctgcgtggtggtcattct |
| | | ctctcacggctgtcaggccagccacctgca |
| | | gttcccaggggctgtctacggcacagatgg |
| | | atgccctgtgtcggtcgagaagattgtgaa |
| | | catcttcaatgggaccagctgccccagcct |
| | | gggagggaagcccaagctctttttcatcca |
| | | ggcctgtggtggggagcagaaagaccatgg |
| | | gtttgaggtggcctccacttcccctgaaga |
| | | cgagtcccctggcagtaaccccgagccaga |
| | | tgccaccccgttccaggaaggtttgaggac |
| | | cttcgaccagctggacgccatatctagttt |
| | | gcccacacccagtgacatctttgtgtccta |
| | | ctctactttcccaggttttgtttcctggag |
| | | ggaccccaagagtggctcctggtacgttga |
| | | gaccctggacgacatctttgagcagtgggc |
| | | tcactctgaagacctgcagtccctcctgct |
| | | tagggtcgctaatgctgtttcggtgaaagg |
| | | gatttataaacagatgcctggttgctttaa |
| | | tttcctccggaaaaaacttttctttaaaac |
| | | atcatga |
| 63 | Nucleic acid | atggccttaccagtgaccgccttgctcctg |
| | sequence of anti- | ccgctggccttgctgctccacgccgccagg |
| | FLT3 CAR with 5 | ccgggatccgacatccagatgacccagagc |
| | amino acid linker | ccctcctctttatccgcctctgtgggcgac |
| | (Signal peptide- | agagtgaccatcacttgtcgtgccagccaa |
| | linker-sAb1978- | gaaatctccggctatctgtcttggctgcag |
| | 30.11 VL-19aa | cagaagcccggtaaggctatcaagagactc |
| | linker-sAb 1978- | atctacgccgccagcactttacagagcgga |
| | 30.11 VH-linker- | gtgcctagcagatttagcggctctcgtagg |
| | CD8ahinge- | gcagcgattatactttaaccatctcctctt |
| | CD8aTM-CD28- | tacagcccgaagactttgctacctactact |
| | 41BB-CD3zeta) | gtttacagtacgccagctaccccttcacct |
| | encoded by | tcggtcaaggtaccaagctggagatcaaag |
| | HG-CAR-TG105- | gtggcggcggctctcaagttaccctcaagg |
| | EF1_CAR3a_5aa | agagcggccccactttagtgaagcctaccc |
| | linker plasmid | agactttaactttaacttgtaccttcagcg |
| | | gcttctctttaagcacctccacaatgggcg |
| | | tgggctggatcagacagcctcccggcaagg |
| | | ctctggagtggctggcccacatcctctgga |
| | | acgacagcaagaggtacaacccctctttaa |
| | | agtctcgtctgaccatcaccaaggacacct |
| | | ccaagaagcaagtigtgctgaccatgacca |
| | | atatggaccccgtggacaccgccacctatt |
| | | actgcgctcgtatcgtgtactactccacct |
| | | acgtgggctacttcgacgtctggggacaag |
| | | gtaccaccgtgaccgtgagctctggatcct |
| | | tcgtgccggtcttcctgccagcgaagccca |
| | | ccacgacgccagcgccgcgaccaccaacac |
| | | cggcgcccaccatcgcgtcgcagcccctgt |
| | | ccctgcgcccagaggcgtgccggccagcgg |
| | | cggggggcgcagtgcacacgagggggctgg |
| | | acttcgcctgtgatatctacatctgggcgc |
| | | ccttggccgggacttgtggggtccttctcc |
| | | tgtcactggttatcaccctttactgcaacc |
| | | acaggaacaggagtaagaggagcaggctcc |
| | | tgcacagtgactacatgaacatgactcccc |
| | | gccgccccgggcccacccgcaagcattacc |
| | | agccctatgccccaccacgcgacttcgcag |
| | | cctatcgctccaaacggggcagaaagaaac |
| | | tcctgtatatattcaaacaaccatttatga |
| | | gaccagtacaaactactcaagaggaagatg |
| | | gctgtagctgccgatttccagaagaagaag |
| | | aaggaggatgtgaactgagagtgaagttca |
| | | gcaggagcgcagacgcccccgcgtaccagc |
| | | agggccagaaccagctctataacgagctca |
| | | atctaggacgaagagaggagtacgatgttt |
| | | tggacaagagacgtggccgggaccctgaga |
| | | tggggggaaagccgagaaggaagaaccctc |
| | | aggaaggcctgtacaatgaactgcagaaag |
| | | ataagatggcggaggcctacagtgagattg |
| | | ggatgaaaggc gagcgccggaggggcaag |
| | | gggcacgatggcctttaccagggtctcagt |
| | | acagccacca aggacacctacgacgccct |
| | | tcacatgcaggccctgccccctcgctga |
| 64 | Nucleic acid | atggccttaccagtgaccgccttgctcctg |
| | sequence of anti- | ccgctggccttgctgctccacgccgccagg |
| | FLT3 CAR with 19 | ccgggatccgacatccagatgacccagagc |
| | amino acid linker | ccctcctctttatccgcctctgtgggcgac |
| | (Signal peptide- | agagtgaccatcacttgtcgtgccagccaa |
| | linker-sAb1978- | gaaatctccggctatctgtcttggctgcag |
| | 30.11 VL-19aa | cagaagcccggtaaggctatcaagagactc |
| | linker-sAb1978- | atctacgccgccagcactttacagagcgga |
| | 30.11 VH-linker- | gtgcctagcagatttagcggctctcgtagc |
| | CD8ahinge- | ggcagcgattatactttaaccatctcctct |
| | CD8aTM-CD28- | ttacagcccgaagactttgctacctactac |
| | 41BB-CD3zeta) | tgtttacagtacgccagctaccccttcacc |
| | encoded by | ttcggtcaaggtaccaagctggagatcaaa |
| | HG-CAR-TG105- | ggtggcggcggctctggcggcggtggctct |
| | EF1_CAR3a_10aa | caagttaccctcaaggagagcggccccact |
| | linker plasmid | ttagtgaagcctacccagactttaacttta |
| | | acttgtaccttcagcggcttctctttaagc |
| | | acctccacaatgggcgtgggctggatcaga |
| | | cagcctcccggcaaggctctggagtggctg |
| | | gcccacatcctctggaacgacagcaagagg |
| | | tacaacccctctttaaagtctcgtctgacc |
| | | atcaccaaggacacctccaagaagcaagti |
| | | gtgctgaccatgaccaatatggaccccgtg |
| | | gacaccgccacctattactgcgctcgtatc |
| | | gtgtactactccacctacgtgggctacttc |
| | | gacgtctggggacaaggtaccaccgtgacc |
| | | gtgagctctggatccttcgtgccggtcttc |
| | | ctgccagcgaagcccaccacgacgccagcg |
| | | ccgcgaccaccaacaccggcgcccaccatc |
| | | gcgtcgcagcccctgtccctgcgcccagag |
| | | gcgtgccggccagcggggggggcgcagtgc |
| | | acacgagggggctggacttcgcctgtgata |
| | | tctacatctgggcgcccttggccgggactt |
| | | gtggggtccttctcctgtcactggttatca |
| | | ccctttactgcaaccacaggaacaggagta |
| | | agaggagcaggctcctgcacagtgactaca |
| | | tgaacatgactccccgccgccccgggccca |
| | | cccgcaagcattaccagccctatgccccac |
| | | cacgcgacttcgcagcctatcgctccaaac |
| | | ggggcagaaagaaactcctgtatatattca |
| | | aacaaccatttatgagaccagtacaaacta |
| | | ctcaagaggaagatggctgtagctgccgat |
| | | ttccagaagaagaagaaggaggatgtgaac |
| | | tgagagtgaagttcagcaggagcgcagacg |
| | | cccccgcgtaccagcagggccagaaccagc |
| | | tctataacgagctcaatctaggacgaagag |
| | | aggagtacgatgttttggacaagagacgtg |
| | | gccgggaccctgagatggggggaaagccga |
| | | gaaggaagaaccctcaggaaggcctgtaca |
| | | atgaactgcagaaagataagatggcggagg |
| | | cctacagtgagattgggatgaaaggcgagc |
| | | gccggaggggcaaggggcacgatggccttt |
| | | accagggtctcagtacagccaccaaggaca |
| | | cctacgacgcccttcacatgcaggccctgc |
| | | cccctcgctga |
| 65 | Nucleic acid | atggccttaccagtgaccgccttgctcctg |
| | sequence of anti- | ccgctggccttgctgctccacgccgccagg |
| | FLT3 CAR with 15 | ccgggatccgacatccagatgacccagagc |
| | amino acid linker | ccctcctctttatccgcctctgtgggcgac |
| | (Signal peptide- | agagtgaccatcacttgtcgtgccagccaa |
| | linker-sAb 1978- | gaaatctccggctatctgtcttggctgcag |
| | 30.11 VL-19aa | cagaagcccggtaaggctatcaagagactc |
| | linker-sAb 1978- | atctacgccgccagcactttacagagcgga |
| | 30.11 VH-linker- | gtgcctagcagatttagcggctctcgtagc |
| | CD8ahinge- | ggcagcgattatactttaaccatctcctct |
| | CD8aTM-CD28- | ttacagcccgaagactttgctacctactac |
| | 41BB-CD3zeta) | tgtttacagtacgccagctaccccttcacc |
| | encoded by | ttcggtcaaggtaccaagctggagatcaaa |
| | HG-CAR-TG105- | ggtggcggcggctctggcggcggtggctct |
| | EF1 CAR3a 15aa | ggcggcggtggctcccaagttaccctcaag |
| | linker plasmid | gagagcggccccactttagtgaagcctacc |
| | | cagactttaactttaacttgtaccttcagc |
| | | ggcttctctttaagcacctccacaatgggc |
| | | gtgggctggatcagacagcctcccggcaag |
| | | gctctggagtggctggcccacatcctctgg |
| | | aacgacagcaagaggtacaacccctcttta |
| | | aagtctcgtctgaccatcaccaaggacacc |
| | | tccaagaagcaagitgtgctgaccatgacc |
| | | aatatggaccccgtggacaccgccacctat |
| | | tactgcgctcgtatgtgtactactccacct |
| | | acgtgggctacttcgacgtctggggacaag |
| | | gtaccaccgtgaccgtgagctctggatcct |
| | | tcgtgccggtcttcctgccagcgaagccca |
| | | ccacgacgccagcgccgcgaccaccaacac |
| | | cggcgcccaccatcgcgtcgcagcccctgt |
| | | ccctgcgcccagaggcgtgccggccagcgg |
| | | cggggggcgcagtgcacacgagggggctgg |
| | | acttcgcctgtgatatctacatctgggcgc |
| | | ccttggccgggacttgtggggtccttctcc |
| | | tgtcactggttatcaccctttactgcaacc |
| | | acaggaacaggagtaagaggagcaggctcc |
| | | tgcacagtgactacatgaacatgactcccc |
| | | gccgccccgggcccacccgcaagcattacc |
| | | agccctatgccccaccacgcgacttcgcag |
| | | cctatcgctccaaacggggcagaaagaaac |
| | | tcctgtatatattcaaacaaccatttatga |
| | | gaccagtacaaactactcaagaggaagatg |
| | | gctgtagctgccgatttccagaagaagaag |
| | | aaggaggatgtgaactgagagtgaagttca |
| | | gcaggagcgcagacgcccccgcgtaccagc |
| | | agggccagaaccagctctataacgagctca |
| | | atctaggacgaagagaggagtacgatgttt |
| | | tggacaagagacgtggccgggaccctgaga |
| | | tggggggaaagccgagaaggaagaaccctc |
| | | aggaaggcctgtacaatgaactgcagaaag |
| | | ataagatggcggaggcctacagtgagattg |
| | | ggatgaaaggcgagcgccggaggggcaagg |
| | | ggcacgatggcctttaccagggtctcagta |
| | | cagccaccaaggacacctacgacgcccttc |
| | | acatgcaggccctgccccctcgctga |
| 66 | Nucleic acid | atggccttaccagtgaccgccttgctcctg |
| | sequence of anti- | ccgctggccttgctgctccacgccgccagg |
| | FLT3 CAR with 20 | ccgggatccgacatccagatgacccagagc |
| | amino acid linker | ccctcctctttatccgcctctgtgggcgac |
| | (Signal peptide- | agagtgaccatcacttgtcgtgccagccaa |
| | linker-sAb1978- | gaaatctccggctatctgtcttggctgcag |
| | 30.11 VL-19aa | cagaagcccggtaaggctatcaagagactc |
| | linker-sAb 1978- | atctacgccgccagcactttacagagcgga |
| | 30.11 VH-linker- | gtgcctagcagatttagcggctctcgtagc |
| | CD8ahinge- | ggcagcgattatactttaaccatctcctct |
| | CD8aTM-CD28- | ttacagcccgaagactttgctacctactac |
| | 41BB-CD3zeta) | tgtttacagtacgccagctaccccttcacc |
| | encoded by | ttcggtcaaggtaccaagctggagatcaaa |
| | HG-CAR-TG105- | ggtggcggcggctctggcggcggtggctct |
| | EF1 CAR3a 20aa | ggcggcggtggctccggcggtggtggtagc |
| | linker plasmid | caagttaccctcaaggagagcggccccact |
| | | ttagtgaagcctacccagactttaacttta |
| | | acttgtaccttcagcggcttctctttaagc |
| | | acctccacaatgggcgtgggctggatcaga |
| | | cagcctcccggcaaggctctggagtggctg |
| | | gcccacatcctctggaacgacagcaagagg |
| | | tacaacccctctttaaagtctcgtctgacc |
| | | atcaccaaggacacctccaagaagcaagti |
| | | gtgctgaccatgaccaatatggaccccgtg |
| | | gacaccgccacctattactgcgctcgtatg |
| | | tgtactactccacctacgtgggctacttcg |
| | | acgtctggggacaaggtaccaccgtgaccg |
| | | tgagctctggatccttcgtgccggtcttcc |
| | | tgccagcgaagcccaccacgacgccagcgc |
| | | cgcgaccaccaacaccggcgcccaccatcg |
| | | cgtcgcagcccctgtccctgcgcccagagg |
| | | cgtgccggccagcggggggggcgcagtgca |
| | | cacgagggggctggacttcgcctgtgatat |
| | | ctacatctgggcgcccttggccgggacttg |
| | | tggggtccttctcctgtcactggttatcac |
| | | cctttactgcaaccacaggaacaggagtaa |
| | | gaggagcaggctcctgcacagtgactacat |
| | | gaacatgactccccgccgccccgggcccac |
| | | ccgcaagcattaccagccctatgccccacc |
| | | acgcgacttcgcagcctatcgctccaaacg |
| | | gggcagaaagaaactcctgtatatattcaa |
| | | acaaccatttatgagaccagtacaaactac |
| | | tcaagaggaagatggctgtagctgccgatt |
| | | tccagaagaagaagaaggaggatgtgaact |
| | | gagagtgaagttcagcaggagcgcagacgc |
| | | ccccgcgtaccagcagggccagaaccagct |
| | | ctataacgagctcaatctaggacgaagaga |
| | | ggagtacgatgttttggacaagagacgtgg |
| | | ccgggaccctgagatggggggaaagccgag |
| | | aaggaagaaccctcaggaaggcctgtacaa |
| | | tgaactgcagaaagataagatggggaggcc |
| | | tacagtgagattgggatgaaaggcgagcgc |
| | | cggaggggcaaggggcacgatggcctttac |
| | | cagggtctcagtacagccaccaaggacacc |
| | | tacgacgcccttcacatgcaggccctgccc |
| | | cctcgctga |
| 67 | Nucleic acid anti- | atggccttaccagtgaccgccttgctcctg |
| | FLT3 CAR with 20 | ccgctggccttgctgctccacgccgccagg |
| | amino acid linker | ccgggatccgacatccagatgacccagagc |
| | (Signal peptide- | ccctcctctttatccgcctctgtgggcgac |
| | linker-sAb 1978- | agagtgaccatcacttgtcgtgccagccaa |
| | 30.11 VL-19aa | gaaatctccggctatctgtcttggctgcag |
| | linker-sAb1978- | cagaagcccggtaaggctatcaagagactc |
| | 30.11 VH-linker- | atctacgccgccagcactttacagagcgga |
| | CD8ahinge- | gtgcctagcagatttagcggctctcgtagc |
| | CD8aTM-CD28- | ggcagcgattatactttaaccatctcctct |
| | CD3zeta) encoded | ttacagcccgaagactttgctacctactac |
| | by | tgtttacagtacgccagctaccccttcacc |
| | CAR3a-20aa linker- | ttcggtcaaggtaccaagctggagatcaaa |
| | NO-41BB plasmid | ggtggcggcggctctggcggcggtggctct |
| | | ggcggcggtggctccggcggtggtggtagc |
| | | caagttaccctcaaggagagcggccccact |
| | | ttagtgaagcctacccagactttaacttta |
| | | acttgtaccttcagcggcttctctttaagc |
| | | acctccacaatgggcgtgggctggatcaga |
| | | cagcctcccggcaaggctctggagtggctg |
| | | gcccacatcctctggaacgacagcaagagg |
| | | tacaacccctctttaaagtctcgtctgacc |
| | | atcaccaaggacacctccaagaagcaagti |
| | | gtgctgaccatgaccaatatggaccccgtg |
| | | gacaccgccacctattactgcgctcgtatc |
| | | gtgtactactccacctacgtgggctacttc |
| | | gacgtctggggacaaggtaccaccgtgacc |
| | | gtgagctctggatccttcgtgccggtcttc |
| | | ctgccagcgaagcccaccacgacgccagcg |
| | | ccgcgaccaccaacaccggcgcccaccatc |
| | | gcgtcgcagcccctgtccctgcgcccagag |
| | | gcgtgccggccagcggcggggggcgcagtg |
| | | cacacgagggggctggacttcgcctgtgat |
| | | atctacatctgggcgcccttggccgggact |
| | | tgtggggtccttctcctgtcactggttatc |
| | | accctttactgcaaccacaggaacaggagt |
| | | aagaggagcaggctcctgcacagtgactac |
| | | atgaacatgactccccgccgccccgggccc |
| | | acccgcaagcattaccagccctatgcccca |
| | | ccacgcgacttcgcagcctatcgctccaga |
| | | gtgaagttcagcaggagcgcagacgccccc |
| | | gcgtaccagcagggccagaaccagctctat |
| | | aacgagctcaatctaggacgaagagaggag |
| | | tacgatgttttggacaagagacgtggccgg |
| | | gaccctgagatggggggaaagccgagaagg |
| | | aagaaccctcaggaaggcctgtacaatgaa |
| | | ctgcagaaagataagatggcggaggcctac |
| | | agtgagattgggatgaaaggcgagcgccgg |
| | | aggggcaaggggcacgatggcctttaccag |
| | | ggtctcagtacagccaccaaggacacctac |
| | | gacgcccttcacatgcaggccctgccccct |
| | | cgctga |
| 68 | Nucleic acid | atggccttaccagtgaccgccttgctcctg |
| | sequence of anti- | ccgctggccttgctgctccacgccgccagg |
| | FLT3 CAR with 19 | ccgggatccgacatccagatgacccagagc |
| | amino acid linker | ccctcctctttatccgcctctgtgggcgac |
| | (Signal peptide- | agagtgaccatcacttgtgtgccagccaag |
| | linker-sAb1978- | aaatctccggctatctgtcttggctgcagc |
| | 30.11 VL-19aa | agaagcccggtaaggctatcaagagactca |
| | linker-sAb1978- | tctacgccgccagcactttacagagcggag |
| | 30.11 VH-linker- | tgcctagcagatttagcggctctcgtagcg |
| | CD8ahinge- | gcagcgattatactttaaccatctcctctt |
| | CD8aTM-41BB- | tacagcccgaagactttgctacctactact |
| | CD3zeta) encoded | gtttacagtacgccagctaccccttcacct |
| | by | tcggtcaaggtaccaagctggagatcaaag |
| | CAR3a-19aalinker- | gtggcggcggctctggcggcggtggctctg |
| | NO-CD28 plasmid | gcggtggctccggcggtggtggtagccaag |
| | | ttaccctcaaggagagcggccccactttag |
| | | tgaagcctacccagactttaactttaactt |
| | | gtaccttcagcggcttctctttaagcacct |
| | | ccacaatgggcgtgggctggatcagacagc |
| | | ctcccggcaaggctctggagtggctggccc |
| | | acatcctctggaacgacagcaagaggtaca |
| | | acccctctttaaagtctcgtctgaccatca |
| | | ccaaggacacctccaagaagcaagttgtgc |
| | | tgaccatgaccaatatggaccccgtggaca |
| | | ccgccacctattactgcgctcgtatcgtgt |
| | | actactccacctacgtgggctacttcgacg |
| | | tctggggacaaggtaccaccgtgaccgtga |
| | | gctctggatccttcgtgccggtcttcctgc |
| | | cagcgaagcccaccacgacgccagcgccgc |
| | | gaccaccaacaccggcgcccaccatcgcgt |
| | | cgcagcccctgtccctgcgcccagaggcgt |
| | | gccggccagcggggggggcgcagtgcacac |
| | | gagggggctggacttcgcctgtgatatcta |
| | | catctgggcgcccttggccgggacttgtgg |
| | | ggtccttctcctgtcactggttatcaccct |
| | | ttactgcaaccacaggaacaaacggggcag |
| | | aaagaaactcctgtatatattcaaacaacc |
| | | atttatgagaccagtacaaactactcaaga |
| | | ggaagatggctgtagctgccgatttccaga |
| | | agaagaagaaggaggatgtgaactgagagt |
| | | gaagttcagcaggagcgcagacgcccccgc |
| | | gtaccagcagggccagaaccagctctataa |
| | | cgagctcaatctaggacgaagagaggagta |
| | | cgatgttttggacaagagacgtggccggga |
| | | ccctgagatggggggaaagccgagaaggaa |
| | | gaaccctcaggaaggcctgtacaatgaact |
| | | gcagaaagataagatggcggaggcctacag |
| | | tgagattgggatgaaaggcgagcgccggag |
| | | gggcaaggggcacgatggcctttaccaggg |
| | | tctcagtacagccaccaaggacacctacga |
| | | cgcccttcacatgcaggccctgccccctcg |
| | | ctga |
| 69 | Nucleic acid | atggccttaccagtgaccgccttgctcctg |
| | sequence of anti- | ccgctggccttgctgctccacgccgccagg |
| | FLT3 CAR with 5 | ccgggatccgacatccagatgacccagagc |
| | amino acid linker | ccctcctctttatccgcctctgtgggcgac |
| | (Signal peptide- | agagtgaccatcacttgtcgtgccagccaa |
| | linker-sAb1978- | gaaatctccggctatctgtcttggctgcag |
| | 30.11 VL-19aa | cagaagcccggtaaggctatcaagagactc |
| | linker-sAb1978- | atctacgccgccagcactttacagagcgga |
| | 30.11 VH-linker- | gtgcctagcagatttagcggctctcgtagc |
| | CD8ahinge- | ggcagcgattatactttaaccatctcctct |
| | CD8aTM-CD28- | ttacagcccgaagactttgctacctactac |
| | CD3zeta) encoded | tgtttacagtacgccagctaccccttcacc |
| | by | ttcggtcaaggtaccaagctggagatcaaa |
| | CAR3a-5aalinker- | ggtggcggcggctctcaagttaccctcaag |
| | NO-41BB | gagagcggccccactttagtgaagcctacc |
| | | cagactttaactttaacttgtaccttcagc |
| | | ggcttctctttaagcacctccacaatgggc |
| | | gtgggctggatcagacagcctcccggcaag |
| | | gctctggagtggctggcccacatcctctgg |
| | | aacgacagcaagaggtacaacccctcttta |
| | | aagtctcgtctgaccatcaccaaggacacc |
| | | tccaagaagcaagtigtgctgaccatgacc |
| | | aatatggaccccgtggacaccgccacctat |
| | | tactgcgctcgtatcgtgtactactccacc |
| | | tacgtgggctacttcgacgtctggggacaa |
| | | ggtaccaccgtgaccgtgagctctggatcc |
| | | ttcgtgccggtcttcctgccagcgaagccc |
| | | accacgacgccagcgccgcgaccaccaaca |
| | | ccggcgcccaccatcgcgtcgcagcccctg |
| | | tccctgcgcccagaggcgtgccggccagcg |
| | | gggggggcgcagtgcacacgagggggctgg |
| | | acttcgcctgtgatatctacatctgggcgc |
| | | ccttggccgggacttgtggggtccttctcc |
| | | tgtcactggttatcaccctttactgcaacc |
| | | acaggaacaggagtaagaggagcaggctcc |
| | | tgcacagtgactacatgaacatgactcccc |
| | | gccgccccgggcccacccgcaagcattacc |
| | | agccctatgccccaccacgcgacttcgcag |
| | | cctatcgctccagagtgaagttcagcagga |
| | | gcgcagacgcccccgcgtaccagcagggcc |
| | | agaaccagctctataacgagctcaatctag |
| | | gacgaagagaggagtacgatgttttggaca |
| | | agagacgtggccgggaccctgagatggggg |
| | | gaaagccgagaaggaagaaccctcaggaag |
| | | gcctgtacaatgaactgcagaaagataaga |
| | | tggcggaggcctacagtgagattgggatga |
| | | aaggcgagcgccggaggggcaaggggcacg |
| | | atggcctttaccagggtctcagtacagcca |
| | | ccaaggacacctacgacgcccttcacatgc |
| | | aggccctgccccctcgctga |
| 70 | Nucleic acid | atggccttaccagtgaccgccttgctcctg |
| | sequence of anti- | ccgctggccttgctgctccacgccgccagg |
| | FLT3 CAR with 19 | ccgggatccgacatccagatgacccagagc |
| | amino acid linker | ccctcctctttatccgcctctgtgggcgac |
| | and GFP (Signal | agagtgaccatcacttgtcgtgccagccaa |
| | peptide-linker- | gaaatctccggctatctgtcttggctgcag |
| | sAb 1978-30.11 | cagaagcccggtaaggctatcaagagactc |
| | VL-19aa linker- | atctacgccgccagcactttacagagcgga |
| | sAb1978-30.11 | gtgcctagcagatttagcggctctcgtagc |
| | VH-linker- | ggcagcgattatactttaaccatctcctct |
| | CD8ahinge- | ttacagcccgaagactttgctacctactac |
| | CD8aTM-CD28- | tgtttacagtacgccagctaccccttcacc |
| | 41BB-CD3zeta- | ttcggtcaaggtaccaagctggagatcaaa |
| | T2A-GFP) encoded | ggtggcggcggctctggcggcggtggctct |
| | by pCDH-EF1- | ggcggtggctccggcggtggtggtagccaa |
| | CAR3a_19aa | gttaccctcaaggagagcggccccacttta |
| | linker_T2A_GFP | gtgaagcctacccagactttaactttaact |
| | | tgtaccttcagcggcttctctttaagcacc |
| | | tccacaatgggcgtgggctggatcagacag |
| | | cctcccggcaaggctctggagtggctggcc |
| | | cacatcctctggaacgacagcaagaggtac |
| | | aacccctctttaaagtctcgtctgaccatc |
| | | accaaggacacctccaagaagcaagttgtg |
| | | ctgaccatgaccaatatggaccccgtggac |
| | | accgccacctattactgcgctcgtatcgtg |
| | | tactactccacctacgtgggctacttcgac |
| | | gtctggggacaaggtaccaccgtgaccgtg |
| | | agctctggatccttcgtgccggtcttcctg |
| | | ccagcgaagcccaccacgacgccagcgccg |
| | | cgaccaccaacaccggcgcccaccatcgcg |
| | | tcgcagcccctgtccctgcgcccagaggcg |
| | | tgccggccagcgggggggggcagtgcacac |
| | | gagggggctggacttcgcctgtgatatcta |
| | | catctgggcgcccttggccgggacttgtgg |
| | | ggtccttctcctgtcactggttatcaccct |
| | | ttactgcaaccacaggaacaggagtaagag |
| | | gagcaggctcctgcacagtgactacatgaa |
| | | catgactccccgccgccccgggcccacccg |
| | | caagcattaccagccctatgccccaccacg |
| | | cgacttcgcagcctatcgctccaaacgggg |
| | | cagaaagaaactcctgtatatattcaaaca |
| | | accatttatgagaccagtacaaactactca |
| | | agaggaagatggctgtagctgccgatttcc |
| | | agaagaagaagaaggaggatgtgaactgag |
| | | agtgaagttcagcaggagcgcagacgcccc |
| | | cgcgtaccagcagggccagaaccagctcta |
| | | taacgagctcaatctaggacgaagagagga |
| | | gtacgatgttttggacaagagacgtggccg |
| | | ggaccctgagatggggggaaagccgagaag |
| | | gaagaaccctcaggaaggcctgtacaatga |
| | | actgcagaaagataagatggcggaggccta |
| | | cagtgagattgggatgaaaggcgagcgccg |
| | | gaggggcaaggggcacgatggcctttacca |
| | | gggtctcagtacagccaccaaggacaccta |
| | | cgacgcccttcacatgcaggccctgccccc |
| | | tcgcGCGGCCGCTGAGGGCAGAGGAAGTCT |
| | | TCTAACATGCGGTGACGTGGAGGAGAATCC |
| | | CGGCCCTTCCGGAATGGAGAGCGACGAGAG |
| | | CGGCCTGCCCGCCATGGAGATCGAGTGCCG |
| | | CATCACCGGCACCCTGAACGGCGTGGAGTT |
| | | CGAGCTGGTGGGCGGCGGAGAGGGCACCCC |
| | | CAAGCAGGGCCGCATGACCAACAAGATGAA |
| | | GAGCACCAAAGGCGCCCTGACCTTCAGCCC |
| | | CTACCTGCTGAGCCACGTGATGGGCTACGG |
| | | CTTCTACCACTTCGGCACCTACCCCAGCGG |
| | | CTACGAGAACCCCTTCCTGCACGCCATCAA |
| | | CAACGGCGGCTACACCAACACCCGCATCGA |
| | | GAAGTACGAGGACGGCGGCGTGCTGCACGT |
| | | GAGCTTCAGCTACCGCTACGAGGCCGGCCG |
| | | CGTGATCGGCGACTTCAAGGTGGTGGGCAC |
| | | CGGCTTCCCCGAGGACAGCGTGATCTTCAC |
| | | CGACAAGATCATCCGCAGCAACGCCACCGT |
| | | GGAGCACCTGCACCCCATGGGCGATAACGT |
| | | GCTGGTGGGCAGCTTCGCCCGCACCTTCAG |
| | | CCTGCGCGACGGCGGCTACTACAGCTTCGT |
| | | GGTGGACAGCCACATGCACTTCAAGAGCGC |
| | | CATCCACCCCAGCATCCTGCAGAACGGGGG |
| | | CCCCATGTTCGCCTTCCGCCGCGTGGAGGA |
| | | GCTGCACAGCAACACCGAGCTGGGCATCGT |
| | | GGAGTACCAGCACGCCTTCAAGACCCCCAT |
| | | CGCCTTCGCCAGATCCCGCGCTCAGTCGTC |
| | | CAATTCTGCCGTGGACGGCACCGCCGGACC |
| | | CGGCTCCACCGGATCTCGCTAG |
| 71 | Amino Acid | MALPVTALLLPLALLLHAARP |
| | sequence of a | |
| | Signal Peptide | |
| 72 | Amino Acid | FVPVFLPAKPTTTPAPRPPTPAPTIASQP |
| | sequence of CD8a | LSLRPEACRPAAGGAVHTRGLDFACD |
| | Hinge region | |
| 73 | Amino acid | IYIWAPLAGTCGVLLLSLVITLYCNHRN |
| | sequence of CD8a | |
| | transmembrane | |
| | domain | |
| 74 | Amino acid CD28 | RSKRSRLLHSDYMNMTPRRPGPTRKHYQPY |
| | co-stimulatory | APPRDFAAYRS |
| | domain | |
| 75 | Amino acid 4-1BB | KRGRKKLLYIFKQPFMRPVQTTQEEDGCSC |
| | co-stimulatory | RFPEEEEGGCEL |
| | domain | |
| 76 | Amino acid | RVKFSRSADAPAYQQGQNQLYNELNLGRR |
| | CD3zeta activation | EEYDVLDKRRGRDPEMGGKPRRKNPQEGL |
| | domain | YNELQKDKMAEAYSEIGMKGERRRGKGHD |
| | | GLYQGLSTATKDTYDALHMQALPPR |
| 77 | Nucleotide | atggccttaccagtgaccgccttgctcct |
| | sequence of Signal | gccgctggccttgctgctccacgccgcca |
| | Peptide | ggccg |
| 78 | Nucleotide | ttcgtgccggtcttcctgccagcgaagccc |
| | sequence of CD8a | accacgacgccagcgccgcgaccaccaaca |
| | Hinge region | ccggcgcccaccatcgcgtcgcagcccctg |
| | | tccctgcgcccagaggcgtgccggccagcg |
| | | gcggggggcgcagtgcacacgagggggctg |
| | | gacttcgcctgtgat |
| 79 | Nucleotide | atctacatctgggcgcccttggccgggact |
| | sequence of CD8a | tgtggggtccttctcctgtcactggttatc |
| | transmembrane | accctttactgcaaccacaggaac |
| | domain | |
| 80 | Nucleotide | aggagtaagaggagcaggctcctgcacagt |
| | sequence of CD28 | gactacatgaacatgactccccgccgcccc |
| | co-stimulatory | gggcccacccgcaagcattaccagccctat |
| | domain | gccccaccacgcgacttcgcagcctatcgc |
| | | tcc |
| 81 | Nucleotide | aaacggggcagaaagaaactcctgtatata |
| | sequence of 4-1BB | ttcaaacaaccatttatgagaccagtacaa |
| | co-stimulatory | actactcaagaggaagatggctgtagctgc |
| | domain | cgatttccagaagaagaagaaggaggatgt |
| | | gaactg |
| 82 | Nucleotide | agagtgaagttcagcaggagcgcagacgcc |
| | sequence of | cccgcgtaccagcagggccagaaccagctc |
| | CD3zeta activation | tataacgagctcaatctaggacgaagagag |
| | domain | gagtacgatgttttggacaagagacgtggc |
| | | cgggaccctgagatggggggaaagccgaga |
| | | aggaagaaccctcaggaaggcctgtacaat |
| | | gaactgcagaaagataagatggcggaggcc |
| | | tacagtgagattgggatgaaaggcgagcgc |
| | | cggaggggcaaggggcacgatggcctttac |
| | | cagggtctcagtacagccaccaaggacacc |
| | | tacgacgcccttcacatgcaggccctgccc |
| | | cctcgc |
| 83 | Nucleotide | ggaagcggagagggcaggggaagtcttcta |
| | sequence of T2A | acatgcggggacgtggaggaaaatcccggc |
| | Cleavage peptide | ccca |
| 84 | Nucleotide | Tgcttctcctggtgacaagccttctgctct |
| | sequence of EGFRt | gtgagttaccacacccagcattcctcctga |
| | | tcccacgcaaagtgtgtaacggaataggta |
| | | ttggtgaatttaaagactcactctccataa |
| | | atgctacgaatattaaacacttcaaaaact |
| | | gcacctccatcagtggcgatctccacatcc |
| | | tgccggtggcatttaggggtgactccttca |
| | | cacatactcctcctctggatccacaggaac |
| | | tggsgatattctgaaaaccgtaaaggaaat |
| | | cacagggtttttgctgattcaggcttggcc |
| | | tgaaaacaggacggacctccatgcctttga |
| | | gaacctagaaatcatacgcggcaggaccaa |
| | | gcaacatggtcagttttctcttgcagtcgt |
| | | cagcctgaacataacatccttgggattacg |
| | | ctccctcaaggagataagTgatggagatgt |
| | | gataatttcaggaaacaaaaatttgtgcta |
| | | tgcaaatacaataaactggaaaaaactgtt |
| | | tgggacctccggtcagaaaaccaaaattat |
| | | aagcaacagaggtgaaaacagctgcaaggc |
| | | cacaggccaggtctgccatgccttgtgctc |
| | | ccccgagggctgctggggcccggagcccag |
| | | ggactgcgtctcttgccggaatgtcagccg |
| | | aggcagggaatgcgtggacaagtgcaacct |
| | | tctggagggtgagccaagggagtttgtgga |
| | | gaactctgagtgcatacagtgccacccaga |
| | | gtgcctgcctcaggccatgaacatcacctg |
| | | cacaggacggggaccagacaactgtatcca |
| | | gtgtgcccactacattgacggcccccactg |
| | | cgtcaagacctgcccggcaggagtcatggg |
| | | agaaaacaacaccctggtctggaagtacgc |
| | | agacgccggccatgtgtgccacctgtgcca |
| | | tccaaactgcacctacggatgcactgggcc |
| | | aggtcttgaaggctgtccaacgaatgggcc |
| | | taagatcccgtccatcgccactgggatggt |
| | | gggggccctcctcttgctgctggtggtggc |
| | | cctggggatcggcctcttcatgtgaa |
| 85 | Nucleotide | ATGGGAGTGCAGGTGGAAACCATCTCCCCA |
| | sequence of iCasp9 | GGAGACGGGCGCACCTTCCCCAAGCGCGGC |
| | | CAGACCTGCGTGGTGCACTACACCGGGATG |
| | | CTTGAAGATGGAAAGAAAGTGGATTCCTCC |
| | | CGGGACAGAAACAAGCCCTTTAAGTTTATG |
| | | CTAGGCAAGCAGGAGGTGATCCGAGGCTGG |
| | | GAAGAAGGGGTTGCCCAGATGAGTGTGGGT |
| | | CAGAGAGCCAAACTGACTATATCTCCAGAT |
| | | TATGCCTATGGTGCCACTGGGCACCCAGGC |
| | | ATCATCCCACCACATGCCACTCTCGTCTTC |
| | | GATGTGGAGCTTCTAAAACTGGAATCTGGA |
| | | GGAGGTTCTACTAACAGGCAAGCAGCAAAG |
| | | TTGTCGAAGCCAACCCTAGAAAACCTTACC |
| | | CCAGTGGTGCTCAGACCAGAGATTCGCAAA |
| | | CCAGAGGTTCTCAGACCGGAAACACCCAGA |
| | | CCAGTGGACATTGGTTCTGGAGGATTTGGT |
| | | GATGTCGGTGCTCTTGAGAGTTTGAGGGGA |
| | | AATGCAGATTTGGCTTACATCCTGAGCATG |
| | | GAGCCCTGTGGCCACTGCCTCATTATCAAC |
| | | AATGTGAACTTCTGCCGTGAGTCCGGGCTC |
| | | CGCACCCGCACTGGCTCCAACATCGACTGT |
| | | GAGAAGTTGCGGCGTCGCTTCTCCTCGCTG |
| | | CATTTCATGGTGGAGGTGAAGGGCGACCTG |
| | | ACTGCCAAGAAAATGGTGCTGGCTTTGCTG |
| | | GAGCTGGCGCAGCAGGACCACGGTGCTCTG |
| | | GACTGCTGCGTGGTGGTCATTCTCTCTCAC |
| | | GGCTGTCAGGCCAGCCACCTGCAGTTCCCA |
| | | GGGGCTGTCTACGGCACAGATGGATGCCCT |
| | | GTGTCGGTCGAGAAGATTGTGAACATCTTC |
| | | AATGGGACCAGCTGCCCCAGCCTGGGAGGG |
| | | AAGCCCAAGCTCTTTTTCATCCAGGCCTGT |
| | | GGTGGGGAGCAGAAAGACCATGGGTTTGAG |
| | | GTGGCCTCCACTTCCCCTGAAGACGAGTCC |
| | | CCTGGCAGTAACCCCGAGCCAGATGCCACC |
| | | CCGTTCCAGGAAGGTTTGAGGACCTTCGAC |
| | | CAGCTGGACGCCATATCTAGTTTGCCCACA |
| | | CCCAGTGACATCTTTGTGTCCTACTCTACT |
| | | TTCCCAGGTTTTGTTTCCTGGAGGGACCCC |
| | | AAGAGTGGCTCCTGGTACGTTGAGACCCTG |
| | | GACGACATCTTTGAGCAGTGGGCTCACTCT |
| | | GAAGACCTGCAGTCCCTCCTGCTTAGGGTC |
| | | GCTAATGCTGTTTCGGTGAAAGGGATTTAT |
| | | AAACAGATGCCTGGTTGCTTTAATTTCCTC |
| | | CGGAAAAAACTTTTCTTTAAAACATCA |
| 86 | Amino acid | QEISGY |
| | sequence of VL | |
| | CDR1 | |
| 87 | Amino acid | AAS |
| | sequence of VL | |
| | CDR2 | |
| 88 | Amino acid | LQYASYPFT |
| | sequence of VL | |
| | CDR3 | |
| 89 | Amino acid | GFSLSTSTMG |
| | sequence of VH | |
| | CDR1 | |
| 90 | Amino acid | ILWNDSK |
| | sequence of VH | |
| | CDR2 | |
| 91 | Amino acid | ARIVYYSTYVGYFDV |
| | sequence of VH | |
| | CDR3 | |
| 92 | Nucleic acid | ACGCGTGTAGTCTTATGCAATACTCTTGTA |
| | sequence of pCDH- | GTCTTGCAACATGGTAACGATGAGTTAGCA |
| | EF1- | ACATGCCTTACAAGGAGAGAAAAAGCACCG |
| | CAR3a_19aalinker_ | TGCATGCCGATTGGTGGAAGTAAGGTGGTA |
| | T2A_GFP | CGATCGTGCCTTATTAGGAAGGCAACAGAC |
| | plasmid | GGGTCTGACATGGATTGGACGAACCACTGA |
| | | ATTGCCGCATTGCAGAGATATTGTATTTAA |
| | | GTGCCTAGCTCGATACATAAACGGGTCTCT |
| | | CTGGTTAGACCAGATCTGAGCCTGGGAGCT |
| | | CTCTGGCTAACTAGGGAACCCACTGCTTAA |
| | | GCCTCAATAAAGCTTGCCTTGAGTGCTTCA |
| | | AGTAGTGTGTGCCCGTCTGTTGTGTGACTC |
| | | TGGTAACTAGAGATCCCTCAGACCCTTTTA |
| | | GTCAGTGTGGAAAATCTCTAGCAGTGGCGC |
| | | CCGAACAGGGACTTGAAAGCGAAAGGGAAA |
| | | CCAGAGGAGCTCTCTCGACGCAGGACTCGG |
| | | CTTGCTGAAGCGCGCACGGCAAGAGGCGAG |
| | | GGGCGGCGACTGGTGAGTACGCCAAAAATT |
| | | TTGACTAGCGGAGGCTAGAAGGAGAGAGAT |
| | | GGGTGCGAGAGCGTCAGTATTAAGCGGGGG |
| | | AGAATTAGATCGCGATGGGAAAAAATTCGG |
| | | TTAAGGCCAGGGGGAAAGAAAAAATATAAA |
| | | TTAAAACATATAGTATGGGCAAGCAGGGAG |
| | | CTAGAACGATTCGCAGTTAATCCTGGCCTG |
| | | TTAGAAACATCAGAAGGCTGTAGACAAATA |
| | | CTGGGACAGCTACAACCATCCCTTCAGACA |
| | | GGATCAGAAGAACTTAGATCATTATATAAT |
| | | ACAGTAGCAACCCTCTATTGTGTGCATCAA |
| | | AGGATAGAGATAAAAGACACCAAGGAAGCT |
| | | TTAGACAAGATAGAGGAAGAGCAAAACAAA |
| | | AGTAAGACCACCGCACAGCAAGCGGCCACT |
| | | GATCTTCAGACCTGGAGGAGGAGATATGAG |
| | | GGACAATTGGAGAAGTGAATTATATAAATA |
| | | TAAAGTAGTAAAAATTGAACCATTAGGAGT |
| | | AGCACCCACCAAGGCAAAGAGAAGAGTGGT |
| | | GCAGAGAGAAAAAAGAGCAGTGGGAATAGG |
| | | AGCTTTGTTCCTTGGGTTCTTGGGAGCAGC |
| | | AGGAAGCACTATGGGCGCAGCGTCAATGAC |
| | | GCTGACGGTACAGGCCAGACAATTATTGTC |
| | | TGGTATAGTGCAGCAGCAGAACAATTTGCT |
| | | GAGGGCTATTGAGGCGCAACAGCATCTGTT |
| | | GCAACTCACAGTCTGGGGCATCAAGCAGCT |
| | | CCAGGCAAGAATCCTGGCTGTGGAAAGATA |
| | | CCTAAAGGATCAACAGCTCCTGGGGATTTG |
| | | GGGTTGCTCTGGAAAACTCATTTGCACCAC |
| | | TGCTGTGCCTTGGAATGCTAGTTGGAGTAA |
| | | TAAATCTCTGGAACAGATTTGGAATCACAC |
| | | GACCTGGATGGAGTGGGACAGAGAAATTAA |
| | | CAATTACACAAGCTTAATACACTCCTTAAT |
| | | TGAAGAATCGCAAAACCAGCAAGAAAAGAA |
| | | TGAACAAGAATTATTGGAATTAGATAAATG |
| | | GGCAAGTTTGTGGAATTGGTTTAACATAAC |
| | | AAATTGGCTGTGGTATATAAAATTATTCAT |
| | | AATGATAGTAGGAGGCTTGGTAGGTTTAAG |
| | | AATAGTTTTTGCTGTACTTTCTATAGTGAA |
| | | TAGAGTTAGGCAGGGATATTCACCATTATC |
| | | GTTTCAGACCCACCTCCCAACCCCGAGGGG |
| | | ACCCGACAGGCCCGAAGGAATAGAAGAAGA |
| | | AGGTGGAGAGAGAGACAGAGACAGATCCAT |
| | | TCGATTAGTGAACGGATCTCGACGGTATCG |
| | | GTTAACTTTTAAAAGAAAAGGGGGGATTGG |
| | | GGGGTACAGTGCAGGGGAAAGAATAGTAGA |
| | | CATAATAGCAACAGACATACAAACTAAAGA |
| | | ATTACAAAAACAAATTACAAAATTCAAAAT |
| | | TTTATCGATACTAGTGGATCTGCGATCGCT |
| | | CCGGTGCCCGTCAGTGGGCAGAGCGCACAT |
| | | CGCCCACAGTCCCCGAGAAGTTGGGGGGAG |
| | | GGGTCGGCAATTGAACGGGTGCCTAGAGAA |
| | | GGTGGCGCGGGGTAAACTGGGAAAGTGATG |
| | | TCGTGTACTGGCTCCGCCTTTTTCCCGAGG |
| | | GTGGGGGAGAACCGTATATAAGTGCAGTAG |
| | | TCGCCGTGAACGTTCTTTTTCGCAACGGGT |
| | | TTGCCGCCAGAACACAGCTGAAGCTTCGAG |
| | | GGGCTCGCATCTCTCCTTCACGCGCCCGCC |
| | | GCCCTACCTGAGGCCGCCATCCACGCCGGT |
| | | TGAGTCGCGTTCTGCCGCCTCCCGCCTGTG |
| | | GTGCCTCCTGAACTGCGTCCGCCGTCTAGG |
| | | TAAGTTTAAAGCTCAGGTCGAGACCGGGCC |
| | | TTTGTCCGGCGCTCCCTTGGAGCCTACCTA |
| | | GACTCAGCCGGCTCTCCACGCTTTGCCTGA |
| | | CCCTGCTTGCTCAACTCTACGTCTTTGTTT |
| | | CGTTTTCTGTTCTGCGCCGTTACAGATCCA |
| | | AGCTGTGACCGGCGCCTACTCTAGAATGGC |
| | | CTTACCAGTGACCGCCTTGCTCCTGCCGCT |
| | | GGCCTTGCTGCTCCACGCCGCCAGGCCGGG |
| | | ATCCGACATCCAGATGACCCAGAGCCCCTC |
| | | CTCTTTATCCGCCTCTGTGGGCGACAGAGT |
| | | GACCATCACTTGTCGTGCCAGCCAAGAAAT |
| | | CTCCGGCTATCTGTCTTGGCTGCAGCAGAA |
| | | GCCCGGTAAGGCTATCAAGAGACTCATCTA |
| | | CGCCGCCAGCACTTTACAGAGCGGAGTGCC |
| | | TAGCAGATTTAGCGGCTCTCGTAGCGGCAG |
| | | CGATTATACTTTAACCATCTCCTCTTTACA |
| | | GCCCGAAGACTTTGCTACCTACTACTGTTT |
| | | ACAGTACGCCAGCTACCCCTTCACCTTCGG |
| | | TCAAGGTACCAAGCTGGAGATCAAAGGTGG |
| | | CGGCGGCTCTGGCGGCGGTGGCTCTGGCGG |
| | | TGGCTCCGGCGGTGGTGGTAGCCAAGTTAC |
| | | CCTCAAGGAGAGCGGCCCCACTTTAGTGAA |
| | | GCCTACCCAGACTTTAACTTTAACTTGTAC |
| | | CTTCAGCGGCTTCTCTTTAAGCACCTCCAC |
| | | AATGGGCGTGGGCTGGATCAGACAGCCTCC |
| | | CGGCAAGGCTCTGGAGTGGCTGGCCCACAT |
| | | CCTCTGGAACGACAGCAAGAGGTACAACCC |
| | | CTCTTTAAAGTCTCGTCTGACCATCACCAA |
| | | GGACACCTCCAAGAAGCAAGTIGTGCTGAC |
| | | CATGACCAATATGGACCCCGTGGACACCGC |
| | | CACCTATTACTGCGCTCGTATCGTGTACTA |
| | | CTCCACCTACGTGGGCTACTTCGACGTCTG |
| | | GGGACAAGGTACCACCGTGACCGTGAGCTC |
| | | TGGATCCTTCGTGCCGGTCTTCCTGCCAGC |
| | | GAAGCCCACCACGACGCCAGCGCCGCGACC |
| | | ACCAACACCGGCGCCCACCATCGCGTCGCA |
| | | GCCCCTGTCCCTGCGCCCAGAGGCGTGCCG |
| | | GCCAGCGGCGGGGGGCGCAGTGCACACGAG |
| | | GGGGCTGGACTTCGCCTGTGATATCTACAT |
| | | CTGGGCGCCCTTGGCCGGGACTTGTGGGGT |
| | | CCTTCTCCTGTCACTGGTTATCACCCTTTA |
| | | CTGCAACCACAGGAACAGGAGTAAGAGGAG |
| | | CAGGCTCCTGCACAGTGACTACATGAACAT |
| | | GACTCCCCGCCGCCCCGGGCCCACCCGCAA |
| | | GCATTACCAGCCCTATGCCCCACCACGCGA |
| | | CTTCGCAGCCTATCGCTCCAAACGGGGCAG |
| | | AAAGAAACTCCTGTATATATTCAAACAACC |
| | | ATTTATGAGACCAGTACAAACTACTCAAGA |
| | | GGAAGATGGCTGTAGCTGCCGATTTCCAGA |
| | | AGAAGAAGAAGGAGGATGTGAACTGAGAGT |
| | | GAAGTTCAGCAGGAGCGCAGACGCCCCCGC |
| | | GTACCAGCAGGGCCAGAACCAGCTCTATAA |
| | | CGAGCTCAATCTAGGACGAAGAGAGGAGTA |
| | | CGATGTTTTGGACAAGAGACGTGGCCGGGA |
| | | CCCTGAGATGGGGGGAAAGCCGAGAAGGAA |
| | | GAACCCTCAGGAAGGCCTGTACAATGAACT |
| | | GCAGAAAGATAAGATGGCGGAGGCCTACAG |
| | | TGAGATTGGGATGAAAGGCGAGCGCCGGAG |
| | | GGGCAAGGGGCACGATGGCCTTTACCAGGG |
| | | TCTCAGTACAGCCACCAAGGACACCTACGA |
| | | CGCCCTTCACATGCAGGCCCTGCCCCCTCG |
| | | CGCGGCCGCTGAGGGCAGAGGAAGTCTTCT |
| | | AACATGCGGTGACGTGGAGGAGAATCCCGG |
| | | CCCTTCCGGAATGGAGAGCGACGAGAGCGG |
| | | CCTGCCCGCCATGGAGATCGAGTGCCGCAT |
| | | CACCGGCACCCTGAACGGCGTGGAGTTCGA |
| | | GCTGGTGGGCGGCGGAGAGGGCACCCCCAA |
| | | GCAGGGCCGCATGACCAACAAGATGAAGAG |
| | | CACCAAAGGCGCCCTGACCTTCAGCCCCTA |
| | | CCTGCTGAGCCACGTGATGGGCTACGGCTT |
| | | CTACCACTTCGGCACCTACCCCAGCGGCTA |
| | | CGAGAACCCCTTCCTGCACGCCATCAACAA |
| | | CGGCGGCTACACCAACACCCGCATCGAGAA |
| | | GTACGAGGACGGCGGCGTGCTGCACGTGAG |
| | | CTTCAGCTACCGCTACGAGGCCGGCCGCGT |
| | | GATCGGCGACTTCAAGGTGGTGGGCACCGG |
| | | CTTCCCCGAGGACAGCGTGATCTTCACCGA |
| | | CAAGATCATCCGCAGCAACGCCACCGTGGA |
| | | GCACCTGCACCCCATGGGCGATAACGTGCT |
| | | GGTGGGCAGCTTCGCCCGCACCTTCAGCCT |
| | | GCGCGACGGCGGCTACTACAGCTTCGTGGT |
| | | GGACAGCCACATGCACTTCAAGAGCGCCAT |
| | | CCACCCCAGCATCCTGCAGAACGGGGGCCC |
| | | CATGTTCGCCTTCCGCCGCGTGGAGGAGCT |
| | | GCACAGCAACACCGAGCTGGGCATCGTGGA |
| | | GTACCAGCACGCCTTCAAGACCCCCATCGC |
| | | CTTCGCCAGATCCCGCGCTCAGTCGTCCAA |
| | | TTCTGCCGTGGACGGCACCGCCGGACCCGG |
| | | CTCCACCGGATCTCGCTAGAGCTGAATCTA |
| | | AGTCGACAATCAACCTCTGGATTACAAAAT |
| | | TTGTGAAAGATTGACTGGTATTCTTAACTA |
| | | TGTTGCTCCTTTTACGCTATGTGGATACGC |
| | | TGCTTTAATGCCTTTGTATCATGCTATTGC |
| | | TTCCCGTATGGCTTTCATTTTCTCCTCCTT |
| | | GTATAAATCCTGGTTGCTGTCTCTTTATGA |
| | | GGAGTTGTGGCCCGTTGTCAGGCAACGTGG |
| | | CGTGGTGTGCACTGTGTTTGCTGACGCAAC |
| | | CCCCACTGGTTGGGGCATTGCCACCACCTG |
| | | TCAGCTCCTTTCCGGGACTTTCGCTTTCCC |
| | | CCTCCCTATTGCCACGGCGGAACTCATCGC |
| | | CGCCTGCCTTGCCCGCTGCTGGACAGGGGC |
| | | TCGGCTGTTGGGCACTGACAATTCCGTGGT |
| | | GTTGTCGGGGAAATCATCGTCCTTTCCTTG |
| | | GCTGCTCGCCTGTGTTGCCACCTGGATTCT |
| | | GCGCGGGACGTCCTTCTGCTACGTCCCTTC |
| | | GGCCCTCAATCCAGCGGACCTTCCTTCCCG |
| | | CGGCCTGCTGCCGGCTCTGCGGCCTCTTCC |
| | | GCGTCTTCGCCTTCGCCCTCAGACGAGTCG |
| | | GATCTCCCTTTGGGCCGCCTCCCCGCCTGG |
| | | TACCTTTAAGACCAATGACTTACAAGGCAG |
| | | CTGTAGATCTTAGCCACTTTTTAAAAGAAA |
| | | AGGGGGGACTGGAAGGGCTAATTCACTCCC |
| | | AACGAAAATAAGATCTGCTTTTTGCTTGTA |
| | | CTGGGTCTCTCTGGTTAGACCAGATCTGAG |
| | | CCTGGGAGCTCTCTGGCTAACTAGGGAACC |
| | | CACTGCTTAAGCCTCAATAAAGCTTGCCTT |
| | | GAGTGCTTCAAGTAGTGTGTGCCCGTCTGT |
| | | TGTGTGACTCTGGTAACTAGAGATCCCTCA |
| | | GACCCTTTTAGTCAGTGTGGAAAATCTCTA |
| | | GCAGTAGTAGTTCATGTCATCTTATTATTC |
| | | AGTATTTATAACTTGCAAAGAAATGAATAT |
| | | CAGAGAGTGAGAGGAACTTGTTTATTGCAG |
| | | CTTATAATGGTTACAAATAAAGCAATAGCA |
| | | TCACAAATTTCACAAATAAAGCATTTTTTT |
| | | CACTGCATTCTAGTTGTGGTTTGTCCAAAC |
| | | TCATCAATGTATCTTATCATGTCTGGCTCT |
| | | AGCTATCCCGCCCCTAACTCCGCCCATCCC |
| | | GCCCCTAACTCCGCCCAGTTCCGCCCATTC |
| | | TCCGCCCCATGGCTGACTAATTTTTTTTAT |
| | | TTATGCAGAGGCCGAGGCCGCCTCGGCCTC |
| | | TGAGCTATTCCAGAAGTAGTGAGGAGGCTT |
| | | TTTTGGAGGCCTAGACTTTTGCAGAGACCA |
| | | AATTCGTAATCATGTCATAGCTGTTTCCTG |
| | | TGTGAAATTGTTATCCGCTCACAATTCCAC |
| | | ACAACATACGAGCCGGAAGCATAAAGTGTA |
| | | AAGCCTGGGGTGCCTAATGAGTGAGCTAAC |
| | | TCACATTAATTGCGTTGCGCTCACTGCCCG |
| | | CTTTCCAGTCGGGAAACCTGTCGTGCCAGC |
| | | TGCATTAATGAATCGGCCAACGCGCGGGGA |
| | | GAGGCGGTTTGCGTATTGGGCGCTCTTCCG |
| | | CTTCCTCGCTCACTGACTCGCTGCGCTCGG |
| | | TCGTTCGGCTGCGGCGAGCGGTATCAGCTC |
| | | ACTCAAAGGCGGTAATACGGTTATCCACAG |
| | | AATCAGGGGATAACGCAGGAAAGAACATGT |
| | | GAGCAAAAGGCCAGCAAAAGGCCAGGAACC |
| | | GTAAAAAGGCCGCGTTGCTGGCGTTTTTCC |
| | | ATAGGCTCCGCCCCCCTGACGAGCATCACA |
| | | AAAATCGACGCTCAAGTCAGAGGTGGCGAA |
| | | ACCCGACAGGACTATAAAGATACCAGGCGT |
| | | TTCCCCCTGGAAGCTCCCTCGTGCGCTCTC |
| | | CTGTTCCGACCCTGCCGCTTACCGGATACC |
| | | TGTCCGCCTTTCTCCCTTCGGGAAGCGTGG |
| | | CGCTTTCTCATAGCTCACGCTGTAGGTATC |
| | | TCAGTTCGGTGTAGGTCGTTCGCTCCAAGC |
| | | TGGGCTGTGTGCACGAACCCCCCGTTCAGC |
| | | CCGACCGCTGCGCCTTATCCGGTAACTATC |
| | | GTCTTGAGTCCAACCCGGTAAGACACGACT |
| | | TATCGCCACTGGCAGCAGCCACTGGTAACA |
| | | GGATTAGCAGAGCGAGGTATGTAGGCGGTG |
| | | CTACAGAGTTCTTGAAGTGGTGGCCTAACT |
| | | ACGGCTACACTAGAAGAACAGTATTTGGTA |
| | | TCTGCGCTCTGCTGAAGCCAGTTACCTTCG |
| | | GAAAAAGAGTTGGTAGCTCTTGATCCGGCA |
| | | AACAAACCACCGCTGGTAGCGGTGGTTTTT |
| | | TTGTTTGCAAGCAGCAGATTACGCGCAGAA |
| | | AAAAAGGATCTCAAGAAGATCCTTTGATCT |
| | | TTTCTACGGGGTCTGACGCTCAGTGGAACG |
| | | AAAACTCACGTTAAGGGATTTTGGTCATGA |
| | | GATTATCAAAAAGGATCTTCACCTAGATCC |
| | | TTTTAAATTAAAAATGAAGTTTTAAATCAA |
| | | TCTAAAGTATATATGAGTAAACTTGGTCTG |
| | | ACAGTTACCAATGCTTAATCAGTGAGGCAC |
| | | CTATCTCAGCGATCTGTCTATTTCGTTCAT |
| | | CCATAGTTGCCTGACTCCCCGTCGTGTAGA |
| | | TAACTACGATACGGGAGGGCTTACCATCTG |
| | | GCCCCAGTGCTGCAATGATACCGCGAGACC |
| | | CACGCTCACCGGCTCCAGATTTATCAGCAA |
| | | TAAACCAGCCAGCCGGAAGGGCCGAGCGCA |
| | | GAAGTGGTCCTGCAACTTTATCCGCCTCCA |
| | | TCCAGTCTATTAATTGTTGCCGGGAAGCTA |
| | | GAGTAAGTAGTTCGCCAGTTAATAGTTTGC |
| | | GCAACGTTGTTGCCATTGCTACAGGCATCG |
| | | TGGTGTCACGCTCGTCGTTTGGTATGGCTT |
| | | CATTCAGCTCCGGTTCCCAACGATCAAGGC |
| | | GAGTTACATGATCCCCCATGTTGTGCAAAA |
| | | AAGCGGTTAGCTCCTTCGGTCCTCCGATCG |
| | | TTGTCAGAAGTAAGTTGGCCGCAGTGTTAT |
| | | CACTCATGGTTATGGCAGCACTGCATAATT |
| | | CTCTTACTGTCATGCCATCCGTAAGATGCT |
| | | TTTCTGTGACTGGTGAGTACTCAACCAAGT |
| | | CATTCTGAGAATAGTGTATGCGGCGACCGA |
| | | GTTGCTCTTGCCCGGCGTCAATACGGGATA |
| | | ATACCGCGCCACATAGCAGAACTTTAAAAG |
| | | TGCTCATCATTGGAAAACGTTCTTCGGGGC |
| | | GAAAACTCTCAAGGATCTTACCGCTGTTGA |
| | | GATCCAGTTCGATGTAACCCACTCGTGCAC |
| | | CCAACTGATCTTCAGCATCTTTTACTTTCA |
| | | CCAGCGTTTCTGGGTGAGCAAAAACAGGAA |
| | | GGCAAAATGCCGCAAAAAAGGGAATAAGGG |
| | | CGACACGGAAATGTTGAATACTCATACTCT |
| | | TCCTTTTTCAATATTATTGAAGCATTTATC |
| | | AGGGTTATTGTCTCATGAGCGGATACATAT |
| | | TTGAATGTATTTAGAAAAATAAACAAATAG |
| | | GGGTTCCGCGCACATTTCCCCGAAAAGTGC |
| | | CACCTGACGTCTAAGAAACCATTATTATCA |
| | | TGACATTAACCTATAAAAATAGGCGTATCA |
| | | CGAGGCCCTTTCGTCTCGCGCGTTTCGGTG |
| | | ATGACGGTGAAAACCTCTGACACATGCAGC |
| | | TCCCGGAGACGGTCACAGCTTGTCTGTAAG |
| | | CGGATGCCGGGAGCAGACAAGCCCGTCAGG |
| | | GCGCGTCAGCGGGTGTTGGCGGGTGTCGGG |
| | | GCTGGCTTAACTATGCGGCATCAGAGCAGA |
| | | TTGTACTGAGAGTGCACCATATGCGGTGTG |
| | | AAATACCGCACAGATGCGTAAGGAGAAAAT |
| | | ACCGCATCAGGCGCCATTCGCCATTCAGGC |
| | | TGCGCAACTGTTGGGAAGGGCGATCGGTGC |
| | | GGGCCTCTTCGCTATTACGCCAGCTGGCGA |
| | | AAGGGGGATGTGCTGCAAGGCGATTAAGTT |
| | | GGGTAACGCCAGGGTTTTCCCAGTCACGAC |
| | | GTTGTAAAACGACGGCCAGTGCCAAGCTG |
| 93 | Nucleic acid | TATAAATATAAAGTAGTAAAAATTGAACCA |
| | sequence of pCDH- | TTAGGAGTAGCACCCACCAAGGCAAAGAGA |
| | EF1- | AGAGTGGTGCAGAGAGAAAAAAGAGCAGTG |
| | CAR3a_19aalinker- | GGAATAGGAGCTTTGTTCCTTGGGTTCTTG |
| | plasmid | GGAGCAGCAGGAAGCACTATGGGCGCAGCC |
| | | TCAATGACGCTGACGGTACAGGCCAGACAA |
| | | TTATTGTCTGGTATAGTGCAGCAGCAGAAC |
| | | AATTTGCTGAGGGCTATTGAGGCGCAACAG |
| | | CATCTGTTGCAACTCACAGTCTGGGGCATC |
| | | AAGCAGCTCCAGGCAAGAATCCTGGCTGTG |
| | | GAAAGATACCTAAAGGATCAACAGCTCCTG |
| | | GGGATTTGGGGTTGCTCTGGAAAACTCATT |
| | | TGCACCACTGCTGTGCCTTGGAATGCTAGT |
| | | TGGAGTAATAAATCTCTGGAACAGATTGGA |
| | | ATCACACGACCTGGATGGAGTGGGACAGAG |
| | | AAATTAACAATTACACAAGCTTAATACACT |
| | | CCTTAATTGAAGAATCGCAAAACCAGCAAG |
| | | AAAAGAATGAACAAGAATTATTGGAATTAG |
| | | ATAAATGGGCAAGTTTGTGGAATTGGTTTA |
| | | ACATAACAAATTGGCTGTGGTATATAAAAT |
| | | TATTCATAATGATAGTAGGAGGCTTGGTAG |
| | | GTTTAAGAATAGTTTTTGCTGTACTTTCTA |
| | | TAGTGAATAGAGTTAGGCAGGGATATTCAC |
| | | CATTATCGTTTCAGACCCACCTCCCAACCC |
| | | CGAGGGGACCCGACAGGCCCGAAGGAATAG |
| | | AAGAAGAAGGTGGAGAGAGAGACAGAGACA |
| | | GATCCATTCGATTAGTGAACGGATCTCGAC |
| | | GGTATCGGTTAACTTTTAAAAGAAAAGGGG |
| | | GGATTGGGGGGTACAGTGCAGGGGAAAGAA |
| | | TAGTAGACATAATAGCAACAGACATACAAA |
| | | CTAAAGAATTACAAAAACAAATTACAAAAA |
| | | TTCAAAATTTTATCGATACTAGTAAGGATC |
| | | TGCGATCGCTCCGGTGCCCGTCAGTGGGCA |
| | | GAGCGCACATCGCCCACAGTCCCCGAGAAG |
| | | TTGGGGGGAGGGGTCGGCAATTGAACGGGT |
| | | GCCTAGAGAAGGTGGCGCGGGGTAAACTGG |
| | | GAAAGTGATGTCGTGTACTGGCTCCGCCTT |
| | | TTTCCCGAGGGTGGGGGAGAACCGTATATA |
| | | AGTGCAGTAGTCGCCGTGAACGTTCTTTTT |
| | | CGCAACGGGTTTGCCGCCAGAACACAGCTG |
| | | AAGCTTCGAGGGGCTCGCATCTCTCCTTCA |
| | | CGCGCCCGCCGCCCTACCTGAGGCCGCCAT |
| | | CCACGCCGGTTGAGTCGCGTTCTGCCGCCT |
| | | CCCGCCTGTGGTGCCTCCTGAACTGCGTCC |
| | | GCCGTCTAGGTAAGTTTAAAGCTCAGGTCG |
| | | AGACCGGGCCTTTGTCCGGCGCTCCCTTGG |
| | | AGCCTACCTAGACTCAGCCGGCTCTCCACG |
| | | CTTTGCCTGACCCTGCTTGCTCAACTCTAC |
| | | GTCTTTGTTTCGTTTTCTGTTCTGCGCCGT |
| | | TACAGATCCAAGCTGTGACCGGCGCCTACT |
| | | CTAGAATGGCCTTACCAGTGACCGCCTTGC |
| | | TCCTGCCGCTGGCCTTGCTGCTCCACGCCG |
| | | CCAGGCCGGGATCCGACATCCAGATGACCC |
| | | AGAGCCCCTCCTCTTTATCCGCCTCTGTGG |
| | | GCGACAGAGTGACCATCACTTGTCGTGCCA |
| | | GCCAAGAAATCTCCGGCTATCTGTCTTGGC |
| | | TGCAGCAGAAGCCCGGTAAGGCTATCAAGA |
| | | GACTCATCTACGCCGCCAGCACTTTACAGA |
| | | GCGGAGTGCCTAGCAGATTTAGCGGCTCTC |
| | | GTAGCGGCAGCGATTATACTTTAACCATCT |
| | | CCTCTTTACAGCCCGAAGACTTTGCTACCT |
| | | ACTACTGTTTACAGTACGCCAGCTACCCCT |
| | | TCACCTTCGGTCAAGGTACCAAGCTGGAGA |
| | | TCAAAGGTGGCGGCGGCTCTGGCGGCGGTG |
| | | GCTCTGGCGGTGGCTCCGGCGGTGGTGGTA |
| | | GCCAAGTTACCCTCAAGGAGAGCGGCCCCA |
| | | CTTTAGTGAAGCCTACCCAGACTTTAACTT |
| | | TAACTTGTACCTTCAGCGGCTTCTCTTTAA |
| | | GCACCTCCACAATGGGCGTGGGCTGGATCA |
| | | GACAGCCTCCCGGCAAGGCTCTGGAGTGGC |
| | | TGGCCCACATCCTCTGGAACGACAGCAAGA |
| | | GGTACAACCCCTCTTTAAAGTCTCGTCTGA |
| | | CCATCACCAAGGACACCTCCAAGAAGCAAG |
| | | TTGTGCTGACCATGACCAATATGGACCCCG |
| | | TGGACACCGCCACCTATTACTGCGCTCGTA |
| | | TCGTGTACTACTCCACCTACGTGGGCTACT |
| | | TCGACGTCTGGGGACAAGGTACCACCGTGA |
| | | CCGTGAGCTCTGGATCCTTCGTGCCGGTCT |
| | | TCCTGCCAGCGAAGCCCACCACGACGCCAG |
| | | CGCCGCGACCACCAACACCGGCGCCCACCA |
| | | TCGCGTCGCAGCCCCTGTCCCTGCGCCCAG |
| | | AGGCGTGCCGGCCAGCGGCGGGGGGCGCAG |
| | | TGCACACGAGGGGGCTGGACTTCGCCTGTG |
| | | ATATCTACATCTGGGCGCCCTTGGCCGGGA |
| | | CTTGTGGGGTCCTTCTCCTGTCACTGGTTA |
| | | TCACCCTTTACTGCAACCACAGGAACAGGA |
| | | GTAAGAGGAGCAGGCTCCTGCACAGTGACT |
| | | ACATGAACATGACTCCCCGCCGCCCCGGGC |
| | | CCACCCGCAAGCATTACCAGCCCTATGCCC |
| | | CACCACGCGACTTCGCAGCCTATCGCTCCA |
| | | AACGGGGCAGAAAGAAACTCCTGTATATAT |
| | | TCAAACAACCATTTATGAGACCAGTACAAA |
| | | CTACTCAAGAGGAAGATGGCTGTAGCTGCC |
| | | GATTTCCAGAAGAAGAAGAAGGAGGATGTG |
| | | AACTGAGAGTGAAGTTCAGCAGGAGCGCAG |
| | | ACGCCCCCGCGTACCAGCAGGGCCAGAACC |
| | | AGCTCTATAACGAGCTCAATCTAGGACGAA |
| | | GAGAGGAGTACGATGTTTTGGACAAGAGAC |
| | | GTGGCCGGGACCCTGAGATGGGGGGAAAGC |
| | | CGAGAAGGAAGAACCCTCAGGAAGGCCTGT |
| | | ACAATGAACTGCAGAAAGATAAGATGGCGG |
| | | AGGCCTACAGTGAGATTGGGATGAAAGGCG |
| | | AGCGCCGGAGGGGCAAGGGGCACGATGGCC |
| | | TTTACCAGGGTCTCAGTACAGCCACCAAGG |
| | | ACACCTACGACGCCCTTCACATGCAGGCCC |
| | | TGCCCCCTCGCGCGGCCGCTAGCTGAATCT |
| | | AAGTCGACAATCAACCTCTGGATTACAAAA |
| | | TTTGTGAAAGATTGACTGGTATTCTTAACT |
| | | ATGTTGCTCCTTTTACGCTATGTGGATACG |
| | | CTGCTTTAATGCCTTTGTATCATGCTATTG |
| | | CTTCCCGTATGGCTTTCATTTTCTCCTCCT |
| | | TGTATAAATCCTGGTTGCTGTCTCTTTATG |
| | | AGGAGTTGTGGCCCGTTGTCAGGCAACGTG |
| | | GCGTGGTGTGCACTGTGTTTGCTGACGCAA |
| | | CCCCCACTGGTTGGGGCATTGCCACCACCT |
| | | GTCAGCTCCTTTCCGGGACTTTCGCTTTCC |
| | | CCCTCCCTATTGCCACGGCGGAACTCATCG |
| | | CCGCCTGCCTTGCCCGCTGCTGGACAGGGG |
| | | CTCGGCTGTTGGGCACTGACAATTCCGTGG |
| | | TGTTGTCGGGGAAATCATCGTCCTTTCCTT |
| | | GGCTGCTCGCCTGTGTTGCCACCTGGATTC |
| | | TGCGCGGGACGTCCTTCTGCTACGTCCCTT |
| | | CGGCCCTCAATCCAGCGGACCTTCCTTCCC |
| | | GCGGCCTGCTGCCGGCTCTGCGGCCTCTTC |
| | | CGCGTCTTCGCCTTCGCCCTCAGACGAGTC |
| | | GGATCTCCCTTTGGGCCGCCTCCCCGCCTG |
| | | GTACCTTTAAGACCAATGACTTACAAGGCA |
| | | GCTGTAGATCTTAGCCACTTTTTAAAAGAA |
| | | AAGGGGGGACTGGAAGGGCTAATTCACTCC |
| | | CAACGAAAATAAGATCTGCTTTTTGCTTGT |
| | | ACTGGGTCTCTCTGGTTAGACCAGATCTGA |
| | | GCCTGGGAGCTCTCTGGCTAACTAGGGAAC |
| | | CCACTGCTTAAGCCTCAATAAAGCTTGCCT |
| | | TGAGTGCTTCAAGTAGTGTGTGCCCGTCTG |
| | | TTGTGTGACTCTGGTAACTAGAGATCCCTC |
| | | AGACCCTTTTAGTCAGTGTGGAAAATCTCT |
| | | AGCAGTAGTAGTTCATGTCATCTTATTATT |
| | | CAGTATTTATAACTTGCAAAGAAATGAATA |
| | | TCAGAGAGTGAGAGGAACTTGTTTATTGCA |
| | | GCTTATAATGGTTACAAATAAAGCAATAGC |
| | | ATCACAAATTTCACAAATAAAGCATTTTTT |
| | | TCACTGCATTCTAGTTGTGGTTTGTCCAAA |
| | | CTCATCAATGTATCTTATCATGTCTGGCTC |
| | | TAGCTATCCCGCCCCTAACTCCGCCCAGTT |
| | | CCGCCCATTCTCCGCCCCATGGCTGACTAA |
| | | TTTTTTTTATTTATGCAGAGGCCGAGGCCG |
| | | CCTCGGCCTCTGAGCTATTCCAGAAGTAGT |
| | | GAGGAGGCTTTTTTGGAGGCCTAGACTTTT |
| | | GCAGAGACGGCCCAAATTCGTAATCATGGT |
| | | CATAGCTGTTTCCTGTGTGAAATTGTTATC |
| | | CGCTCACAATTCCACACAACATACGAGCCG |
| | | GAAGCATAAAGTGTAAAGCCTGGGGTGCCT |
| | | AATGAGTGAGCTAACTCACATTAATTGCGT |
| | | TGCGCTCACTGCCCGCTTTCCAGTCGGGAA |
| | | ACCTGTCGTGCCAGCTGCATTAATGAATCG |
| | | GCCAACGCGCGGGGAGAGGCGGTTTGCGTA |
| | | TTGGGCGCTCTTCCGCTTCCTCGCTCACTG |
| | | ACTCGCTGCGCTCGGTCGTTCGGCTGCGGC |
| | | GAGCGGTATCAGCTCACTCAAAGGCGGTAA |
| | | TACGGTTATCCACAGAATCAGGGGATAACG |
| | | CAGGAAAGAACATGTGAGCAAAAGGCCAGC |
| | | AAAAGGCCAGGAACCGTAAAAAGGCCGCGT |
| | | TGCTGGCGTTTTTCCATAGGCTCCGCCCCC |
| | | CTGACGAGCATCACAAAAATCGACGCTCAA |
| | | GTCAGAGGTGGCGAAACCCGACAGGACTAT |
| | | AAAGATACCAGGCGTTTCCCCCTGGAAGCT |
| | | CCCTCGTGCGCTCTCCTGTTCCGACCCTGC |
| | | CGCTTACCGGATACCTGTCCGCCTTTCTCC |
| | | CTTCGGGAAGCGTGGCGCTTTCTCATAGCT |
| | | CACGCTGTAGGTATCTCAGTTCGGTGTAGG |
| | | TCGTTCGCTCCAAGCTGGGCTGTGTGCACG |
| | | AACCCCCCGTTCAGCCCGACCGCTGCGCCT |
| | | TATCCGGTAACTATCGTCTTGAGTCCAACC |
| | | CGGTAAGACACGACTTATCGCCACTGGCAG |
| | | CAGCCACTGGTAACAGGATTAGCAGAGCGA |
| | | GGTATGTAGGCGGTGCTACAGAGTTCTTGA |
| | | AGTGGTGGCCTAACTACGGCTACACTAGAA |
| | | GGACAGTATTTGGTATCTGCGCTCTGCTGA |
| | | AGCCAGTTACCTTCGGAAAAAGAGTTGGTA |
| | | GCTCTTGATCCGGCAAACAAACCACCGCTG |
| | | GTAGCGGTGGTTTTTTTGTTTGCAAGCAGC |
| | | AGATTACGCGCAGAAAAAAAGGATCTCAAG |
| | | AAGATCCTTTGATCTTTTCTACGGGGTCTG |
| | | ACGCTCAGTGGAACGAAAACTCACGTTAAG |
| | | GGATTTTGGTCATGAGATTATCAAAAAGGA |
| | | TCTTCACCTAGATCCTTTTAAATTAAAAAT |
| | | GAAGTTTTAAATCAATCTAAAGTATATATG |
| | | AGTAAACTTGGTCTGACAGTTACCAATGCT |
| | | TAATCAGTGAGGCACCTATCTCAGCGATCT |
| | | GTCTATTTCGTTCATCCATAGTTGCCTGAC |
| | | TCCCCGTCGTGTAGATAACTACGATACGGG |
| | | AGGGCTTACCATCTGGCCCCAGTGCTGCAA |
| | | TGATACCGCGAGACCCACGCTCACCGGCTC |
| | | CAGATTTATCAGCAATAAACCAGCCAGCCG |
| | | GAAGGGCCGAGCGCAGAAGTGGTCCTGCAA |
| | | CTTTATCCGCCTCCATCCAGTCTATTAATT |
| | | GTTGCCGGGAAGCTAGAGTAAGTAGTTCGC |
| | | CAGTTAATAGTTTGCGCAACGTTGTTGCCA |
| | | TTGCTACAGGCATCGTGGTGTCACGCTCGT |
| | | CGTTTGGTATGGCTTCATTCAGCTCCGGTT |
| | | CCCAACGATCAAGGCGAGTTACATGATCCC |
| | | CCATGTTGTGCAAAAAAGCGGTTAGCTCCT |
| | | TCGGTCCTCCGATCGTTGTCAGAAGTAAGT |
| | | TGGCCGCAGTGTTATCACTCATGGTTATGG |
| | | CAGCACTGCATAATTCTCTTACTGTCATGC |
| | | CATCCGTAAGATGCTTTTCTGTGACTGGTG |
| | | AGTACTCAACCAAGTCATTCTGAGAATAGT |
| | | GTATGCGGCGACCGAGTTGCTCTTGCCCGG |
| | | CGTCAATACGGGATAATACCGCGCCACATA |
| | | GCAGAACTTTAAAAGTGCTCATCATTGGAA |
| | | AACGTTCTTCGGGGCGAAAACTCTCAAGGA |
| | | TCTTACCGCTGTTGAGATCCAGTTCGATGT |
| | | AACCCACTCGTGCACCCAACTGATCTTCAG |
| | | CATCTTTTACTTTCACCAGCGTTTCTGGGT |
| | | GAGCAAAAACAGGAAGGCAAAATGCCGCAA |
| | | AAAAGGGAATAAGGGCGACACGGAAATGTT |
| | | GAATACTCATACTCTTCCTTTTTCAATATT |
| | | ATTGAAGCATTTATCAGGGTTATTGTCTCA |
| | | TGAGCGGATACATATTTGAATGTATTTAGA |
| | | AAAATAAACAAATAGGGGTTCCGCGCACAT |
| | | TTCCCCGAAAAGTGCCACCTGACGTCTAAG |
| | | AAACCATTATTATCATGACATTAACCTATA |
| | | AAAATAGGCGTATCACGAGGCCCTTTCGTC |
| | | TCGCGCGTTTCGGTGATGACGGTGAAAACC |
| | | TCTGACACATGCAGCTCCCGGAGACGGTCA |
| | | CAGCTTGTCTGTAAGCGGATGCCGGGAGCA |
| | | GACAAGCCCGTCAGGGCGCGTCAGCGGGTG |
| | | TTGGCGGGTGTCGGGGCTGGCTTAACTATG |
| | | CGGCATCAGAGCAGATTGTACTGAGAGTGC |
| | | ACCATATGCGGTGTGAAATACCGCACAGAT |
| | | GCGTAAGGAGAAAATACCGCATCAGGCGCC |
| | | ATTCGCCATTCAGGCTGCGCAACTGTTGGG |
| | | AAGGGCGATCGGTGCGGGCCTCTTCGCTAT |
| | | TACGCCAGCTGGCGAAAGGGGGATGTGCTG |
| | | CAAGGCGATTAAGTTGGGTAACGCCAGGGT |
| | | TTTCCCAGTCACGACGTTGTAAAACGACGG |
| | | CCAGTGCCAAGCTG |
| 94 | Nucleic Acid | aatgtagtcttatgcaatactcttgtagtc |
| | sequence of | ttgcaacatggtaacgatgagttagcaaca |
| | EF1_CAR3a_19aa | tgccttacaaggagagaaaaagcaccgtgc |
| | linkerT2Aicasp9_V | atgccgattggtggaagtaaggtggtacga |
| | B200623-2571wvv | tcgtgccttattaggaaggcaacagacggg |
| | Plasmid | tctgacatggattggacgaaccactgaatt |
| | | gccgcattgcagagatattgtatttaagtg |
| | | cctagctcgatacataaacgggtctctctg |
| | | gttagaccagatctgagcctgggagctctc |
| | | tggctaactagggaacccactgcttaagcc |
| | | tcaataaagcttgccttgagtgcttcaagt |
| | | agtgtgtgcccgtctgttgtgtgactctgg |
| | | taactagagatccctcagacccttttagtc |
| | | agtgtggaaaatctctagcagtggcgcccg |
| | | aacagggacttgaaagcgaaagggaaacca |
| | | gaggagctctctcgacgcaggactcggctt |
| | | gctgaagcgcgcacggcaagaggcgagggg |
| | | cggcgactggtgagtacgccaaaaattttg |
| | | actagcggaggctagaaggagagagatggg |
| | | tgcgagagcgtcagtattaagcgggggaga |
| | | attagatcgcgatgggaaaaaattcggtta |
| | | aggccagggggaaagaaaaaatataaatta |
| | | aaacatatagtatgggcaagcagggagcta |
| | | gaacgattcgcagttaatcctggcctgtta |
| | | gaaacatcagaaggctgtagacaaatactg |
| | | ggacagctacaaccatcccttcagacagga |
| | | tcagaagaacttagatcattatataataca |
| | | gtagcaaccctctattgtgtgcatcaaagg |
| | | atagagataaaagacaccaaggaagcttta |
| | | gacaagatagaggaagagcaaaacaaaagt |
| | | aagaccaccgcacagcaagcggccgctgat |
| | | cttcagacctggaggaggagatatgaggga |
| | | caattggagaagtgaattatataaatataa |
| | | agtagtaaaaattgaaccattaggagtagc |
| | | acccaccaaggcaaagagaagagtggtgca |
| | | gagagaaaaaagagcagtgggaataggagc |
| | | tttgttccttgggttcttgggagcagcagg |
| | | aagcactatgggcgcagcgtcaatgacgct |
| | | gacggtacaggccagacaattattgtctgg |
| | | tatagtgcagcagcagaacaatttgctgag |
| | | ggctattgaggcgcaacagcatctgttgca |
| | | actcacagtctggggcatcaagcagctcca |
| | | ggcaagaatcctggctgtggaaagatacct |
| | | aaaggatcaacagctcctggggatttgggg |
| | | ttgctctggaaaactcatttgcaccactgc |
| | | tgtgccttggaatgctagttggagtaataa |
| | | atctctggaacagatttggaatcacacgac |
| | | ctggatggagtgggacagagaaattaacaa |
| | | ttacacaagcttaatacactccttaattga |
| | | agaatcgcaaaaccagcaagaaaagaatga |
| | | acaagaattattggaattagataaatgggc |
| | | aagtttgtggaattggtttaacataacaaa |
| | | ttggctgtggtatataaaattattcataat |
| | | gatagtaggaggcttggtaggtttaagaat |
| | | agtttttgctgtactttctatagtgaatag |
| | | agttaggcagggatattcaccattatcgtt |
| | | tcagacccacctcccaaccccgaggggacc |
| | | cgacaggcccgaaggaatagaagaagaagg |
| | | tggagagagagacagagacagatccattcg |
| | | attagtgaacggatctcgacggtatcgcta |
| | | gcttttaaaagaaaaggggggattgggggg |
| | | tacagtgcaggggaaagaatagtagacata |
| | | atagcaacagacatacaaactaaagaatta |
| | | caaaaacaaattacaaaaattcaaaatttt |
| | | actagtgattatcggatcaactttgtatag |
| | | aaaagttgggctccggtgcccgtcagtggg |
| | | cagagcgcacatcgcccacagtccccgaga |
| | | agttggggggaggggtcggcaattgaaccg |
| | | gtgcctagagaaggtggcgcggggtaaact |
| | | gggaaagtgatgtcgtgtactggctccgcc |
| | | tttttcccgaggggggggagaaccgtatat |
| | | aagtgcagtagtcgccgtgaacgttctttt |
| | | tcgcaacgggtttgccgccagaacacaggt |
| | | aagtgccgtgtgtggttcccgcgggcctgg |
| | | cctctttacgggttatggcccttgcgtgcc |
| | | ttgaattacttccacctggctgcagtacgt |
| | | gattcttgatcccgagcttcgggttggaag |
| | | tgggtgggagagttcgaggccttgcgctta |
| | | aggagccccttcgcctcgtgcttgagttga |
| | | ggcctggcctgggcgctggggccgccgcgt |
| | | gcgaatctggtggcaccttcgcgcctgtct |
| | | cgctgctttcgataagtctctagccattta |
| | | aaatttttgatgacctgctgcgacgctttt |
| | | tttctggcaagatagtcttgtaaatgcggg |
| | | ccaagatctgcacactggtatttcggtttt |
| | | tggggccgcgggcggcgacggggcccgtgc |
| | | gtcccagcgcacatgttcggcgaggggggc |
| | | ctgcgagcgcggccaccgagaatcggacgg |
| | | gggtagtctcaagctggccggcctgctctg |
| | | gtgcctggtctcgcgccgccgtgtatcgcc |
| | | ccgccctgggcggcaaggctggcccggtcg |
| | | gcaccagttgcgtgagcggaaagatggccg |
| | | cttcccggccctgctgcagggagctcaaaa |
| | | tggaggacgcggcgctcgggagagcggggg |
| | | gtgagtcacccacacaaaggaaaagggcct |
| | | ttccgtcctcagccgtcgcttcatgtgact |
| | | ccacggagtaccggggccgtccaggcacct |
| | | cgattagttctcgagcttttggagtacgtc |
| | | gtctttaggttggggggaggggttttatgc |
| | | gatggagtttccccacactgagtgggtgga |
| | | gactgaagttaggccagcttggcacttgat |
| | | gtaattctccttggaatttgccctttttga |
| | | gtttggatcttggttcattctcaagcctca |
| | | gacagtggttcaaagtttttttcttccatt |
| | | tcaggtgtcgtgacaagtttgtacaaaaaa |
| | | gcaggctgccaccatggccttaccagtgac |
| | | cgccttgctcctgccgctggccttgctgct |
| | | ccacgccgccaggccgggatccgacatcca |
| | | gatgacccagagcccctcctctttatccgc |
| | | ctctgtgggcgacagagtgaccatcacttg |
| | | tcgtgccagccaagaaatctccggctatct |
| | | gtcttggctgcagcagaagcccggtaaggc |
| | | tatcaagagactcatctacgccgccagcac |
| | | tttacagagcggagtgcctagcagatttag |
| | | cggctctcgtagcggcagcgattatacttt |
| | | aaccatctcctctttacagcccgaagactt |
| | | tgctacctactactgtttacagtacgccag |
| | | ctaccccttcaccttcggtcaaggtaccaa |
| | | gctggagatcaaaggtggcggcggctctgg |
| | | cggcggtggctctggcggtggctccggcgg |
| | | tggtggtagccaagttaccctcaaggagag |
| | | cggccccactttagtgaagcctacccagac |
| | | tttaactttaacttgtaccttcagcggctt |
| | | ctctttaagcacctccacaatgggcgtggg |
| | | ctggatcagacagcctcccggcaaggctct |
| | | ggagtggctggcccacatcctctggaacga |
| | | cagcaagaggtacaacccctctttaaagtc |
| | | tcgtctgaccatcaccaaggacacctccaa |
| | | gaagcaagttgtgctgaccatgaccaatat |
| | | ggaccccgtggacaccgccacctattactg |
| | | cgctcgtatcgtgtactactccacctacgt |
| | | gggctacttcgacgtctggggacaaggtac |
| | | caccgtgaccgtgagctctggatccttcgt |
| | | gccggtcttcctgccagcgaagcccaccac |
| | | gacgccagcgccgcgaccaccaacaccggc |
| | | gcccaccatcgcgtcgcagcccctgtccct |
| | | gcgcccagaggcgtgccggccagcgggggg |
| | | ggcgcagtgcacacgagggggctggacttc |
| | | gcctgtgatatctacatctgggcgcccttg |
| | | gccgggacttgtggggtccttctcctgtca |
| | | ctggttatcaccctttactgcaaccacagg |
| | | aacaggagtaagaggagcaggctcctgcac |
| | | agtgactacatgaacatgactccccgccgc |
| | | cccgggcccacccgcaagcattaccagccc |
| | | tatgccccaccacgcgacttcgcagcctat |
| | | cgctccaaacggggcagaaagaaactcctg |
| | | tatatattcaaacaaccatttatgagacca |
| | | gtacaaactactcaagaggaagatggctgt |
| | | agctgccgatttccagaagaagaagaagga |
| | | ggatgtgaactgagagtgaagttcagcagg |
| | | agcgcagacgcccccgcgtaccagcagggc |
| | | cagaaccagctctataacgagctcaatcta |
| | | ggacgaagagaggagtacgatgttttggac |
| | | aagagacgtggccgggaccctgagatgggg |
| | | ggaaagccgagaaggaagaaccctcaggaa |
| | | ggcctgtacaatgaactgcagaaagataag |
| | | atggcggaggcctacagtgagattgggatg |
| | | aaaggcgagcgccggaggggcaaggggcac |
| | | gatggcctttaccagggtctcagtacagcc |
| | | accaaggacacctacgacgcccttcacatg |
| | | caggccctgccccctcgcggaagcggagag |
| | | ggcaggggaagtcttctaacatgcggggac |
| | | gtggaggaaaatcccggccccatgggagtg |
| | | caggtggaaaccatctccccaggagacggg |
| | | cgcaccttccccaagcgcggccagacctgc |
| | | gtggtgcactacaccgggatgcttgaagat |
| | | ggaaagaaagtggattcctcccgggacaga |
| | | aacaagccctttaagtttatgctaggcaag |
| | | caggaggtgatccgaggctgggaagaaggg |
| | | gttgcccagatgagtgtgggtcagagagcc |
| | | aaactgactatatctccagattatgcctat |
| | | ggtgccactgggcacccaggcatcatccca |
| | | ccacatgccactctcgtcttcgatgtggag |
| | | cttctaaaactggaatctggaggaggttct |
| | | actaacaggcaagcagcaaagttgtcgaag |
| | | ccaaccctagaaaaccttaccccagtggtg |
| | | ctcagaccagagattcgcaaaccagaggtt |
| | | ctcagaccggaaacacccagaccagtggac |
| | | attggttctggaggatttggtgatgtcggt |
| | | gctcttgagagtttgaggggaaatgcagat |
| | | ttggcttacatcctgagcatggagccctgt |
| | | ggccactgcctcattatcaacaatgtgaac |
| | | ttctgccgtgagtccgggctccgcacccgc |
| | | actggctccaacatcgactgtgagaagttg |
| | | cggcgtcgcttctcctcgctgcatttcatg |
| | | gtggaggtgaagggcgacctgactgccaag |
| | | aaaatggtgctggctttgctggagctggcg |
| | | cagcaggaccacggtgctctggactgctgc |
| | | gtggtggtcattctctctcacggctgtcag |
| | | gccagccacctgcagttcccaggggctgtc |
| | | tacggcacagatggatgccctgtgtcggtc |
| | | gagaagattgtgaacatcttcaatgggacc |
| | | agctgccccagcctgggagggaagcccaag |
| | | ctctttttcatccaggcctgtggtggggag |
| | | cagaaagaccatgggtttgaggtggcctcc |
| | | acttcccctgaagacgagtcccctggcagt |
| | | aaccccgagccagatgccaccccgttccag |
| | | gaaggtttgaggaccttcgaccagctggac |
| | | gccatatctagtttgcccacacccagtgac |
| | | atctttgtgtcctactctactttcccaggt |
| | | tttgtttcctggagggaccccaagagtggc |
| | | tcctggtacgttgagaccctggacgacatc |
| | | tttgagcagtgggctcactctgaagacctg |
| | | cagtccctcctgcttagggtcgctaatgct |
| | | gtttcggtgaaagggatttataaacagatg |
| | | cctggttgctttaatttcctccggaaaaaa |
| | | cttttctttaaaacatcatgaacccagctt |
| | | tcttgtacaaagtggtgataatcgaattcc |
| | | gataatcaacctctggattacaaaatttgt |
| | | gaaagattgactggtattcttaactatgtt |
| | | gctccttttacgctatgtggatacgctgct |
| | | ttaatgcctttgtatcatgctattgcttcc |
| | | cgtatggctttcattttctcctccttgtat |
| | | aaatcctggttgctgtctctttatgaggag |
| | | ttgtggcccgttgtcaggcaacgtggcgtg |
| | | gtgtgcactgtgtttgctgacgcaaccccc |
| | | actggttggggcattgccaccacctgtcag |
| | | ctcctttccgggactttcgctttccccctc |
| | | cctattgccacggcggaactcatcgccgcc |
| | | tgccttgcccgctgctggacaggggctcgg |
| | | ctgttgggcactgacaattccgtggtgttg |
| | | tcggggaagctgacgtcctttccatggctg |
| | | ctcgcctgtgttgccacctggattctgcgc |
| | | gggacgtccttctgctacgtcccttcggcc |
| | | ctcaatccagcggaccttccttcccgcggc |
| | | ctgctgccggctctgcggcctcttccgcgt |
| | | cttcgccttcgccctcagacgagtcggatc |
| | | tccctttgggccgcctccccgcatcgggaa |
| | | ttcccgcggttcgctttaagaccaatgact |
| | | tacaaggcagctgtagatcttagccacttt |
| | | ttaaaagaaaaggggggactggaagggcta |
| | | attcactcccaacgaagacaagatctgctt |
| | | tttgcttgtactgggtctctctggttagac |
| | | cagatctgagcctgggagctctctggctaa |
| | | ctagggaacccactgcttaagcctcaataa |
| | | agcttgccttgagtgcttcaagtagtgtgt |
| | | gcccgtctgttgtgtgactctggtaactag |
| | | agatccctcagacccttttagtcagtgtgg |
| | | aaaatctctagcagtagtagttcatgtcat |
| | | cttattattcagtatttataacttgcaaag |
| | | aaatgaatatcagagagtgagaggaacttg |
| | | tttattgcagcttataatggttacaaataa |
| | | agcaatagcatcacaaatttcacaaataaa |
| | | gcatttttttcactgcattctagttgtggt |
| | | ttgtccaaactcatcaatgtatcttatcat |
| | | gtctggctctagctatcccgcccctaactc |
| | | cgcccatcccgcccctaactccgcccagtt |
| | | ccgcccattctccgccccatggctgactaa |
| | | ttttttttatttatgcagaggccgaggccg |
| | | cctcggcctctgagctattccagaagtagt |
| | | gaggaggcttttttggaggcctagggacgt |
| | | acccaattcgccctatagtgagtcgtatta |
| | | cgcgcgctcactggccgtcgttttacaacg |
| | | tcgtgactgggaaaaccctggcgttaccca |
| | | acttaatcgccttgcagcacatcccccttt |
| | | cgccagctggcgtaatagcgaagaggcccg |
| | | caccgatcgcccttcccaacagttgcgcag |
| | | cctgaatggcgaatgggacgcgccctgtag |
| | | cggcgcattaagcgcggcgggtgtggtggt |
| | | tacgcgcagcgtgaccgctacacttgccag |
| | | cgccctagcgcccgctcctttcgctttctt |
| | | cccttcctttctcgccacgttcgccggctt |
| | | tccccgtcaagctctaaatcgggggctccc |
| | | tttagggttccgatttagtgctttacggca |
| | | cctcgaccccaaaaaacttgattagggtga |
| | | tggttcacgtagtgggccatcgccctgata |
| | | gacggtttttcgccctttgacgttggagtc |
| | | cacgttctttaatagtggactcttgttcca |
| | | aactggaacaacactcaaccctatctcggt |
| | | ctattcttttgatttataagggattttgcc |
| | | gatttcggcctattggttaaaaaatgagct |
| | | gatttaacaaaaatttaacgcgaattttaa |
| | | caaaatattaacgcttacaatttaggtggc |
| | | acttttcggggaaatgtgcgcggaacccct |
| | | atttgtttatttttctaaatacattcaaat |
| | | atgtatccgctcatgagacaataaccctga |
| | | taaatgcttcaataatattgaaaaaggaag |
| | | agtatgagtattcaacatttccgtgtcgcc |
| | | cttattcccttttttgcggcattttgcctt |
| | | cctgtttttgctcacccagaaacgctggtg |
| | | aaagtaaaagatgctgaagatcagttgggt |
| | | gcacgagtgggttacatcgaactggatctc |
| | | aacagcggtaagatccttgagagttttcgc |
| | | cccgaagaacgttttccaatgatgagcact |
| | | tttaaagttctgctatgtggcgcggtatta |
| | | tcccgtattgacgccgggcaagagcaactc |
| | | ggtcgccgcatacactattctcagaatgac |
| | | ttggttgagtactcaccagtcacagaaaag |
| | | catcttacggatggcatgacagtaagagaa |
| | | ttatgcagtgctgccataaccatgagtgat |
| | | aacactgcggccaacttacttctgacaacg |
| | | atcggaggaccgaaggagctaaccgctttt |
| | | ttgcacaacatgggggatcatgtaactcgc |
| | | cttgatcgttgggaaccggagctgaatgaa |
| | | gccataccaaacgacgagcgtgacaccacg |
| | | atgcctgtagcaatggcaacaacgttgcgc |
| | | aaactattaactggcgaactacttactcta |
| | | gcttcccggcaacaattaatagactggatg |
| | | gaggcggataaagttgcaggaccacttctg |
| | | cgctcggcccttccggctggctggtttatt |
| | | gctgataaatctggagccggtgagcgtggg |
| | | tctcgcggtatcattgcagcactggggcca |
| | | gatggtaagccctcccgtatcgtagttatc |
| | | tacacgacggggagtcaggcaactatggat |
| | | gaacgaaatagacagatcgctgagataggt |
| | | gcctcactgattaagcattggtaactgtca |
| | | gaccaagtttactcatatatactttagatt |
| | | gatttaaaacttcatttttaatttaaaagg |
| | | atctaggtgaagatcctttttgataatctc |
| | | atgaccaaaatcccttaacgtgagttttcg |
| | | ttccactgagcgtcagaccccgtagaaaag |
| | | atcaaaggatcttcttgagatccttttttt |
| | | ctgcgcgtaatctgctgcttgcaaacaaaa |
| | | aaaccaccgctaccagcggtggtttgtttg |
| | | ccggatcaagagctaccaactctttttccg |
| | | aaggtaactggcttcagcagagcgcagata |
| | | ccaaatactgttcttctagtgtagccgtag |
| | | ttaggccaccacttcaagaactctgtagca |
| | | ccgcctacatacctcgctctgctaatcctg |
| | | ttaccagtggctgctgccagtggcgataag |
| | | tcgtgtcttaccgggttggactcaagacga |
| | | tagttaccggataaggcgcagcggtcgggc |
| | | tgaacggggggttcgtgcacacagcccagc |
| | | ttggagcgaacgacctacaccgaactgaga |
| | | tacctacagcgtgagctatgagaaagcgcc |
| | | acgcttcccgaagagagaaaggcggacagg |
| | | tatccggtaagcggcagggtcggaacagga |
| | | gagcgcacgagggagcttccagggggaaac |
| | | gcctggtatctttatagtcctgtcgggttt |
| | | cgccacctctgacttgagcgtcgatttttg |
| | | tgatgctcgtcaggggggcggagcctatgg |
| | | aaaaacgccagcaacgcggcctttttacgg |
| | | ttcctggccttttgctggccttttgctcac |
| | | atgttctttcctgcgttatcccctgattct |
| | | gtggataaccgtattaccgcctttgagtga |
| | | gctgataccgctcgccgcagccgaacgacc |
| | | gagcgcagcgagtcagtgagcgaggaagcg |
| | | gaagagcgcccaatacgcaaaccgcctctc |
| | | cccgcgcgttggccgattcattaatgcagc |
| | | tggcacgacaggtttcccgactggaaaggg |
| | | gcagtgagcgcaacgcaattaatgtgagtt |
| | | agctcactcattaggcaccccaggctttac |
| | | actttatgcttccggctcgtatgttgtgtg |
| | | gaattgtgagcggataacaatttcacacag |
| | | gaaacagctatgaccatgattacgccaagc |
| | | gcgcaattaaccctcactaaagggaacaaa |
| | | agctggagctgcaagctt |
| 95 | Nucleic Acid | aatgtagtcttatgcaatactcttgtagtc |
| | sequence of | ttgcaacatggtaacgatgagttagcaaca |
| | EF1_CAR3a_19aa | tgccttacaaggagagaaaaagcaccgtgc |
| | linkerT2Aegfit_VB | atgccgattggtggaagtaaggtggtacga |
| | 200623-1259qkz- | tcgtgccttattaggaaggcaacagacggg |
| | Plasmid | tctgacatggattggacgaaccactgaatt |
| | | gccgcattgcagagatattgtatttaagtg |
| | | cctagctcgatacataaacgggtctctctg |
| | | gttagaccagatctgagcctgggagctctc |
| | | tggctaactagggaacccactgcttaagcc |
| | | tcaataaagcttgccttgagtgcttcaagt |
| | | agtgtgtgcccgtctgttgtgtgactctgg |
| | | taactagagatccctcagacccttttagtc |
| | | agtgtggaaaatctctagcagtggcgcccg |
| | | aacagggacttgaaagcgaaagggaaacca |
| | | gaggagctctctcgacgcaggactcggctt |
| | | gctgaagcgcgcacggcaagaggcgagggg |
| | | cggcgactggtgagtacgccaaaaattttg |
| | | actagcggaggctagaaggagagagatggg |
| | | tgcgagagcgtcagtattaagcgggggaga |
| | | attagatcgcgatgggaaaaaattcggtta |
| | | aggccagggggaaagaaaaaatataaatta |
| | | aaacatatagtatgggcaagcagggagcta |
| | | gaacgattcgcagttaatcctggcctgtta |
| | | gaaacatcagaaggctgtagacaaatactg |
| | | ggacagctacaaccatcccttcagacagga |
| | | tcagaagaacttagatcattatataataca |
| | | gtagcaaccctctattgtgtgcatcaaagg |
| | | atagagataaaagacaccaaggaagcttta |
| | | gacaagatagaggaagagcaaaacaaaagt |
| | | aagaccaccgcacagcaagcggccgctgat |
| | | cttcagacctggaggaggagatatgaggga |
| | | caattggagaagtgaattatataaatataa |
| | | agtagtaaaaattgaaccattaggagtagc |
| | | acccaccaaggcaaagagaagagtggtgca |
| | | gagagaaaaaagagcagtgggaataggagc |
| | | tttgttccttgggttcttgggagcagcagg |
| | | aagcactatgggcgcagcgtcaatgacgct |
| | | gacggtacaggccagacaattattgtctgg |
| | | tatagtgcagcagcagaacaatttgctgag |
| | | ggctattgaggcgcaacagcatctgttgca |
| | | actcacagtctggggcatcaagcagctcca |
| | | ggcaagaatcctggctgtggaaagatacct |
| | | aaaggatcaacagctcctggggatttgggg |
| | | ttgctctggaaaactcatttgcaccactgc |
| | | tgtgccttggaatgctagttggagtaataa |
| | | atctctggaacagatttggaatcacacgac |
| | | ctggatggagtgggacagagaaattaacaa |
| | | ttacacaagcttaatacactccttaattga |
| | | agaatcgcaaaaccagcaagaaaagaatga |
| | | acaagaattattggaattagataaatgggc |
| | | aagtttgtggaattggtttaacataacaaa |
| | | ttggctgtggtatataaaattattcataat |
| | | gatagtaggaggcttggtaggtttaagaat |
| | | agtttttgctgtactttctatagtgaatag |
| | | agttaggcagggatattcaccattatcgtt |
| | | tcagacccacctcccaaccccgaggggacc |
| | | cgacaggcccgaaggaatagaagaagaagg |
| | | tggagagagagacagagacagatccattcg |
| | | attagtgaacggatctcgacggtatcgcta |
| | | gcttttaaaagaaaaggggggattgggggg |
| | | tacagtgcaggggaaagaatagtagacata |
| | | atagcaacagacatacaaactaaagaatta |
| | | caaaaacaaattacaaaaattcaaaatttt |
| | | actagtgattatcggatcaactttgtatag |
| | | aaaagttgggctccggtgcccgtcagtggg |
| | | cagagcgcacatcgcccacagtccccgaga |
| | | agttggggggaggggtcggcaattgaaccg |
| | | gtgcctagagaaggtggcgcggggtaaact |
| | | gggaaagtgatgtcgtgtactggctccgcc |
| | | tttttcccgaggggggggagaaccgtatat |
| | | aagtgcagtagtcgccgtgaacgttctttt |
| | | tcgcaacgggtttgccgccagaacacaggt |
| | | aagtgccgtgtgtggttcccgcgggcctgg |
| | | cctctttacgggttatggcccttgcgtgcc |
| | | ttgaattacttccacctggctgcagtacgt |
| | | gattcttgatcccgagcttcgggttggaag |
| | | tgggtgggagagttcgaggccttgcgctta |
| | | aggagccccttcgcctcgtgcttgagttga |
| | | ggcctggcctgggcgctggggccgccgcgt |
| | | gcgaatctggtggcaccttcgcgcctgtct |
| | | cgctgctttcgataagtctctagccattta |
| | | aaatttttgatgacctgctgcgacgctttt |
| | | tttctggcaagatagtcttgtaaatgcggg |
| | | ccaagatctgcacactggtatttcggtttt |
| | | tggggccgcgggcggcgacggggcccgtgc |
| | | gtcccagcgcacatgttcggcgaggcgggg |
| | | cctgcgagcgcggccaccgagaatcggacg |
| | | ggggtagtctcaagctggccggcctgctct |
| | | ggtgcctggtctcgcgccgccgtgtatcgc |
| | | cccgccctgggcggcaaggctggcccggtc |
| | | ggcaccagttgcgtgagcggaaagatggcc |
| | | gcttcccggccctgctgcagggagctcaaa |
| | | atggaggacgcggcgctcgggagagcgggg |
| | | ggtgagtcacccacacaaaggaaaagggcc |
| | | tttccgtcctcagccgtcgcttcatgtgac |
| | | tccacggagtaccgggcgccgtccaggcac |
| | | ctcgattagttctcgagcttttggagtacg |
| | | tcgtctttaggttggggggaggggttttat |
| | | gcgatggagtttccccacactgagtgggtg |
| | | gagactgaagttaggccagcttggcacttg |
| | | atgtaattctccttggaatttgcccttttt |
| | | gagtttggatcttggttcattctcaagcct |
| | | cagacagtggttcaaagtttttttcttcca |
| | | tttcaggtgtcgtgacaagtttgtacaaaa |
| | | aagcaggctgccaccatggccttaccagtg |
| | | accgccttgctcctgccgctggccttgctg |
| | | ctccacgccgccaggccgggatccgacatc |
| | | cagatgacccagagcccctcctctttatcc |
| | | gcctctgtgggcgacagagtgaccatcact |
| | | tgtcgtgccagccaagaaatctccggctat |
| | | ctgtcttggctgcagcagaagcccggtaag |
| | | gctatcaagagactcatctacgccgccagc |
| | | actttacagagcggagtgcctagcagattt |
| | | agcggctctcgtagcggcagcgattatact |
| | | ttaaccatctcctctttacagcccgaagac |
| | | tttgctacctactactgtttacagtacgcc |
| | | agctaccccttcaccttcggtcaaggtacc |
| | | aagctggagatcaaaggtggcggcggctct |
| | | ggcggcggtggctctggcggtggctccggc |
| | | ggtggtggtagccaagttaccctcaaggag |
| | | agcggccccactttagtgaagcctacccag |
| | | actttaactttaacttgtaccttcagcggc |
| | | ttctctttaagcacctccacaatgggcgtg |
| | | ggctggatcagacagcctcccggcaaggct |
| | | ctggagtggctggcccacatcctctggaac |
| | | gacagcaagaggtacaacccctctttaaag |
| | | tctcgtctgaccatcaccaaggacacctcc |
| | | aagaagcaagttgtgctgaccatgaccaat |
| | | atggaccccgtggacaccgccacctattac |
| | | tgcgctcgtatcgtgtactactccacctac |
| | | gtgggctacttcgacgtctggggacaaggt |
| | | accaccgtgaccgtgagctctggatccttc |
| | | gtgccggtcttcctgccagcgaagcccacc |
| | | acgacgccagcgccgcgaccaccaacaccg |
| | | gcgcccaccatcgcgtcgcagcccctgtcc |
| | | ctgcgcccagaggcgtgccggccagcgggg |
| | | ggggcgcagtgcacacgagggggctggact |
| | | tcgcctgtgatatctacatctgggcgccct |
| | | tggccgggacttgtggggtccttctcctgt |
| | | cactggttatcaccctttactgcaaccaca |
| | | ggaacaggagtaagaggagcaggctcctgc |
| | | acagtgactacatgaacatgactccccgcc |
| | | gccccgggcccacccgcaagcattaccagc |
| | | cctatgccccaccacgcgacttcgcagcct |
| | | atcgctccaaacggggcagaaagaaactcc |
| | | tgtatatattcaaacaaccatttatgagac |
| | | cagtacaaactactcaagaggaagatggct |
| | | gtagctgccgatttccagaagaagaagaag |
| | | gaggatgtgaactgagagtgaagttcagca |
| | | ggagcgcagacgcccccgcgtaccagcagg |
| | | gccagaaccagctctataacgagctcaatc |
| | | taggacgaagagaggagtacgatgttttgg |
| | | acaagagacgtggccgggaccctgagatgg |
| | | ggggaaagccgagaaggaagaaccctcagg |
| | | aaggcctgtacaatgaactgcagaaagata |
| | | agatggcggaggcctacagtgagattggga |
| | | tgaaaggcgagcgccggaggggcaaggggc |
| | | acgatggcctttaccagggtctcagtacag |
| | | ccaccaaggacacctacgacgcccttcaca |
| | | tgcaggccctgccccctcgcggaagcggag |
| | | agggcaggggaagtcttctaacatgcgggg |
| | | acgtggaggaaaatcccggccccatgcttc |
| | | tcctggtgacaagccttctgctctgtgagt |
| | | taccacacccagcattcctcctgatcccac |
| | | gcaaagtgtgtaacggaataggtattggtg |
| | | aatttaaagactcactctccataaatgcta |
| | | cgaatattaaacacttcaaaaactgcacct |
| | | ccatcagtggcgatctccacatcctgccgg |
| | | tggcatttaggggtgactccttcacacata |
| | | ctcctcctctggatccacaggaactggata |
| | | ttctgaaaaccgtaaaggaaatcacagggt |
| | | ttttgctgattcaggcttggcctgaaaaca |
| | | ggacggacctccatgcctttgagaacctag |
| | | aaatcatacgcggcaggaccaagcaacatg |
| | | gtcagttttctcttgcagtcgtcagcctga |
| | | acataacatccttgggattacgctccctca |
| | | aggagataagtgatggagatgtgataattt |
| | | caggaaacaaaaatttgtgctatgcaaata |
| | | caataaactggaaaaaactgtttgggacct |
| | | ccggtcagaaaaccaaaattataagcaaca |
| | | gaggtgaaaacagctgcaaggccacaggcc |
| | | aggtctgccatgccttgtgctcccccgagg |
| | | gctgctggggcccggagcccagggactgcg |
| | | tctcttgccggaatgtcagccgaggcaggg |
| | | aatgcgtggacaagtgcaaccttctggagg |
| | | gtgagccaagggagtttgtggagaactctg |
| | | agtgcatacagtgccacccagagtgcctgc |
| | | ctcaggccatgaacatcacctgcacaggac |
| | | ggggaccagacaactgtatccagtgtgccc |
| | | actacattgacggcccccactgcgtcaaga |
| | | cctgcccggcaggagtcatgggagaaaaca |
| | | acaccctggtctggaagtacgcagacgccg |
| | | gccatgtgtgccacctgtgccatccaaact |
| | | gcacctacggatgcactgggccaggtcttg |
| | | aaggctgtccaacgaatgggcctaagatcc |
| | | cgtccatcgccactgggatggtgggggccc |
| | | tcctcttgctgctggtggtggccctgggga |
| | | tcggcctcttcatgtgaacccagctttctt |
| | | gtacaaagtggtgataatcgaattccgata |
| | | atcaacctctggattacaaaatttgtgaaa |
| | | gattgactggtattcttaactatgttgctc |
| | | cttttacgctatgtggatacgctgctttaa |
| | | tgcctttgtatcatgctattgcttcccgta |
| | | tggctttcattttctcctccttgtataaat |
| | | cctggttgctgtctctttatgaggagttgt |
| | | ggcccgttgtcaggcaacgtggcgtggtgt |
| | | gcactgtgtttgctgacgcaacccccactg |
| | | gttggggcattgccaccacctgtcagctcc |
| | | tttccgggactttcgctttccccctcccta |
| | | ttgccacggcggaactcatcgccgcctgcc |
| | | ttgcccgctgctggacaggggctcggctgt |
| | | tgggcactgacaattccgtggtgttgtcgg |
| | | ggaagctgacgtcctttccatggctgctcg |
| | | cctgtgttgccacctggattctgcgcggga |
| | | cgtccttctgctacgtcccttcggccctca |
| | | atccagcggaccttccttcccgcggcctgc |
| | | tgccggctctgcggcctcttccgcgtcttc |
| | | gccttcgccctcagacgagtcggatctccc |
| | | tttgggccgcctccccgcatcgggaattcc |
| | | cgcggttcgctttaagaccaatgacttaca |
| | | aggcagctgtagatcttagccactttttaa |
| | | aagaaaaggggggactggaagggctaattc |
| | | actcccaacgaagacaagatctgctttttg |
| | | cttgtactgggtctctctggttagaccaga |
| | | tctgagcctgggagctctctggctaactag |
| | | ggaacccactgcttaagcctcaataaagct |
| | | tgccttgagtgcttcaagtagtgtgtgccc |
| | | gtctgttgtgtgactctggtaactagagat |
| | | ccctcagacccttttagtcagtgtggaaaa |
| | | tctctagcagtagtagttcatgtcatctta |
| | | ttattcagtatttataacttgcaaagaaat |
| | | gaatatcagagagtgagaggaacttgttta |
| | | ttgcagcttataatggttacaaataaagca |
| | | atagcatcacaaatttcacaaataaagcat |
| | | ttttttcactgcattctagttgtggtttgt |
| | | ccaaactcatcaatgtatcttatcatgtct |
| | | ggctctagctatcccgcccctaactccgcc |
| | | catcccgcccctaactccgcccagttccgc |
| | | ccattctccgccccatggctgactaatttt |
| | | ttttatttatgcagaggccgaggccgcctc |
| | | ggcctctgagctattccagaagtagtgagg |
| | | aggcttttttggaggcctagggacgtaccc |
| | | aattcgccctatagtgagtcgtattacgcg |
| | | cgctcactggccgtcgttttacaacgtcgt |
| | | gactgggaaaaccctggcgttacccaactt |
| | | aatcgccttgcagcacatccccctttcgcc |
| | | agctggcgtaatagcgaagaggcccgcacc |
| | | gatcgcccttcccaacagttgcgcagcctg |
| | | aatggcgaatgggacgcgccctgtagcggc |
| | | gcattaagcgcggcgggtgtggtggttacg |
| | | cgcagcgtgaccgctacacttgccagcgcc |
| | | ctagcgcccgctcctttcgctttcttccct |
| | | tcctttctcgccacgttcgccggctttccc |
| | | cgtcaagctctaaatcgggggctcccttta |
| | | gggttccgatttagtgctttacggcacctc |
| | | gaccccaaaaaacttgattagggtgatggt |
| | | tcacgtagtgggccatcgccctgatagacg |
| | | gtttttcgccctttgacgttggagtccacg |
| | | ttctttaatagtggactcttgttccaaact |
| | | ggaacaacactcaaccctatctcggtctat |
| | | tcttttgatttataagggattttgccgatt |
| | | tcggcctattggttaaaaaatgagctgatt |
| | | taacaaaaatttaacgcgaattttaacaaa |
| | | atattaacgcttacaatttaggtggcactt |
| | | ttcggggaaatgtgcgcggaacccctattt |
| | | gtttatttttctaaatacattcaaatatgt |
| | | atccgctcatgagacaataaccctgataaa |
| | | tgcttcaataatattgaaaaaggaagagta |
| | | tgagtattcaacatttccgtgtcgccctta |
| | | ttcccttttttgcggcattttgccttcctg |
| | | tttttgctcacccagaaacgctggtgaaag |
| | | taaaagatgctgaagatcagttgggtgcac |
| | | gagtgggttacatcgaactggatctcaaca |
| | | gcggtaagatccttgagagttttcgccccg |
| | | aagaacgttttccaatgatgagcactttta |
| | | aagttctgctatgtggcgcggtattatccc |
| | | gtattgacgccgggcaagagcaactcggtc |
| | | gccgcatacactattctcagaatgacttgg |
| | | ttgagtactcaccagtcacagaaaagcatc |
| | | ttacggatggcatgacagtaagagaattat |
| | | gcagtgctgccataaccatgagtgataaca |
| | | ctgcggccaacttacttctgacaacgatcg |
| | | gaggaccgaaggagctaaccgcttttttgc |
| | | acaacatgggggatcatgtaactcgccttg |
| | | atcgttgggaaccggagctgaatgaagcca |
| | | taccaaacgacgagcgtgacaccacgatgc |
| | | ctgtagcaatggcaacaacgttgcgcaaac |
| | | tattaactggcgaactacttactctagctt |
| | | cccggcaacaattaatagactggatggagg |
| | | cggataaagttgcaggaccacttctgcgct |
| | | cggcccttccggctggctggtttattgctg |
| | | ataaatctggagccggtgagcgtgggtctc |
| | | gcggtatcattgcagcactggggccagatg |
| | | gtaagccctcccgtatcgtagttatctaca |
| | | cgacggggagtcaggcaactatggatgaac |
| | | gaaatagacagatcgctgagataggtgcct |
| | | cactgattaagcattggtaactgtcagacc |
| | | aagtttactcatatatactttagattgatt |
| | | taaaacttcatttttaatttaaaaggatct |
| | | aggtgaagatcctttttgataatctcatga |
| | | ccaaaatcccttaacgtgagttttcgttcc |
| | | actgagcgtcagaccccgtagaaaagatca |
| | | aaggatcttcttgagatcctttttttctgc |
| | | gcgtaatctgctgcttgcaaacaaaaaaac |
| | | caccgctaccagcggtggtttgtttgccgg |
| | | atcaagagctaccaactctttttccgaagg |
| | | taactggcttcagcagagcgcagataccaa |
| | | atactgttcttctagtgtagccgtagttag |
| | | gccaccacttcaagaactctgtagcaccgc |
| | | ctacatacctcgctctgctaatcctgttac |
| | | cagtggctgctgccagtggcgataagtcgt |
| | | gtcttaccgggttggactcaagacgatagt |
| | | taccggataaggcgcagcggtcgggctgaa |
| | | cggggggttcgtgcacacagcccagcttgg |
| | | agcgaacgacctacaccgaactgagatacc |
| | | tacagcgtgagctatgagaaagcgccacgc |
| | | ttcccgaagagagaaaggcggacaggtatc |
| | | cggtaagcggcagggtcggaacaggagagc |
| | | gcacgagggagcttccagggggaaacgcct |
| | | ggtatctttatagtcctgtcgggtttcgcc |
| | | acctctgacttgagcgtcgatttttgtgat |
| | | gctcgtcaggggggcggagcctatggaaaa |
| | | acgccagcaacgcggcctttttacggttcc |
| | | tggccttttgctggccttttgctcacatgt |
| | | tctttcctgcgttatcccctgattctgtgg |
| | | ataaccgtattaccgcctttgagtgagctg |
| | | ataccgctcgccgcagccgaacgaccgagc |
| | | gcagcgagtcagtgagcgaggaagcggaag |
| | | agcgcccaatacgcaaaccgcctctccccg |
| | | cgcgttggccgattcattaatgcagctggc |
| | | acgacaggtttcccgactggaaagcgggca |
| | | gtgagcgcaacgcaattaatgtgagttagc |
| | | tcactcattaggcaccccaggctttacact |
| | | ttatgcttccggctcgtatgttgtgtggaa |
| | | ttgtgagcggataacaatttcacacaggaa |
| | | acagctatgaccatgattacgccaagcgcg |
| | | caattaaccctcactaaagggaacaaaagc |
| | | tggagctgcaagctt |
| 96 | Nucleic Acid | aatgtagtcttatgcaatactcttgtagtc |
| | Sequence of HG- | ttgcaacatggtaacgatgagttagcaaca |
| | CAR-TG102- | tgccttacaaggagagaaaaagcaccgtgc |
| | EF1_CAR3a_5aa | atgccgattggtggaagtaaggtggtacga |
| | linker-Plasmid | tcgtgccttattaggaaggcaacagacggg |
| | | tctgacatggattggacgaaccactgaatt |
| | | gccgcattgcagagatattgtatttaagtg |
| | | cctagctcgatacataaacgggtctctctg |
| | | gttagaccagatctgagcctgggagctctc |
| | | tggctaactagggaacccactgcttaagcc |
| | | tcaataaagcttgccttgagtgcttcaagt |
| | | agtgtgtgcccgtctgttgtgtgactctgg |
| | | taactagagatccctcagacccttttagtc |
| | | agtgtggaaaatctctagcagtggcgcccg |
| | | aacagggacttgaaagcgaaagggaaacca |
| | | gaggagctctctcgacgcaggactcggctt |
| | | gctgaagcgcgcacggcaagaggcgagggg |
| | | cggcgactggtgagtacgccaaaaattttg |
| | | actagcggaggctagaaggagagagatggg |
| | | tgcgagagcgtcagtattaagcgggggaga |
| | | attagatcgcgatgggaaaaaattcggtta |
| | | aggccagggggaaagaaaaaatataaatta |
| | | aaacatatagtatgggcaagcagggagcta |
| | | gaacgattcgcagttaatcctggcctgtta |
| | | gaaacatcagaaggctgtagacaaatactg |
| | | ggacagctacaaccatcccttcagacagga |
| | | tcagaagaacttagatcattatataataca |
| | | gtagcaaccctctattgtgtgcatcaaagg |
| | | atagagataaaagacaccaaggaagcttta |
| | | gacaagatagaggaagagcaaaacaaaagt |
| | | aagaccaccgcacagcaagcggccgctgat |
| | | cttcagacctggaggaggagatatgaggga |
| | | caattggagaagtgaattatataaatataa |
| | | agtagtaaaaattgaaccattaggagtagc |
| | | acccaccaaggcaaagagaagagtggtgca |
| | | gagagaaaaaagagcagtgggaataggagc |
| | | tttgttccttgggttcttgggagcagcagg |
| | | aagcactatgggcgcagcgtcaatgacgct |
| | | gacggtacaggccagacaattattgtctgg |
| | | tatagtgcagcagcagaacaatttgctgag |
| | | ggctattgaggcgcaacagcatctgttgca |
| | | actcacagtctggggcatcaagcagctcca |
| | | ggcaagaatcctggctgtggaaagatacct |
| | | aaaggatcaacagctcctggggatttgggg |
| | | ttgctctggaaaactcatttgcaccactgc |
| | | tgtgccttggaatgctagttggagtaataa |
| | | atctctggaacagatttggaatcacacgac |
| | | ctggatggagtgggacagagaaattaacaa |
| | | ttacacaagcttaatacactccttaattga |
| | | agaatcgcaaaaccagcaagaaaagaatga |
| | | acaagaattattggaattagataaatgggc |
| | | aagtttgtggaattggtttaacataacaaa |
| | | ttggctgtggtatataaaattattcataat |
| | | gatagtaggaggcttggtaggtttaagaat |
| | | agtttttgctgtactttctatagtgaatag |
| | | agttaggcagggatattcaccattatcgtt |
| | | tcagacccacctcccaaccccgaggggacc |
| | | cgacaggcccgaaggaatagaagaagaagg |
| | | tggagagagagacagagacagatccattcg |
| | | attagtgaacggatctcgacggtatcgcta |
| | | gcttttaaaagaaaaggggggattgggggg |
| | | tacagtgcaggggaaagaatagtagacata |
| | | atagcaacagacatacaaactaaagaatta |
| | | caaaaacaaattacaaaaattcaaaatttt |
| | | actagtgattatcggatcaactttgtatag |
| | | aaaagttgggctccggtgcccgtcagtggg |
| | | cagagcgcacatcgcccacagtccccgaga |
| | | agttggggggaggggtcggcaattgaaccg |
| | | gtgcctagagaaggtggcgcggggtaaact |
| | | gggaaagtgatgtcgtgtactggctccgcc |
| | | tttttcccgaggggggggagaaccgtatat |
| | | aagtgcagtagtcgccgtgaacgttctttt |
| | | tcgcaacgggtttgccgccagaacacaggt |
| | | aagtgccgtgtgtggttcccgcgggcctgg |
| | | cctctttacgggttatggcccttgcgtgcc |
| | | ttgaattacttccacctggctgcagtacgt |
| | | gattcttgatcccgagcttcgggttggaag |
| | | tggggggagagttcgaggccttgcgcttaa |
| | | ggagccccttcgcctcgtgcttgagttgag |
| | | gcctggcctgggcgctggggccgccgcgtg |
| | | cgaatctggtggcaccttcgcgcctgtctc |
| | | gctgctttcgataagtctctagccatttaa |
| | | aatttttgatgacctgctgcgacgcttttt |
| | | ttctggcaagatagtcttgtaaatgcgggc |
| | | caagatctgcacactggtatttcggttttt |
| | | ggggccgcgggcggcgacggggcccgtgcg |
| | | tcccagcgcacatgttcggcgaggcggggc |
| | | ctgcgagcgcggccaccgagaatcggacgg |
| | | gggtagtctcaagctggccggcctgctctg |
| | | gtgcctggtctcgcgccgccgtgtatcgcc |
| | | ccgccctgggcggcaaggctggcccggtcg |
| | | gcaccagttgcgtgagcggaaagatggccg |
| | | cttcccggccctgctgcagggagctcaaaa |
| | | tggaggacgcggcgctcgggagagcggggg |
| | | gtgagtcacccacacaaaggaaaagggcct |
| | | ttccgtcctcagccgtcgcttcatgtgact |
| | | ccacggagtaccgggcgccgtccaggcacc |
| | | tcgattagttctcgagcttttggagtacgt |
| | | cgtctttaggttggggggaggggttttatg |
| | | cgatggagtttccccacactgagtgggtgg |
| | | agactgaagttaggccagcttggcacttga |
| | | tgtaattctccttggaatttgccctttttg |
| | | agtttggatcttggttcattctcaagcctc |
| | | agacagtggttcaaagtttttttcttccat |
| | | ttcaggtgtcgtgacaagtttgtacaaaaa |
| | | agcaggctgccaccatggccttaccagtga |
| | | ccgccttgctcctgccgctggccttgctgc |
| | | tccacgccgccaggccgggatccgacatcc |
| | | agatgacccagagcccctcctctttatccg |
| | | cctctgtgggcgacagagtgaccatcactt |
| | | gtcgtgccagccaagaaatctccggctatc |
| | | tgtcttggctgcagcagaagcccggtaagg |
| | | ctatcaagagactcatctacgccgccagca |
| | | ctttacagagcggagtgcctagcagattta |
| | | gcggctctcgtagcggcagcgattatactt |
| | | taaccatctcctctttacagcccgaagact |
| | | ttgctacctactactgtttacagtacgcca |
| | | gctaccccttcaccttcggtcaaggtacca |
| | | agctggagatcaaaggtggcggcggctctc |
| | | aagttaccctcaaggagagcggccccactt |
| | | tagtgaagcctacccagactttaactttaa |
| | | cttgtaccttcagcggcttctctttaagca |
| | | cctccacaatgggcgtgggctggatcagac |
| | | agcctcccggcaaggctctggagtggctgg |
| | | cccacatcctctggaacgacagcaagaggt |
| | | acaacccctctttaaagtctcgtctgacca |
| | | tcaccaaggacacctccaagaagcaagttg |
| | | tgctgaccatgaccaatatggaccccgtgg |
| | | acaccgccacctattactgcgctcgtatcg |
| | | tgtactactccacctacgtgggctacttcg |
| | | acgtctggggacaaggtaccaccgtgaccg |
| | | tgagctctggatccttcgtgccggtcttcc |
| | | tgccagcgaagcccaccacgacgccagcgc |
| | | cgcgaccaccaacaccggcgcccaccatcg |
| | | cgtcgcagcccctgtccctgcgcccagagg |
| | | cgtgccggccagcggggggggcgcagtgca |
| | | cacgagggggctggacttcgcctgtgatat |
| | | ctacatctgggcgcccttggccgggacttg |
| | | tggggtccttctcctgtcactggttatcac |
| | | cctttactgcaaccacaggaacaggagtaa |
| | | gaggagcaggctcctgcacagtgactacat |
| | | gaacatgactccccgccgccccgggcccac |
| | | ccgcaagcattaccagccctatgccccacc |
| | | acgcgacttcgcagcctatcgctccaaacg |
| | | gggcagaaagaaactcctgtatatattcaa |
| | | acaaccatttatgagaccagtacaaactac |
| | | tcaagaggaagatggctgtagctgccgatt |
| | | tccagaagaagaagaaggaggatgtgaact |
| | | gagagtgaagttcagcaggagcgcagacgc |
| | | ccccgcgtaccagcagggccagaaccagct |
| | | ctataacgagctcaatctaggacgaagaga |
| | | ggagtacgatgttttggacaagagacgtgg |
| | | ccgggaccctgagatggggggaaagccgag |
| | | aaggaagaaccctcaggaaggcctgtacaa |
| | | tgaactgcagaaagataagatggcggaggc |
| | | ctacagtgagattgggatgaaaggcgagcg |
| | | ccggaggggcaaggggcacgatggccttta |
| | | ccagggtctcagtacagccaccaaggacac |
| | | ctacgacgcccttcacatgcaggccctgcc |
| | | ccctcgctgaacccagctttcttgtacaaa |
| | | gtggtgataatcgaattccgataatcaacc |
| | | tctggattacaaaatttgtgaaagattgac |
| | | tggtattcttaactatgttgctccttttac |
| | | gctatgtggatacgctgctttaatgccttt |
| | | gtatcatgctattgcttcccgtatggcttt |
| | | cattttctcctccttgtataaatcctggtt |
| | | gctgtctctttatgaggagttgtggcccgt |
| | | tgtcaggcaacgtggcgtggtgtgcactgt |
| | | gtttgctgacgcaacccccactggttgggg |
| | | cattgccaccacctgtcagctcctttccgg |
| | | gactttcgctttccccctccctattgccac |
| | | ggcggaactcatcgccgcctgccttgcccg |
| | | ctgctggacaggggctcggctgttgggcac |
| | | tgacaattccgtggtgttgtcggggaagct |
| | | gacgtcctttccatggctgctcgcctgtgt |
| | | tgccacctggattctgcgcgggacgtcctt |
| | | ctgctacgtcccttcggccctcaatccagc |
| | | ggaccttccttcccgcggcctgctgccggc |
| | | tctgcggcctcttccgcgtcttcgccttcg |
| | | ccctcagacgagtcggatctccctttgggc |
| | | cgcctccccgcatcgggaattcccgcggtt |
| | | cgctttaagaccaatgacttacaaggcagc |
| | | tgtagatcttagccactttttaaaagaaaa |
| | | ggggggactggaagggctaattcactccca |
| | | acgaagacaagatctgctttttgcttgtac |
| | | tgggtctctctggttagaccagatctgagc |
| | | ctgggagctctctggctaactagggaaccc |
| | | actgcttaagcctcaataaagcttgccttg |
| | | agtgcttcaagtagtgtgtgcccgtctgtt |
| | | gtgtgactctggtaactagagatccctcag |
| | | acccttttagtcagtgtggaaaatctctag |
| | | cagtagtagttcatgtcatcttattattca |
| | | gtatttataacttgcaaagaaatgaatatc |
| | | agagagtgagaggaacttgtttattgcagc |
| | | ttataatggttacaaataaagcaatagcat |
| | | cacaaatttcacaaataaagcatttttttc |
| | | actgcattctagttgtggtttgtccaaact |
| | | catcaatgtatcttatcatgtctggctcta |
| | | gctatcccgcccctaactccgcccatcccg |
| | | cccctaactccgcccagttccgcccattct |
| | | ccgccccatggctgactaattttttttatt |
| | | tatgcagaggccgaggccgcctcggcctct |
| | | gagctattccagaagtagtgaggaggcttt |
| | | tttggaggcctagggacgtacccaattcgc |
| | | cctatagtgagtgtattacgcgcgctcact |
| | | ggccgtcgttttacaacgtcgtgactggga |
| | | aaaccctggcgttacccaacttaatcgcct |
| | | tgcagcacatccccctttcgccagctggcg |
| | | taatagcgaagaggcccgcaccgatcgccc |
| | | ttcccaacagttgcgcagcctgaatggcga |
| | | atgggacgcgccctgtagcggcgcattaag |
| | | cgcggcgggtgtggtggttacgcgcagcgt |
| | | gaccgctacacttgccagcgccctagcgcc |
| | | cgctcctttcgctttcttcccttcctttct |
| | | cgccacgttcgccggctttccccgtcaagc |
| | | tctaaatcgggggctccctttagggttccg |
| | | atttagtgctttacggcacctcgaccccaa |
| | | aaaacttgattagggtgatggttcacgtag |
| | | tgggccatcgccctgatagacggtttttcg |
| | | ccctttgacgttggagtccacgttctttaa |
| | | tagtggactcttgttccaaactggaacaac |
| | | actcaaccctatctcggtctattcttttga |
| | | tttataagggattttgccgatttcggccta |
| | | ttggttaaaaaatgagctgatttaacaaaa |
| | | atttaacgcgaattttaacaaaatattaac |
| | | gcttacaatttaggtggcacttttcgggga |
| | | aatgtgcgcggaacccctatttgtttattt |
| | | ttctaaatacattcaaatatgtatccgctc |
| | | atgagacaataaccctgataaatgcttcaa |
| | | taatattgaaaaaggaagagtatgagtatt |
| | | caacatttccgtgtcgcccttattcccttt |
| | | tttgcggcattttgccttcctgtttttgct |
| | | cacccagaaacgctggtgaaagtaaaagat |
| | | gctgaagatcagttgggtgcacgagtgggt |
| | | tacatcgaactggatctcaacagcggtaag |
| | | atccttgagagttttcgccccgaagaacgt |
| | | tttccaatgatgagcacttttaaagttctg |
| | | ctatgtggcgcggtattatcccgtattgac |
| | | gccgggcaagagcaactcggtcgccgcata |
| | | cactattctcagaatgacttggttgagtac |
| | | tcaccagtcacagaaaagcatcttacggat |
| | | ggcatgacagtaagagaattatgcagtgct |
| | | gccataaccatgagtgataacactgggcca |
| | | acttacttctgacaacgatcggaggaccga |
| | | aggagctaaccgcttttttgcacaacatgg |
| | | gggatcatgtaactcgccttgatcgttggg |
| | | aaccggagctgaatgaagccataccaaacg |
| | | acgagcgtgacaccacgatgcctgtagcaa |
| | | tggcaacaacgttgcgcaaactattaactg |
| | | gcgaactacttactctagcttcccggcaac |
| | | aattaatagactggatggaggcggataaag |
| | | ttgcaggaccacttctgcgctcggcccttc |
| | | cggctggctggtttattgctgataaatctg |
| | | gagccggtgagcgtgggtctcgcggtatca |
| | | ttgcagcactggggccagatggtaagccct |
| | | cccgtatcgtagttatctacacgacgggga |
| | | gtcaggcaactatggatgaacgaaatagac |
| | | agatcgctgagataggtgcctcactgatta |
| | | agcattggtaactgtcagaccaagtttact |
| | | catatatactttagattgatttaaaacttc |
| | | atttttaatttaaaaggatctaggtgaaga |
| | | tcctttttgataatctcatgaccaaaatcc |
| | | cttaacgtgagttttcgttccactgagcgt |
| | | cagaccccgtagaaaagatcaaaggatctt |
| | | cttgagatcctttttttctgcgcgtaatct |
| | | gctgcttgcaaacaaaaaaaccaccgctac |
| | | cagcggtggtttgtttgccggatcaagagc |
| | | taccaactctttttccgaaggtaactggct |
| | | tcagcagagcgcagataccaaatactgttc |
| | | ttctagtgtagccgtagttaggccaccact |
| | | tcaagaactctgtagcaccgcctacatacc |
| | | tcgctctgctaatcctgttaccagtggctg |
| | | ctgccagtggcgataagtcgtgtcttaccg |
| | | ggttggactcaagacgatagttaccggata |
| | | aggcgcagcggtcgggctgaacggggggtt |
| | | cgtgcacacagcccagcttggagcgaacga |
| | | cctacaccgaactgagatacctacagcgtg |
| | | agctatgagaaagcgccacgcttcccgaag |
| | | agagaaaggcggacaggtatccggtaagcg |
| | | gcagggtcggaacaggagagcgcacgaggg |
| | | agcttccagggggaaacgcctggtatcttt |
| | | atagtcctgtcgggtttcgccacctctgac |
| | | ttgagcgtcgatttttgtgatgctcgtcag |
| | | gggggcggagcctatggaaaaacgccagca |
| | | acgcggcctttttacggttcctggcctttt |
| | | gctggccttttgctcacatgttctttcctg |
| | | cgttatcccctgattctgtggataaccgta |
| | | ttaccgcctttgagtgagctgataccgctc |
| | | gccgcagccgaacgaccgagcgcagcgagt |
| | | cagtgagcgaggaagcggaagagcgcccaa |
| | | tacgcaaaccgcctctccccgcgcgttggc |
| | | cgattcattaatgcagctggcacgacaggt |
| | | ttcccgactggaaagcgggcagtgagcgca |
| | | acgcaattaatgtgagttagctcactcatt |
| | | aggcaccccaggctttacactttatgcttc |
| | | cggctcgtatgttgtgtggaattgtgagcg |
| | | gataacaatttcacacaggaaacagctatg |
| | | accatgattacgccaagcgcgcaattaacc |
| | | ctcactaaagggaacaaaagctggagctgc |
| | | aagctt |
| 97 | Nucleic Acid | aatgtagtcttatgcaatactcttgtagtc |
| | Sequence of HG- | ttgcaacatggtaacgatgagttagcaaca |
| | CAR-TG103- | tgccttacaaggagagaaaaagcaccgtgc |
| | EF1_CAR3a_10aa | atgccgattggtggaagtaaggtggtacga |
| | linker plasmid | tcgtgccttattaggaaggcaacagacggg |
| | | tctgacatggattggacgaaccactgaatt |
| | | gccgcattgcagagatattgtatttaagtg |
| | | cctagctcgatacataaacgggtctctctg |
| | | gttagaccagatctgagcctgggagctctc |
| | | tggctaactagggaacccactgcttaagcc |
| | | tcaataaagcttgccttgagtgcttcaagt |
| | | agtgtgtgcccgtctgttgtgtgactctgg |
| | | taactagagatccctcagacccttttagtc |
| | | agtgtggaaaatctctagcagtggcgcccg |
| | | aacagggacttgaaagcgaaagggaaacca |
| | | gaggagctctctcgacgcaggactcggctt |
| | | gctgaagcgcgcacggcaagaggcgagggg |
| | | cggcgactggtgagtacgccaaaaattttg |
| | | actagcggaggctagaaggagagagatggg |
| | | tgcgagagcgtcagtattaagcgggggaga |
| | | attagatcgcgatgggaaaaaattcggtta |
| | | aggccagggggaaagaaaaaatataaatta |
| | | aaacatatagtatgggcaagcagggagcta |
| | | gaacgattcgcagttaatcctggcctgtta |
| | | gaaacatcagaaggctgtagacaaatactg |
| | | ggacagctacaaccatcccttcagacagga |
| | | tcagaagaacttagatcattatataataca |
| | | gtagcaaccctctattgtgtgcatcaaagg |
| | | atagagataaaagacaccaaggaagcttta |
| | | gacaagatagaggaagagcaaaacaaaagt |
| | | aagaccaccgcacagcaagcggccgctgat |
| | | cttcagacctggaggaggagatatgaggga |
| | | caattggagaagtgaattatataaatataa |
| | | agtagtaaaaattgaaccattaggagtagc |
| | | acccaccaaggcaaagagaagagtggtgca |
| | | gagagaaaaaagagcagtgggaataggagc |
| | | tttgttccttgggttcttgggagcagcagg |
| | | aagcactatgggcgcagcgtcaatgacgct |
| | | gacggtacaggccagacaattattgtctgg |
| | | tatagtgcagcagcagaacaatttgctgag |
| | | ggctattgaggcgcaacagcatctgttgca |
| | | actcacagtctggggcatcaagcagctcca |
| | | ggcaagaatcctggctgtggaaagatacct |
| | | aaaggatcaacagctcctggggatttgggg |
| | | ttgctctggaaaactcatttgcaccactgc |
| | | tgtgccttggaatgctagttggagtaataa |
| | | atctctggaacagatttggaatcacacgac |
| | | ctggatggagtgggacagagaaattaacaa |
| | | ttacacaagcttaatacactccttaattga |
| | | agaatcgcaaaaccagcaagaaaagaatga |
| | | acaagaattattggaattagataaatgggc |
| | | aagtttgtggaattggtttaacataacaaa |
| | | ttggctgtggtatataaaattattcataat |
| | | gatagtaggaggcttggtaggtttaagaat |
| | | agtttttgctgtactttctatagtgaatag |
| | | agttaggcagggatattcaccattatcgtt |
| | | tcagacccacctcccaaccccgaggggacc |
| | | cgacaggcccgaaggaatagaagaagaagg |
| | | tggagagagagacagagacagatccattcg |
| | | attagtgaacggatctcgacggtatcgcta |
| | | gcttttaaaagaaaaggggggattgggggg |
| | | tacagtgcaggggaaagaatagtagacata |
| | | atagcaacagacatacaaactaaagaatta |
| | | caaaaacaaattacaaaaattcaaaatttt |
| | | actagtgattatcggatcaactttgtatag |
| | | aaaagttgggctccggtgcccgtcagtggg |
| | | cagagcgcacatcgcccacagtccccgaga |
| | | agttggggggaggggtcggcaattgaaccg |
| | | gtgcctagagaaggtggcgcggggtaaact |
| | | gggaaagtgatgtcgtgtactggctccgcc |
| | | tttttcccgaggggggggagaaccgtatat |
| | | aagtgcagtagtcgccgtgaacgttctttt |
| | | tcgcaacgggtttgccgccagaacacaggt |
| | | aagtgccgtgtgtggttcccgcgggcctgg |
| | | cctctttacgggttatggcccttgcgtgcc |
| | | ttgaattacttccacctggctgcagtacgt |
| | | gattcttgatcccgagcttcgggttggaag |
| | | tggggggagagttcgaggccttgcgcttaa |
| | | ggagccccttcgcctcgtgcttgagttgag |
| | | gcctggcctgggcgctggggccgccgcgtg |
| | | cgaatctggtggcaccttcgcgcctgtctc |
| | | gctgctttcgataagtctctagccatttaa |
| | | aatttttgatgacctgctgcgacgcttttt |
| | | ttctggcaagatagtcttgtaaatgcgggc |
| | | caagatctgcacactggtatttcggttttt |
| | | ggggccgcgggcggcgacggggcccgtgcg |
| | | tcccagcgcacatgttcggcgaggcggggc |
| | | ctgcgagcgcggccaccgagaatcggacgg |
| | | gggtagtctcaagctggccggcctgctctg |
| | | gtgcctggtctcgcgccgccgtgtatcgcc |
| | | ccgccctgggcggcaaggctggcccggtcg |
| | | gcaccagttgcgtgagcggaaagatggccg |
| | | cttcccggccctgctgcagggagctcaaaa |
| | | tggaggacgcggcgctcgggagagcgggcg |
| | | ggtgagtcacccacacaaaggaaaagggcc |
| | | tttccgtcctcagccgtcgcttcatgtgac |
| | | tccacggagtaccgggcgccgtccaggcac |
| | | ctcgattagttctcgagcttttggagtacg |
| | | tcgtctttaggttggggggaggggttttat |
| | | gcgatggagtttccccacactgagtgggtg |
| | | gagactgaagttaggccagcttggcacttg |
| | | atgtaattctccttggaatttgcccttttt |
| | | gagtttggatcttggttcattctcaagcct |
| | | cagacagtggttcaaagtttttttcttcca |
| | | tttcaggtgtcgtgacaagtttgtacaaaa |
| | | aagcaggctgccaccatggccttaccagtg |
| | | accgccttgctcctgccgctggccttgctg |
| | | ctccacgccgccaggccgggatccgacatc |
| | | cagatgacccagagcccctcctctttatcc |
| | | gcctctgtgggcgacagagtgaccatcact |
| | | tgtcgtgccagccaagaaatctccggctat |
| | | ctgtcttggctgcagcagaagcccggtaag |
| | | gctatcaagagactcatctacgccgccagc |
| | | actttacagagcggagtgcctagcagattt |
| | | agcggctctcgtagcggcagcgattatact |
| | | ttaaccatctcctctttacagcccgaagac |
| | | tttgctacctactactgtttacagtacgcc |
| | | agctaccccttcaccttcggtcaaggtacc |
| | | aagctggagatcaaaggtggcggcggctct |
| | | ggcggcggtggctctcaagttaccctcaag |
| | | gagagcggccccactttagtgaagcctacc |
| | | cagactttaactttaacttgtaccttcagc |
| | | ggcttctctttaagcacctccacaatgggc |
| | | gtgggctggatcagacagcctcccggcaag |
| | | gctctggagtggctggcccacatcctctgg |
| | | aacgacagcaagaggtacaacccctcttta |
| | | aagtctcgtctgaccatcaccaaggacacc |
| | | tccaagaagcaagttgtgctgaccatgacc |
| | | aatatggaccccgtggacaccgccacctat |
| | | tactgcgctcgtatcgtgtactactccacc |
| | | tacgtgggctacttcgacgtctggggacaa |
| | | ggtaccaccgtgaccgtgagctctggatcc |
| | | ttcgtgccggtcttcctgccagcgaagccc |
| | | accacgacgccagcgccgcgaccaccaaca |
| | | ccggcgcccaccatcgcgtcgcagcccctg |
| | | tccctgcgcccagaggcgtgccggccagcg |
| | | gcggggggcgcagtgcacacgagggggctg |
| | | gacttcgcctgtgatatctacatctgggcg |
| | | cccttggccgggacttgtggggtccttctc |
| | | ctgtcactggttatcaccctttactgcaac |
| | | cacaggaacaggagtaagaggagcaggctc |
| | | ctgcacagtgactacatgaacatgactccc |
| | | cgccgccccgggcccacccgcaagcattac |
| | | cagccctatgccccaccacgcgacttcgca |
| | | gcctatcgctccaaacggggcagaaagaaa |
| | | ctcctgtatatattcaaacaaccatttatg |
| | | agaccagtacaaactactcaagaggaagat |
| | | ggctgtagctgccgatttccagaagaagaa |
| | | gaaggaggatgtgaactgagagtgaagttc |
| | | agcaggagcgcagacgcccccgcgtaccag |
| | | cagggccagaaccagctctataacgagctc |
| | | aatctaggacgaagagaggagtacgatgtt |
| | | ttggacaagagacgtggccgggaccctgag |
| | | atggggggaaagccgagaaggaagaaccct |
| | | caggaaggcctgtacaatgaactgcagaaa |
| | | gataagatggcggaggcctacagtgagatt |
| | | gggatgaaaggcgagcgccggaggggcaag |
| | | gggcacgatggcctttaccagggtctcagt |
| | | acagccaccaaggacacctacgacgccctt |
| | | cacatgcaggccctgccccctcgctgaacc |
| | | cagctttcttgtacaaagtggtgataatcg |
| | | aattccgataatcaacctctggattacaaa |
| | | atttgtgaaagattgactggtattcttaac |
| | | tatgttgctccttttacgctatgtggatac |
| | | gctgctttaatgcctttgtatcatgctatt |
| | | gcttcccgtatggctttcattttctcctcc |
| | | ttgtataaatcctggttgctgtctctttat |
| | | gaggagttgtggcccgttgtcaggcaacgt |
| | | ggcgtggtgtgcactgtgtttgctgacgca |
| | | acccccactggttggggcattgccaccacc |
| | | tgtcagctcctttccgggactttcgctttc |
| | | cccctccctattgccacggcggaactcatc |
| | | gccgcctgccttgcccgctgctggacaggg |
| | | gctcggctgttgggcactgacaattccgtg |
| | | gtgttgtcggggaagctgacgtcctttcca |
| | | tggctgctcgcctgtgttgccacctggatt |
| | | ctgcgcgggacgtccttctgctacgtccct |
| | | tcggccctcaatccagcggaccttccttcc |
| | | cgcggcctgctgccggctctgcggcctctt |
| | | ccgcgtcttcgccttcgccctcagacgagt |
| | | cggatctccctttgggccgcctccccgcat |
| | | cgggaattcccgcggttcgctttaagacca |
| | | atgacttacaaggcagctgtagatcttagc |
| | | cactttttaaaagaaaaggggggactggaa |
| | | gggctaattcactcccaacgaagacaagat |
| | | ctgctttttgcttgtactgggtctctctgg |
| | | ttagaccagatctgagcctgggagctctct |
| | | ggctaactagggaacccactgcttaagcct |
| | | caataaagcttgccttgagtgcttcaagta |
| | | gtgtgtgcccgtctgttgtgtgactctggt |
| | | aactagagatccctcagacccttttagtca |
| | | gtgtggaaaatctctagcagtagtagttca |
| | | tgtcatcttattattcagtatttataactt |
| | | gcaaagaaatgaatatcagagagtgagagg |
| | | aacttgtttattgcagcttataatggttac |
| | | aaataaagcaatagcatcacaaatttcaca |
| | | aataaagcatttttttcactgcattctagt |
| | | tgtggtttgtccaaactcatcaatgtatct |
| | | tatcatgtctggctctagctatcccgcccc |
| | | taactccgcccatcccgcccctaactccgc |
| | | ccagttccgcccattctccgccccatggct |
| | | gactaattttttttatttatgcagaggccg |
| | | aggccgcctcggcctctgagctattccaga |
| | | agtagtgaggaggcttttttggaggcctag |
| | | ggacgtacccaattcgccctatagtgagtc |
| | | gtattacgcgcgctcactggccgtcgtttt |
| | | acaacgtcgtgactgggaaaaccctggcgt |
| | | tacccaacttaatcgccttgcagcacatcc |
| | | ccctttcgccagctggcgtaatagcgaaga |
| | | ggcccgcaccgatcgcccttcccaacagtt |
| | | gcgcagcctgaatggcgaatgggacgcgcc |
| | | ctgtagcggcgcattaagcgcggcgggtgt |
| | | ggtggttacgcgcagcgtgaccgctacact |
| | | tgccagcgccctagcgcccgctcctttcgc |
| | | tttcttcccttcctttctcgccacgttcgc |
| | | cggctttccccgtcaagctctaaatcgggg |
| | | gctccctttagggttccgatttagtgcttt |
| | | acggcacctcgaccccaaaaaacttgatta |
| | | gggtgatggttcacgtagtgggccatcgcc |
| | | ctgatagacggtttttcgccctttgacgtt |
| | | ggagtccacgttctttaatagtggactctt |
| | | gttccaaactggaacaacactcaaccctat |
| | | ctcggtctattcttttgatttataagggat |
| | | tttgccgatttcggcctattggttaaaaaa |
| | | tgagctgatttaacaaaaatttaacgcgaa |
| | | ttttaacaaaatattaacgcttacaattta |
| | | ggtggcacttttcggggaaatgtgcgcgga |
| | | acccctatttgtttatttttctaaatacat |
| | | tcaaatatgtatccgctcatgagacaataa |
| | | ccctgataaatgcttcaataatattgaaaa |
| | | aggaagagtatgagtattcaacatttccgt |
| | | gtcgcccttattcccttttttgcggcattt |
| | | tgccttcctgtttttgctcacccagaaacg |
| | | ctggtgaaagtaaaagatgctgaagatcag |
| | | ttgggtgcacgagtgggttacatcgaactg |
| | | gatctcaacagcggtaagatccttgagagt |
| | | tttcgccccgaagaacgttttccaatgatg |
| | | agcacttttaaagttctgctatgtggcgcg |
| | | gtattatcccgtattgacgccgggcaagag |
| | | caactcggtcgccgcatacactattctcag |
| | | aatgacttggttgagtactcaccagtcaca |
| | | gaaaagcatcttacggatggcatgacagta |
| | | agagaattatgcagtgctgccataaccatg |
| | | agtgataacactgcggccaacttacttctg |
| | | acaacgatcggaggaccgaaggagctaacc |
| | | gcttttttgcacaacatgggggatcatgta |
| | | actcgccttgatcgttgggaaccggagctg |
| | | aatgaagccataccaaacgacgagcgtgac |
| | | accacgatgcctgtagcaatggcaacaacg |
| | | ttgcgcaaactattaactggcgaactactt |
| | | actctagcttcccggcaacaattaatagac |
| | | tggatggaggcggataaagttgcaggacca |
| | | cttctgcgctcggcccttccggctggctgg |
| | | tttattgctgataaatctggagccggtgag |
| | | cgtgggtctcgcggtatcattgcagcactg |
| | | gggccagatggtaagccctcccgtatcgta |
| | | gttatctacacgacggggagtcaggcaact |
| | | atggatgaacgaaatagacagatcgctgag |
| | | ataggtgcctcactgattaagcattggtaa |
| | | ctgtcagaccaagtttactcatatatactt |
| | | tagattgatttaaaacttcatttttaattt |
| | | aaaaggatctaggtgaagatcctttttgat |
| | | aatctcatgaccaaaatcccttaacgtgag |
| | | ttttcgttccactgagcgtcagaccccgta |
| | | gaaaagatcaaaggatcttcttgagatcct |
| | | ttttttctgcgcgtaatctgctgcttgcaa |
| | | acaaaaaaaccaccgctaccagcggtggtt |
| | | tgtttgccggatcaagagctaccaactctt |
| | | tttccgaaggtaactggcttcagcagagcg |
| | | cagataccaaatactgttcttctagtgtag |
| | | ccgtagttaggccaccacttcaagaactct |
| | | gtagcaccgcctacatacctcgctctgcta |
| | | atcctgttaccagtggctgctgccagtggc |
| | | gataagtcgtgtcttaccgggttggactca |
| | | agacgatagttaccggataaggcgcagcgg |
| | | tcgggctgaacggggggttcgtgcacacag |
| | | cccagcttggagcgaacgacctacaccgaa |
| | | ctgagatacctacagcgtgagctatgagaa |
| | | agcgccacgcttcccgaagagagaaaggcg |
| | | gacaggtatccggtaagcggcagggtcgga |
| | | acaggagagcgcacgagggagcttccaggg |
| | | ggaaacgcctggtatctttatagtcctgtc |
| | | gggtttcgccacctctgacttgagcgtcga |
| | | tttttgtgatgctcgtcaggggggcggagc |
| | | ctatggaaaaacgccagcaacgcggccttt |
| | | ttacggttcctggccttttgctggcctttt |
| | | gctcacatgttctttcctgcgttatcccct |
| | | gattctgtggataaccgtattaccgccttt |
| | | gagtgagctgataccgctcgccgcagccga |
| | | acgaccgagcgcagcgagtcagtgagcgag |
| | | gaagcggaagagcgcccaatacgcaaaccg |
| | | cctctccccgcgcgttggccgattcattaa |
| | | tgcagctggcacgacaggtttcccgactgg |
| | | aaagcgggcagtgagcgcaacgcaattaat |
| | | gtgagttagctcactcattaggcaccccag |
| | | gctttacactttatgcttccggctcgtatg |
| | | ttgtgtggaattgtgagcggataacaattt |
| | | cacacaggaaacagctatgaccatgattac |
| | | gccaagcgcgcaattaaccctcactaaagg |
| | | gaacaaaagctggagctgcaagctt |
| 98 | Nucleic Acid | aatgtagtcttatgcaatactcttgtagtc |
| | sequence of HG- | ttgcaacatggtaacgatgagttagcaaca |
| | CAR-TG104- | tgccttacaaggagagaaaaagcaccgtgc |
| | EF1_CAR3a_15aa | atgccgattggtggaagtaaggggtacgat |
| | linker-plasmid | cgtgccttattaggaaggcaacagacgggt |
| | | ctgacatggattggacgaaccactgaattg |
| | | ccgcattgcagagatattgtatttaagtgc |
| | | ctagctcgatacataaacgggtctctctgg |
| | | ttagaccagatctgagcctgggagctctct |
| | | ggctaactagggaacccactgcttaagcct |
| | | caataaagcttgccttgagtgcttcaagta |
| | | gtgtgtgcccgtctgttgtgtgactctggt |
| | | aactagagatccctcagacccttttagtca |
| | | gtgtggaaaatctctagcagtggcgcccga |
| | | acagggacttgaaagcgaaagggaaaccag |
| | | aggagctctctcgacgcaggactcggcttg |
| | | ctgaagcgcgcacggcaagaggcgaggggc |
| | | ggcgactggtgagtacgccaaaaattttga |
| | | ctagcggaggctagaaggagagagatgggt |
| | | gcgagagcgtcagtattaagcgggggagaa |
| | | ttagatcgcgatgggaaaaaattcggttaa |
| | | ggccagggggaaagaaaaaatataaattaa |
| | | aacatatagtatgggcaagcagggagctag |
| | | aacgattcgcagttaatcctggcctgttag |
| | | aaacatcagaaggctgtagacaaatactgg |
| | | gacagctacaaccatcccttcagacaggat |
| | | cagaagaacttagatcattatataatacag |
| | | tagcaaccctctattgtgtgcatcaaagga |
| | | tagagataaaagacaccaaggaagctttag |
| | | acaagatagaggaagagcaaaacaaaagta |
| | | agaccaccgcacagcaagcggccgctgatc |
| | | ttcagacctggaggaggagatatgagggac |
| | | aattggagaagtgaattatataaatataaa |
| | | gtagtaaaaattgaaccattaggagtagca |
| | | cccaccaaggcaaagagaagagtggtgcag |
| | | agagaaaaaagagcagtgggaataggagct |
| | | ttgttccttgggttcttgggagcagcagga |
| | | agcactatgggcgcagcgtcaatgacgctg |
| | | acggtacaggccagacaattattgtctggt |
| | | atagtgcagcagcagaacaatttgctgagg |
| | | gctattgaggcgcaacagcatctgttgcaa |
| | | ctcacagtctggggcatcaagcagctccag |
| | | gcaagaatcctggctgtggaaagataccta |
| | | aaggatcaacagctcctggggatttggggt |
| | | tgctctggaaaactcatttgcaccactgct |
| | | gtgccttggaatgctagttggagtaataaa |
| | | tctctggaacagatttggaatcacacgacc |
| | | tggatggagtgggacagagaaattaacaat |
| | | tacacaagcttaatacactccttaattgaa |
| | | gaatcgcaaaaccagcaagaaaagaatgaa |
| | | caagaattattggaattagataaatgggca |
| | | agtttgtggaattggtttaacataacaaat |
| | | tggctgtggtatataaaattattcataatg |
| | | atagtaggaggcttggtaggtttaagaata |
| | | gtttttgctgtactttctatagtgaataga |
| | | gttaggcagggatattcaccattatcgttt |
| | | cagacccacctcccaaccccgaggggaccc |
| | | gacaggcccgaaggaatagaagaagaaggt |
| | | ggagagagagacagagacagatccattcga |
| | | ttagtgaacggatctcgacggtatcgctag |
| | | cttttaaaagaaaaggggggattggggggt |
| | | acagtgcaggggaaagaatagtagacataa |
| | | tagcaacagacatacaaactaaagaattac |
| | | aaaaacaaattacaaaaattcaaaatttta |
| | | ctagtgattatcggatcaactttgtataga |
| | | aaagttgggctccggtgcccgtcagtgggc |
| | | agagcgcacatcgcccacagtccccgagaa |
| | | gttggggggaggggtcggcaattgaaccgg |
| | | tgcctagagaaggtggcgcggggtaaactg |
| | | ggaaagtgatgtcgtgtactggctccgcct |
| | | ttttcccgaggggggggagaaccgtatata |
| | | agtgcagtagtcgccgtgaacgttcttttt |
| | | cgcaacgggtttgccgccagaacacaggta |
| | | agtgccgtgtgtggttcccgcgggcctggc |
| | | ctctttacgggttatggcccttgcgtgcct |
| | | tgaattacttccacctggctgcagtacgtg |
| | | attcttgatcccgagcttcgggttggaagt |
| | | ggggggagagttcgaggccttgcgcttaag |
| | | gagccccttcgcctcgtgcttgagttgagg |
| | | cctggcctgggcgctggggccgccgcgtgc |
| | | gaatctggtggcaccttcgcgcctgtctcg |
| | | ctgctttcgataagtctctagccatttaaa |
| | | atttttgatgacctgctgcgacgctttttt |
| | | tctggcaagatagtcttgtaaatgcgggcc |
| | | aagatctgcacactggtatttcggtttttg |
| | | gggccgcgggcggcgacggggcccgtgcgt |
| | | cccagcgcacatgttcggcgaggcggggcc |
| | | tgcgagcgcggccaccgagaatcggacggg |
| | | ggtagtctcaagctggccggcctgctctgg |
| | | tgcctggtctcgcgccgccgtgtatcgccc |
| | | cgccctgggcggcaaggctggcccggtcgg |
| | | caccagttgcgtgagcggaaagatggccgc |
| | | ttcccggccctgctgcagggagctcaaaat |
| | | ggaggacgcggcgctcgggagagcgggcgg |
| | | gtgagtcacccacacaaaggaaaagggcct |
| | | ttccgtcctcagccgtcgcttcatgtgact |
| | | ccacggagtaccgggcgccgtccaggcacc |
| | | tcgattagttctcgagcttttggagtacgt |
| | | cgtctttaggttggggggaggggttttatg |
| | | cgatggagtttccccacactgagtgggtgg |
| | | agactgaagttaggccagcttggcacttga |
| | | tgtaattctccttggaatttgccctttttg |
| | | agtttggatcttggttcattctcaagcctc |
| | | agacagtggttcaaagtttttttcttccat |
| | | ttcaggtgtcgtgacaagtttgtacaaaaa |
| | | agcaggctgccaccatggccttaccagtga |
| | | ccgccttgctcctgccgctggccttgctgc |
| | | tccacgccgccaggccgggatccgacatcc |
| | | agatgacccagagcccctcctctttatccg |
| | | cctctgtgggcgacagagtgaccatcactt |
| | | gtcgtgccagccaagaaatctccggctatc |
| | | tgtcttggctgcagcagaagcccggtaagg |
| | | ctatcaagagactcatctacgccgccagca |
| | | ctttacagagcggagtgcctagcagattta |
| | | gcggctctcgtagcggcagcgattatactt |
| | | taaccatctcctctttacagcccgaagact |
| | | ttgctacctactactgtttacagtacgcca |
| | | gctaccccttcaccttcggtcaaggtacca |
| | | agctggagatcaaaggtggcggcggctctg |
| | | gcggcggtggctctggcggcggtggctccc |
| | | aagttaccctcaaggagagcggccccactt |
| | | tagtgaagcctacccagactttaactttaa |
| | | cttgtaccttcagcggcttctctttaagca |
| | | cctccacaatgggcgtgggctggatcagac |
| | | agcctcccggcaaggctctggagtggctgg |
| | | cccacatcctctggaacgacagcaagaggt |
| | | acaacccctctttaaagtctcgtctgacca |
| | | tcaccaaggacacctccaagaagcaagttg |
| | | tgctgaccatgaccaatatggaccccgtgg |
| | | acaccgccacctattactgcgctcgtatcg |
| | | tgtactactccacctacgtgggctacttcg |
| | | acgtctggggacaaggtaccaccgtgaccg |
| | | tgagctctggatccttcgtgccggtcttcc |
| | | tgccagcgaagcccaccacgacgccagcgc |
| | | cgcgaccaccaacaccggcgcccaccatcg |
| | | cgtcgcagcccctgtccctgcgcccagagg |
| | | cgtgccggccagcggcggggggcgcagtgc |
| | | acacgagggggctggacttcgcctgtgata |
| | | tctacatctgggcgcccttggccgggactt |
| | | gtggggtccttctcctgtcactggttatca |
| | | ccctttactgcaaccacaggaacaggagta |
| | | agaggagcaggctcctgcacagtgactaca |
| | | tgaacatgactccccgccgccccgggccca |
| | | cccgcaagcattaccagccctatgccccac |
| | | cacgcgacttcgcagcctatcgctccaaac |
| | | ggggcagaaagaaactcctgtatatattca |
| | | aacaaccatttatgagaccagtacaaacta |
| | | ctcaagaggaagatggctgtagctgccgat |
| | | ttccagaagaagaagaaggaggatgtgaac |
| | | tgagagtgaagttcagcaggagcgcagacg |
| | | cccccgcgtaccagcagggccagaaccagc |
| | | tctataacgagctcaatctaggacgaagag |
| | | aggagtacgatgttttggacaagagacgtg |
| | | gccgggaccctgagatggggggaaagccga |
| | | gaaggaagaaccctcaggaaggcctgtaca |
| | | atgaactgcagaaagataagatggcggagg |
| | | cctacagtgagattgggatgaaaggcgagc |
| | | gccggaggggcaaggggcacgatggccttt |
| | | accagggtctcagtacagccaccaaggaca |
| | | cctacgacgcccttcacatgcaggccctgc |
| | | cccctcgctgaacccagctttcttgtacaa |
| | | agtggtgataatcgaattccgataatcaac |
| | | ctctggattacaaaatttgtgaaagattga |
| | | ctggtattcttaactatgttgctcctttta |
| | | cgctatgtggatacgctgctttaatgcctt |
| | | tgtatcatgctattgcttcccgtatggctt |
| | | tcattttctcctccttgtataaatcctggt |
| | | tgctgtctctttatgaggagttgtggcccg |
| | | ttgtcaggcaacgtggcgtggtgtgcactg |
| | | tgtttgctgacgcaacccccactggttggg |
| | | gcattgccaccacctgtcagctcctttccg |
| | | ggactttcgctttccccctccctattgcca |
| | | cggcggaactcatcgccgcctgccttgccc |
| | | gctgctggacaggggctcggctgttgggca |
| | | ctgacaattccgtggtgttgtcggggaagc |
| | | tgacgtcctttccatggctgctcgcctgtg |
| | | ttgccacctggattctgcgcgggacgtcct |
| | | tctgctacgtcccttcggccctcaatccag |
| | | cggaccttccttcccgcggcctgctgccgg |
| | | ctctgcggcctcttccgcgtcttcgccttc |
| | | gccctcagacgagtcggatctccctttggg |
| | | ccgcctccccgcatcgggaattcccgggtt |
| | | cgctttaagaccaatgacttacaaggcagc |
| | | tgtagatcttagccactttttaaaagaaaa |
| | | ggggggactggaagggctaattcactccca |
| | | acgaagacaagatctgctttttgcttgtac |
| | | tgggtctctctggttagaccagatctgagc |
| | | ctgggagctctctggctaactagggaaccc |
| | | actgcttaagcctcaataaagcttgccttg |
| | | agtgcttcaagtagtgtgtgcccgtctgtt |
| | | gtgtgactctggtaactagagatccctcag |
| | | acccttttagtcagtgtggaaaatctctag |
| | | cagtagtagttcatgtcatcttattattca |
| | | gtatttataacttgcaaagaaatgaatatc |
| | | agagagtgagaggaacttgtttattgcagc |
| | | ttataatggttacaaataaagcaatagcat |
| | | cacaaatttcacaaataaagcatttttttc |
| | | actgcattctagttgtggtttgtccaaact |
| | | catcaatgtatcttatcatgtctggctcta |
| | | gctatcccgcccctaactccgcccatcccg |
| | | cccctaactccgcccagttccgcccattct |
| | | ccgccccatggctgactaattttttttatt |
| | | tatgcagaggccgaggccgcctcggcctct |
| | | gagctattccagaagtagtgaggaggcttt |
| | | tttggaggcctagggacgtacccaattcgc |
| | | cctatagtgagtcgtattacgcgcgctcac |
| | | tggccgtcgttttacaacgtcgtgactggg |
| | | aaaaccctggcgttacccaacttaatcgcc |
| | | ttgcagcacatccccctttcgccagctggc |
| | | gtaatagcgaagaggcccgcaccgatcgcc |
| | | cttcccaacagttgcgcagcctgaatggcg |
| | | aatgggacgcgccctgtagcggcgcattaa |
| | | gcgcggcgggtgtggtggttacgcgcagcg |
| | | tgaccgctacacttgccagcgccctagcgc |
| | | ccgctcctttcgctttcttcccttcctttc |
| | | tcgccacgttcgccggctttccccgtcaag |
| | | ctctaaatcgggggctccctttagggttcc |
| | | gatttagtgctttacggcacctcgacccca |
| | | aaaaacttgattagggtgatggttcacgta |
| | | gtgggccatcgccctgatagacggtttttc |
| | | gccctttgacgttggagtccacgttcttta |
| | | atagtggactcttgttccaaactggaacaa |
| | | cactcaaccctatctcggtctattcttttg |
| | | atttataagggattttgccgatttcggcct |
| | | attggttaaaaaatgagctgatttaacaaa |
| | | aatttaacgcgaattttaacaaaatattaa |
| | | cgcttacaatttaggtggcacttttcgggg |
| | | aaatgtgcgcggaacccctatttgtttatt |
| | | tttctaaatacattcaaatatgtatccgct |
| | | catgagacaataaccctgataaatgcttca |
| | | ataatattgaaaaaggaagagtatgagtat |
| | | tcaacatttccgtgtcgcccttattccctt |
| | | ttttgcggcattttgccttcctgtttttgc |
| | | tcacccagaaacgctggtgaaagtaaaaga |
| | | tgctgaagatcagttgggtgcacgagtggg |
| | | ttacatcgaactggatctcaacagcggtaa |
| | | gatccttgagagttttcgccccgaagaacg |
| | | ttttccaatgatgagcacttttaaagttct |
| | | gctatgtggcgcggtattatcccgtattga |
| | | cgccgggcaagagcaactcggtcgccgcat |
| | | acactattctcagaatgacttggttgagta |
| | | ctcaccagtcacagaaaagcatcttacgga |
| | | tggcatgacagtaagagaattatgcagtgc |
| | | tgccataaccatgagtgataacactgcggc |
| | | caacttacttctgacaacgatcggaggacc |
| | | gaaggagctaaccgcttttttgcacaacat |
| | | gggggatcatgtaactcgccttgatcgttg |
| | | ggaaccggagctgaatgaagccataccaaa |
| | | cgacgagcgtgacaccacgatgcctgtagc |
| | | aatggcaacaacgttgcgcaaactattaac |
| | | tggcgaactacttactctagcttcccggca |
| | | acaattaatagactggatggaggcggataa |
| | | agttgcaggaccacttctgcgctcggccct |
| | | tccggctggctggtttattgctgataaatc |
| | | tggagccggtgagcgtgggtctcgcggtat |
| | | cattgcagcactggggccagatggtaagcc |
| | | ctcccgtatcgtagttatctacacgacggg |
| | | gagtcaggcaactatggatgaacgaaatag |
| | | acagatcgctgagataggtgcctcactgat |
| | | taagcattggtaactgtcagaccaagttta |
| | | ctcatatatactttagattgatttaaaact |
| | | tcatttttaatttaaaaggatctaggtgaa |
| | | gatcctttttgataatctcatgaccaaaat |
| | | cccttaacgtgagttttcgttccactgagc |
| | | gtcagaccccgtagaaaagatcaaaggatc |
| | | ttcttgagatcctttttttctgcgcgtaat |
| | | ctgctgcttgcaaacaaaaaaaccaccgct |
| | | accagcggtggtttgtttgccggatcaaga |
| | | gctaccaactctttttccgaaggtaactgg |
| | | cttcagcagagcgcagataccaaatactgt |
| | | tcttctagtgtagccgtagttaggccacca |
| | | cttcaagaactctgtagcaccgcctacata |
| | | cctcgctctgctaatcctgttaccagtggc |
| | | tgctgccagtggcgataagtcgtgtcttac |
| | | cgggttggactcaagacgatagttaccgga |
| | | taaggcgcagcggtcgggctgaacgggggg |
| | | ttcgtgcacacagcccagcttggagcgaac |
| | | gacctacaccgaactgagatacctacagcg |
| | | tgagctatgagaaagcgccacgcttcccga |
| | | agagagaaaggcggacaggtatccggtaag |
| | | cggcagggtcggaacaggagagcgcacgag |
| | | ggagcttccagggggaaacgcctggtatct |
| | | ttatagtcctgtcgggtttcgccacctctg |
| | | acttgagcgtcgatttttgtgatgctcgtc |
| | | aggggggcggagcctatggaaaaacgccag |
| | | caacgcggcctttttacggttcctggcctt |
| | | ttgctggccttttgctcacatgttctttcc |
| | | tgcgttatcccctgattctgtggataaccg |
| | | tattaccgcctttgagtgagctgataccgc |
| | | tcgccgcagccgaacgaccgagcgcagcga |
| | | gtcagtgagcgaggaagcggaagagcgccc |
| | | aatacgcaaaccgcctctccccgcgcgttg |
| | | gccgattcattaatgcagctggcacgacag |
| | | gtttcccgactggaaagcgggcagtgagcg |
| | | caacgcaattaatgtgagttagctcactca |
| | | ttaggcaccccaggctttacactttatgct |
| | | tccggctcgtatgttgtgtggaattgtgag |
| | | cggataacaatttcacacaggaaacagcta |
| | | tgaccatgattacgccaagcgcgcaattaa |
| | | ccctcactaaagggaacaaaagctggagct |
| | | gcaagctt |
| 99 | Nucleic Acid | aatgtagtcttatgcaatactcttgtagtc |
| | sequence of HG- | ttgcaacatggtaacgatgagttagcaaca |
| | CAR-TG105- | tgccttacaaggagagaaaaagcaccgtgc |
| | EF1_CAR3a_20aa | atgccgattggtggaagtaaggtggtacga |
| | linker-plasmid | tcgtgccttattaggaaggcaacagacggg |
| | | tctgacatggattggacgaaccactgaatt |
| | | gccgcattgcagagatattgtatttaagtg |
| | | cctagctcgatacataaacgggtctctctg |
| | | gttagaccagatctgagcctgggagctctc |
| | | tggctaactagggaacccactgcttaagcc |
| | | tcaataaagcttgccttgagtgcttcaagt |
| | | agtgtgtgcccgtctgttgtgtgactctgg |
| | | taactagagatccctcagacccttttagtc |
| | | agtgtggaaaatctctagcagtggcgcccg |
| | | aacagggacttgaaagcgaaagggaaacca |
| | | gaggagctctctcgacgcaggactcggctt |
| | | gctgaagcgcgcacggcaagaggcgagggg |
| | | ggcgactggtgagtacgccaaaaattttga |
| | | ctagcggaggctagaaggagagagatgggt |
| | | gcgagagcgtcagtattaagcgggggagaa |
| | | ttagatcgcgatgggaaaaaattcggttaa |
| | | ggccagggggaaagaaaaaatataaattaa |
| | | aacatatagtatgggcaagcagggagctag |
| | | aacgattcgcagttaatcctggcctgttag |
| | | aaacatcagaaggctgtagacaaatactgg |
| | | gacagctacaaccatcccttcagacaggat |
| | | cagaagaacttagatcattatataatacag |
| | | tagcaaccctctattgtgtgcatcaaagga |
| | | tagagataaaagacaccaaggaagctttag |
| | | acaagatagaggaagagcaaaacaaaagta |
| | | agaccaccgcacagcaagcggccgctgatc |
| | | ttcagacctggaggaggagatatgagggac |
| | | aattggagaagtgaattatataaatataaa |
| | | gtagtaaaaattgaaccattaggagtagca |
| | | cccaccaaggcaaagagaagagtggtgcag |
| | | agagaaaaaagagcagtgggaataggagct |
| | | ttgttccttgggttcttgggagcagcagga |
| | | agcactatgggcgcagcgtcaatgacgctg |
| | | acggtacaggccagacaattattgtctggt |
| | | atagtgcagcagcagaacaatttgctgagg |
| | | gctattgaggcgcaacagcatctgttgcaa |
| | | ctcacagtctggggcatcaagcagctccag |
| | | gcaagaatcctggctgtggaaagataccta |
| | | aaggatcaacagctcctggggatttggggt |
| | | tgctctggaaaactcatttgcaccactgct |
| | | gtgccttggaatgctagttggagtaataaa |
| | | tctctggaacagatttggaatcacacgacc |
| | | tggatggagtgggacagagaaattaacaat |
| | | tacacaagcttaatacactccttaattgaa |
| | | gaatcgcaaaaccagcaagaaaagaatgaa |
| | | caagaattattggaattagataaatgggca |
| | | agtttgtggaattggtttaacataacaaat |
| | | tggctgtggtatataaaattattcataatg |
| | | atagtaggaggcttggtaggtttaagaata |
| | | gtttttgctgtactttctatagtgaataga |
| | | gttaggcagggatattcaccattatcgttt |
| | | cagacccacctcccaaccccgaggggaccc |
| | | gacaggcccgaaggaatagaagaagaaggt |
| | | ggagagagagacagagacagatccattcga |
| | | ttagtgaacggatctcgacggtatcgctag |
| | | cttttaaaagaaaaggggggattggggggt |
| | | acagtgcaggggaaagaatagtagacataa |
| | | tagcaacagacatacaaactaaagaattac |
| | | aaaaacaaattacaaaaattcaaaatttta |
| | | ctagtgattatcggatcaactttgtataga |
| | | aaagttgggctccggtgcccgtcagtgggc |
| | | agagcgcacatcgcccacagtccccgagaa |
| | | gttggggggaggggtcggcaattgaaccgg |
| | | tgcctagagaaggtggcgcggggtaaactg |
| | | ggaaagtgatgtcgtgtactggctccgcct |
| | | ttttcccgaggggggggagaaccgtatata |
| | | agtgcagtagtcgccgtgaacgttcttttt |
| | | cgcaacgggtttgccgccagaacacaggta |
| | | agtgccgtgtgtggttcccgcgggcctggc |
| | | ctctttacgggttatggcccttgcgtgcct |
| | | tgaattacttccacctggctgcagtacgtg |
| | | attcttgatcccgagcttcgggttggaagt |
| | | gggtgggagagttcgaggccttgcgcttaa |
| | | ggagccccttcgcctcgtgcttgagttgag |
| | | gcctggcctgggcgctggggccgccgcgtg |
| | | cgaatctggtggcaccttcgcgcctgtctc |
| | | gctgctttcgataagtctctagccatttaa |
| | | aatttttgatgacctgctgcgacgcttttt |
| | | ttctggcaagatagtcttgtaaatgcgggc |
| | | caagatctgcacactggtatttcggttttt |
| | | ggggccgcgggcggcgacggggcccgtgcg |
| | | tcccagcgcacatgttcggcgaggcggggc |
| | | ctgcgagcgcggccaccgagaatcggacgg |
| | | gggtagtctcaagctggccggcctgctctg |
| | | gtgcctggtctcgcgccgccgtgtatcgcc |
| | | ccgccctgggcggcaaggctggcccggtcg |
| | | gcaccagttgcgtgagcggaaagatggccg |
| | | cttcccggccctgctgcagggagctcaaaa |
| | | tggaggacgcggcgctcgggagagcggggg |
| | | gtgagtcacccacacaaaggaaaagggcct |
| | | ttccgtcctcagccgtcgcttcatgtgact |
| | | ccacggagtaccgggcgccgtccaggcacc |
| | | tcgattagttctcgagcttttggagtacgt |
| | | cgtctttaggttggggggaggggttttatg |
| | | cgatggagtttccccacactgagtgggtgg |
| | | agactgaagttaggccagcttggcacttga |
| | | tgtaattctccttggaatttgccctttttg |
| | | agtttggatcttggttcattctcaagcctc |
| | | agacagtggttcaaagtttttttcttccat |
| | | ttcaggtgtcgtgacaagtttgtacaaaaa |
| | | agcaggctgccaccatggccttaccagtga |
| | | ccgccttgctcctgccgctggccttgctgc |
| | | tccacgccgccaggccgggatccgacatcc |
| | | agatgacccagagcccctcctctttatccg |
| | | cctctgtgggcgacagagtgaccatcactt |
| | | gtcgtgccagccaagaaatctccggctatc |
| | | tgtcttggctgcagcagaagcccggtaagg |
| | | ctatcaagagactcatctacgccgccagca |
| | | ctttacagagcggagtgcctagcagattta |
| | | gcggctctcgtagcggcagcgattatactt |
| | | taaccatctcctctttacagcccgaagact |
| | | ttgctacctactactgtttacagtacgcca |
| | | gctaccccttcaccttcggtcaaggtacca |
| | | agctggagatcaaaggtggcggcggctctg |
| | | gcggcggtggctctggcggcggtggctccg |
| | | gcggtggtggtagccaagttaccctcaagg |
| | | agagcggccccactttagtgaagcctaccc |
| | | agactttaactttaacttgtaccttcagcg |
| | | gcttctctttaagcacctccacaatgggcg |
| | | tgggctggatcagacagcctcccggcaagg |
| | | ctctggagtggctggcccacatcctctgga |
| | | acgacagcaagaggtacaacccctctttaa |
| | | agtctcgtctgaccatcaccaaggacacct |
| | | ccaagaagcaagttgtgctgaccatgacca |
| | | atatggaccccgtggacaccgccacctatt |
| | | actgcgctcgtatcgtgtactactccacct |
| | | acgtgggctacttcgacgtctggggacaag |
| | | gtaccaccgtgaccgtgagctctggatcct |
| | | tcgtgccggtcttcctgccagcgaagccca |
| | | ccacgacgccagcgccgcgaccaccaacac |
| | | cggcgcccaccatcgcgtcgcagcccctgt |
| | | ccctgcgcccagaggcgtgccggccagcgg |
| | | ggggggcgcagtgcacacgagggggctgga |
| | | cttcgcctgtgatatctacatctgggcgcc |
| | | cttggccgggacttgtggggtccttctcct |
| | | gtcactggttatcaccctttactgcaacca |
| | | caggaacaggagtaagaggagcaggctcct |
| | | gcacagtgactacatgaacatgactccccg |
| | | ccgccccgggcccacccgcaagcattacca |
| | | gccctatgccccaccacgcgacttcgcagc |
| | | ctatcgctccaaacggggcagaaagaaact |
| | | cctgtatatattcaaacaaccatttatgag |
| | | accagtacaaactactcaagaggaagatgg |
| | | ctgtagctgccgatttccagaagaagaaga |
| | | aggaggatgtgaactgagagtgaagttcag |
| | | caggagcgcagacgcccccgcgtaccagca |
| | | gggccagaaccagctctataacgagctcaa |
| | | tctaggacgaagagaggagtacgatgtttt |
| | | ggacaagagacgtggccgggaccctgagat |
| | | ggggggaaagccgagaaggaagaaccctca |
| | | ggaaggcctgtacaatgaactgcagaaaga |
| | | taagatggcggaggcctacagtgagattgg |
| | | gatgaaaggcgagcgccggaggggcaaggg |
| | | gcacgatggcctttaccagggtctcagtac |
| | | agccaccaaggacacctacgacgcccttca |
| | | catgcaggccctgccccctcgctgaaccca |
| | | gctttcttgtacaaagtggtgataatcgaa |
| | | ttccgataatcaacctctggattacaaaat |
| | | ttgtgaaagattgactggtattcttaacta |
| | | tgttgctccttttacgctatgtggatacgc |
| | | tgctttaatgcctttgtatcatgctattgc |
| | | ttcccgtatggctttcattttctcctcctt |
| | | gtataaatcctggttgctgtctctttatga |
| | | ggagttgtggcccgttgtcaggcaacgtgg |
| | | cgtggtgtgcactgtgtttgctgacgcaac |
| | | ccccactggttggggcattgccaccacctg |
| | | tcagctcctttccgggactttcgctttccc |
| | | cctccctattgccacggcggaactcatcgc |
| | | cgcctgccttgcccgctgctggacaggggc |
| | | tcggctgttgggcactgacaattccgtggt |
| | | gttgtcggggaagctgacgtcctttccatg |
| | | gctgctcgcctgtgttgccacctggattct |
| | | gcgcgggacgtccttctgctacgtcccttc |
| | | ggccctcaatccagcggaccttccttcccg |
| | | cggcctgctgccggctctgcggcctcttcc |
| | | gcgtcttcgccttcgccctcagacgagtcg |
| | | gatctccctttgggccgcctccccgcatcg |
| | | ggaattcccgcggttcgctttaagaccaat |
| | | gacttacaaggcagctgtagatcttagcca |
| | | ctttttaaaagaaaaggggggactggaagg |
| | | gctaattcactcccaacgaagacaagatct |
| | | gctttttgcttgtactgggtctctctggtt |
| | | agaccagatctgagcctgggagctctctgg |
| | | ctaactagggaacccactgcttaagcctca |
| | | ataaagcttgccttgagtgcttcaagtagt |
| | | gtgtgcccgtctgttgtgtgactctggtaa |
| | | ctagagatccctcagacccttttagtcagt |
| | | gtggaaaatctctagcagtagtagttcatg |
| | | tcatcttattattcagtatttataacttgc |
| | | aaagaaatgaatatcagagagtgagaggaa |
| | | cttgtttattgcagcttataatggttacaa |
| | | ataaagcaatagcatcacaaatttcacaaa |
| | | taaagcatttttttcactgcattctagttg |
| | | tggtttgtccaaactcatcaatgtatctta |
| | | tcatgtctggctctagctatcccgccccta |
| | | actccgcccatcccgcccctaactccgccc |
| | | agttccgcccattctccgccccatggctga |
| | | ctaattttttttatttatgcagaggccgag |
| | | gccgcctcggcctctgagctattccagaag |
| | | tagtgaggaggcttttttggaggcctaggg |
| | | acgtacccaattcgccctatagtgagtcgt |
| | | attacgcgcgctcactggccgtcgttttac |
| | | aacgtcgtgactgggaaaaccctggcgtta |
| | | cccaacttaatcgccttgcagcacatcccc |
| | | ctttcgccagctggcgtaatagcgaagagg |
| | | cccgcaccgatcgcccttcccaacagttgc |
| | | gcagcctgaatggcgaatgggacgcgccct |
| | | gtagcggcgcattaagcgcggcgggtgtgg |
| | | tggttacgcgcagcgtgaccgctacacttg |
| | | ccagcgccctagcgcccgctcctttcgctt |
| | | tcttcccttcctttctcgccacgttcgccg |
| | | gctttccccgtcaagctctaaatcgggggc |
| | | tccctttagggttccgatttagtgctttac |
| | | ggcacctcgaccccaaaaaacttgattagg |
| | | gtgatggttcacgtagtgggccatcgccct |
| | | gatagacggtttttcgccctttgacgttgg |
| | | agtccacgttctttaatagtggactcttgt |
| | | tccaaactggaacaacactcaaccctatct |
| | | cggtctattcttttgatttataagggattt |
| | | tgccgatttcggcctattggttaaaaaatg |
| | | agctgatttaacaaaaatttaacgcgaatt |
| | | ttaacaaaatattaacgcttacaatttagg |
| | | tggcacttttcggggaaatgtgcgcggaac |
| | | ccctatttgtttatttttctaaatacattc |
| | | aaatatgtatccgctcatgagacaataacc |
| | | ctgataaatgcttcaataatattgaaaaag |
| | | gaagagtatgagtattcaacatttccgtgt |
| | | cgcccttattcccttttttgcggcattttg |
| | | ccttcctgtttttgctcacccagaaacgct |
| | | ggtgaaagtaaaagatgctgaagatcagtt |
| | | gggtgcacgagtgggttacatcgaactgga |
| | | tctcaacagcggtaagatccttgagagttt |
| | | tcgccccgaagaacgttttccaatgatgag |
| | | cacttttaaagttctgctatgtggcgcggt |
| | | attatcccgtattgacgccgggcaagagca |
| | | actcggtcgccgcatacactattctcagaa |
| | | tgacttggttgagtactcaccagtcacaga |
| | | aaagcatcttacggatggcatgacagtaag |
| | | agaattatgcagtgctgccataaccatgag |
| | | tgataacactgcggccaacttacttctgac |
| | | aacgatcggaggaccgaaggagctaaccgc |
| | | ttttttgcacaacatgggggatcatgtaac |
| | | tcgccttgatcgttgggaaccggagctgaa |
| | | tgaagccataccaaacgacgagcgtgacac |
| | | cacgatgcctgtagcaatggcaacaacgtt |
| | | gcgcaaactattaactggcgaactacttac |
| | | tctagcttcccggcaacaattaatagactg |
| | | gatggaggcggataaagttgcaggaccact |
| | | tctgcgctcggcccttccggctggctggtt |
| | | tattgctgataaatctggagccggtgagcg |
| | | tgggtctcgcggtatcattgcagcactggg |
| | | gccagatggtaagccctcccgtatcgtagt |
| | | tatctacacgacggggagtcaggcaactat |
| | | ggatgaacgaaatagacagatcgctgagat |
| | | aggtgcctcactgattaagcattggtaact |
| | | gtcagaccaagtttactcatatatacttta |
| | | gattgatttaaaacttcatttttaatttaa |
| | | aaggatctaggtgaagatcctttttgataa |
| | | tctcatgaccaaaatcccttaacgtgagtt |
| | | ttcgttccactgagcgtcagaccccgtaga |
| | | aaagatcaaaggatcttcttgagatccttt |
| | | ttttctgcgcgtaatctgctgcttgcaaac |
| | | aaaaaaaccaccgctaccagcggtggtttg |
| | | tttgccggatcaagagctaccaactctttt |
| | | tccgaaggtaactggcttcagcagagcgca |
| | | gataccaaatactgttcttctagtgtagcc |
| | | gtagttaggccaccacttcaagaactctgt |
| | | agcaccgcctacatacctcgctctgctaat |
| | | cctgttaccagtggctgctgccagtggcga |
| | | taagtcgtgtcttaccgggttggactcaag |
| | | acgatagttaccggataaggcgcagcggtc |
| | | gggctgaacggggggttcgtgcacacagcc |
| | | cagcttggagcgaacgacctacaccgaact |
| | | gagatacctacagcgtgagctatgagaaag |
| | | cgccacgcttcccgaagagagaaaggcgga |
| | | caggtatccggtaagcggcagggtcggaac |
| | | aggagagcgcacgagggagcttccaggggg |
| | | aaacgcctggtatctttatagtcctgtcgg |
| | | gtttcgccacctctgacttgagcgtcgatt |
| | | tttgtgatgctcgtcaggggggcggagcct |
| | | atggaaaaacgccagcaacgcggccttttt |
| | | acggttcctggccttttgctggccttttgc |
| | | tcacatgttctttcctgcgttatcccctga |
| | | ttctgtggataaccgtattaccgcctttga |
| | | gtgagctgataccgctcgccgcagccgaac |
| | | gaccgagcgcagcgagtcagtgagcgagga |
| | | agcggaagagcgcccaatacgcaaaccgcc |
| | | tctccccgcgcgttggccgattcattaatg |
| | | cagctggcacgacaggtttcccgactggaa |
| | | agcgggcagtgagcgcaacgcaattaatgt |
| | | gagttagctcactcattaggcaccccaggc |
| | | tttacactttatgcttccggctcgtatgtt |
| | | gtgtggaattgtgagcggataacaatttca |
| | | cacaggaaacagctatgaccatgattacgc |
| | | caagcgcgcaattaaccctcactaaaggga |
| | | acaaaagctggagctgcaagctt |
| 100 | Nucleic Acid | aatgtagtcttatgcaatactcttgtagtc |
| | sequence of | ttgcaacatggtaacgatgagttagcaaca |
| | CAR3a-5aalinker- | tgccttacaaggagagaaaaagcaccgtgc |
| | NO-41BB-plsmid | atgccgattggtggaagtaaggtggtacga |
| | | tcgtgccttattaggaaggcaacagacggg |
| | | tctgacatggattggacgaaccactgaatt |
| | | gccgcattgcagagatattgtatttaagtg |
| | | cctagctcgatacataaacgggtctctctg |
| | | gttagaccagatctgagcctgggagctctc |
| | | tggctaactagggaacccactgcttaagcc |
| | | tcaataaagcttgccttgagtgcttcaagt |
| | | agtgtgtgcccgtctgttgtgtgactctgg |
| | | taactagagatccctcagacccttttagtc |
| | | agtgtggaaaatctctagcagtggcgcccg |
| | | aacagggacttgaaagcgaaagggaaacca |
| | | gaggagctctctcgacgcaggactcggctt |
| | | gctgaagcgcgcacggcaagaggcgagggg |
| | | cggcgactggtgagtacgccaaaaattttg |
| | | actagcggaggctagaaggagagagatggg |
| | | tgcgagagcgtcagtattaagcgggggaga |
| | | attagatcgcgatgggaaaaaattcggtta |
| | | aggccagggggaaagaaaaaatataaatta |
| | | aaacatatagtatgggcaagcagggagcta |
| | | gaacgattcgcagttaatcctggcctgtta |
| | | gaaacatcagaaggctgtagacaaatactg |
| | | ggacagctacaaccatcccttcagacagga |
| | | tcagaagaacttagatcattatataataca |
| | | gtagcaaccctctattgtgtgcatcaaagg |
| | | atagagataaaagacaccaaggaagcttta |
| | | gacaagatagaggaagagcaaaacaaaagt |
| | | aagaccaccgcacagcaagcggccgctgat |
| | | cttcagacctggaggaggagatatgaggga |
| | | caattggagaagtgaattatataaatataa |
| | | agtagtaaaaattgaaccattaggagtagc |
| | | acccaccaaggcaaagagaagagtggtgca |
| | | gagagaaaaaagagcagtgggaataggagc |
| | | tttgttccttgggttcttgggagcagcagg |
| | | aagcactatgggcgcagcgtcaatgacgct |
| | | gacggtacaggccagacaattattgtctgg |
| | | tatagtgcagcagcagaacaatttgctgag |
| | | ggctattgaggcgcaacagcatctgttgca |
| | | actcacagtctggggcatcaagcagctcca |
| | | ggcaagaatcctggctgtggaaagatacct |
| | | aaaggatcaacagctcctggggatttgggg |
| | | ttgctctggaaaactcatttgcaccactgc |
| | | tgtgccttggaatgctagttggagtaataa |
| | | atctctggaacagatttggaatcacacgac |
| | | ctggatggagtgggacagagaaattaacaa |
| | | ttacacaagcttaatacactccttaattga |
| | | agaatcgcaaaaccagcaagaaaagaatga |
| | | acaagaattattggaattagataaatgggc |
| | | aagtttgtggaattggtttaacataacaaa |
| | | ttggctgtggtatataaaattattcataat |
| | | gatagtaggaggcttggtaggtttaagaat |
| | | agtttttgctgtactttctatagtgaatag |
| | | agttaggcagggatattcaccattatcgtt |
| | | tcagacccacctcccaaccccgaggggacc |
| | | cgacaggcccgaaggaatagaagaagaagg |
| | | tggagagagagacagagacagatccattcg |
| | | attagtgaacggatctcgacggtatcgcta |
| | | gcttttaaaagaaaaggggggattgggggg |
| | | tacagtgcaggggaaagaatagtagacata |
| | | atagcaacagacatacaaactaaagaatta |
| | | caaaaacaaattacaaaaattcaaaatttt |
| | | actagtgattatcggatcaactttgtatag |
| | | aaaagttgggctccggtgcccgtcagtggg |
| | | cagagcgcacatcgcccacagtccccgaga |
| | | agttggggggaggggtcggcaattgaaccg |
| | | gtgcctagagaaggtggcgcggggtaaact |
| | | gggaaagtgatgtcgtgtactggctccgcc |
| | | tttttcccgagggtgggggagaaccgtata |
| | | taagtgcagtagtcgccgtgaacgttcttt |
| | | ttcgcaacgggtttgccgccagaacacagg |
| | | taagtgccgtgtgtggttcccgcgggcctg |
| | | gcctctttacgggttatggcccttgcgtgc |
| | | cttgaattacttccacctggctgcagtacg |
| | | tgattcttgatcccgagcttcgggttggaa |
| | | gtgggtgggagagttcgaggccttgcgctt |
| | | aaggagccccttcgcctcgtgcttgagttg |
| | | aggcctggcctgggcgctggggccgccgcg |
| | | tgcgaatctggtggcaccttcgcgcctgtc |
| | | tcgctgctttcgataagtctctagccattt |
| | | aaaatttttgatgacctgctgcgacgcttt |
| | | ttttctggcaagatagtcttgtaaatgcgg |
| | | gccaagatctgcacactggtatttcggttt |
| | | ttggggccgcgggcggcgacggggcccgtg |
| | | cgtcccagcgcacatgttcggcgaggcggg |
| | | gcctgcgagcgcggccaccgagaatcggac |
| | | gggggtagtctcaagctggccggcctgctc |
| | | tggtgcctggtctcgcgccgccgtgtatcg |
| | | ccccgccctgggcggcaaggctggcccggt |
| | | cggcaccagttgcgtgagcggaaagatggc |
| | | cgcttcccggccctgctgcagggagctcaa |
| | | aatggaggacgcggcgctcgggagagcggg |
| | | gggtgagtcacccacacaaaggaaaagggc |
| | | ctttccgtcctcagccgtcgcttcatgtga |
| | | ctccacggagtaccgggcgccgtccaggca |
| | | cctcgattagttctcgagcttttggagtac |
| | | gtcgtctttaggttggggggaggggtttta |
| | | tgcgatggagtttccccacactgagtgggt |
| | | ggagactgaagttaggccagcttggcactt |
| | | gatgtaattctccttggaatttgccctttt |
| | | tgagtttggatcttggttcattctcaagcc |
| | | tcagacagtggttcaaagtttttttcttcc |
| | | atttcaggtgtcgtgacaagtttgtacaaa |
| | | aaagcaggctgccaccatggccttaccagt |
| | | gaccgccttgctcctgccgctggccttgct |
| | | gctccacgccgccaggccgggatccgacat |
| | | ccagatgacccagagcccctcctctttatc |
| | | cgcctctgtgggcgacagagtgaccatcac |
| | | ttgtcgtgccagccaagaaatctccggcta |
| | | tctgtcttggctgcagcagaagcccggtaa |
| | | ggctatcaagagactcatctacgccgccag |
| | | cactttacagagcggagtgcctagcagatt |
| | | tagcggctctcgtagcggcagcgattatac |
| | | tttaaccatctcctctttacagcccgaaga |
| | | ctttgctacctactactgtttacagtacgc |
| | | cagctaccccttcaccttcggtcaaggtac |
| | | caagctggagatcaaaggtggggggctctc |
| | | aagttaccctcaaggagagcggccccactt |
| | | tagtgaagcctacccagactttaactttaa |
| | | cttgtaccttcagcggcttctctttaagca |
| | | cctccacaatgggcgtgggctggatcagac |
| | | agcctcccggcaaggctctggagtggctgg |
| | | cccacatcctctggaacgacagcaagaggt |
| | | acaacccctctttaaagtctcgtctgacca |
| | | tcaccaaggacacctccaagaagcaagttg |
| | | tgctgaccatgaccaatatggaccccgtgg |
| | | acaccgccacctattactgcgctcgtatcg |
| | | tgtactactccacctacgtgggctacttcg |
| | | acgtctggggacaaggtaccaccgtgaccg |
| | | tgagctctggatccttcgtgccggtcttcc |
| | | tgccagcgaagcccaccacgacgccagcgc |
| | | cgcgaccaccaacaccggcgcccaccatcg |
| | | cgtcgcagcccctgtccctgcgcccagagg |
| | | cgtgccggccagcggggggggcgcagtgca |
| | | cacgagggggctggacttcgcctgtgatat |
| | | ctacatctgggcgcccttggccgggacttg |
| | | tggggtccttctcctgtcactggttatcac |
| | | cctttactgcaaccacaggaacaggagtaa |
| | | gaggagcaggctcctgcacagtgactacat |
| | | gaacatgactccccgccgccccgggcccac |
| | | ccgcaagcattaccagccctatgccccacc |
| | | acgcgacttcgcagcctatcgctccagagt |
| | | gaagttcagcaggagcgcagacgcccccgc |
| | | gtaccagcagggccagaaccagctctataa |
| | | cgagctcaatctaggacgaagagaggagta |
| | | cgatgttttggacaagagacgtggccggga |
| | | ccctgagatggggggaaagccgagaaggaa |
| | | gaaccctcaggaaggcctgtacaatgaact |
| | | gcagaaagataagatggcggaggcctacag |
| | | tgagattgggatgaaaggcgagcgccggag |
| | | gggcaaggggcacgatggcctttaccaggg |
| | | tctcagtacagccaccaaggacacctacga |
| | | cgcccttcacatgcaggccctgccccctcg |
| | | ctgaacccagctttcttgtacaaagtggtg |
| | | ataatcgaattccgataatcaacctctgga |
| | | ttacaaaatttgtgaaagattgactggtat |
| | | tcttaactatgttgctccttttacgctatg |
| | | tggatacgctgctttaatgcctttgtatca |
| | | tgctattgcttcccgtatggctttcatttt |
| | | ctcctccttgtataaatcctggttgctgtc |
| | | tctttatgaggagttgtggcccgttgtcag |
| | | gcaacgtggcgtggtgtgcactgtgtttgc |
| | | tgacgcaacccccactggttggggcattgc |
| | | caccacctgtcagctcctttccgggacttt |
| | | cgctttccccctccctattgccacggcgga |
| | | actcatcgccgcctgccttgcccgctgctg |
| | | gacaggggctcggctgttgggcactgacaa |
| | | ttccgtggtgttgtcggggaagctgacgtc |
| | | ctttccatggctgctcgcctgtgttgccac |
| | | ctggattctgcgcgggacgtccttctgcta |
| | | cgtcccttcggccctcaatccagcggacct |
| | | tccttcccgcggcctgctgccggctctgcg |
| | | gcctcttccgcgtcttcgccttcgccctca |
| | | gacgagtcggatctccctttgggccgcctc |
| | | cccgcatcgggaattcccgcggttcgcttt |
| | | aagaccaatgacttacaaggcagctgtaga |
| | | tcttagccactttttaaaagaaaagggggg |
| | | actggaagggctaattcactcccaacgaag |
| | | acaagatctgctttttgcttgtactgggtc |
| | | tctctggttagaccagatctgagcctggga |
| | | gctctctggctaactagggaacccactgct |
| | | taagcctcaataaagcttgccttgagtgct |
| | | tcaagtagtgtgtgcccgtctgttgtgtga |
| | | ctctggtaactagagatccctcagaccctt |
| | | ttagtcagtgtggaaaatctctagcagtag |
| | | tagttcatgtcatcttattattcagtattt |
| | | ataacttgcaaagaaatgaatatcagagag |
| | | tgagaggaacttgtttattgcagcttataa |
| | | tggttacaaataaagcaatagcatcacaaa |
| | | tttcacaaataaagcatttttttcactgca |
| | | ttctagttgtggtttgtccaaactcatcaa |
| | | tgtatcttatcatgtctggctctagctatc |
| | | ccgcccctaactccgcccatcccgccccta |
| | | actccgcccagttccgcccattctccgccc |
| | | catggctgactaattttttttatttatgca |
| | | gaggccgaggccgcctcggcctctgagcta |
| | | ttccagaagtagtgaggaggcttttttgga |
| | | ggcctagggacgtacccaattcgccctata |
| | | gtgagtcgtattacgcgcgctcactggccg |
| | | tcgttttacaacgtcgtgactgggaaaacc |
| | | ctggcgttacccaacttaatcgccttgcag |
| | | cacatccccctttcgccagctggcgtaata |
| | | gcgaagaggcccgcaccgatcgcccttccc |
| | | aacagttgcgcagcctgaatggcgaatggg |
| | | acgcgccctgtagcggcgcattaagcgcgg |
| | | cgggtgtggtggttacgcgcagcgtgaccg |
| | | ctacacttgccagcgccctagcgcccgctc |
| | | ctttcgctttcttcccttcctttctcgcca |
| | | cgttcgccggctttccccgtcaagctctaa |
| | | atcgggggctccctttagggttccgattta |
| | | gtgctttacggcacctcgaccccaaaaaac |
| | | ttgattagggtgatggttcacgtagtgggc |
| | | catcgccctgatagacggtttttcgccctt |
| | | tgacgttggagtccacgttctttaatagtg |
| | | gactcttgttccaaactggaacaacactca |
| | | accctatctcggtctattcttttgatttat |
| | | aagggattttgccgatttcggcctattggt |
| | | taaaaaatgagctgatttaacaaaaattta |
| | | acgcgaattttaacaaaatattaacgctta |
| | | caatttaggtggcacttttcggggaaatgt |
| | | gcgcggaacccctatttgtttatttttcta |
| | | aatacattcaaatatgtatccgctcatgag |
| | | acaataaccctgataaatgcttcaataata |
| | | ttgaaaaaggaagagtatgagtattcaaca |
| | | tttccgtgtcgcccttattcccttttttgc |
| | | ggcattttgccttcctgtttttgctcaccc |
| | | agaaacgctggtgaaagtaaaagatgctga |
| | | agatcagttgggtgcacgagtgggttacat |
| | | cgaactggatctcaacagcggtaagatcct |
| | | tgagagttttcgccccgaagaacgttttcc |
| | | aatgatgagcacttttaaagttctgctatg |
| | | tggcgcggtattatcccgtattgacgccgg |
| | | gcaagagcaactcggtcgccgcatacacta |
| | | ttctcagaatgacttggttgagtactcacc |
| | | agtcacagaaaagcatcttacggatggcat |
| | | gacagtaagagaattatgcagtgctgccat |
| | | aaccatgagtgataacactgcggccaactt |
| | | acttctgacaacgatcggaggaccgaagga |
| | | gctaaccgcttttttgcacaacatggggga |
| | | tcatgtaactcgccttgatcgttgggaacc |
| | | ggagctgaatgaagccataccaaacgacga |
| | | gcgtgacaccacgatgcctgtagcaatggc |
| | | aacaacgttgcgcaaactattaactggcga |
| | | actacttactctagcttcccggcaacaatt |
| | | aatagactggatggaggcggataaagttgc |
| | | aggaccacttctgcgctcggcccttccggc |
| | | tggctggtttattgctgataaatctggagc |
| | | cggtgagcgtgggtctcgcggtatcattgc |
| | | agcactggggccagatggtaagccctcccg |
| | | tatcgtagttatctacacgacggggagtca |
| | | ggcaactatggatgaacgaaatagacagat |
| | | cgctgagataggtgcctcactgattaagca |
| | | ttggtaactgtcagaccaagtttactcata |
| | | tatactttagattgatttaaaacttcattt |
| | | ttaatttaaaaggatctaggtgaagatcct |
| | | ttttgataatctcatgaccaaaatccctta |
| | | acgtgagttttcgttccactgagcgtcaga |
| | | ccccgtagaaaagatcaaaggatcttcttg |
| | | agatcctttttttctgcgcgtaatctgctg |
| | | cttgcaaacaaaaaaaccaccgctaccagc |
| | | ggtggtttgtttgccggatcaagagctacc |
| | | aactctttttccgaaggtaactggcttcag |
| | | cagagcgcagataccaaatactgttcttct |
| | | agtgtagccgtagttaggccaccacttcaa |
| | | gaactctgtagcaccgcctacatacctcgc |
| | | tctgctaatcctgttaccagtggctgctgc |
| | | cagtggcgataagtcgtgtcttaccgggtt |
| | | ggactcaagacgatagttaccggataaggc |
| | | gcagcggtcgggctgaacggggggttcgtg |
| | | cacacagcccagcttggagcgaacgaccta |
| | | caccgaactgagatacctacagcgtgagct |
| | | atgagaaagcgccacgcttcccgaagagag |
| | | aaaggcggacaggtatccggtaagcggcag |
| | | ggtcggaacaggagagcgcacgagggagct |
| | | tccagggggaaacgcctggtatctttatag |
| | | tcctgtcgggtttcgccacctctgacttga |
| | | gcgtcgatttttgtgatgctcgtcaggggg |
| | | gggagcctatggaaaaacgccagcaacgcg |
| | | gcctttttacggttcctggccttttgctgg |
| | | ccttttgctcacatgttctttcctgcgtta |
| | | tcccctgattctgtggataaccgtattacc |
| | | gcctttgagtgagctgataccgctcgccgc |
| | | agccgaacgaccgagcgcagcgagtcagtg |
| | | agcgaggaagcggaagagcgcccaatacgc |
| | | aaaccgcctctccccgcgcgttggccgatt |
| | | cattaatgcagctggcacgacaggtttccc |
| | | gactggaaagcgggcagtgagcgcaacgca |
| | | attaatgtgagttagctcactcattaggca |
| | | ccccaggctttacactttatgcttccggct |
| | | cgtatgttgtgtggaattgtgagcggataa |
| | | caatttcacacaggaaacagctatgaccat |
| | | gattacgccaagcgcgcaattaaccctcac |
| | | taaagggaacaaaagctggagctgcaagct |
| | | t |
| 101 | Nucleic Acid | aatgtagtcttatgcaatactcttgtagtc |
| | sequence of | ttgcaacatggtaacgatgagttagcaaca |
| | CAR3a-20aalinker- | tgccttacaaggagagaaaaagcaccgtgc |
| | NO-41BB plasmid | atgccgattggtggaagtaaggtggtacga |
| | | tcgtgccttattaggaaggcaacagacggg |
| | | tctgacatggattggacgaaccactgaatt |
| | | gccgcattgcagagatattgtatttaagtg |
| | | cctagctcgatacataaacgggtctctctg |
| | | gttagaccagatctgagcctgggagctctc |
| | | tggctaactagggaacccactgcttaagcc |
| | | tcaataaagcttgccttgagtgcttcaagt |
| | | agtgtgtgcccgtctgttgtgtgactctgg |
| | | taactagagatccctcagacccttttagtc |
| | | agtgtggaaaatctctagcagtggcgcccg |
| | | aacagggacttgaaagcgaaagggaaacca |
| | | gaggagctctctcgacgcaggactcggctt |
| | | gctgaagcgcgcacggcaagaggcgagggg |
| | | cggcgactggtgagtacgccaaaaattttg |
| | | actagcggaggctagaaggagagagatggg |
| | | tgcgagagcgtcagtattaagcgggggaga |
| | | attagatcgcgatgggaaaaaattcggtta |
| | | aggccagggggaaagaaaaaatataaatta |
| | | aaacatatagtatgggcaagcagggagcta |
| | | gaacgattcgcagttaatcctggcctgtta |
| | | gaaacatcagaaggctgtagacaaatactg |
| | | ggacagctacaaccatcccttcagacagga |
| | | tcagaagaacttagatcattatataataca |
| | | gtagcaaccctctattgtgtgcatcaaagg |
| | | atagagataaaagacaccaaggaagcttta |
| | | gacaagatagaggaagagcaaaacaaaagt |
| | | aagaccaccgcacagcaagcggccgctgat |
| | | cttcagacctggaggaggagatatgaggga |
| | | caattggagaagtgaattatataaatataa |
| | | agtagtaaaaattgaaccattaggagtagc |
| | | acccaccaaggcaaagagaagagtggtgca |
| | | gagagaaaaaagagcagtgggaataggagc |
| | | tttgttccttgggttcttgggagcagcagg |
| | | aagcactatgggcgcagcgtcaatgacgct |
| | | gacggtacaggccagacaattattgtctgg |
| | | tatagtgcagcagcagaacaatttgctgag |
| | | ggctattgaggcgcaacagcatctgttgca |
| | | actcacagtctggggcatcaagcagctcca |
| | | ggcaagaatcctggctgtggaaagatacct |
| | | aaaggatcaacagctcctggggatttgggg |
| | | ttgctctggaaaactcatttgcaccactgc |
| | | tgtgccttggaatgctagttggagtaataa |
| | | atctctggaacagatttggaatcacacgac |
| | | ctggatggagtgggacagagaaattaacaa |
| | | ttacacaagcttaatacactccttaattga |
| | | agaatcgcaaaaccagcaagaaaagaatga |
| | | acaagaattattggaattagataaatgggc |
| | | aagtttgtggaattggtttaacataacaaa |
| | | ttggctgtggtatataaaattattcataat |
| | | gatagtaggaggcttggtaggtttaagaat |
| | | agtttttgctgtactttctatagtgaatag |
| | | agttaggcagggatattcaccattatcgtt |
| | | tcagacccacctcccaaccccgaggggacc |
| | | cgacaggcccgaaggaatagaagaagaagg |
| | | tggagagagagacagagacagatccattcg |
| | | attagtgaacggatctcgacggtatcgcta |
| | | gcttttaaaagaaaaggggggattgggggg |
| | | tacagtgcaggggaaagaatagtagacata |
| | | atagcaacagacatacaaactaaagaatta |
| | | caaaaacaaattacaaaaattcaaaatttt |
| | | actagtgattatcggatcaactttgtatag |
| | | aaaagttgggctccggtgcccgtcagtggg |
| | | cagagcgcacatcgcccacagtccccgaga |
| | | agttggggggaggggtcggcaattgaaccg |
| | | gtgcctagagaaggtggcgcggggtaaact |
| | | gggaaagtgatgtcgtgtactggctccgcc |
| | | tttttcccgaggggggggagaaccgtatat |
| | | aagtgcagtagtcgccgtgaacgttctttt |
| | | tcgcaacgggtttgccgccagaacacaggt |
| | | aagtgccgtgtgtggttcccgcgggcctgg |
| | | cctctttacgggttatggcccttgcgtgcc |
| | | ttgaattacttccacctggctgcagtacgt |
| | | gattcttgatcccgagcttcgggttggaag |
| | | tgggtgggagagttcgaggccttgcgctta |
| | | aggagccccttcgcctcgtgcttgagttga |
| | | ggcctggcctgggcgctggggccgccgcgt |
| | | gcgaatctggtggcaccttcgcgcctgtct |
| | | cgctgctttcgataagtctctagccattta |
| | | aaatttttgatgacctgctgcgacgctttt |
| | | tttctggcaagatagtcttgtaaatgcggg |
| | | ccaagatctgcacactggtatttcggtttt |
| | | tggggccgcgggcggcgacggggcccgtgc |
| | | gtcccagcgcacatgttcggcgaggcgggg |
| | | cctgcgagcgcggccaccgagaatcggacg |
| | | ggggtagtctcaagctggccggcctgctct |
| | | ggtgcctggtctcgcgccgccgtgtatcgc |
| | | cccgccctgggcggcaaggctggcccggtc |
| | | ggcaccagttgcgtgagcggaaagatggcc |
| | | gcttcccggccctgctgcagggagctcaaa |
| | | atggaggacgcggcgctcgggagagcgggg |
| | | ggtgagtcacccacacaaaggaaaagggcc |
| | | tttccgtcctcagccgtcgcttcatgtgac |
| | | tccacggagtaccgggcgccgtccaggcac |
| | | ctcgattagttctcgagcttttggagtacg |
| | | tcgtctttaggttggggggaggggttttat |
| | | gcgatggagtttccccacactgagtgggtg |
| | | gagactgaagttaggccagcttggcacttg |
| | | atgtaattctccttggaatttgcccttttt |
| | | gagtttggatcttggttcattctcaagcct |
| | | cagacagtggttcaaagtttttttcttcca |
| | | tttcaggtgtcgtgacaagtttgtacaaaa |
| | | aagcaggctgccaccatggccttaccagtg |
| | | accgccttgctcctgccgctggccttgctg |
| | | ctccacgccgccaggccgggatccgacatc |
| | | cagatgacccagagcccctcctctttatcc |
| | | gcctctgtgggcgacagagtgaccatcact |
| | | tgtcgtgccagccaagaaatctccggctat |
| | | ctgtcttggctgcagcagaagcccggtaag |
| | | gctatcaagagactcatctacgccgccagc |
| | | actttacagagcggagtgcctagcagattt |
| | | agcggctctcgtagcggcagcgattatact |
| | | ttaaccatctcctctttacagcccgaagac |
| | | tttgctacctactactgtttacagtacgcc |
| | | agctaccccttcaccttcggtcaaggtacc |
| | | aagctggagatcaaaggtggcggcggctct |
| | | ggcggcggtggctctggggggggctccggc |
| | | ggtggtggtagccaagttaccctcaaggag |
| | | agcggccccactttagtgaagcctacccag |
| | | actttaactttaacttgtaccttcagcggc |
| | | ttctctttaagcacctccacaatgggcgtg |
| | | ggctggatcagacagcctcccggcaaggct |
| | | ctggagtggctggcccacatcctctggaac |
| | | gacagcaagaggtacaacccctctttaaag |
| | | tctcgtctgaccatcaccaaggacacctcc |
| | | aagaagcaagttgtgctgaccatgaccaat |
| | | atggaccccgtggacaccgccacctattac |
| | | tgcgctcgtatcgtgtactactccacctac |
| | | gtgggctacttcgacgtctggggacaaggt |
| | | accaccgtgaccgtgagctctggatccttc |
| | | gtgccggtcttcctgccagcgaagcccacc |
| | | acgacgccagcgccgcgaccaccaacaccg |
| | | gcgcccaccatcgcgtcgcagcccctgtcc |
| | | ctgcgcccagaggcgtgccggccagcggcg |
| | | gggggcgcagtgcacacgagggggctggac |
| | | ttcgcctgtgatatctacatctgggcgccc |
| | | ttggccgggacttgtggggtccttctcctg |
| | | tcactggttatcaccctttactgcaaccac |
| | | aggaacaggagtaagaggagcaggctcctg |
| | | cacagtgactacatgaacatgactccccgc |
| | | cgccccgggcccacccgcaagcattaccag |
| | | ccctatgccccaccacgcgacttcgcagcc |
| | | tatcgctccagagtgaagttcagcaggagc |
| | | gcagacgcccccgcgtaccagcagggccag |
| | | aaccagctctataacgagctcaatctagga |
| | | cgaagagaggagtacgatgttttggacaag |
| | | agacgtggccgggaccctgagatgggggga |
| | | aagccgagaaggaagaaccctcaggaaggc |
| | | ctgtacaatgaactgcagaaagataagatg |
| | | gcggaggcctacagtgagattgggatgaaa |
| | | ggcgagcgccggaggggcaaggggcacgat |
| | | ggcctttaccagggtctcagtacagccacc |
| | | aaggacacctacgacgcccttcacatgcag |
| | | gccctgccccctcgctgaacccagctttct |
| | | tgtacaaagtggtgataatcgaattccgat |
| | | aatcaacctctggattacaaaatttgtgaa |
| | | agattgactggtattcttaactatgttgct |
| | | ccttttacgctatgtggatacgctgcttta |
| | | atgcctttgtatcatgctattgcttcccgt |
| | | atggctttcattttctcctccttgtataaa |
| | | tcctggttgctgtctctttatgaggagttg |
| | | tggcccgttgtcaggcaacgtggcgtggtg |
| | | tgcactgtgtttgctgacgcaacccccact |
| | | ggttggggcattgccaccacctgtcagctc |
| | | ctttccgggactttcgctttccccctccct |
| | | attgccacggcggaactcatcgccgcctgc |
| | | cttgcccgctgctggacaggggctcggctg |
| | | ttgggcactgacaattccgtggtgttgtcg |
| | | gggaagctgacgtcctttccatggctgctc |
| | | gcctgtgttgccacctggattctgcgcggg |
| | | acgtccttctgctacgtcccttcggccctc |
| | | aatccagcggaccttccttcccgcggcctg |
| | | ctgccggctctgcggcctcttccgcgtctt |
| | | cgccttcgccctcagacgagtcggatctcc |
| | | ctttgggccgcctccccgcatcgggaattc |
| | | ccgcggttcgctttaagaccaatgacttac |
| | | aaggcagctgtagatcttagccacttttta |
| | | aaagaaaaggggggactggaagggctaatt |
| | | cactcccaacgaagacaagatctgcttttt |
| | | gcttgtactgggtctctctggttagaccag |
| | | atctgagcctgggagctctctggctaacta |
| | | gggaacccactgcttaagcctcaataaagc |
| | | ttgccttgagtgcttcaagtagtgtgtgcc |
| | | cgtctgttgtgtgactctggtaactagaga |
| | | tccctcagacccttttagtcagtgtggaaa |
| | | atctctagcagtagtagttcatgtcatctt |
| | | attattcagtatttataacttgcaaagaaa |
| | | tgaatatcagagagtgagaggaacttgttt |
| | | attgcagcttataatggttacaaataaagc |
| | | aatagcatcacaaatttcacaaataaagca |
| | | tttttttcactgcattctagttgtggtttg |
| | | tccaaactcatcaatgtatcttatcatgtc |
| | | tggctctagctatcccgcccctaactccgc |
| | | ccatcccgcccctaactccgcccagttccg |
| | | cccattctccgccccatggctgactaattt |
| | | tttttatttatgcagaggccgaggccgcct |
| | | cggcctctgagctattccagaagtagtgag |
| | | gaggcttttttggaggcctagggacgtacc |
| | | caattcgccctatagtgagtcgtattacgc |
| | | gcgctcactggccgtcgttttacaacgtcg |
| | | tgactgggaaaaccctggcgttacccaact |
| | | taatcgccttgcagcacatccccctttcgc |
| | | cagctggcgtaatagcgaagaggcccgcac |
| | | cgatcgcccttcccaacagttgcgcagcct |
| | | gaatggcgaatgggacgcgccctgtagcgg |
| | | cgcattaagcgcggcgggtgtggtggttac |
| | | gcgcagcgtgaccgctacacttgccagcgc |
| | | cctagcgcccgctcctttcgctttcttccc |
| | | ttcctttctcgccacgttcgccggctttcc |
| | | ccgtcaagctctaaatcgggggctcccttt |
| | | agggttccgatttagtgctttacggcacct |
| | | cgaccccaaaaaacttgattagggtgatgg |
| | | ttcacgtagtgggccatcgccctgatagac |
| | | ggtttttcgccctttgacgttggagtccac |
| | | gttctttaatagtggactcttgttccaaac |
| | | tggaacaacactcaaccctatctcggtcta |
| | | ttcttttgatttataagggattttgccgat |
| | | ttcggcctattggttaaaaaatgagctgat |
| | | ttaacaaaaatttaacgcgaattttaacaa |
| | | aatattaacgcttacaatttaggtggcact |
| | | tttcggggaaatgtgcgcggaacccctatt |
| | | tgtttatttttctaaatacattcaaatatg |
| | | tatccgctcatgagacaataaccctgataa |
| | | atgcttcaataatattgaaaaaggaagagt |
| | | atgagtattcaacatttccgtgtcgccctt |
| | | attcccttttttgcggcattttgccttcct |
| | | gtttttgctcacccagaaacgctggtgaaa |
| | | gtaaaagatgctgaagatcagttgggtgca |
| | | cgagtgggttacatcgaactggatctcaac |
| | | agcggtaagatccttgagagttttcgcccc |
| | | gaagaacgttttccaatgatgagcactttt |
| | | aaagttctgctatgtggcgcggtattatcc |
| | | cgtattgacgccgggcaagagcaactcggt |
| | | cgccgcatacactattctcagaatgacttg |
| | | gttgagtactcaccagtcacagaaaagcat |
| | | cttacggatggcatgacagtaagagaatta |
| | | tgcagtgctgccataaccatgagtgataac |
| | | actgcggccaacttacttctgacaacgatc |
| | | ggaggaccgaaggagctaaccgcttttttg |
| | | cacaacatgggggatcatgtaactcgcctt |
| | | gatcgttgggaaccggagctgaatgaagcc |
| | | ataccaaacgacgagcgtgacaccacgatg |
| | | cctgtagcaatggcaacaacgttgcgcaaa |
| | | ctattaactggcgaactacttactctagct |
| | | tcccggcaacaattaatagactggatggag |
| | | gcggataaagttgcaggaccacttctgcgc |
| | | tcggcccttccggctggctggtttattgct |
| | | gataaatctggagccggtgagcgtgggtct |
| | | cgcggtatcattgcagcactggggccagat |
| | | ggtaagccctcccgtatcgtagttatctac |
| | | acgacggggagtcaggcaactatggatgaa |
| | | cgaaatagacagatcgctgagataggtgcc |
| | | tcactgattaagcattggtaactgtcagac |
| | | caagtttactcatatatactttagattgat |
| | | ttaaaacttcatttttaatttaaaaggatc |
| | | taggtgaagatcctttttgataatctcatg |
| | | accaaaatcccttaacgtgagttttcgttc |
| | | cactgagcgtcagaccccgtagaaaagatc |
| | | aaaggatcttcttgagatcctttttttctg |
| | | cgcgtaatctgctgcttgcaaacaaaaaaa |
| | | ccaccgctaccagcggtggtttgtttgccg |
| | | gatcaagagctaccaactctttttccgaag |
| | | gtaactggcttcagcagagcgcagatacca |
| | | aatactgttcttctagtgtagccgtagtta |
| | | ggccaccacttcaagaactctgtagcaccg |
| | | cctacatacctcgctctgctaatcctgtta |
| | | ccagtggctgctgccagtggcgataagtcg |
| | | tgtcttaccgggttggactcaagacgatag |
| | | ttaccggataaggcgcagcggtcgggctga |
| | | acggggggttcgtgcacacagcccagcttg |
| | | gagcgaacgacctacaccgaactgagatac |
| | | ctacagcgtgagctatgagaaagcgccacg |
| | | cttcccgaagagagaaaggcggacaggtat |
| | | ccggtaagcggcagggtcggaacaggagag |
| | | cgcacgagggagcttccagggggaaacgcc |
| | | tggtatctttatagtcctgtcgggtttcgc |
| | | cacctctgacttgagcgtcgatttttgtga |
| | | tgctcgtcaggggggcggagcctatggaaa |
| | | aacgccagcaacgcggcctttttacggttc |
| | | ctggccttttgctggccttttgctcacatg |
| | | ttctttcctgcgttatcccctgattctgtg |
| | | gataaccgtattaccgcctttgagtgagct |
| | | gataccgctcgccgcagccgaacgaccgag |
| | | cgcagcgagtcagtgagcgaggaagcggaa |
| | | gagcgcccaatacgcaaaccgcctctcccc |
| | | gcgcgttggccgattcattaatgcagctgg |
| | | cacgacaggtttcccgactggaaagcgggc |
| | | agtgagcgcaacgcaattaatgtgagttag |
| | | ctcactcattaggcaccccaggctttacac |
| | | tttatgcttccggctcgtatgttgtgtgga |
| | | attgtgagcggataacaatttcacacagga |
| | | aacagctatgaccatgattacgccaagcgc |
| | | gcaattaaccctcactaaagggaacaaaag |
| | | ctggagctgcaagctt |
| 102 | Nucleic acid | aatgtagtcttatgcaatactcttgtagtc |
| | sequence of Car3a- | ttgcaacatggtaacgatgagttagcaaca |
| | 19aalinker-NO- | tgccttacaaggagagaaaaagcaccgtgc |
| | CD28 plasmid | atgccgattggtggaagtaaggtggtacga |
| | | tcgtgccttattaggaaggcaacagacggg |
| | | tctgacatggattggacgaaccactgaatt |
| | | gccgcattgcagagatattgtatttaagtg |
| | | cctagctcgatacataaacgggtctctctg |
| | | gttagaccagatctgagcctgggagctctc |
| | | tggctaactagggaacccactgcttaagcc |
| | | tcaataaagcttgccttgagtgcttcaagt |
| | | agtgtgtgcccgtctgttgtgtgactctgg |
| | | taactagagatccctcagacccttttagtc |
| | | agtgtggaaaatctctagcagtggcgcccg |
| | | aacagggacttgaaagcgaaagggaaacca |
| | | gaggagctctctcgacgcaggactcggctt |
| | | gctgaagcgcgcacggcaagaggcgagggg |
| | | cggcgactggtgagtacgccaaaaattttg |
| | | actagcggaggctagaaggagagagatggg |
| | | tgcgagagcgtcagtattaagcgggggaga |
| | | attagatcgcgatgggaaaaaattcggtta |
| | | aggccagggggaaagaaaaaatataaatta |
| | | aaacatatagtatgggcaagcagggagcta |
| | | gaacgattcgcagttaatcctggcctgtta |
| | | gaaacatcagaaggctgtagacaaatactg |
| | | ggacagctacaaccatcccttcagacagga |
| | | tcagaagaacttagatcattatataataca |
| | | gtagcaaccctctattgtgtgcatcaaagg |
| | | atagagataaaagacaccaaggaagcttta |
| | | gacaagatagaggaagagcaaaacaaaagt |
| | | aagaccaccgcacagcaagcggccgctgat |
| | | cttcagacctggaggaggagatatgaggga |
| | | caattggagaagtgaattatataaatataa |
| | | agtagtaaaaattgaaccattaggagtagc |
| | | acccaccaaggcaaagagaagagtggtgca |
| | | gagagaaaaaagagcagtgggaataggagc |
| | | tttgttccttgggttcttgggagcagcagg |
| | | aagcactatgggcgcagcgtcaatgacgct |
| | | gacggtacaggccagacaattattgtctgg |
| | | tatagtgcagcagcagaacaatttgctgag |
| | | ggctattgaggcgcaacagcatctgttgca |
| | | actcacagtctggggcatcaagcagctcca |
| | | ggcaagaatcctggctgtggaaagatacct |
| | | aaaggatcaacagctcctggggatttgggg |
| | | ttgctctggaaaactcatttgcaccactgc |
| | | tgtgccttggaatgctagttggagtaataa |
| | | atctctggaacagatttggaatcacacgac |
| | | ctggatggagtgggacagagaaattaacaa |
| | | ttacacaagcttaatacactccttaattga |
| | | agaatcgcaaaaccagcaagaaaagaatga |
| | | acaagaattattggaattagataaatgggc |
| | | aagtttgtggaattggtttaacataacaaa |
| | | ttggctgtggtatataaaattattcataat |
| | | gatagtaggaggcttggtaggtttaagaat |
| | | agtttttgctgtactttctatagtgaatag |
| | | agttaggcagggatattcaccattatcgtt |
| | | tcagacccacctcccaaccccgaggggacc |
| | | cgacaggcccgaaggaatagaagaagaagg |
| | | tggagagagagacagagacagatccattcg |
| | | attagtgaacggatctcgacggtatcgcta |
| | | gcttttaaaagaaaaggggggattgggggg |
| | | tacagtgcaggggaaagaatagtagacata |
| | | atagcaacagacatacaaactaaagaatta |
| | | caaaaacaaattacaaaaattcaaaatttt |
| | | actagtgattatcggatcaactttgtatag |
| | | aaaagttgggctccggtgcccgtcagtggg |
| | | cagagcgcacatcgcccacagtccccgaga |
| | | agttggggggaggggtcggcaattgaaccg |
| | | gtgcctagagaaggtggcgcggggtaaact |
| | | gggaaagtgatgtcgtgtactggctccgcc |
| | | tttttcccgagggtgggggagaaccgtata |
| | | taagtgcagtagtcgccgtgaacgttcttt |
| | | ttcgcaacgggtttgccgccagaacacagg |
| | | taagtgccgtgtgtggttcccgcgggcctg |
| | | gcctctttacgggttatggcccttgcgtgc |
| | | cttgaattacttccacctggctgcagtacg |
| | | tgattcttgatcccgagcttcgggttggaa |
| | | gtgggtgggagagttcgaggccttgcgctt |
| | | aaggagccccttcgcctcgtgcttgagttg |
| | | aggcctggcctgggcgctggggccgccgcg |
| | | tgcgaatctggtggcaccttcgcgcctgtc |
| | | tcgctgctttcgataagtctctagccattt |
| | | aaaatttttgatgacctgctgcgacgcttt |
| | | ttttctggcaagatagtcttgtaaatgcgg |
| | | gccaagatctgcacactggtatttcggttt |
| | | ttggggccgcgggcggcgacggggcccgtg |
| | | cgtcccagcgcacatgttcggcgagggggg |
| | | cctgcgagcgcggccaccgagaatcggacg |
| | | ggggtagtctcaagctggccggcctgctct |
| | | ggtgcctggtctcgcgccgccgtgtatcgc |
| | | cccgccctgggcggcaaggctggcccggtc |
| | | ggcaccagttgcgtgagcggaaagatggcc |
| | | gcttcccggccctgctgcagggagctcaaa |
| | | atggaggacgcggcgctcgggagagcgggg |
| | | ggtgagtcacccacacaaaggaaaagggcc |
| | | tttccgtcctcagccgtcgcttcatgtgac |
| | | tccacggagtaccgggcgccgtccaggcac |
| | | ctcgattagttctcgagcttttggagtacg |
| | | tcgtctttaggttggggggaggggttttat |
| | | gcgatggagtttccccacactgagtgggtg |
| | | gagactgaagttaggccagcttggcacttg |
| | | atgtaattctccttggaatttgcccttttt |
| | | gagtttggatcttggttcattctcaagcct |
| | | cagacagtggttcaaagtttttttcttcca |
| | | tttcaggtgtcgtgacaagtttgtacaaaa |
| | | aagcaggctgccaccatggccttaccagtg |
| | | accgccttgctcctgccgctggccttgctg |
| | | ctccacgccgccaggccgggatccgacatc |
| | | cagatgacccagagcccctcctctttatcc |
| | | gcctctgtgggcgacagagtgaccatcact |
| | | tgtcgtgccagccaagaaatctccggctat |
| | | ctgtcttggctgcagcagaagcccggtaag |
| | | gctatcaagagactcatctacgccgccagc |
| | | actttacagagcggagtgcctagcagattt |
| | | agcggctctcgtagcggcagcgattatact |
| | | ttaaccatctcctctttacagcccgaagac |
| | | tttgctacctactactgtttacagtacgcc |
| | | agctaccccttcaccttcggtcaaggtacc |
| | | aagctggagatcaaaggtggcggcggctct |
| | | ggcggcggtggctctggcggtggctccggc |
| | | ggtggtggtagccaagttaccctcaaggag |
| | | agggccccactttagtgaagcctacccaga |
| | | ctttaactttaacttgtaccttcagcggct |
| | | tctctttaagcacctccacaatgggcgtgg |
| | | gctggatcagacagcctcccggcaaggctc |
| | | tggagtggctggcccacatcctctggaacg |
| | | acagcaagaggtacaacccctctttaaagt |
| | | ctcgtctgaccatcaccaaggacacctcca |
| | | agaagcaagttgtgctgaccatgaccaata |
| | | tggaccccgtggacaccgccacctattact |
| | | gcgctcgtatcgtgtactactccacctacg |
| | | tgggctacttcgacgtctggggacaaggta |
| | | ccaccgtgaccgtgagctctggatccttcg |
| | | tgccggtcttcctgccagcgaagcccacca |
| | | cgacgccagcgccgcgaccaccaacaccgg |
| | | cgcccaccatcgcgtcgcagcccctgtccc |
| | | tgcgcccagaggcgtgccggccagcggggg |
| | | gggcgcagtgcacacgagggggctggactt |
| | | cgcctgtgatatctacatctgggcgccctt |
| | | ggccgggacttgtggggtccttctcctgtc |
| | | actggttatcaccctttactgcaaccacag |
| | | gaacaaacggggcagaaagaaactcctgta |
| | | tatattcaaacaaccatttatgagaccagt |
| | | acaaactactcaagaggaagatggctgtag |
| | | ctgccgatttccagaagaagaagaaggagg |
| | | atgtgaactgagagtgaagttcagcaggag |
| | | cgcagacgcccccgcgtaccagcagggcca |
| | | gaaccagctctataacgagctcaatctagg |
| | | acgaagagaggagtacgatgttttggacaa |
| | | gagacgtggccgggaccctgagatgggggg |
| | | aaagccgagaaggaagaaccctcaggaagg |
| | | cctgtacaatgaactgcagaaagataagat |
| | | ggcggaggcctacagtgagattgggatgaa |
| | | aggcgagcgccggaggggcaaggggcacga |
| | | tggcctttaccagggtctcagtacagccac |
| | | caaggacacctacgacgcccttcacatgca |
| | | ggccctgccccctcgctgaacccagctttc |
| | | ttgtacaaagtggtgataatcgaattccga |
| | | taatcaacctctggattacaaaatttgtga |
| | | aagattgactggtattcttaactatgttgc |
| | | tccttttacgctatgtggatacgctgcttt |
| | | aatgcctttgtatcatgctattgcttcccg |
| | | tatggctttcattttctcctccttgtataa |
| | | atcctggttgctgtctctttatgaggagtt |
| | | gtggcccgttgtcaggcaacgtggcgtggt |
| | | gtgcactgtgtttgctgacgcaacccccac |
| | | tggttggggcattgccaccacctgtcagct |
| | | cctttccgggactttcgctttccccctccc |
| | | tattgccacggcggaactcatcgccgcctg |
| | | ccttgcccgctgctggacaggggctcggct |
| | | gttgggcactgacaattccgtggtgttgtc |
| | | ggggaagctgacgtcctttccatggctgct |
| | | cgcctgtgttgccacctggattctgcgcgg |
| | | gacgtccttctgctacgtcccttcggccct |
| | | caatccagcggaccttccttcccgcggcct |
| | | gctgccggctctgcggcctcttccgcgtct |
| | | tcgccttcgccctcagacgagtcggatctc |
| | | cctttgggccgcctccccgcatcgggaatt |
| | | cccgcggttcgctttaagaccaatgactta |
| | | caaggcagctgtagatcttagccacttttt |
| | | aaaagaaaaggggggactggaagggctaat |
| | | tcactcccaacgaagacaagatctgctttt |
| | | tgcttgtactgggtctctctggttagacca |
| | | gatctgagcctgggagctctctggctaact |
| | | agggaacccactgcttaagcctcaataaag |
| | | cttgccttgagtgcttcaagtagtgtgtgc |
| | | ccgtctgttgtgtgactctggtaactagag |
| | | atccctcagacccttttagtcagtgtggaa |
| | | aatctctagcagtagtagttcatgtcatct |
| | | tattattcagtatttataacttgcaaagaa |
| | | atgaatatcagagagtgagaggaacttgtt |
| | | tattgcagcttataatggttacaaataaag |
| | | caatagcatcacaaatttcacaaataaagc |
| | | atttttttcactgcattctagttgtggttt |
| | | gtccaaactcatcaatgtatcttatcatgt |
| | | ctggctctagctatcccgcccctaactccg |
| | | cccatcccgcccctaactccgcccagttcc |
| | | gcccattctccgccccatggctgactaatt |
| | | ttttttatttatgcagaggccgaggccgcc |
| | | tcggcctctgagctattccagaagtagtga |
| | | ggaggcttttttggaggcctagggacgtac |
| | | ccaattcgccctatagtgagtcgtattacg |
| | | cgcgctcactggccgtcgttttacaacgtc |
| | | gtgactgggaaaaccctggcgttacccaac |
| | | ttaatcgccttgcagcacatccccctttcg |
| | | ccagctggcgtaatagcgaagaggcccgca |
| | | ccgatcgcccttcccaacagttgcgcagcc |
| | | tgaatggcgaatgggacgcgccctgtagcg |
| | | gcgcattaagcgcggcgggtgtggtggtta |
| | | cgcgcagcgtgaccgctacacttgccagcg |
| | | ccctagcgcccgctcctttcgctttcttcc |
| | | cttcctttctcgccacgttcgccggctttc |
| | | cccgtcaagctctaaatcgggggctccctt |
| | | tagggttccgatttagtgctttacggcacc |
| | | tcgaccccaaaaaacttgattagggtgatg |
| | | gttcacgtagtgggccatcgccctgataga |
| | | cggtttttcgccctttgacgttggagtcca |
| | | cgttctttaatagtggactcttgttccaaa |
| | | ctggaacaacactcaaccctatctcggtct |
| | | attcttttgatttataagggattttgccga |
| | | tttcggcctattggttaaaaaatgagctga |
| | | tttaacaaaaatttaacgcgaattttaaca |
| | | aaatattaacgcttacaatttaggtggcac |
| | | ttttcggggaaatgtgcgcggaacccctat |
| | | ttgtttatttttctaaatacattcaaatat |
| | | gtatccgctcatgagacaataaccctgata |
| | | aatgcttcaataatattgaaaaaggaagag |
| | | tatgagtattcaacatttccgtgtcgccct |
| | | tattcccttttttgcggcattttgccttcc |
| | | tgtttttgctcacccagaaacgctggtgaa |
| | | agtaaaagatgctgaagatcagttgggtgc |
| | | acgagtgggttacatcgaactggatctcaa |
| | | cagcggtaagatccttgagagttttcgccc |
| | | cgaagaacgttttccaatgatgagcacttt |
| | | taaagttctgctatgtggcgcggtattatc |
| | | ccgtattgacgccgggcaagagcaactcgg |
| | | tcgccgcatacactattctcagaatgactt |
| | | ggttgagtactcaccagtcacagaaaagca |
| | | tcttacggatggcatgacagtaagagaatt |
| | | atgcagtgctgccataaccatgagtgataa |
| | | cactgcggccaacttacttctgacaacgat |
| | | cggaggaccgaaggagctaaccgctttttt |
| | | gcacaacatgggggatcatgtaactcgcct |
| | | tgatcgttgggaaccggagctgaatgaagc |
| | | cataccaaacgacgagcgtgacaccacgat |
| | | gcctgtagcaatggcaacaacgttgcgcaa |
| | | actattaactggcgaactacttactctagc |
| | | ttcccggcaacaattaatagactggatgga |
| | | ggcggataaagttgcaggaccacttctgcg |
| | | ctcggcccttccggctggctggtttattgc |
| | | tgataaatctggagccggtgagcgtgggtc |
| | | tcgcggtatcattgcagcactggggccaga |
| | | tggtaagccctcccgtatcgtagttatcta |
| | | cacgacggggagtcaggcaactatggatga |
| | | acgaaatagacagatcgctgagataggtgc |
| | | ctcactgattaagcattggtaactgtcaga |
| | | ccaagtttactcatatatactttagattga |
| | | tttaaaacttcatttttaatttaaaaggat |
| | | ctaggtgaagatcctttttgataatctcat |
| | | gaccaaaatcccttaacgtgagttttcgtt |
| | | ccactgagcgtcagaccccgtagaaaagat |
| | | caaaggatcttcttgagatcctttttttct |
| | | gcgcgtaatctgctgcttgcaaacaaaaaa |
| | | accaccgctaccagcggtggtttgtttgcc |
| | | ggatcaagagctaccaactctttttccgaa |
| | | ggtaactggcttcagcagagcgcagatacc |
| | | aaatactgttcttctagtgtagccgtagtt |
| | | aggccaccacttcaagaactctgtagcacc |
| | | gcctacatacctcgctctgctaatcctgtt |
| | | accagtggctgctgccagtggcgataagtc |
| | | gtgtcttaccgggttggactcaagacgata |
| | | gttaccggataaggcgcagcggtcgggctg |
| | | aacggggggttcgtgcacacagcccagctt |
| | | ggagcgaacgacctacaccgaactgagata |
| | | cctacagcgtgagctatgagaaagcgccac |
| | | gcttcccgaagagagaaaggcggacaggta |
| | | tccggtaagcggcagggtcggaacaggaga |
| | | gcgcacgagggagcttccagggggaaacgc |
| | | ctggtatctttatagtcctgtcgggtttcg |
| | | ccacctctgacttgagcgtcgatttttgtg |
| | | atgctcgtcaggggggcggagcctatggaa |
| | | aaacgccagcaacgcggcctttttacggtt |
| | | cctggccttttgctggccttttgctcacat |
| | | gttctttcctgcgttatcccctgattctgt |
| | | ggataaccgtattaccgcctttgagtgagc |
| | | tgataccgctcgccgcagccgaacgaccga |
| | | gcgcagcgagtcagtgagcgaggaagcgga |
| | | agagcgcccaatacgcaaaccgcctctccc |
| | | cgcgcgttggccgattcattaatgcagctg |
| | | gcacgacaggtttcccgactggaaagcggg |
| | | cagtgagcgcaacgcaattaatgtgagtta |
| | | gctcactcattaggcaccccaggctttaca |
| | | ctttatgcttccggctcgtatgttgtgtgg |
| | | aattgtgagcggataacaatttcacacagg |
| | | aaacagctatgaccatgattacgccaagcg |
| | | cgcaattaaccctcactaaagggaacaaaa |
| | | gctggagctgcaagctt |
| 103 | Amino acid | EGRGSLLTCGDVEENPGP |
| | sequence of T2A | |
| | cleavage peptide | |
| 104 | Amino acid | MLLLVTSLLLCELPHPAFLLIPRKVCNGIG |
| | sequence of EGFRt | IGEFKDSLSINATNIKHFKNCTSISGDLHI |
| | | LPVAFRGDSFTHTPPLDPQELDILKTVKEI |
| | | TGFLLIQAWPENRTDLHAFENLEIIRGRTK |
| | | QHGQFSLAVVSLNITSLGLRSLKEISDGDV |
| | | IISGNKNLCYANTINWKKLFGTSGQKTKII |
| | | SNRGENSCKATGQVCHALCSPEGCWGPEPR |
| | | DCVSCRNVSRGRECVDKCNLLEGEPREFVE |
| | | NSECIQCHPECLPQAMNITCTGRGPDNCIQ |
| | | CAHYIDGPHCVKTCPAGVMGENNTLVWKYA |
| | | DAGHVCHLCHPNCTYGCTGPGLEGCPTNGP |
| | | KIPSIATGMVGALLLLLVVALGIGLFM |
| 105 | Amino acid | QVETISPGDGRTFPKRGQTCVVHYTGMLED |
| | sequence of iCasp9 | GKKVDSSRDRNKPFKFMLGKQEVIRGWEEG |
| | | VAQMSVGQRAKLTISPDYAYGATGHPGIIP |
| | | PHATLVFDVELLKLESGGGSTNRQAAKLSK |
| | | PTLENLTPVVLRPEIRKPEVLRPETPRPVD |
| | | IGSGGFGDVGALESLRGNADLAYILSMEPC |
| | | GHCLIINNVNFCRESGLRTRTGSNIDCEKL |
| | | RRRFSSLHFMVEVKGDLTAKKMVLALLELA |
| | | QQDHGALDCCVVVILSHGCQASHLQFPGAV |
| | | YGTDGCPVSVEKIVNIFNGTSCPSLGGKPK |
| | | LFFIQACGGEQKDHGFEVASTSPEDESPGS |
| | | NPEPDATPFQEGLRTFDQLDAISSLPTPSD |
| | | IFVSYSTFPGFVSWRDPKSGSWYVETLDDI |
| | | FEQWAHSEDLQSLLLRVANAVSVKGIYKQM |
| | | PGCFNFLRKKLFFKTS |

### INCORPORATION BY REFERENCE

Various references such as patents, patent applications, and publications are cited herein, the disclosures of which are hereby incorporated by reference herein in their entireties.

What is claimed is:

1. A humanized antibody or antigen binding fragment thereof that binds to human FLT3, wherein the antibody or fragment comprises:
i. a light chain variable region (VL) comprising an amino acid sequence with at least 95% identity to any one of the sequences selected from the group consisting of: SEQ ID NO:1, SEQ ID NO:2, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, and SEQ ID NO:38; and/or
ii. a heavy chain variable region (VH) comprising an amino acid sequence with at least 95% identity to any one of the sequences selected from the group consisting of: SEQ ID NO:3, SEQ ID NO: 18, SEQ ID NO:19, SEQ ID NO:20, SEQ ID NO:21, SEQ ID NO:22, SEQ ID NO:23, SEQ ID NO;24, SEQ ID NO:25, SEQ ID NO:26, and SEQ ID NO:27.

2. The humanized antibody or fragment of claim 1, wherein (i) the VL comprises complementarity determining regions (CDRs) having at least 97%, 98%, 99% or 100% identity to the amino acid sequences of CDR-L1 of SEQ ID NO:86, CDR-L2 of SEQ ID NO: 87, and CDR-L3 of SEQ ID NO: 88, and (ii) the VH comprises CDRs having at least 97%, 98%, 99% or 100% identity to the amino acid sequences of CDR-H1 of SEQ ID NO: 89, CDR-H2 of SEQ ID NO: 90, and CDR-L3 of SEQ ID NO:91.

3. The humanized antibody or fragment of claim 1, wherein the VL comprises the amino acid sequence of SEQ ID NO:1, and the VH comprises the amino acid sequence of SEQ ID NO:3.

4. The humanized antibody or fragment of claim 1, wherein the VL comprises the amino acid sequence of SEQ ID NO:2, and the VH comprises the amino acid sequence of SEQ ID NO:3.

5. A single chain variable domain (scFv) comprising an antigen binding fragment of any one of claims 1-4.

6. The scFv of claim 5, which comprises a linker between the VL and the VH, wherein the linker has the formula (Gly₃₋₄-Ser)₁₋₄.

7. The scFv of claim 5, wherein the scFv has the amino acid sequence selected from the group consisting of: SEQ ID NO:4, SEQ ID NO:5, SEQ ID NO:44, SEQ ID NO:45, SEQ ID NO:46, SEQ ID NO:47, and SEQ ID NO:49.

8. The scFv of claim 7, wherein the scFv has the amino acid sequence of SEQ ID NO:4.

9. The scFv of claim 7, wherein the scFv has the amino acid sequence of SEQ ID NO:5.

10. A chimeric antigen receptor (CAR), wherein the CAR comprises: (i) an extracellular domain comprising (a) an antibody or fragment of any one of claims 1-4, or (b) an scFv of any one of claims 5-9; (ii) a transmembrane domain; and (iii) an intracellular domain.

11. The CAR of claim 10, wherein the transmembrane domain is a CD3 transmembrane domain, a CD4 transmembrane domain, a CD8 transmembrane domain, or a CD28 transmembrane domain.

12. The CAR of claim 10 or 11, wherein the intracellular domain comprises an activation domain, wherein, when the CAR is expressed in a T cell, the activation domain transmits an activation signal after the extracellular domain binds FLT3.

13. The CAR of claim 10 or 11, wherein the intracellular domain comprises an activation domain, wherein the activation domain comprises an intracellular signaling domain of CD3zeta, CD3epsilon, or FcRgamma.

14. The CAR of claim 12 or 13, wherein the intracellular domain further comprises one or more co-stimulatory domains.

15. The CAR of claim 14, wherein the one or more co-stimulatory domains are from one or more of: CD28, 4-1BB, CD27, OX40 or ICOS.

16. The CAR of claim 15, wherein the one or more co-stimulatory domains are from CD28 and/or 4-1BB.

17. The CAR of any one of claims 10-16, wherein the CAR comprises a spacer or hinge region between the extracellular domain and the transmembrane domain.

18. The CAR of claim 17, wherein the spacer or hinge region is from the extracellular domain of CD8.

19. The CAR of any one of claims 10-18, wherein the extracellular domain further comprises a cleavable signal peptide.

20. The CAR of claim 10, wherein the extracellular domain comprises an scFv comprising the amino acid sequence of SEQ ID NO:4; the transmembrane domain comprises a CD8 transmembrane domain; and the intracellular domain comprises an intracellular signaling domain of CD3zeta and a co-stimulatory domain of CD28 and/or 4-1BB.

21. The CAR of claim 10, where the CAR comprises the amino acid sequence selected from the group consisting of: SEQ ID NO:6, SEQ ID NO:9, SEQ ID NO:10, SEQ ID NO:11, SEQ ID NO:12, SEQ ID NO:13, SEQ ID NO:14, and SEQ ID NO:15.

22. The CAR of any one of claims 10-20, which further comprises a safety switch polypeptide, wherein the safety switch polypeptide is bound to the CAR by a self-cleaving peptide.

23. The CAR of claim 22, wherein the safety switch polypeptide is iCasp9 or EGFRt, and wherein the self-cleaving peptide is T2A, P2A, E2A, F2A or IRES.

24. The CAR of any one of claims 10-23, wherein a T cell expressing the CAR is activated or stimulated to proliferate when the extracellular domain binds to FLT3.

25. The CAR of any one of claims 10-24, wherein the CAR, when expressed on the surface of a T cell, directs the T cell to kill a cell expressing FLT3.

26. An immune cell expressing a CAR of any one of claims 10-25 or comprising a nucleic acid encoding a CAR of any one of claims 10-25.

27. The immune cell of claim 26, wherein the immune cell is a T cell, a NK cell, a macrophage or a monocyte.

28. The immune cell of claim 26 or 27, wherein the immune cell is a T cell.

29. The immune cell of any one of claims 26-28, wherein the immune cell comprises a nucleic acid, wherein the nucleic acid comprises a sequence selected from the group consisting of: SEQ ID NO:60, SEQ ID NO:63, SEQ ID NO:64, SEQ ID NO:65, SEQ ID NO:66, SEQ ID NO:67, SEQ ID NO:68, and SEQ ID NO:69.

30. The immune cell of any one of claims 26-29, wherein the immune cell has been derived from a subject before introducing the CAR or the nucleic acid.

31. The immune cell of claim 30, wherein the subject is a human.

32. The immune cell of any one of claims 26-31, where the immune cell expressing the CAR or comprising the nucleic acid is further expanded to generate a population of cells.

33. A population of immune cells expressing a CAR of any one of claims 10-25 or comprising a nucleic acid encoding a CAR of any one of claims 10-25.

34. A pharmaceutical composition comprising (i) a humanized antibody or fragment of any one of claims 1-4, an scFv of any one of claims 5-9, an immune cell of any one of claims 26-32, or a population of immune cells of claim 33, and (ii) a pharmaceutically acceptable carrier.

35. A method of treating a hematologic cancer in a subject in need thereof, wherein the method comprises administering to the subject a therapeutically effective amount of: (i) an immune cell of any one of claims 26-32, (ii) a population of immune cells of claim 33, or (ii) a pharmaceutical composition of claim 34.

36. The method of claim 35, where the hematologic cancer is acute myeloid leukemia (AML), acute lymphoblastic leukemia (ALL), chronic myeloid leukemia (CML), chronic lymphocytic leukemia (CLL), blastic plasmacytoid dendritic cell neoplasm (BPDCN), peripheral T cell lymphoma, follicular lymphoma, diffuse large B cell lymphoma, Hodgkin lymphoma, non-Hodgkin lymphoma, neuroblastoma, a non-malignant inherited or acquired marrow disorder, multiple myeloma, or a dendritic cell neoplasm.

37. The method of claim 36, wherein the hematologic cancer is AML.

38. The method of claim 36, wherein the hematologic cancer is ALL.

39. The method of claim 36, wherein the hematologic cancer is a dendritic cell neoplasm.

40. A method for preparing or conditioning a subject in need thereof for hematopoietic cell transplantation, wherein the method comprises administering to the subject a therapeutically effective amount of: (i) an immune cell of any one of claims 26-32, (ii) a population of immune cells of claim 33, or (ii) a pharmaceutical composition of claim 34.

41. The method of claim 40, wherein the therapeutically effective amount reduces the cell population expressing FLT3 by at least 90% in the subject.

42. The method of claim 40 or 41, wherein the subject in need thereof has a hematologic cancer.

43. The method of claim 42, wherein the hematologic cancer is acute myeloid leukemia (AML), acute lymphoblastic leukemia (ALL), chronic myeloid leukemia (CML), chronic lymphocytic leukemia (CLL), blastic plasmacytoid dendritic cell neoplasm (BPDCN), peripheral T cell lymphoma, follicular lymphoma, diffuse large B cell lymphoma, Hodgkin lymphoma, non-Hodgkin lymphoma, neuroblastoma, a non-malignant inherited or acquired marrow disorder, multiple myeloma, or a dendritic cell neoplasm.

44. The method of claim 43, wherein the hematologic cancer is AML.

45. The method of claim 43, wherein the hematologic cancer is ALL.

46. The method of claim 43, wherein the hematologic cancer is a dendritic cell neoplasm.

47. The method of any one of claims 35-46, wherein the administering reduces circulating myeloid lineages in the subject, optionally wherein the administering reduces circulating myeloid lineages by at least 60%, at least 65%, at least 70%, at least 75%, at least 80% or at least 85% relative to baseline levels.

48. The method of any one of claims 35-47, wherein the administering reduces bone marrow lineage frequencies and numbers in the subject, optionally wherein the administering reduces bone marrow frequencies and/or numbers by at least 50%, at least 55% or at least 60% relative to baseline levels.

49. The method of any one of claims 35-48, wherein the administering specifically targets human CD34⁺ hematopoietic stem cells and/or hematopoietic progenitor cells.

50. The method of claim 49, wherein the administering reduces human CD34⁺CD38⁺ cell population in bone marrow mononuclear cells of the subject by at least 50%, at least 55%, at least 60% or at least 65% relative to baseline levels, and/or reduces human CD34⁺CD38⁻ cell population in bone marrow mononuclear cells of the subject by at least 60%, at least 65%, at least 70%, at least 75%, at least 80% or at least 85% relative to baseline levels.

51. The method of any one of claims 35-50, which further comprises performing hematopoietic cell transplantation to the subject after the administering.

52. The method of claim 51, wherein the hematopoietic cell transplantation comprises transplantation to the subject of hematopoietic stem cells and/or hematopoietic progenitor cells.

53. The method of claim 51 or 52, wherein the performing of the hematopoietic cell transplantation occurs 5 days to 6 weeks after the administering.

54. The method of claim 53, wherein the performing of the hematopoietic cell transplantation occurs about 2 to 3 weeks after the administering.

55. The method of any one of claims 35-54, wherein the therapeutically effective amount of the immune cells or the population of immune cells is a dose from about 50,000,000 to 10,000,000,000 cells.

56. The method of claim 55, wherein the therapeutically effective amount of the immune cells or the population of immune cells is a dose from about 100,000,000 to 2,000,000,000 cells.

57. The method of any one of claims 35-56, wherein the administration is intravenous.

58. The method of claim 57, wherein the intravenous administration is by infusion into the subject.

59. The method of any one of claims 35-58, wherein the administering occurs once.

60. The method of any one of claims 35-58, wherein the administering is every 3-7 days for 2 to 3 weeks.

61. The method of any one of claims 35-60, wherein the method comprises the following steps prior to the administering step:
(v) collecting of blood from the subject;
(vi) isolating immune cells from the blood;
(vii) introducing a nucleic acid encoding a CAR of any one of claims 10-25 into the isolated immune cells; and
(viii) expanding the isolated immune cells obtained in step (iii), wherein the expanding yields the immune cells or the population of immune cells administered during the administering step.

62. The method of any one of claims 35-61, which further comprises administering a checkpoint inhibitor.

63. The method of claim 62, wherein the checkpoint inhibitor is an antagonist of PD1, PD-L1 or CTLA4.

64. The method of claim 63, wherein the antagonist is an antagonistic antibody.

65. The method of any one of claims 35-64, wherein the subject is a human.


SEQUENCE LISTING (105 sequences)

SEQ ID NO: 1 - protein, 107 aa, synthetic construct
DIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIK

SEQ ID NO: 2 - protein, 107 aa, synthetic construct
DIVMTQSPDSLAVSLGERATINCKASQEISGYLSWLQQKPGQPIKRLIYAASTRESGVPDRFSGSRSGSDYTLTISSLQAEDVAVYYCLQYASYPFTFGQGTKLEIK

SEQ ID NO: 3 - protein, 123 aa, synthetic construct
QVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSS

SEQ ID NO: 4 - protein, 249 aa, synthetic construct
DIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSS

SEQ ID NO: 5 - protein, 249 aa, synthetic construct
DIVMTQSPDSLAVSLGERATINCKASQEISGYLSWLQQKPGQPIKRLIYAASTRESGVPDRFSGSRSGSDYTLTISSLQAEDVAVYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSS

SEQ ID NO: 6 - protein, 552 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 930 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 8 - protein, 1012 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 9 - protein, 538 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 543 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 548 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 553 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 511 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 511 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 496 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 827 aa, synthetic construct
MALPVTALLLPLALLLHAARPGSDIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSSGSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAAAEGRGSLLTCGDVEENPGPSGMESDESGLPAMEIECRITGTLNGVEFELVGGGEGTPKQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAQSSNSAVDGTAGPGSTGSR

SEQ ID NO: 17 - protein, 123 aa, synthetic construct
QVTLKESGPGILQPSQTLSLTCSFSGFSLSTSTMGVGWIRQPSGKGLEWLLHILWNDSKYYNPALKSRLTISKDTYNKQVFLKIANVDTADTATYYCARIVYYSTYVGYFDVWGAGTTVTVSS

SEQ ID NO: 18 - protein, 123 aa, synthetic construct; identical to SEQ ID NO: 3

SEQ ID NO: 19 - protein, 123 aa, synthetic construct
QITLKESGPTLVKPTQTLTLTCTFSGFSLSTSTMGVGWIRQPPGKALEWLAHILWNDDKRYGPSLKSRLTITKDTSKKQVVLTMTNMDPVDTATYYCARIVYYSTYVGYFDVWGQGTTVTVSS

SEQ ID NO: 20 - protein, 123 aa, synthetic construct; identical to SEQ ID NO: 19

SEQ ID NO: 21 - protein, 123 aa, synthetic construct; identical to SEQ ID NO: 19

SEQ ID NO: 22 - protein, 123 aa, synthetic construct
QVQLQESGPGLVKPSETLSLTCTFSGFSLSTSTMGVGWIRQPPGKGLEWIGHILWNDSKYYNPSLKSRVTISKDTSKKQFSLKLSSVTAADTAVYYCARIVYYSTYVGYFDVWGQGTTVTVSS

SEQ ID NO: 23 - protein, 123 aa, synthetic construct; identical to SEQ ID NO: 22

SEQ ID NO: 24 - protein, 123 aa, synthetic construct; identical to SEQ ID NO: 22

SEQ ID NO: 25 - protein, 123 aa, synthetic construct
QVQLQQSGPGLVKPSQTLSLTCAFSGFSLSTSTMGVGWIRQSPSRGLEWLGHILWNDSKDYNPSVKSRITINKDTSKKQFSLQLNSVTPEDTAVYYCARIVYYSTYVGYFDVWGQGTTVTVSS

SEQ ID NO: 26 - protein, 123 aa, synthetic construct; identical to SEQ ID NO: 25

SEQ ID NO: 27 - protein, 123 aa, synthetic construct; identical to SEQ ID NO: 25

SEQ ID NO: 28 - protein, 107 aa, synthetic construct
DIQMTQSPSSLSASLGERVSLTCRASQEISGYLSWLQQKPDGTIKRLIYAASTLHSGVPKRFSGSRSGSDYSLTISRLESEDVADYYCLQYASYPFTFGSGTKLEIK

SEQ ID NO: 29 - protein, 107 aa, synthetic construct
EIVMTQSPGTLSLSPGERATLSCRASQEISGYLSWLQQKPGQAIRRLIYAASTRATGIPDRFSGSRSGSDYTLTISRLEPEDFAVYYCLQYASYPFTFGQGTKLEIK

SEQ ID NO: 30 - protein, 107 aa, synthetic construct; identical to SEQ ID NO: 1

SEQ ID NO: 31 - protein, 106 aa, synthetic construct
EIVMTQSPGTLSLSPGERATLSCRASQEISGYLSWLQQKPGQAIRRLIYAASTRATGIPDRFSGSRSGSDYTLTISRLEPEDFAVYYCLQYASYPFTFGQGTKLEI

SEQ ID NO: 32 - protein, 107 aa, synthetic construct; identical to SEQ ID NO: 2

SEQ ID NO: 33 - protein, 107 aa, synthetic construct; identical to SEQ ID NO: 1

SEQ ID NO: 34 - protein, 107 aa, synthetic construct; identical to SEQ ID NO: 29

SEQ ID NO: 35 - protein, 107 aa, synthetic construct; identical to SEQ ID NO: 2

SEQ ID NO: 36 - protein, 107 aa, synthetic construct; identical to SEQ ID NO: 1

SEQ ID NO: 37 - protein, 107 aa, synthetic construct; identical to SEQ ID NO: 29

SEQ ID NO: 38 - protein, 107 aa, synthetic construct; identical to SEQ ID NO: 2

SEQ ID NO: 39 - protein, 249 aa, synthetic construct
DIQMTQSPSSLSASLGERVSLTCRASQEISGYLSWLQQKPDGTIKRLIYAASTLHSGVPKRFSGSRSGSDYSLTISRLESEDVADYYCLQYASYPFTFGSGTKLEIKGGGGSGGGGSGGGSGGGGSQVTLKESGPGILQPSQTLSLTCSFSGFSLSTSTMGVGWIRQPSGKGLEWLLHILWNDSKYYNPALKSRLTISKDTYNKQVFLKIANVDTADTATYYCARIVYYSTYVGYFDVWGAGTTVTVSS

SEQ ID NO: 40 - protein, 249 aa, synthetic construct
EIVMTQSPGTLSLSPGERATLSCRASQEISGYLSWLQQKPGQAIRRLIYAASTRATGIPDRFSGSRSGSDYTLTISRLEPEDFAVYYCLQYASYPFTFGQGTKLEIKGGGGSGGGGSGGGSGGGGSQVTLKESGPTLVKPTQTLTLTCTFSGFSLSTSTMGVGWIRQPPGKALEWLAHILWNDSKRYNPSLKSRLTITKDTSKKQVVLTMTNMDPVDTATYYCARIVYYSTYVGYFDVWGQGTTVTVSS

SEQ ID NO: 41 - protein, 249 aa, synthetic construct
DIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQITLKESGPTLVKPTQTLTLTCTFSGFSLSTSTMGVGWIRQPPGKALEWLAHILWNDDKRYGPSLKSRLTITKDTSKKQVVLTMTNMDPVDTATYYCARIVYYSTYVGYFDVWGQGTTVTVSS

SEQ ID NO: 42 - protein, 249 aa, synthetic construct
EIVMTQSPGTLSLSPGERATLSCRASQEISGYLSWLQQKPGQAIRRLIYAASTRATGIPDRFSGSRSGSDYTLTISRLEPEDFAVYYCLQYASYPFTFGQGTKLEIKGGGGSGGGGSGGGSGGGGSQITLKESGPTLVKPTQTLTLTCTFSGFSLSTSTMGVGWIRQPPGKALEWLAHILWNDDKRYGPSLKSRLTITKDTSKKQVVLTMTNMDPVDTATYYCARIVYYSTYVGYFDVWGQGTTVTVSS

SEQ ID NO: 43 - protein, 249 aa, synthetic construct
DIVMTQSPDSLAVSLGERATINCKASQEISGYLSWLQQKPGQPIKRLIYAASTRESGVPDRFSGSRSGSDYTLTISSLQAEDVAVYYCLQYASYPFTFGQGTKLEIKGGGGSGGGGSGGGSGGGGSQITLKESGPTLVKPTQTLTLTCTFSGFSLSTSTMGVGWIRQPPGKALEWLAHILWNDDKRYGPSLKSRLTITKDTSKKQVVLTMTNMDPVDTATYYCARIVYYSTYVGYFDVWGQGTTVTVSS

SEQ ID NO: 44 - protein, 249 aa, synthetic construct
DIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVQLQESGPGLVKPSETLSLTCTFSGFSLSTSTMGVGWIRQPPGKGLEWIGHILWNDSKYYNPSLKSRVTISKDTSKKQFSLKLSSVTAADTAVYYCARIVYYSTYVGYFDVWGQGTTVTVSS

SEQ ID NO: 45 - protein, 249 aa, synthetic construct
EIVMTQSPGTLSLSPGERATLSCRASQEISGYLSWLQQKPGQAIRRLIYAASTRATGIPDRFSGSRSGSDYTLTISRLEPEDFAVYYCLQYASYPFTFGQGTKLEIKGGGGSGGGGSGGGSGGGGSQVQLQESGPGLVKPSETLSLTCTFSGFSLSTSTMGVGWIRQPPGKGLEWIGHILWNDSKYYNPSLKSRVTISKDTSKKQFSLKLSSVTAADTAVYYCARIVYYSTYVGYFDVWGQGTTVTVSS

SEQ ID NO: 46 - protein, 249 aa, synthetic construct
DIVMTQSPDSLAVSLGERATINCKASQEISGYLSWLQQKPGQPIKRLIYAASTRESGVPDRFSGSRSGSDYTLTISSLQAEDVAVYYCLQYASYPFTFGQGTKLEIKGGGGSGGGGSGGGSGGGGSQVQLQESGPGLVKPSETLSLTCTFSGFSLSTSTMGVGWIRQPPGKGLEWIGHILWNDSKYYNPSLKSRVTISKDTSKKQFSLKLSSVTAADTAVYYCARIVYYSTYVGYFDVWGQGTTVTVSS

SEQ ID NO: 47 - protein, 249 aa, synthetic construct
DIQMTQSPSSLSASVGDRVTITCRASQEISGYLSWLQQKPGKAIKRLIYAASTLQSGVPSRFSGSRSGSDYTLTISSLQPEDFATYYCLQYASYPFTFGQGTKLEIKGGGGSGGGGSGGGSGGGGSQVQLQQSGPGLVKPSQTLSLTCAFSGFSLSTSTMGVGWIRQSPSRGLEWLGHILWNDSKDYNPSVKSRITINKDTSKKQFSLQLNSVTPEDTAVYYCARIVYYSTYVGYFDVWGQGTTVTVSS

SEQ ID NO: 48 - protein, 249 aa, synthetic construct
EIVMTQSPGTLSLSPGERATLSCRASQEISGYLSWLQQKPGQAIRRLIYAASTRATGIPDRFSGSRSGSDYTLTISRLEPEDFAVYYCLQYASYPFTFGQGTKLEIKGGGGSGGGGSGGGSGGGGSQVQLQQSGPGLVKPSQTLSLTCAFSGFSLSTSTMGVGWIRQSPSRGLEWLGHILWNDSKDYNPSVKSRITINKDTSKKQFSLQLNSVTPEDTAVYYCARIVYYSTYVGYFDVWGQGTTVTVSS

SEQ ID NO: 49 - protein, 249 aa, synthetic construct
DIVMTQSPDSLAVSLGERATINCKASQEISGYLSWLQQKPGQPIKRLIYAASTRESGVPDRFSGSRSGSDYTLTISSLQAEDVAVYYCLQYASYPFTFGQGTKLEIKGGGGSGGGGSGGGSGGGGSQVQLQQSGPGLVKPSQTLSLTCAFSGFSLSTSTMGVGWIRQSPSRGLEWLGHILWNDSKDYNPSVKSRITINKDTSKKQFSLQLNSVTPEDTAVYYCARIVYYSTYVGYFDVWGQGTTVTVSS

SEQ ID NO: 50 - protein, 5 aa, synthetic construct
GGGGS

SEQ ID NO: 51 - protein, 10 aa, synthetic construct
GGGGSGGGGS

SEQ ID NO: 52 - protein, 15 aa, synthetic construct
GGGGSGGGGSGGGGS

SEQ ID NO: 53 - protein, 19 aa, synthetic construct
GGGGSGGGGSGGGSGGGGS

SEQ ID NO: 54 - protein, 20 aa, synthetic construct
GGGGSGGGGSGGGGSGGGGS

SEQ ID NO: 55 - DNA, 15 bp, synthetic construct
  source 1..15: mol_type = other DNA
ggtggcggcggctct

SEQ ID NO: 56 - DNA, 30 bp, synthetic construct
  source 1..30: mol_type = other DNA
ggtggcggcggctctggcggcggtggctct

SEQ ID NO: 57 - DNA, 45 bp, synthetic construct
  source 1..45: mol_type = other DNA
ggtggcggcggctctggcggcggtggctctggcggcggtggctcc

SEQ ID NO: 58 - DNA, 57 bp, synthetic construct
  source 1..57: mol_type = other DNA
ggtggcggcggctctggcggcggtggctctggcggtggctccggcggtggtggtagc

SEQ ID NO: 59 - DNA, 59 bp, synthetic construct
  source 1..59: mol_type = other DNA
gtggcggcggctctggcggcggtggctctggcggcggtggctccggcggtggtggtagc

SEQ ID NO: 60 - DNA, 1659 bp, synthetic construct
  source 1..1659: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 61 - DNA, 2793 bp, synthetic construct
  source 1..2793: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagagggcaggggaagtcttctaacatgcggggacgtggaggaaaatcccggccccatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtga

SEQ ID NO: 62 - DNA, 3039 bp, synthetic construct
  source 1..3039: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagagggcaggggaagtcttctaacatgcggggacgtggaggaaaatcccggccccatgggagtgcaggtggaaaccatctccccaggagacgggcgcaccttccccaagcgcggccagacctgcgtggtgcactacaccgggatgcttgaagatggaaagaaagtggattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggaggaggttctactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcatga

SEQ ID NO: 63 - DNA, 1617 bp, synthetic construct
  source 1..1617: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctcaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 64 - DNA, 1632 bp, synthetic construct
  source 1..1632: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctcaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 65 - DNA, 1647 bp, synthetic construct
  source 1..1647: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggcggtggctcccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 66 - DNA, 1662 bp, synthetic construct
  source 1..1662: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 67 - DNA, 1536 bp, synthetic construct
  source 1..1536: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 68 - DNA, 1536 bp, synthetic construct
  source 1..1536: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 69 - DNA, 1491 bp, synthetic construct
  source 1..1491: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctcaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 70 - DNA, 2484 bp, synthetic construct
  source 1..2484: mol_type = other DNA
atggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctag

SEQ ID NO: 71 - protein, 21 aa, Homo sapiens
MALPVTALLLPLALLLHAARP

SEQ ID NO: 72 - protein, 55 aa, Homo sapiens
FVPVFLPAKPTTTPAPRPPTPAPTIASQPLSLRPEACRPAAGGAVHTRGLDFACD

SEQ ID NO: 73 - protein, 28 aa, Homo sapiens
IYIWAPLAGTCGVLLLSLVITLYCNHRN

SEQ ID NO: 74 - protein, 41 aa, Homo sapiens
RSKRSRLLHSDYMNMTPRRPGPTRKHYQPYAPPRDFAAYRS

SEQ ID NO: 75 - protein, 42 aa, Homo sapiens
KRGRKKLLYIFKQPFMRPVQTTQEEDGCSCRFPEEEEGGCEL

SEQ ID NO: 76 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - DNA, 63 bp, Homo sapiens
  source 1..63: mol_type = genomic DNA
atggccttaccagtgaccgccttgctcctgccgctggccttgctgctccacgccgccaggccg

SEQ ID NO: 78 - DNA, 165 bp, Homo sapiens
  source 1..165: mol_type = genomic DNA
ttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 79 - DNA, 84 bp, Homo sapiens
  source 1..84: mol_type = genomic DNA
atctacatctgggcgcccttggccgggacttgtggggtccttctcctgtcactggttatcaccctttactgcaaccacaggaac

SEQ ID NO: 80 - DNA, 123 bp, Homo sapiens
  source 1..123: mol_type = genomic DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 81 - DNA, 126 bp, Homo sapiens
  source 1..126: mol_type = genomic DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 82 - DNA, 336 bp, Homo sapiens
  source 1..336: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 83 - DNA, 64 bp, unidentified
  source 1..64: mol_type = other DNA
ggaagcggagagggcaggggaagtcttctaacatgcggggacgtggaggaaaatcccggcccca

SEQ ID NO: 84 - DNA, 1074 bp, synthetic construct
  source 1..1074: mol_type = other DNA
tgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgaa

SEQ ID NO: 85 - DNA, 1317 bp, synthetic construct
  source 1..1317: mol_type = other DNA
atgggagtgcaggtggaaaccatctccccaggagacgggcgcaccttccccaagcgcggccagacctgcgtggtgcactacaccgggatgcttgaagatggaaagaaagtggattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggaggaggttctactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 86 - protein, 6 aa, synthetic construct
QEISGY

SEQ ID NO: 87 - skipped

SEQ ID NO: 88 - protein, 9 aa, synthetic construct
LQYASYPFT

SEQ ID NO: 89 - protein, 10 aa, synthetic construct
GFSLSTSTMG

SEQ ID NO: 90 - protein, 7 aa, synthetic construct
ILWNDSK

SEQ ID NO: 91 - protein, 15 aa, synthetic construct
ARIVYYSTYVGYFDV

SEQ ID NO: 92 - DNA, 8909 bp, synthetic construct
  source 1..8909: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagaatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagacttttgcagagaccaaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 93 - DNA, 7094 bp, synthetic construct
  source 1..7094: mol_type = other DNA
tataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatactagtaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctactctagaatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcggccgctagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 94 - DNA, 10404 bp, synthetic construct
  source 1..10404: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagagggcaggggaagtcttctaacatgcggggacgtggaggaaaatcccggccccatgggagtgcaggtggaaaccatctccccaggagacgggcgcaccttccccaagcgcggccagacctgcgtggtgcactacaccgggatgcttgaagatggaaagaaagtggattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggaggaggttctactaacaggcaagcagcaaagttgtcgaagccaaccctagaaaaccttaccccagtggtgctcagaccagagattcgcaaaccagaggttctcagaccggaaacacccagaccagtggacattggttctggaggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcagcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcatgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 95 - DNA, 10158 bp, synthetic construct
  source 1..10158: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagagggcaggggaagtcttctaacatgcggggacgtggaggaaaatcccggccccatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 96 - DNA, 8982 bp, synthetic construct
  source 1..8982: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctcaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 97 - DNA, 8997 bp, synthetic construct
  source 1..8997: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctcaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 98 - DNA, 9012 bp, synthetic construct
  source 1..9012: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggcggtggctcccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 99 - DNA, 9027 bp, synthetic construct
  source 1..9027: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 100 - DNA, 8856 bp, synthetic construct
  source 1..8856: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctcaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 101 - DNA, 8901 bp, synthetic construct
  source 1..8901: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 102 - DNA, 8901 bp, synthetic construct
  source 1..8901: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatggccttaccagtgaccgccttgctcctgccgctggccttgctgctccacgccgccaggccgggatccgacatccagatgacccagagcccctcctctttatccgcctctgtgggcgacagagtgaccatcacttgtcgtgccagccaagaaatctccggctatctgtcttggctgcagcagaagcccggtaaggctatcaagagactcatctacgccgccagcactttacagagcggagtgcctagcagatttagcggctctcgtagcggcagcgattatactttaaccatctcctctttacagcccgaagactttgctacctactactgtttacagtacgccagctaccccttcaccttcggtcaaggtaccaagctggagatcaaaggtggcggcggctctggcggcggtggctctggcggtggctccggcggtggtggtagccaagttaccctcaaggagagcggccccactttagtgaagcctacccagactttaactttaacttgtaccttcagcggcttctctttaagcacctccacaatgggcgtgggctggatcagacagcctcccggcaaggctctggagtggctggcccacatcctctggaacgacagcaagaggtacaacccctctttaaagtctcgtctgaccatcaccaaggacacctccaagaagcaagttgtgctgaccatgaccaatatggaccccgtggacaccgccacctattactgcgctcgtatcgtgtactactccacctacgtgggctacttcgacgtctggggacaaggtaccaccgtgaccgtgagctctggatccttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctgaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 103 - protein, 18 aa, unidentified
EGRGSLLTCGDVEENPGP

SEQ ID NO: 104 - protein, 357 aa, synthetic construct
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 105 - protein, 436 aa, synthetic construct
QVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS